(19) United States
(12) Patent Application Publication
(10) Pub. No.: US 2025/0122488 A1
(43) Pub. Date: Apr. 17, 2025

(54) ENGINEERED ADENYLATE KINASE VARIANTS

(71) Applicant: Codexis, Inc., Redwood City, CA (US)

(72) Inventors: Stephanie Marie Forget, Redwood City, CA (US); Anders Matthew Knight, Mountain View, CA (US); Marissa Greene MacAvoy, Emerald Hills, CA (US); Simon Ng, San Francisco, CA (US); Nicholas Porter, Redwood City, CA (US); Jonathan Vroom, South San Francisco, CA (US)

(21) Appl. No.: 18/913,751

(22) Filed: Oct. 11, 2024

Related U.S. Application Data
(60) Provisional application No. 63/589,828, filed on Oct. 12, 2023.
(60) Provisional application No. 63/661,366, filed on Jun. 18, 2024.

(51) Int. Cl. C12N 9/12; C12N 9/08; C12N 15/11

(52) U.S. Cl. CPC C12N 9/1229; C12N 9/0065; C12N 15/11; C12N 2310/321; C12N 2310/33; C12Y 111/01006; C12Y 207/04003

(57) ABSTRACT

The present disclosure provides engineered adenylate kinases, and recombinant polynucleotides encoding the engineered adenylate kinases. The present disclosure further provides uses of the engineered adenylate kinases for converting NMP to NDP.

### CROSS REFERENCE TO RELATED APPLICATIONS

This application claims the benefit of U.S. Provisional Application No. 63/589,828, filed Oct. 12, 2023, and U.S. Provisional Application No. 63/661,366, filed Jun. 18, 2024, each of which is hereby incorporated by reference herein in its entirety for all purposes.

### REFERENCE TO SEQUENCE LISTING, TABLE OR COMPUTER PROGRAM

The Sequence Listing concurrently submitted herewith as file name CX10-258WO3_ST26.xml, created on Oct. 10, 2024, with a file size of 5,616,375 bytes, is part of the specification and is hereby incorporated by reference herein in its entirety.

### TECHNICAL FIELD

The present disclosure relates to engineered adenylate kinase variants, polynucleotides encoding the engineered adenylate kinase variants, and uses of the engineered enzymes.

### BACKGROUND

Adenylate kinase (AK or AdyK) is an ATP:AMP phosphotransferase, an enzyme that belongs to the nucleoside monophosphate (NMPs) kinase family, and mediates the interconversion of adenine nucleotides (ATP+AMP↔2 ADP). The enzyme is ubiquitous in nature, being present in archaea, bacteria, and as well eukarya, with the exception of sulfur bacteria, and is a critical enzyme in energy metabolism and equilibrating adenine nucleotides in vivo. Although ATP is the substrate for most AKs, the human AK isoenzyme 3 uses GTP instead (GTP:AMP phosphotransferase).

Adenylate kinase can be used as an ATP regenerating system in presence of excess ADP to convert two ADP molecules, one as a phosphate acceptor and the other as a phosphate donor, to produce ATP+AMP. Adenylate kinase is also adaptable for use in enzyme cascades for the conversion of AMP to ADP as well as other NDPs. However, adenylate kinases can display bias in substrate specificity and have different activities towards certain NDP substrates.

### SUMMARY

The present disclosure provides engineered adenylate kinase enzymes, polynucleotides encoding the engineered adenylate kinase enzymes, and method of using the adenylate kinase enzymes. In some embodiments, the adenylate kinases have been engineered to have an improved property as compared to a reference adenylate kinase.

In one aspect, the present disclosure provides an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12-231 of an even-numbered SEQ ID NO. of SEQ ID NOs: 2-1302, 1356-2652, and 2676-3192, or to a reference sequence corresponding to an even-numbered SEQ ID NO. of SEQ ID NOs: 2-1302, 1356-2652, and 2676-3192, wherein the amino acid sequence comprises one or more substitutions relative to a reference sequence corresponding to amino acid residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or relative to a reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or to a reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, wherein the amino acid sequence comprises one or more substitutions relative to the reference sequence corresponding to amino acid residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12-231 of SEQ ID NO: 2, or to the reference sequence corresponding to SEQ ID NO: 2, wherein the amino acid sequence comprises one or more substitutions relative to the reference sequence corresponding to amino acid residues 12-231 of SEQ ID NO: 2, or relative to the reference sequence corresponding to SEQ ID NO: 2.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, wherein the amino acid sequence comprises one or more substitutions relative to the reference sequence corresponding to amino acid residues 12-231 of SEQ ID NO: 2, or relative to the reference sequence corresponding to SEQ ID NO: 2.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12-231 of an even-numbered SEQ ID NO. of SEQ ID NOs: 4-1302, 1356-2652, and 2676-3192, or to a reference sequence corresponding to an even-numbered SEQ ID NO. of SEQ ID NOs: 4-1302, 1356-2652, and 2676-3192, wherein the amino acid sequence comprises one or more substitutions relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 11, 13, 14, 15, 16, 18, 20, 21, 22, 26, 27, 29, 30, 31, 32, 34, 35, 36, 37, 38, 39, 40, 42, 43, 44, 46, 48, 50, 51, 52, 53, 54, 55, 56, 57, 58, 59, 60, 61, 62, 63, 64, 65, 66, 67, 68, 69, 70, 71, 73, 74, 75, 76, 77, 78, 79, 80, 81, 82, 83, 84, 87, 88, 89, 90, 91, 92, 93, 94, 97, 98, 99, 100, 101, 102, 104, 105, 108, 109, 110, 111, 112, 113, 115, 116, 117, 118, 119, 120, 122, 123, 124, 125, 126, 127, 128, 129, 131, 132, 133, 134, 135, 136, 137, 138, 139, 140, 141, 142, 143, 146, 148, 149, 150, 151, 152, 153, 154, 155, 156, 157, 161, 162, 163, 166, 168, 169, 170, 172, 173, 175, 178, 179, 180, 181, 182, 183, 184, 186, 187, 188, 190, 191, 192, 194, 195, 196, 197, 198, 199, 200, 201, 202, 203, 204, 205, 206, 207, 208, 210, 211, 212, 213, 214, 215, 216, 217, 218, 219, 220, 221, 222, 223, 224, 225, 226, 227, 228, 229, 230, or 231, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 13, 18, 29, 30, 31, 32, 34, 35, 48, 50, 53, 55, 59, 60, 61, 62, 66, 68, 77, 78, 80, 83, 87, 100, 104, 105, 109, 118, 119, 127, 128, 133, 136, 139, 142, 143, 150, 151, 155, 157, 168, 170, 175, 184, 186, 190, 201, 207, 212, 213, 214, 216, 217, 218, 224, or 226, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution or substitution set at amino acid position(s) 29/30/128/142/186/212/213, 18/142/212/213, 29/30/128/142/186, 40/135/214, 18/29/30/127/150, 82, 69, 118, 29/30, 206, 32, 16, 104, 111/135/136/214, 29/128/175, 178, 18/128/142, 32/108/111/156/191, 18/29/128/129/142/186, 32/133, 18/29/30/212/213/222, 29/65/127, 32/40/43/52/155/156/217, 18/29/128/129, 80, 32/43/138/152/191/214/215, 183, 18/175/186/212/213, 129, 18/29/30/65/129/142/150/186, 127/142/212/213, 30/65/142/222, 40/43/215, 136, 29/150/211/212/213/221, 43/111/136/219, 226, 88, 140/215/216, 32/133/134, or 18/29/128/186, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least one substitution set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution or substitution set of an engineered adenylate kinase variant set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to amino acid residues 12-231 of SEQ ID NO: 2, or relative to the reference sequence corresponding to SEQ ID NO: 2.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference amino acid sequence comprising a substitution or substitution set of an engineered adenylate kinase variant set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to amino acid residues 12-231 of SEQ ID NO: 2, or relative to the reference sequence corresponding to SEQ ID NO: 2.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12-231 of an even-numbered SEQ ID NO. of SEQ ID NOs: 4-1302, 1356-2652, and 2676-3192, or to a reference sequence corresponding to an even-numbered SEQ ID NO. of SEQ ID NOs: 4-1302, 1356-2652, and 2676-3192.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, wherein the amino acid sequence comprises one or more substitutions relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 4-1302, 1356-2652, and 2676-3192, or to the reference sequence corresponding to an even-numbered SEQ ID NO. of SEQ ID NOs: 4-1302, 1356-2652, and 2676-3192, wherein the amino acid sequence comprises one or more substitutions relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the amino acid sequence of the engineered adenylate kinase comprises at least a substitution at amino acid position 11, 13, 14, 15, 16, 18, 20, 21, 22, 26, 27, 29, 30, 31, 32, 34, 35, 36, 37, 38, 39, 40, 42, 43, 44, 46, 48, 50, 51, 52, 53, 54, 55, 56, 57, 58, 59, 60, 61, 62, 63, 64, 65, 66, 67, 68, 69, 70, 71, 73, 74, 75, 76, 77, 78, 79, 80, 81, 82, 83, 84, 87, 88, 89, 90, 91, 92, 93, 94, 97, 98, 99, 100, 101, 102, 104, 105, 108, 109, 110, 111, 112, 113, 115, 116, 117, 118, 119, 120, 122, 123, 124, 125, 126, 127, 128, 129, 131, 132, 133, 134, 135, 136, 137, 138, 139, 140, 141, 142, 143, 146, 148, 149, 150, 151, 152, 153, 154, 155, 156, 157, 161, 162, 163, 166, 168, 169, 170, 172, 173, 175, 178, 179, 180, 181, 182, 183, 184, 186, 187, 188, 190, 191, 192, 194, 195, 196, 197, 198, 199, 200, 201, 202, 203, 204, 205, 206, 207, 208, 210, 211, 212, 213, 214, 215, 216, 217, 218, 219, 220, 221, 222, 223, 224, 225, 226, 227, 228, 229, 230, or 231, or combinations thereof, wherein the amino acid positions are relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the amino acid sequence of the engineered adenylate kinase comprises at least a substitution at amino acid position 13, 18, 29, 30, 31, 32, 34, 35, 48, 50, 53, 55, 59, 60, 61, 62, 66, 68, 77, 78, 80, 83, 87, 100, 104, 105, 109, 118, 119, 127, 128, 133, 136, 139, 142, 143, 150, 151, 155, 157, 168, 170, 175, 184, 186, 190, 201, 207, 212, 213, 214, 216, 217, 218, 224, or 226, or combinations thereof, wherein the amino acid positions are relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 4, or to the reference sequence corresponding to SEQ ID NO: 4, wherein the amino acid sequence comprises one or more substitutions relative to the reference sequence corresponding to residues 12-231 of SEQ ID NO: 4, or relative to the reference sequence corresponding to SEQ ID NO: 4.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 86-372, or to the reference sequence corresponding to an even-numbered SEQ ID NO. of SEQ ID NOs: 86-372, wherein the amino acid sequence comprises one or more substitutions relative to the reference sequence corresponding to residues 12-231 of SEQ ID NO: 4, or relative to the reference sequence corresponding to SEQ ID NO: 4.

In some embodiments, the amino acid sequence of the engineered adenylate kinase comprises at least a substitution or substitution set at amino acid position(s) 100/101, 94, 179, 198, 57, 78, 68, 93, 77, 62, 14, 51, 118, 91, 163, 65, 173, 224, 66, 109, 60, 40/88, 191, 63, 68/170, 100, 108, 58, 180, 178, 199, 40, 80/203, 40/43, 39, 55, 110, 51/59, 155/226, 80, 219, 166, 202, 59, 38, 90, 80/184/203, 35, 222, 54, 111/153/155, 112, 175, 74, 105, 170, 117, 128, 61, 119, 155, 79, 220, 226, 108/155, 108/111, 111, 69/88, 43, 43/69/138, 40/43/88/134/178, 214, 80/82/184/219, 88/136, 16/40/43/88, 40/134, 88, 111/155, 223, 115, 190, 201, 126, 56, 216, 76, 69, 120, 116, 203, 194, or 138, wherein the amino acid sequence comprises one or more substitutions relative to the reference sequence corresponding to residues 12-231 of SEQ ID NO: 4, or relative to the reference sequence corresponding to SEQ ID NO: 4.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 172, or to the reference sequence corresponding to SEQ ID NO: 172, wherein the amino acid sequence comprises one or more substitutions relative to the reference sequence corresponding to residues 12-231 of SEQ ID NO: 172, or relative to the reference sequence corresponding to SEQ ID NO: 172.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 374-444, or to the reference sequence corresponding to an even-numbered SEQ ID NO. of SEQ ID NOs: 374-444, wherein the amino acid sequence comprises one or more substitutions relative to the reference sequence corresponding to residues 12-231 of SEQ ID NO: 172, or relative to the reference sequence corresponding to SEQ ID NO: 172.

In some embodiments, the amino acid sequence of the engineered adenylate kinase comprises at least a substitution or substitution set at amino acid position(s) 66/78, 78/80/224, 78, 65/66, 78/224, 78/80/109, 40/60/94/226, 66/68, 78/80, 94/117/118/226, 68/109, 68, 94/179, 51/66/68/224, 94/117, 51/66/68/78/224, 66, 118, 93/94/180, 109, 68/224, 40/62/93, 93, 51/66, 94/117/118, 198/220, 93/117, 117/118, 93/198, 68/173, 40/117, 224, 40/118/226, 93/94, or 40/62/118, wherein the amino acid sequence comprises one or more substitutions relative to the reference sequence corresponding to residues 12-231 of SEQ ID NO: 172, or relative to the reference sequence corresponding to SEQ ID NO: 172.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 376, or to the reference sequence corresponding to SEQ ID NO: 376, wherein the amino acid sequence comprises one or more substitutions relative to the reference sequence corresponding to residues 12-231 of SEQ ID NO: 376, or relative to the reference sequence corresponding to SEQ ID NO: 376.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 446-510, or to the reference sequence corresponding to an even-numbered SEQ ID NO. of SEQ ID NOs: 446-510, wherein the amino acid sequence comprises one or more substitutions relative to the reference sequence corresponding to residues 12-231 of SEQ ID NO: 376, or relative to the reference sequence corresponding to SEQ ID NO: 376.

In some embodiments, the amino acid sequence of the engineered adenylate kinase comprises at least a substitution or substitution set at amino acid position(s) 13, 60, 126, 104, 201, 59, 55, 133, 61, 53, 190, 57, 62, 181, 168, 54, 173, 170, 34, 183, 14, 60/62/124/170, 60/170, or 60/62, wherein the amino acid positions are relative to the reference sequence corresponding to residues 12-231 of SEQ ID NO: 376, or relative to the reference sequence corresponding to SEQ ID NO: 376.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 508, or to the reference sequence corresponding to SEQ ID NO: 508, wherein the amino acid sequence comprises one or more substitutions relative to the reference sequence corresponding to residues 12-231 of SEQ ID NO: 508, or relative to the reference sequence corresponding to SEQ ID NO: 508.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 512-580, or to the reference sequence corresponding to an even-numbered SEQ ID NO. of SEQ ID NOs: 512-580, wherein the amino acid sequence comprises one or more substitutions relative to the reference sequence corresponding to residues 12-231 of SEQ ID NO: 508, or relative to the reference sequence corresponding to SEQ ID NO: 508.

In some embodiments, the amino acid sequence of the engineered adenylate kinase comprises at least a substitution or substitution set at amino acid position(s) 15, 15/68, 15/181, 13/15, 13/15/201, 54/55, 55/133, 55/133/197, 54/55/133/197, 68, 54/133/197, 54/197, 13/15/68, 34, 54, 133, 120, 34/133, 13/15/181, 55, 15/59, 68/181, 201, 181, 66, 15/59/181, 197, 13/15/59, 61, 34/61, 13, 34/69, or 163, wherein the amino acid positions are relative to the reference sequence corresponding to residues 12-231 of SEQ ID NO: 508, or relative to the reference sequence corresponding to SEQ ID NO: 508.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 524, or to the reference sequence corresponding to SEQ ID NO: 524, wherein the amino acid sequence comprises one or more substitutions relative to the reference sequence corresponding to residues 12-231 of SEQ ID NO: 524, or relative to the reference sequence corresponding to SEQ ID NO: 524.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 582-608, or to the reference sequence corresponding to an even-numbered SEQ ID NO. of SEQ ID NOs: 582-608, wherein the amino acid sequence comprises one or more substitutions relative to the reference sequence corresponding to residues 12-231 of SEQ ID NO: 524, or relative to the reference sequence corresponding to SEQ ID NO: 524.

In some embodiments, the amino acid sequence of the engineered adenylate kinase comprises at least a substitution or substitution set at amino acid position(s) 120, 61/201, 34, 36, 105, 112, 31, or 146, wherein the amino acid positions are relative to the reference sequence corresponding to residues 12-231 of SEQ ID NO: 524, or relative to the reference sequence corresponding to SEQ ID NO: 524.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 584, or to the reference sequence corresponding to SEQ ID NO: 584, wherein the amino acid sequence comprises one or more substitutions relative to the reference sequence corresponding to residues 12-231 of SEQ ID NO: 584, or relative to the reference sequence corresponding to SEQ ID NO: 584.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 610-666, or to the reference sequence corresponding to an even-numbered SEQ ID NO. of SEQ ID NOs: 610-666, wherein the amino acid sequence comprises one or more substitutions relative to the reference sequence corresponding to residues 12-231 of SEQ ID NO: 584, or relative to the reference sequence corresponding to SEQ ID NO: 584.

In some embodiments, the amino acid sequence of the engineered adenylate kinase comprises at least a substitution or substitution set at amino acid position(s) 13/30/31/53/109/119/168, 36/126/190, 126, 36/39/126, 126/190, 36/126, 36/190, 39/126, 13/109/119, 109, 36/120/190, 126/148, 190, 108/126, 30/31, 13/118/182, 31/53, 39, 119, 13/119/168, 53/168, 34, 34/65/146, 36/39, 119/168, 34/112, or 173, wherein the amino acid positions are relative to the reference sequence corresponding to residues 12-231 of SEQ ID NO: 584, or relative to the reference sequence corresponding to SEQ ID NO: 584.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 610, or to the reference sequence corresponding to SEQ ID NO: 610, wherein the amino acid sequence comprises one or more substitutions relative to the reference sequence corresponding to residues 12-231 of SEQ ID NO: 610, or relative to the reference sequence corresponding to SEQ ID NO: 610.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 668-780, or to the reference sequence corresponding to an even-numbered SEQ ID NO. of SEQ ID NOs: 668-780, wherein the amino acid sequence comprises one or more substitutions relative to the reference sequence corresponding to residues 12-231 of SEQ ID NO: 610, or relative to the reference sequence corresponding to SEQ ID NO: 610.

In some embodiments, the amino acid sequence of the engineered adenylate kinase comprises at least a substitution or substitution set at amino acid position(s) 190, 115/126/190, 126/190, 34/35/105, 126, 59/126, 34/105/116/146, 48, 66, 59, 148, 82, 100, 27, 135, 77, 138, 52, 68, 139, 184, 81, 65, 153, 50, 181, 214, 172, 69, or 154, wherein the amino acid positions are relative to the reference sequence corresponding to residues 12-231 of SEQ ID NO: 610, or relative to the reference sequence corresponding to SEQ ID NO: 610.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 674, or to the reference sequence corresponding to SEQ ID NO: 674, wherein the amino acid sequence comprises one or more substitutions relative to the reference sequence corresponding to residues 12-231 of SEQ ID NO: 674, or relative to the reference sequence corresponding to SEQ ID NO: 674.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 782-868, or to the reference sequence corresponding to an even-numbered SEQ ID NO. of SEQ ID NOs: 782-868, wherein the amino acid sequence comprises one or more substitutions relative to the reference sequence corresponding to residues 12-231 of SEQ ID NO: 674, or relative to the reference sequence corresponding to SEQ ID NO: 674.

In some embodiments, the amino acid sequence of the engineered adenylate kinase comprises at least a substitution or substitution set at amino acid position(s) 81/148/219, 68/148/219, 68/81/219, 148/219, 81/219, 136/138/139, 68/219, 66/68/219, 66/148/219, 66/68/148, 59, 59/135, 219, 59/139, 138, 126/148/219, 59/181, 81, 66/81/219, 126/219, 66/68/81/148/219, 181, 27/148, 135/136/139, 148, 59/135/138/181, 59/135/136/138/139, 59/135/136/138/181, 27, 27/126/148, 135/136/138, 68/81/126/148/219, 136, 68, 126/148, or 138/139, wherein the amino acid positions are relative to the reference sequence corresponding to residues 12-231 of SEQ ID NO: 674, or relative to the reference sequence corresponding to SEQ ID NO: 674.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 816, or to the reference sequence corresponding to SEQ ID NO: 816, wherein the amino acid sequence comprises one or more substitutions relative to the reference sequence corresponding to residues 12-231 of SEQ ID NO: 816, or relative to the reference sequence corresponding to SEQ ID NO: 816.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 870-1014, or to the reference sequence corresponding to an even-numbered SEQ ID NO. of SEQ ID NOs: 870-1014, wherein the amino acid sequence comprises one or more substitutions relative to the reference sequence corresponding to residues 12-231 of SEQ ID NO: 816, or relative to the reference sequence corresponding to SEQ ID NO: 816.

In some embodiments, the amino acid sequence of the engineered adenylate kinase comprises at least a substitution or substitution set at amino acid position(s) 48/100/148, 48/66/148/219, 48/100, 48/100/135/136, 48/66/77/100/135/136/148, 48/100/136, 48/135/148, 48/148, 48/136/148, 48/135, 48/135/136, 66/100, 48/66/219, 48/135/219, 100/148, 48/136/219, 48/219, 48/135/148/219, 100/135/136, 100/136, 66/77/100/136/219, 100/135, 48/77/100/135/136, 100, 77/100/135, 48/66/100/135/136/148/219, 52/184, 66, 52/68, 136/148, 126/184, 148, 184, 52, 135/136/148, 126/138/184, 27/184, 27/126, 27/126/148, 27, 27/126/184, 66/100/135/219, 27/68, 52/68/81/126, 48/66/136/148, 66/136/148, 66/135, 66/136, 66/135/219, 136, 135/148/219, 68/126, 66/148/219, 27/68/126, 27/68/184, 126, 68/81/126, 77/135, 135/136, 77/136, 77/148, 82, 138/184, 77/135/136, 27/138, 81, 81/126, 27/126/138, 68, or 81/138, wherein the amino acid positions are relative to the reference sequence corresponding to residues 12-231 of SEQ ID NO: 816, or relative to the reference sequence corresponding to SEQ ID NO: 816.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 874, or to the reference sequence corresponding to SEQ ID NO: 874, wherein the amino acid sequence comprises one or more substitutions relative to the reference sequence corresponding to residues 12-231 of SEQ ID NO: 874, or relative to the reference sequence corresponding to SEQ ID NO: 874.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1016-1302, or to the reference sequence corresponding to an even-numbered SEQ ID NO. of SEQ ID NOs: 1016-1302, wherein the amino acid sequence comprises one or more substitutions relative to the reference sequence corresponding to residues 12-231 of SEQ ID NO: 874, or relative to the reference sequence corresponding to SEQ ID NO: 874.

In some embodiments, the amino acid sequence of the engineered adenylate kinase comprises at least a substitution or substitution set at amino acid position(s) 32/60/135/175/225, 32/81/127/128/135/175/225, 83/175/225, 135/175/225, 54/84/190, 55/190/212, 32/60/81/83/127/128/135/225, 175/225, 32/127/175, 84/190/212, 32/128/175, 81/175, 21/54, 54/55/212, 32/175/225, 128/175/225, 62/84/190, 81/135/225, 21/54/62/190/212, 60/81/83/175, 55/84/190, 32/81/83/135/225, 175, 62/212, 32/81/83, 32/81/225, 54/55/84/212, 55/190, 62/179, 54/84/179/212, 54/55/62/84, 81/135, 32/81/135, 179/190/212, 55/62, 100, 21/54/62/190, 190/212, 128/175, 21/62/190, 54/179/190, 54/190, 21/55/190, 21/54/62, 21/84, 21/55, 21/62, 21/179, 55/62/190/212, 54/62/84, 127/128/175/225, 54/62, 100/104, 54/55, 21/55/212, 62, 55/84, 55/62/212, 21/84/190, 225, 21/54/179/190, 83/135, 32/175, 21/54/78/190, 55/62/179/190/212, 21/54/190, 21/55/84/190, 62/84, 21/55/62, 21/62/84, 135, 32/60/81/127, 21/54/55/62/78, 54/84, 55/62/179/190, 54/55/62/84/179/190/212, 190, 83, 78, 54/62/190, 21/55/62/179/190, 32/60/127, 54/62/84/190/212, 21/62/179, 127/128/225, 32/225, 32/60/81, 81, 54, 21, 60/81/128/175, 84, 21/190/212, 60/128/225, 179, 21/55/62/190, 128, 60, 212, 55, 127, 32, 80, 220, 62/78/179, 55/62/84, 21/55/190/212, 32/127/128/225, 21/54/55/84/179, 21/190, 32/60/81/83/128/135, 127/128, or 21/55/179, wherein the amino acid positions are relative to the reference sequence corresponding to residues 12-231 of SEQ ID NO: 874, or relative to the reference sequence corresponding to SEQ ID NO: 874.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1032, or to the reference sequence corresponding to SEQ ID NO: 1032, wherein the amino acid sequence comprises one or more substitutions relative to the reference sequence corresponding to residues 12-231 of SEQ ID NO: 1032, or relative to the reference sequence corresponding to SEQ ID NO: 1032.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1356-1552, or to the reference sequence corresponding to an even-numbered SEQ ID NO. of SEQ ID NOs: 1356-1552, wherein the amino acid sequence comprises one or more substitutions relative to the reference sequence corresponding to residues 12-231 of SEQ ID NO: 1032, or relative to the reference sequence corresponding to SEQ ID NO: 1032.

In some embodiments, the amino acid sequence of the engineered adenylate kinase comprises at least a substitution or substitution set at amino acid position(s) 54/80/225, 55/80/180, 54/55/80/208/225, 84/128/155/190/212, 62/155/190, 83/84/155/190, 62/128/135/190, 225, 84/155/190, 128/190, 190/212, 83/84/155, 54/208/225, 84/155/190, 84/212, 84/123/155/212, 62/128/190, 55/80/208/225, 155/190, 83/84/190/212, 83/128/155/190/212, 55/180/225, 84/190/212, 54/80/180/208/225, 80/208, 135/155/190, 212, 80/180/208, 128/135/190/212, 128/155/212, 80/208/225, 83/212, 62/84/155/190, 54/179/225, 84, 83/84/190, 128/212, 54/55/80/208, 55, 62/128, 54/80/208/225, 62/83/84/190, 179/225, 190, 80/169/208, 62/128/212, 80, 62, 54/55/80/225, 54/80/180/208, 83/84/128/190/212, 62/83/190, 62/190, 80/179, 80/180, 54/80, 62/83/84/128/190/212, 83/84/135/212, 54/80/179/208, 62/84/128, 11/62/84/128/190/212, 80/179/208, 80/225, 62/84/128/190/212, 62/84/212, 80/179/180/208/225, 62/135, 55/208, 62/84, 128/135/190, 55/208/225, 84/128/190, 62/84/128/190, 62/135/212, 54/179/180/225, 62/84/135/212, 135, 54/55/225, 55/80/179/180, 84/135/190, 84/128, 54, 84/128/135, 54/179/208/225, 55/179/225, 128, 62/83/84/155/190, 84/135, 84/128/155, 55/179/180, 83/84/128/190, or 54/55/179/225, wherein the amino acid positions are relative to the reference sequence corresponding to residues 12-231 of SEQ ID NO: 1032, or relative to the reference sequence corresponding to SEQ ID NO: 1032.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1388, or to the reference sequence corresponding to SEQ ID NO: 1388, wherein the amino acid sequence comprises one or more substitutions relative to the reference sequence corresponding to residues 12-231 of SEQ ID NO: 1388, or relative to the reference sequence corresponding to SEQ ID NO: 1388.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1554-1786, or to the reference sequence corresponding to an even-numbered SEQ ID NO. of SEQ ID NOs: 1554-1786, wherein the amino acid sequence comprises one or more substitutions relative to the reference sequence corresponding to residues 12-231 of SEQ ID NO: 1388, or relative to the reference sequence corresponding to SEQ ID NO: 1388.

In some embodiments, the amino acid sequence of the engineered adenylate kinase comprises at least a substitution or substitution set at amino acid position(s) 143, 228, 136, 82, 156, 205, 226, 150, 219, 217, 218, 68, 142, 212, 213, 79, 230, 207, 155, 227, 129, 210, 133, 151, 43, 204, 182, 82/136/150/217, 136/150, 79/81/143/156/212/228, 82/136, 79/81/143/212, 79/133/228, 81/143/156/212, 136/219, 82/136/226, 136/150/217, 133/156, 133/143/228, 150/217, 82/136/150, 81, 81/143, 81/133/228, 143/228, 212/228, 79/81, 133/228, 133/212, 136/217, 216, 215, or 127, wherein the amino acid positions are relative to the reference sequence corresponding to residues 12-231 of SEQ ID NO: 1388, or relative to the reference sequence corresponding to SEQ ID NO: 1388.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1708, or to the reference sequence corresponding to SEQ ID NO: 1708, wherein the amino acid sequence comprises one or more substitutions relative to the reference sequence corresponding to residues 12-231 of SEQ ID NO: 1708, or relative to the reference sequence corresponding to SEQ ID NO: 1708.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1788-1968, or to the reference sequence corresponding to an even-numbered SEQ ID NO. of SEQ ID NOs: 1788-1968, wherein the amino acid sequence comprises one or more substitutions relative to the reference sequence corresponding to residues 12-231 of SEQ ID NO: 1708, or relative to the reference sequence corresponding to SEQ ID NO: 1708.

In some embodiments, the amino acid sequence of the engineered adenylate kinase comprises at least a substitution or substitution set at amino acid position(s) 127, 143/155/219/228, 219, 127/142, 142/151/207/218, 143/213, 151/218, 127/142/218, 142/151, 143/155/205/213/228, 127/142/207, 127/216/218, 142/151/207, 143/219, 127/207, 127/218, 143/219/228, 127/216, 68/127/207, 143/155/212/228, 151, 143/213/228, 143/155/205, 151/207/216, 143/228, 68/127/207/218, 143/155, 143, 205/213/219, 155/212/219/228, 205/219/228, 143/205, 142/207, 143/205/219, 143/212/213, 127/207/218, 142, 127/142/151/207/218, 143/205/219/228, 205/213/228, 68/151/207, 68/127/142/207, 68/127, 218, 127/142/196, 142/207/218, 68/142/216, 68/127/216/218, 68/127/218, 151/216/218, 143/212/228, 127/142/151, 151/207, 127/142/216, 129/207/218, 68/142/151/207, 68/142/151, 151/207/218, 228, 155/213, 127/142/151/218, 127/151, 155/228, 68/142/207, 80/151/207, 205/228, 68/142/207/216/218, 68/218, 68/207, 216/218, 68/142, 212, 205, or 142/207/216/218, wherein the amino acid positions are relative to the reference sequence corresponding to residues 12-231 of SEQ ID NO: 1708, or relative to the reference sequence corresponding to SEQ ID NO: 1708.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1952, or to the reference sequence corresponding to SEQ ID NO: 1952, wherein the amino acid sequence comprises one or more substitutions relative to the reference sequence corresponding to residues 12-231 of SEQ ID NO: 1952, or relative to the reference sequence corresponding to SEQ ID NO: 1952.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1970-2032, or to the reference sequence corresponding to an even-numbered SEQ ID NO. of SEQ ID NOs: 1970-2032, wherein the amino acid sequence comprises one or more substitutions relative to the reference sequence corresponding to residues 12-231 of SEQ ID NO: 1952, or relative to the reference sequence corresponding to SEQ ID NO: 1952.

In some embodiments, the amino acid sequence of the engineered adenylate kinase comprises at least a substitution or substitution set at amino acid position(s) 18/118, 87/119/192, 119/173/192, 118, 87/119, 18/118/170, 18, 119, 192, 87, 89, 59, 170, 64, 112, 93, 184, 146, 51, 125, 192, 191, 117, 172, or 163, wherein the amino acid positions are relative to the reference sequence corresponding to residues 12-231 of SEQ ID NO: 1952, or relative to the reference sequence corresponding to SEQ ID NO: 1952.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1980, or to the reference sequence corresponding to SEQ ID NO: 1980, wherein the amino acid sequence comprises one or more substitutions relative to the reference sequence corresponding to residues 12-231 of SEQ ID NO: 1980, or relative to the reference sequence corresponding to SEQ ID NO: 1980.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2034-2134, or to the reference sequence corresponding to an even-numbered SEQ ID NO. of SEQ ID NOs: 2034-2134, wherein the amino acid sequence comprises one or more substitutions relative to the reference sequence corresponding to residues 12-231 of SEQ ID NO: 1980, or relative to the reference sequence corresponding to SEQ ID NO: 1980.

In some embodiments, the amino acid sequence of the engineered adenylate kinase comprises at least a substitution or substitution set at amino acid position(s) 87/119/192, 87/192, 87/119, 119/192, 192, 119, 146/192, 87, 59/87, 59/87/112/146, 146, 87/112/119, 74, 75, 66, 181, 81, 73, 77, 188, 216, 82, 136, 214, 21, 217, 154, 133, 71, 212, or 228, wherein the amino acid positions are relative to the reference sequence corresponding to residues 12-231 of SEQ ID NO: 1980, or relative to the reference sequence corresponding to SEQ ID NO: 1980.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2072, or to the reference sequence corresponding to SEQ ID NO: 2072, wherein the amino acid sequence comprises one or more substitutions relative to the reference sequence corresponding to residues 12-231 of SEQ ID NO: 2072, or relative to the reference sequence corresponding to SEQ ID NO: 2072.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2136-2264, or to the reference sequence corresponding to an even-numbered SEQ ID NO. of SEQ ID NOs: 2136-2264, wherein the amino acid sequence comprises one or more substitutions relative to the reference sequence corresponding to residues 12-231 of SEQ ID NO: 2072, or relative to the reference sequence corresponding to SEQ ID NO: 2072.

In some embodiments, the amino acid sequence of the engineered adenylate kinase comprises at least a substitution or substitution set at amino acid position(s) 87, 194, 197, 93, 202, 38, 91, 183, 39, 146, 203, 37, 92, 36, 89, 131, 132, 90, 148, 35/197, 231, or 56, wherein the amino acid positions are relative to the reference sequence corresponding to residues 12-231 of SEQ ID NO: 2072, or relative to the reference sequence corresponding to SEQ ID NO: 2072.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2138, or to the reference sequence corresponding to SEQ ID NO: 2138, wherein the amino acid sequence comprises one or more substitutions relative to the reference sequence corresponding to residues 12-231 of SEQ ID NO: 2138, or relative to the reference sequence corresponding to SEQ ID NO: 2138.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2266-2366, or to the reference sequence corresponding to an even-numbered SEQ ID NO. of SEQ ID NOs: 2266-2366, wherein the amino acid sequence comprises one or more substitutions relative to the reference sequence corresponding to residues 12-231 of SEQ ID NO: 2138, or relative to the reference sequence corresponding to SEQ ID NO: 2138.

In some embodiments, the amino acid sequence of the engineered adenylate kinase comprises at least a substitution or substitution set at amino acid position(s) 161, 162, 153, 216, 212, 156, 214, 229, 77/143/214/216, 143/156/216, 77/143, 77/143/216, 77/214/216, 77/216, 143/156, 156/216, 143, 77, 77/214, 77/143/161/162/212/214, 77/162/214/216, 77/143/156/214/216, 77/143/156/162/212/216, 77/143/156/161/214/216, 77/156/162/216, 156/161/214/216, 77/143/162/214/216, 143/216, 214/216, 143/156/214/216, 143/214/216, 77/143/212/216, 143/156/161/162/216, 156/162/214, 77/143/156/161/162, 143/161/214/216, 77/161/216, or 143/212/214/216, wherein the amino acid positions are relative to the reference sequence corresponding to residues 12-231 of SEQ ID NO: 2138, or relative to the reference sequence corresponding to SEQ ID NO: 2138.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2294, or to the reference sequence corresponding to SEQ ID NO: 2294, wherein the amino acid sequence comprises one or more substitutions relative to the reference sequence corresponding to residues 12-231 of SEQ ID NO: 2294, or relative to the reference sequence corresponding to SEQ ID NO: 2294.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2368-2594, or to the reference sequence corresponding to an even-numbered SEQ ID NO. of SEQ ID NOs: 2368-2594, wherein the amino acid sequence comprises one or more substitutions relative to the reference sequence corresponding to residues 12-231 of SEQ ID NO: 2294, or relative to the reference sequence corresponding to SEQ ID NO: 2294.

In some embodiments, the amino acid sequence of the engineered adenylate kinase comprises at least a substitution or substitution set at amino acid position(s) 80, 105, 228, 50, 175, 170, 82, 173, 65, 53, 184, 122, 195, 68, 89, 34, 118, 119, 87, 179, 117, 113, 54, 190, 216, 88, 36, 166, 38, 169, 66, 142, 136, 131, 187, 40, 74, 127, 55, 148, 215, 64, 155, 116, 90, 132, 60, 94, 112, 120, 37, 108, 35, 51, 61, 212, 172, 59, or 56, wherein the amino acid positions are relative to the reference sequence corresponding to residues 12-231 of SEQ ID NO: 2294, or relative to the reference sequence corresponding to SEQ ID NO: 2294.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2368, or to the reference sequence corresponding to SEQ ID NO: 2368, wherein the amino acid sequence comprises one or more substitutions relative to the reference sequence corresponding to residues 12-231 of SEQ ID NO: 2368, or relative to the reference sequence corresponding to SEQ ID NO: 2368.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2596-2652 or 2676-2714, or to the reference sequence corresponding to an even-numbered SEQ ID NO. of SEQ ID NOs: 2596-2652 or 2676-2714, wherein the amino acid sequence comprises one or more substitutions relative to the reference sequence corresponding to residues 12-231 of SEQ ID NO: 2368, or relative to the reference sequence corresponding to SEQ ID NO: 2368.

In some embodiments, the amino acid sequence of the engineered adenylate kinase comprises at least a substitution or substitution set at amino acid position(s) 20, 21, 22, 43, 44, 46, 48, 138, 181, 182, 136/228, 82/142/173/184/216, 184/216, 50/184/216, 127/173/184, 105/136/170/175/228, 105/175, 50/184, 105/170/175/228, 50/127/173/184, 50/142/184/216, 50/82/127/216, 50, 50/82/173/216, 50/127/142/184/216, 50/82/127, 105/136, 105/228, 105/136/175/228, 173/216, 50/173, 228, 127/173, 105/136/170/195, 50/82/216, 175/228, or 50/82/127/142/184/216, wherein the amino acid positions are relative to the reference sequence corresponding to residues 12-231 of SEQ ID NO: 2368, or relative to the reference sequence corresponding to SEQ ID NO: 2368.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2602, or to the reference sequence corresponding to SEQ ID NO: 2602, wherein the amino acid sequence comprises one or more substitutions relative to the reference sequence corresponding to residues 12-231 of SEQ ID NO: 2602, or relative to the reference sequence corresponding to SEQ ID NO: 2602.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2716-2988, or to the reference sequence corresponding to an even-numbered SEQ ID NO. of SEQ ID NOs: 2716-2988, wherein the amino acid sequence comprises one or more substitutions relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution or substitution set at amino acid position(s) 18, 18/56, 20, 21, 26, 27, 29, 30, 32, 42, 43, 50, 66, 67, 69, 70, 80, 98, 99, 102, 135, 137, 139, 141, 142, 143, 148, 149, 152, 153, 156, 173, 184, or 216, wherein the amino acid positions are relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution or substitution set at amino acid position(s) 65, 65/104, 65/104/105/116/127, 65/104/105/127/136, 65/104/136, 65/104/136/170, 65/105/116/136/170, 65/105/127/136, 65/105/127/136/175, 65/105/127/170, 65/105/127/175, 65/105/136, 65/105/136/170, 65/105/170, 65/116, 65/127/136, 65/127/136/170, 65/127/170, 94/173, 94/187, 104/105, 104/105/127, 104/105/127/136/175/195, 104/105/136/170, 104/127, 104/127/136/170, 104/127/136/175, 105, 105/116/127/136/170/175, 105/127/136, 105/127/136/195, 105/136, 105/136/195, 116/127/136/175, 127/195, 132, 136, or 170, wherein the amino acid positions are relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution at amino acid position 36, 37, 40, 50, 59, 89, 117, 120, 128, 168, or 203, or combinations thereof, wherein the amino acid positions are relative to the reference sequence corresponding to residues 12-231 of SEQ ID NO: 2602, or relative to the reference sequence corresponding to SEQ ID NO: 2602.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2832, or to the reference sequence corresponding to SEQ ID NO: 2832, wherein the amino acid sequence comprises one or more substitutions relative to the reference sequence corresponding to residues 12-231 of SEQ ID NO: 2832, or relative to the reference sequence corresponding to SEQ ID NO: 2832.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2990-3032, or to the reference sequence corresponding to an even-numbered SEQ ID NO. of SEQ ID NOs: 2990-3032, wherein the amino acid sequence comprises one or more substitutions relative to the reference sequence corresponding to residues 12-231 of SEQ ID NO: 2832, or relative to the reference sequence corresponding to SEQ ID NO: 2832.

In some embodiments, the amino acid sequence of the engineered adenylate kinase comprises an amino acid sequence of the engineered adenylate kinase comprises at least a substitution or substitution set at amino acid position(s) 36, 36/50, 36/50/89/93/110, 36/50/89/93/139, 36/50/89/139/170, 36/50/89/172, 36/89, 36/170, 50, 50/89/93, 50/89/93/139, 50/93, 50/170, 89/170/172, 90/104, 90/151/157, 104/151/154, 104/151/154/157, 104/154/157/216, 151/157, 151/216, or 216, wherein the amino acid positions are relative to the reference sequence corresponding to residues 12-231 of SEQ ID NO: 2832, or relative to the reference sequence corresponding to SEQ ID NO: 2832.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2994, or to the reference sequence corresponding to SEQ ID NO: 2994, wherein the amino acid sequence comprises one or more substitutions relative to the reference sequence corresponding to residues 12-231 of SEQ ID NO: 2994, or relative to the reference sequence corresponding to SEQ ID NO: 2994.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 3034-3150, or to the reference sequence corresponding to an even-numbered SEQ ID NO. of SEQ ID NOs: 3034-3150, wherein the amino acid sequence comprises one or more substitutions relative to the reference sequence corresponding to residues 12-231 of SEQ ID NO: 2994, or relative to the reference sequence corresponding to SEQ ID NO: 2994.

In some embodiments, the amino acid sequence of the engineered adenylate kinase comprises an amino acid sequence of the engineered adenylate kinase comprises at least a substitution or substitution set at amino acid position(s) 50, 58, 59, 68, 74, 76, 79, 80, 83, 90, 112, 113, 119, 157, 170, 172, 182, 184, 217, 224, 226, 228, 229, 230, or 231, or combinations thereof, wherein the amino acid positions are relative to the reference sequence corresponding to residues 12-231 of SEQ ID NO: 2994, or relative to the reference sequence corresponding to SEQ ID NO: 2994.

In some embodiments, the amino acid sequence of the engineered adenylate kinase comprises an amino acid sequence of the engineered adenylate kinase comprises at least a substitution or substitution set at amino acid position(s) 79/104/157/228, 79/157/228/229, 79/157/229, 79/228, 83, 83/104, 83/104/151/168/173/190, 83/113, 83/173/190, 83/173/190/201, 83/173/201, 83/190, 83/190/201/216, 83/216, 104/157, 157, 157/173/190/216, 157/183, 157/190, 157/190/216, 157/228, 173/216, 201/216, or 229, wherein the amino acid positions are relative to the reference sequence corresponding to residues 12-231 of SEQ ID NO: 2994, or relative to the reference sequence corresponding to SEQ ID NO: 2994.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 3104, or to the reference sequence corresponding to SEQ ID NO: 3104, wherein the amino acid sequence comprises one or more substitutions relative to the reference sequence corresponding to residues 12-231 of SEQ ID NO: 3104, or relative to the reference sequence corresponding to SEQ ID NO: 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 3152-3192, or to the reference sequence corresponding to an even-numbered SEQ ID NO. of SEQ ID NOs: 3152-3192, wherein the amino acid sequence comprises one or more substitutions relative to the reference sequence corresponding to residues 12-231 of SEQ ID NO: 3104, or relative to the reference sequence corresponding to SEQ ID NO: 3104.

In some embodiments, the amino acid sequence of the engineered adenylate kinase comprises an amino acid sequence of the engineered adenylate kinase comprises at least a substitution or substitution set at amino acid position(s) 104, 104/157, 104/157/190/228, 104/170/228, 104/157/228/229, 104/157/229, 104/170/190, 104/170/190/228, 104/190, 104/190/201, 104/190/228, 104/201/228, 104/228/229, 157/228, 157/228/229, 170/190/228, 170/190/228/229, 190, 190/229, 201/228, or 228, wherein the amino acid positions are relative to the reference sequence corresponding to residues 12-231 of SEQ ID NO: 3104, or relative to the reference sequence corresponding to SEQ ID NO: 3104.

In some embodiments, the amino acid sequence of the engineered adenylate kinase comprises at least one substitution set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the amino acid sequence of the engineered adenylate kinase comprises at least a substitution or substitution set of an engineered adenylate kinase variant set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference amino acid sequence comprising a substitution or substitution set of an engineered adenylate kinase variant set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence comprising residues 12-231 of an even-numbered SEQ ID NO. of SEQ ID NOs: 4-1302, 1356-2652, and 2676-3192, or comprises an even-numbered SEQ ID NO. of SEQ ID NOs: 4-1302, 1356-2652, and 2676-3192, optionally wherein the engineered adenylate kinase has 1, 2, 3, 4, 5, 6, 7, 8, 9, or up to 10 substitutions in the amino acid sequence.

In some embodiments, the engineered adenylate kinase comprises an amino acid sequence comprising residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or wherein the amino acid sequence comprises SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, optionally wherein the engineered adenylate kinase has 1, 2, 3, 4, 5, 6, 7, 8, 9, or up to 10 substitutions in the amino acid sequence.

In some embodiments, the engineered adenylate kinase has adenylate kinase activity and at least one improved property as compared to a reference adenylate kinase. In some embodiments, the engineered adenylate kinase has an improved property selected from i) increased activity on unmodified nucleoside monophosphate (NMP), ii) increased stability, iii) increased thermostability, iv) increased activity on 2′-fluoro modified nucleoside monophosphate, and v) increased activity on 2′-O-methyl modified nucleoside monophosphate, or any combinations of i), ii), iii), iv), and v) as compared to a reference adenylate kinase. In some embodiments, the reference adenylate kinase has an amino acid sequence corresponding to residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or an amino acid sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104. In some embodiments, the reference adenylate kinase has an amino acid sequence corresponding to residues 12-231 of SEQ ID NO: 2, or an amino acid sequence corresponding to SEQ ID NO: 2.

In some embodiments, the engineered adenylate kinase is purified. In some embodiments, the engineered adenylate kinase is immobilized on a support medium.

In a further aspect, the present disclosure provides a recombinant polynucleotide comprising a polynucleotide sequence encoding an engineered adenylate kinase described herein.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to nucleotide residues 34-693 of SEQ ID NO: 1, 3, 171, 375, 507, 523, 583, 609, 673, 815, 873, 1031, 1387, 1707, 1951, 1979, 2071, 2137, 2293, 2367, 2601, 2831, 2993, or3103, or to a reference polynucleotide sequence corresponding to SEQ ID NO: 1, 3, 171, 375, 507, 523, 583, 609, 673, 815, 873, 1031, 1387, 1707, 1951, 1979, 2071, 2137, 2293, 2367, 2601, 2831, 2993, or 3103, wherein the recombinant polynucleotide encodes an adenylate kinase.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to nucleotide residues 34-693 of an odd-numbered SEQ ID NO. of SEQ ID NOs: 3-1301, 1355-2651, and 2675-3191, or to a reference polynucleotide sequence corresponding to an odd-numbered SEQ ID NO. of SEQ ID NOs: 3-1301, 1355-2651, and 2675-3191, wherein the recombinant polynucleotide encodes an engineered adenylate kinase.

In some embodiments, the polynucleotide sequence encoding the engineered adenylate kinase has preferred codons, e.g., is codon optimized. In some embodiments, the polynucleotide sequence is codon optimized for expression in a bacterial cell, fungal cell, or a mammalian cell.

In some embodiments, the polynucleotide sequence comprises nucleotide residues 34-693 of an odd-numbered SEQ ID NO. of SEQ ID NOs: 3-1301, 1355-2651, and 2675-3191, or comprises an odd-numbered SEQ ID NO. of SEQ ID NOs: 3-1301, 1355-2651, and 2675-3191.

In some embodiments, the polynucleotide sequence comprises nucleotide residues 34-693 of SEQ ID NO: 1, 3, 171, 375, 507, 523, 583, 609, 673, 815, or 873, 1031, 1387, 1707, 1951, 1979, 2071, 2137, 2293, 2367, 2601, 2831, 2993, or 3103 or comprises SEQ ID NO: 1, 3, 171, 375, 507, 523, 583, 609, 673, 815, or 873, 1031, 1387, 1707, 1951, 1979, 2071, 2137, 2293, 2367, 2601, 2831, 2993, or 3103.

In another aspect, the present disclosure provides an expression vector comprising a recombinant polynucleotide encoding an engineered adenylate kinase described herein. In some embodiments, the recombinant polynucleotide of the expression vector is operably linked to a control sequence.

In a further aspect, the present disclosure also provides a host cell comprising an expression vector comprising a recombinant polynucleotide encoding an engineered adenylate kinase. In some embodiments, the host cell is a bacterial cell, fungal cell, insect cell, or mammalian cell.

In a further aspect, the host cells are used for producing an engineered adenylate kinase. In some embodiments, a method of producing an engineered adenylate kinase polypeptide comprises culturing a host cell comprising an expression vector described herein under suitable culture conditions such that the engineered adenylate kinase polypeptide is produced. In some embodiments, the method further comprises recovering or isolating the adenylate kinase polypeptide from the culture and/or host cells. In some embodiments, the method further comprises purifying the adenylate kinase polypeptide.

In a further aspect, the engineered adenylate kinase is provided as a composition. In some embodiments, the composition further comprises a nucleoside monophosphate substrate. In some embodiments, the nucleoside in the composition is a modified nucleoside monophosphate.

In a further aspect, the engineered adenylate kinase is used for converting a nucleoside monophosphate to the corresponding nucleoside diphosphate. In some embodiments, a method of converting a nucleoside monophosphate (NMP) to the nucleoside diphosphate (NDP), comprises contacting a nucleoside monophosphate with an engineered adenylate kinase described herein in the presence of substrate NTP under suitable reaction conditions to convert the nucleoside monophosphate to the corresponding nucleoside diphosphate.

In some embodiments of the method, the substrate nucleoside monophosphate is an unmodified nucleoside monophosphate. In some embodiments of the method, the substrate nucleoside monophosphate is a modified nucleoside monophosphate. In some embodiments, the modified nucleoside monophosphate comprises a modified sugar moiety, modified nucleobase, or modified phosphate, or any combination thereof.

### DETAILED DESCRIPTION

The present disclosure provides engineered adenylate kinases for the interconversion of ATP:AMP, and recombinant polynucleotides encoding the engineered adenylate kinases. In some embodiments, the engineered adenylate kinases has adenylate kinase activity, and one or more improved properties as compared to the parent wild-type enzyme, including increased activity on NDPs, including NDPs other than ADP, and increased activity on modified NDPs.

### Abbreviations and Definitions

In reference to the present invention, the technical and scientific terms used in the descriptions herein will have the meanings commonly understood by one of ordinary skill in the art, unless specifically defined otherwise. Accordingly, the following terms are intended to have the following meanings.

As used herein, the singular forms “a”, “an” and “the” include plural referents unless the context clearly indicates otherwise. Thus, for example, reference to “a polypeptide” includes more than one polypeptide.

Similarly, “comprise,” “comprises,” “comprising” “include,” “includes,” and “including” are interchangeable and not intended to be limiting. Thus, as used herein, the term “comprising” and its cognates are used in their inclusive sense (i.e., equivalent to the term “including” and its corresponding cognates).

It is to be further understood that where descriptions of various embodiments use the term “comprising,” those skilled in the art would understand that in some specific instances, an embodiment can be alternatively described using language “consisting essentially of” or “consisting of.”

“About” means an acceptable error for a particular value. In some instances, “about” means within 0.05%, 0.5%, 1.0%, or 2.0%, of a given value range. In some instances, “about” means within 1, 2, 3, or 4 standard deviations of a given value.

“EC” number refers to the Enzyme Nomenclature of the Nomenclature Committee of the International Union of Biochemistry and Molecular Biology (NC-IUBMB). The IUBMB biochemical classification is a numerical classification system for enzymes based on the chemical reactions they catalyze.

“ATCC” refers to the American Type Culture Collection whose biorepository collection includes genes and strains.

“NCBI” refers to National Center for Biological Information and the sequence databases provided therein.

“Adenylate kinase,” “AdyK,” or “AK” refers to an enzyme that catalyzes the interconversion of ATP, ADP, and AMP through transfer of phosphoryl groups. In some embodiments, adenylate kinase also includes enzymes capable of catalyzing the interconversion of NTP, NDP and NMP. In some embodiments, adenylate kinase are enzymes classified in EC 2.7.3.4.

“Creatine kinase” refers to an enzyme that catalyzes the reversible interconversion of creatine:ATP to creatine phosphate:ADP.

“Polyphosphate kinase” refers to an enzyme that catalyzes the transfer of phosphate group(s) from high-energy, phosphate-donating molecules, such as polyphosphate (PolyPn), to specific substrates/molecules. Two families of polyphosphate kinases, PPKK1 and PPK2, are known. PPK1s preferentially synthesize polyphosphate from NTP and the corresponding reverse reaction, and PPK2s preferentially consume polyphosphate to phosphorylate nucleoside mono- or diphosphates, and the corresponding reverse reactions. In some embodiments, polyphosphate kinase includes enzymes classified in EC 2.7.4.1.

“Acetate kinase (“AcK”) refers to enzymes that are capable of catalyzing the phosphorylation of nucleoside diphosphates or analogues thereof, to nucleoside triphosphates or the corresponding analogues, using acetyl phosphate or another phosphoryl group donor. Acetate kinases as used herein includes naturally occurring, wild-type enzymes or engineered enzymes. In some embodiments, acetate kinases are naturally occurring, wild-type basic metabolic enzymes found primarily in prokaryotes that catalyze the following reaction: acetate+ATP↔acetyl phosphate+ADP. Acetyl phosphate is an intermediate in the formation of acetyl-CoA. In some embodiments, acetate kinases are derived from the naturally occurring, wild-type enzymes.

“Adenosine kinase,” “AdoK,” or “Adk,” refer to an enzyme that catalyzes the phosphorylation of adenosine (A or ADO) to adenosine-5′-monophosphate (AMP). In some embodiments, adenosine kinase is classified in EC 2.7.1.20. Although the primary substrate is adenosine, adenosine kinases as used herein include enzymes that act on other nucleosides.

“Pyruvate oxidase” or “Pox” refers to an enzyme that catalyzes the catalyzes the reaction between pyruvate, inorganic phosphate, and oxygen to generate acetyl phosphate and H₂O₂. In some embodiments, pyruvate oxidase include enzymes classified in EC 1.2. 3.3.

“Catalase” refers to an enzyme that converts hydrogen peroxide (H₂O₂) to H₂O and O₂. Catalase can be used to remove residual hydrogen peroxide in applications where hydrogen peroxide is present or is a product in a process. In some embodiments, catalases includes enzymes classified in EC 1.11.1.6.

“Protein,” “polypeptide,” and “peptide” are used interchangeably herein to denote a polymer of at least two amino acids covalently linked by an amide bond, regardless of length or post-translational modification (e.g., glycosylation or phosphorylation). Included within this definition are D- and L-amino acids, and mixtures of D- and L-amino acids, as well as polymers comprising D- and L-amino acids, and mixtures of D- and L-amino acids.

“Fusion protein,” and “chimeric protein” and “chimera” refer to hybrid proteins created through the joining of two or more polynucleotides that originally encode separate proteins. In some embodiments, fusion proteins are created by recombinant technology (e.g., molecular biology techniques known in the art).

“Amino acids” are referred to herein by either their commonly known three-letter symbols or by the one-letter symbols recommended by IUPAC-IUB Biochemical Nomenclature Commission. Nucleotides, likewise, may be referred to by their commonly accepted single letter codes. The abbreviations used for the genetically encoded amino acids are conventional and are as follows: alanine (Ala or A), arginine (Arg or R), asparagine (Asn or N), aspartate (Asp or D), cysteine (Cys or C), glutamate (Glu or E), glycine (Gly or G), glutamine (Gln or Q), histidine (His or H), isoleucine (Ile or I), leucine (Leu or L), lysine (Lys or K), methionine (Met or M), phenylalanine (Phe or F), proline (Pro or P), serine (Ser or S), threonine (Thr or T), tryptophan (Trp or W), tyrosine (Tyr or Y), and valine (Val or V). When the three-letter abbreviations are used, unless specifically preceded by an “L” or a “D” or clear from the context in which the abbreviation is used, the amino acid may be in either the L- or D-configuration about α-carbon (Cα). For example, whereas “Ala” designates alanine without specifying the configuration about the α-carbon, “D-Ala” and “L-Ala” designate D-alanine and L-alanine, respectively. When the one-letter abbreviations are used, upper case letters designate amino acids in the L-configuration about the α-carbon and lower case letters designate amino acids in the D-configuration about the α-carbon. For example, “A” designates L-alanine and “a” designates D-alanine. When polypeptide sequences are presented as a string of one-letter or three-letter abbreviations (or mixtures thereof), the sequences are presented in the amino (N) to carboxy (C) direction in accordance with common convention.

“Polynucleotide” and “nucleic acid’ refer to two or more nucleotides that are covalently linked together. The polynucleotide may be wholly comprised of ribonucleotides (i.e., RNA), wholly comprised of 2′ deoxyribonucleotides (i.e., DNA), or comprised of mixtures of ribo- and 2′ deoxyribonucleotides. While the nucleosides will typically be linked together via standard phosphodiester linkages, the polynucleotides may include one or more non-standard linkages. The polynucleotide may be single-stranded or double-stranded, or may include both single-stranded regions and double-stranded regions. Moreover, while a polynucleotide will typically be composed of the naturally occurring encoding nucleobases (i.e., adenine, guanine, uracil, thymine and cytosine), it may include one or more modified and/or synthetic nucleobases, such as, for example, inosine, xanthine, hypoxanthine, etc. In some embodiments, such modified or synthetic nucleobases are nucleobases encoding amino acid sequences.

The abbreviations used for the genetically encoding nucleosides are conventional and are as follows: adenosine (A); guanosine (G); cytidine (C); thymidine (T); and uridine (U). Unless specifically delineated, the abbreviated nucleosides may be either ribonucleosides or 2′-deoxyribonucleosides. The nucleosides may be specified as being either ribonucleosides or 2′-deoxyribonucleosides on an individual basis or on an aggregate basis. When nucleic acid sequences are presented as a string of one-letter abbreviations, the sequences are presented in the 5′ to 3′ direction in accordance with common convention, and the phosphates are not indicated.

“Nucleobase” or “base” refers to those naturally occurring and synthetic heterocyclic moieties commonly known to those who utilize nucleic acid or polynucleotide technology to thereby generate polymers that can hybridize to polynucleotides in a sequence-specific manner. Non-limiting examples of nucleobases include, among others, adenine, cytosine, guanine, thymine, uracil, 5-propynyl-uracil, 2-thio-5-propynyl-uracil, 5-methylcytosine, pseudoisocytosine, pseudoisouridine, 2-thiouracil and 2-thiothymine, 2-aminopurine, N9-(2-amino-6-chloropurine), N9-(2,6-diaminopurine), hypoxanthine, N9-(7-deaza-guanine), N9-(7-deaza-8-aza-guanine) and N8-(7-deaza-8-aza-adenine).

“Nucleoside” refers to glycosylamines comprising a nucleobase, and a 5-carbon sugar (e.g., ribose, deoxyribose, or arabinose). Non-limiting examples of nucleosides include cytidine, uridine, adenosine, guanosine, thymidine, and inosine. In contrast, the term “nucleotide” refers to the glycosylamines comprising a nucleobase, a 5-carbon sugar, and one or more phosphate groups, as further described herein. In some embodiments, nucleosides can be phosphorylated by kinases to produce nucleotides.

“Nucleoside monophosphate” or “NMP” refers to glycosylamines comprising a nucleobase, a 5-carbon sugar (e.g., ribose, deoxyribose, or arabinose), and a phosphate moiety at the 5′-position. In some embodiments herein, “nucleoside monophosphate” is abbreviated as “NMP”. Non-limiting examples of nucleoside monophosphates include cytidine monophosphate (CMP), uridine monophosphate (UMP), adenosine monophosphate (AMP), guanosine monophosphate (GMP), thymidine monophosphate (TMP), and inosine monophosphate (IMP). In some embodiments, “nucleoside monophosphate” may refer to a non-natural nucleoside monophosphate. The terms “nucleoside” and “nucleotide” may be used interchangeably in some contexts.

“Nucleoside diphosphate” or “NDP” refers to glycosylamines comprising a nucleobase, a 5-carbon sugar (e.g., ribose, deoxyribose, or arabinose), and a diphosphate (i.e., pyrophosphate) moiety at the 5′-position. In some embodiments herein, “nucleoside diphosphate” is abbreviated as “NDP”. Non-limiting examples of nucleoside diphosphates include cytidine diphosphate (CDP), uridine diphosphate (UDP), adenosine diphosphate (ADP), guanosine diphosphate (GDP), thymidine diphosphate (TDP), and inosine diphosphate (IDP). In some embodiments, “nucleoside diphosphate” may refer to a non-natural nucleoside diphosphate. The terms “nucleoside” and “nucleotide” may be used interchangeably in some contexts.

“Nucleoside triphosphate” or “NTP” refers to glycosylamines comprising a nucleobase, a 5-carbon sugar (e.g., ribose, deoxyribose, or arabinose), and a triphosphate moiety at the 5′-position. In some embodiments herein, “nucleoside triphosphate” is abbreviated as “NTP”. Non-limiting examples of nucleoside triphosphates include cytidine triphosphate (CTP), uridine triphosphate (UTP), adenosine triphosphate (ATP), guanosine triphosphate (GTP), thymidine triphosphate (TTP), and inosine triphosphate (ITP). In some embodiments, “nucleoside triphosphate” may refer to a non-natural nucleoside triphosphate. The terms “nucleoside” and “nucleotide” may be used interchangeably in some contexts.

“Modified” or “non-natural” in context of a nucleoside or nucleotide refers to a nucleoside or nucleotide that has been altered to a non-naturally occurring nucleoside or nucleotide. In some embodiments, modifications to nucleoside or nucleotides include modifications to the nucleobase, sugar moiety, and/or phosphate. In some embodiments, the common modifications of the 2′-position of the sugar residue with fluoro (F) or —O—CH₃ is denoted by “fN” and “mN”, respectively, where N denotes the nucleobase on the nucleoside or nucleotide. In some embodiments, the presence of a 5′-thiophosphate is denoted by “*N”.

“Locked nucleoside” or “locked nucleotide” refers to nucleoside or nucleotide, respectively, in which the ribose moiety is modified with a bridge connecting the 2′ oxygen and 4′ carbon (see, e.g., Obika et al., Tetrahedron Letters, 1997, 38(50):8735-8738; Orum et al., Current Pharmaceutical Design, 2008, 14(11):1138-1142). In some embodiments, the bridge is a methylene or ethylene bridge. In some embodiments, the ribose moiety of the locked nucleoside or locked nucleotide is in the C3′-endo (beta-D) or C2′-endo (alpha-L) conformation.

“Biocatalysis,” “biocatalytic,” “biotransformation,” and “biosynthesis” refer to the use of enzymes to perform chemical reactions on organic compounds.

“Wild-type” and “naturally occurring” refer to the form found in nature. For example, a wild-type polypeptide or polynucleotide sequence is a sequence present in an organism that can be isolated from a source in nature and which has not been intentionally modified by human manipulation.

“Recombinant,” “engineered,” “variant,” “non-natural,” and “non-naturally occurring” when used with reference to a cell, nucleic acid, or polypeptide, refers to a material, or a material corresponding to the natural or native form of the material, which has been modified in a manner that would not otherwise exist in nature. In some embodiments, the cell, nucleic acid or polypeptide is identical a naturally occurring cell, nucleic acid or polypeptide, but is produced or derived from synthetic materials and/or by manipulation using recombinant techniques. Non-limiting examples include, among others, recombinant cells expressing genes that are not found within the native (non-recombinant) form of the cell or express native genes that are otherwise expressed at a different level.

“Percent (%) sequence identity” is used herein to refer to comparisons among polynucleotides or polypeptides, and are determined by comparing two optimally aligned sequences over a comparison window, wherein the portion of the polynucleotide or polypeptide sequence in the comparison window may comprise additions or deletions (i.e., gaps) as compared to the reference sequence for optimal alignment of the two sequences. The percentage may be calculated by determining the number of positions at which the identical nucleic acid base or amino acid residue occurs in both sequences to yield the number of matched positions, dividing the number of matched positions by the total number of positions in the window of comparison and multiplying the result by 100 to yield the percentage of sequence identity. Alternatively, the percentage may be calculated by determining the number of positions at which either the identical nucleic acid base or amino acid residue occurs in both sequences or a nucleic acid base or amino acid residue is aligned with a gap to yield the number of matched positions, dividing the number of matched positions by the total number of positions in the window of comparison and multiplying the result by 100 to yield the percentage of sequence identity. Those of skill in the art appreciate that there are many established algorithms available to align two sequences. Optimal alignment of sequences for comparison can be conducted by any suitable method, including, but not limited to the local homology algorithm of Smith and Waterman (Smith and Waterman, Adv. Appl. Math., 1981, 2:482), by the homology alignment algorithm of Needleman and Wunsch (Needleman and Wunsch, J. Mol. Biol., 1970, 48:443), by the search for similarity method of Pearson and Lipman (Pearson and Lipman, Proc. Natl. Acad. Sci. USA., 1988, 85:2444), by computerized implementations of these algorithms (e.g., GAP, BESTFIT, FASTA, and TFASTA in the GCG Wisconsin Software Package), or by visual inspection, as known in the art. Examples of algorithms that are suitable for determining percent sequence identity and sequence similarity include, but are not limited to the BLAST and BLAST 2.0 algorithms, which are described by Altschul et al. (See Altschul et al., J. Mol. Biol., 1990, 215:403-410; and Altschul et al., Nucl. Acids Res., 1977, 25(17):3389-3402, respectively). Software for performing BLAST analyses is publicly available through the National Center for Biotechnology Information website. This algorithm involves first identifying high scoring sequence pairs (HSPs) by identifying short words of length W in the query sequence, which either match or satisfy some positive-valued threshold score T when aligned with a word of the same length in a database sequence. T is referred to as, the neighborhood word score threshold (See, Altschul et al., supra). These initial neighborhood word hits act as seeds for initiating searches to find longer HSPs containing them. The word hits are then extended in both directions along each sequence for as far as the cumulative alignment score can be increased. Cumulative scores are calculated using, for nucleotide sequences, the parameters M (reward score for a pair of matching residues; always >0) and N (penalty score for mismatching residues; always <0). For amino acid sequences, a scoring matrix is used to calculate the cumulative score. Extension of the word hits in each direction are halted when: the cumulative alignment score falls off by the quantity X from its maximum achieved value; the cumulative score goes to zero or below, due to the accumulation of one or more negative-scoring residue alignments; or the end of either sequence is reached. The BLAST algorithm parameters W, T, and X determine the sensitivity and speed of the alignment. The BLASTN program (for nucleotide sequences) uses as defaults a wordlength (W) of 11, an expectation (E) of 10, M=5, N=−4, and a comparison of both strands. For amino acid sequences, the BLASTP program uses as defaults a word length (W) of 3, an expectation (E) of 10, and the BLOSUM62 scoring matrix (See, Henikoff and Henikoff, Proc. Natl. Acad. Sci. USA, 1989, 89:10915). Exemplary determination of sequence alignment and % sequence identity can employ the BESTFIT or GAP programs in the GCG Wisconsin Software package (Accelrys, Madison WI), using default parameters provided.

“Substantial identity” refers to a polynucleotide or polypeptide sequence that has at least 80 percent sequence identity, at least 85 percent identity, at least between 89 to 95 percent sequence identity, or more usually, at least 99 percent sequence identity as compared to a reference sequence over a comparison window of at least 20 residue positions, frequently over a window of at least 30-50 residues, wherein the percentage of sequence identity is calculated by comparing the reference sequence to a sequence that includes deletions or additions which total 20 percent or less of the reference sequence over the window of comparison. In some specific embodiments applied to polypeptides, the term “substantial identity” means that two polypeptide sequences, when optimally aligned, such as by the programs GAP or BESTFIT using default gap weights, share at least 80 percent sequence identity, preferably at least 89 percent sequence identity, at least 95 percent sequence identity or more (e.g., 99 percent sequence identity). In some embodiments, residue positions that are not identical in sequences being compared differ by conservative amino acid substitutions.

“Reference sequence” refers to a defined sequence used as a basis for a sequence and/or activity comparison. A reference sequence may be a subset of a larger sequence, for example, a segment of a full-length gene or polypeptide sequence. Generally, a reference sequence is at least 20 nucleotide or amino acid residues in length, at least 25 residues in length, at least 50 residues in length, at least 100 residues in length or the full length of the nucleic acid or polypeptide. Since two polynucleotides or polypeptides may each (1) comprise a sequence (i.e., a portion of the complete sequence) that is similar between the two sequences, and (2) may further comprise a sequence that is divergent between the two sequences, sequence comparisons between two (or more) polynucleotides or polypeptides are typically performed by comparing sequences of the two polynucleotides or polypeptides over a “comparison window” to identify and compare local regions of sequence similarity. In some embodiments, a “reference sequence” can be based on a primary amino acid sequence, where the reference sequence is a sequence that can have one or more changes in the primary sequence.

“Comparison window” refers to a conceptual segment of contiguous nucleotide positions or amino acids residues wherein a sequence may be compared to a reference sequence. In some embodiments, the comparison window is at least 15 to 20 contiguous nucleotides or amino acids and wherein the portion of the sequence in the comparison window may comprise additions or deletions (i.e., gaps) of 20 percent or less as compared to the reference sequence (which does not comprise additions or deletions) for optimal alignment of the two sequences. In some embodiments, the comparison window can be longer than 15-20 contiguous residues, and includes, optionally 30, 40, 50, 100, or longer windows.

“Corresponding to,” “reference to,” and “relative to” when used in the context of the numbering of a given amino acid or polynucleotide sequence refer to the numbering of the residues of a specified reference sequence when the given amino acid or polynucleotide sequence is compared to the reference sequence. In other words, the residue number or residue position of a given polymer is designated with respect to the reference sequence rather than by the actual numerical position of the residue within the given amino acid or polynucleotide sequence. For example, a given amino acid sequence, such as that of an engineered adenylate kinase, can be aligned to a reference sequence by introducing gaps to optimize residue matches between the two sequences. In these cases, although the gaps are present, the numbering of the residue in the given amino acid or polynucleotide sequence is made with respect to the reference sequence to which it has been aligned.

“Amino acid difference”, “residue difference” and “substitution” refer to a difference in the amino acid residue at a position of a polypeptide sequence relative to the amino acid residue at a corresponding position in a reference sequence. The positions of amino acid differences generally are referred to herein as “Xn,” where n refers to the corresponding position in the reference sequence upon which the residue difference is based. For example, a “residue difference at position X13 as compared to SEQ ID NO: 2” refers to a difference of the amino acid residue at the polypeptide position corresponding to position 13 of SEQ ID NO: 2. Thus, if the reference polypeptide of SEQ ID NO: 2 has a methionine at position 13, then a “residue difference at position X13 as compared to SEQ ID NO: 2” refers to an amino acid substitution of any residue other than methionine at the position of the polypeptide corresponding to position 13 of SEQ ID NO: 2. In most instances herein, the specific amino acid residue difference at a position is indicated as “XnY” where “Xn” specified the corresponding position as described above, and “Y” is the single letter identifier of the amino acid found in the engineered polypeptide (i.e., the different residue than in the reference polypeptide). In some instances (e.g., in the Tables presented in the Examples), the present invention also provides specific amino acid differences denoted by the conventional notation “AnB”, where A is the single letter identifier of the residue in the reference sequence, “n” is the number of the residue position in the reference sequence, and B is the single letter identifier of the residue substitution in the sequence of the engineered polypeptide. In some instances, an amino acid residue difference or substitution may be a deletion and may be denoted by a “−”. In some instances, a polypeptide of the present invention can include one or more amino acid residue differences relative to a reference sequence, which is indicated by a list of the specified positions where residue differences are present relative to the reference sequence. In some embodiments, the amino acid difference, e.g., a substitution, is denoted by the abbreviation “nB,” without the identifier for the residue in the reference sequence. In some embodiments, the phrase “an amino acid residue nB” denotes the presence of the amino residue in the engineered polypeptide, which may or may not be a substitution in context of a reference polypeptide or amino acid sequence.

In some instances, a polypeptide of the present disclosure can include one or more amino acid residue differences relative to a reference sequence, which is indicated by a list of the specified positions where residue differences are present relative to the reference sequence. In some embodiments, where more than one amino acid can be used in a specific residue position of a polypeptide, the various amino acid residues that can be used are separated by a “/” (e.g., X13G/X13S, X13G/S, or 13G/S).

“Amino acid substitution set” or “substitution set” refers to a group of amino acid substitutions in a polypeptide sequence, as compared to a reference sequence. A substitution set can have 2, 3, 4, 5, 6, 7, 8, 9, 10, 11, 12, 13, 14, 15, or more amino acid substitutions. In some embodiments, a substitution set refers to the set of amino acid substitutions that is present in any of the variant adenylate kinases listed in the Tables provided in the Examples. In these substitution sets, the individual substitutions are separated by a semicolon (“;”; e.g., Y29Q;A30S) or slash (“/”; e.g., Y29Q/A30S or 29Q/30S).

“Conservative amino acid substitution” refers to a substitution of a residue with a different residue having a similar side chain, and thus typically involves substitution of the amino acid in the polypeptide with amino acids within the same or similar defined class of amino acids. By way of example and not limitation, in some embodiments, an amino acid with an aliphatic side chain is substituted with another aliphatic amino acid (e.g., alanine, valine, leucine, and isoleucine); an amino acid with an hydroxyl side chain is substituted with another amino acid with an hydroxyl side chain (e.g., serine and threonine); an amino acids having aromatic side chains is substituted with another amino acid having an aromatic side chain (e.g., phenylalanine, tyrosine, tryptophan, and histidine); an amino acid with a basic side chain is substituted with another amino acid with a basis side chain (e.g., lysine and arginine); an amino acid with an acidic side chain is substituted with another amino acid with an acidic side chain (e.g., aspartic acid or glutamic acid); and/or a hydrophobic or hydrophilic amino acid is replaced with another hydrophobic or hydrophilic amino acid, respectively.

“Non-conservative substitution” refers to substitution of an amino acid in the polypeptide with an amino acid with significantly differing side chain properties. Non-conservative substitutions may use amino acids between, rather than within, the defined groups and affects (a) the structure of the peptide backbone in the area of the substitution (e.g., proline for glycine) (b) the charge or hydrophobicity, or (c) the bulk of the side chain. By way of example and not limitation, an exemplary non-conservative substitution can be an acidic amino acid substituted with a basic or aliphatic amino acid; an aromatic amino acid substituted with a small amino acid; and a hydrophilic amino acid substituted with a hydrophobic amino acid.

“Deletion” refers to modification to the polypeptide by removal of one or more amino acids from the reference polypeptide. Deletions can comprise removal of 1 or more amino acids, 2 or more amino acids, 5 or more amino acids, 10 or more amino acids, 15 or more amino acids, or 20 or more amino acids, up to 10% of the total number of amino acids, or up to 20% of the total number of amino acids making up the reference enzyme while retaining enzymatic activity and/or retaining the improved properties of an engineered adenylate kinase enzyme. Deletions can be directed to the internal portions and/or terminal portions of the polypeptide. In various embodiments, the deletion can comprise a continuous segment or can be discontinuous. Deletions are typically indicated by “−” in amino acid sequences.

“Insertion” refers to modification to the polypeptide by addition of one or more amino acids to the reference polypeptide. Insertions can be in the internal portions of the polypeptide, or to the carboxy or amino terminus. Insertions as used herein include fusion proteins as is known in the art. The insertion can be a contiguous segment of amino acids or separated by one or more of the amino acids in the naturally occurring polypeptide.

“Functional fragment” and “biologically active fragment” are used interchangeably herein to refer to a polypeptide that has an amino-terminal and/or carboxy-terminal deletion(s) and/or internal deletions, but where the remaining amino acid sequence is identical to the corresponding positions in the sequence to which it is being compared (e.g., a full-length engineered adenylate kinase of the present invention) and that retains substantially all of the activity of the full-length polypeptide.

“Isolated polypeptide” refers to a polypeptide which is substantially separated from other contaminants that naturally accompany it (e.g., protein, lipids, and polynucleotides). The term embraces polypeptides which have been removed or purified from their naturally occurring environment or expression system (e.g., within a host cell or via in vitro synthesis). The recombinant adenylate kinase polypeptides may be present within a cell, present in the cellular medium, or prepared in various forms, such as lysates or isolated preparations. As such, in some embodiments, the recombinant adenylate kinase polypeptides can be an isolated polypeptide.

“Substantially pure polypeptide” or “purified protein” refers to a composition in which the polypeptide species is the predominant species present (i.e., on a molar or weight basis it is more abundant than any other individual macromolecular species in the composition), and is generally a substantially purified composition when the object species comprises at least about 50 percent of the macromolecular species present by mole or % weight. However, in some embodiments, the composition comprising adenylate kinase comprises adenylate kinase that is less than 50% pure (e.g., about 10%, about 20%, about 30%, about 40%, or about 50%). Generally, a substantially pure adenylate kinase composition comprises about 60% or more, about 70% or more, about 80% or more, about 90% or more, about 95% or more, and about 98% or more of all macromolecular species by mole or % weight present in the composition. In some embodiments, the object species is purified to essential homogeneity (i.e., contaminant species cannot be detected in the composition by conventional detection methods) wherein the composition consists essentially of a single macromolecular species. Solvent species, small molecules (<500 Daltons), and elemental ion species are not considered macromolecular species. In some embodiments, the isolated recombinant adenylate kinase polypeptides are substantially pure polypeptide compositions.

“Improved enzyme property” refers to at least one improved property of an enzyme. In some embodiments, the present invention provides engineered adenylate kinase polypeptides that exhibit an improvement in any enzyme property as compared to a reference adenylate kinase polypeptide and/or a wild-type adenylate kinase polypeptide, and/or another engineered adenylate kinase polypeptide. Thus, the level of “improvement” can be determined and compared between various adenylate kinase polypeptides, including wild-type, as well as engineered adenylate kinases. Improved properties include, but are not limited, to such properties as increased protein expression, increased thermoactivity, increased thermostability, increased pH activity, increased stability, increased enzymatic activity, increased substrate specificity or affinity, increased specific activity, increased resistance to substrate or end-product inhibition, increased chemical stability, improved chemoselectivity, improved solvent stability, increased tolerance to acidic pH, increased tolerance to proteolytic activity (i.e., reduced sensitivity to proteolysis), reduced aggregation, increased solubility, and altered temperature profile. In additional embodiments, the term is used in reference to the at least one improved property of adenylate kinase enzymes. In some embodiments, the present invention provides adenylate kinase polypeptides that exhibit an improvement in any enzyme property as compared to a reference adenylate kinase polypeptide and/or a wild-type adenylate kinase polypeptide, and/or another engineered adenylate kinase polypeptide. Thus, the level of “improvement” can be determined and compared between various adenylate kinase polypeptides, including wild-type, as well as engineered adenylate kinases.

“Increased enzymatic activity” and “enhanced catalytic activity” refer to an improved property of the engineered polypeptides, which can be represented by an increase in specific activity (e.g., product produced/time/weight protein) or an increase in percent conversion of the substrate to the product (e.g., percent conversion of starting amount of substrate to product in a specified time period using a specified amount of enzyme) as compared to the reference enzyme. In some embodiments, the terms refer to an improved property of engineered adenylate kinase polypeptides provided herein, which can be represented by an increase in specific activity (e.g., product produced/time/weight protein) or an increase in percent conversion of the substrate to the product (e.g., percent conversion of starting amount of substrate to product in a specified time period using a specified amount of adenylate kinase) as compared to the reference adenylate kinase enzyme. In some embodiments, the terms are used in reference to improved adenylate kinase enzymes provided herein. Exemplary methods to determine enzyme activity of the engineered adenylate kinases of the present invention are provided in the Examples. Any property relating to enzyme activity may be affected, including the classical enzyme properties of Kₘ, Vₘₐₓ or k_{cat}, changes of which can lead to increased enzymatic activity. For example, improvements in enzyme activity can be from about 1.1 fold the enzymatic activity of the corresponding wild-type enzyme, to as much as 2-fold, 5-fold, 10-fold, 20-fold, 25-fold, 50-fold, 75-fold, 100-fold, 150-fold, 200-fold or more enzymatic activity than the naturally occurring adenylate kinase or another engineered adenylate kinase from which the adenylate kinase polypeptides were derived.

“Conversion” refers to the enzymatic conversion (or biotransformation) of a substrate(s) to the corresponding product(s). “Percent conversion” refers to the percent of the substrate that is converted to the product within a period of time under specified conditions. Thus, the “enzymatic activity” or “activity” of an adenylate kinase polypeptide can be expressed as “percent conversion” of the substrate to the product in a specific period of time.

“Stringent hybridization conditions” is used herein to refer to conditions under which nucleic acid hybrids are stable. As known to those of skill in the art, the stability of hybrids is reflected in the melting temperature (Tm) of the hybrids. In general, the stability of a hybrid is a function of ion strength, temperature, G/C content, and the presence of chaotropic agents. The T. values for polynucleotides can be calculated using known methods for predicting melting temperatures (See e.g., Baldino et al., Meth. Enzymol., 1989, 168:761-777; Bolton et al., Proc. Natl. Acad. Sci. USA., 1962, 48:1390; Bresslauer et al., Proc. Natl. Acad. Sci. USA, 1986, 83:8893-8897; Freier et al., Proc. Natl. Acad. Sci. USA., 1986, 83:9373-9377; Kierzek et al., Biochem., 1986, 25:7840-7846; Rychlik et al., Nucl. Acids Res., 1990, 18:6409-6412 (erratum, Nucl. Acids Res., 1991, 19:698); Sambrook et al., supra); Suggs et al., 1981, in “Developmental Biology Using Purified Genes,” Brown et al. [eds.], pp. 683-693, Academic Press, Cambridge, MA (1981); and Wetmur, Crit. Rev. Biochem. Mol. Biol., 1991, 26:227-259). In some embodiments, the polynucleotide encodes the polypeptide disclosed herein and hybridizes under defined conditions, such as moderately stringent or highly stringent conditions, to the complement of a sequence encoding an engineered adenylate kinase enzyme of the present invention.

“Hybridization stringency” relates to hybridization conditions, such as washing conditions, in the hybridization of nucleic acids. Generally, hybridization reactions are performed under conditions of lower stringency, followed by washes of varying but higher stringency (see, e.g., Sambrook et al., Molecular Cloning: A Laboratory Manual, Cold Spring Harbor Laboratory Press, New York, 2001; Ausubel et al., Current Protocols in Molecular Biology, John Wiley & Sons, 2003). The term “moderately stringent hybridization” refers to conditions that permit target-DNA to bind a complementary nucleic acid that has about 60% identity, preferably about 75% identity, about 85% identity to the target DNA, with greater than about 90% identity to target-polynucleotide. Exemplary moderately stringent conditions are conditions equivalent to hybridization in 50% formamide, 5×Denhart's solution, 5×SSPE, 0.2% SDS at 42° C., followed by washing in 0.2×SSPE, 0.2% SDS, at 42° C. “High stringency hybridization” refers generally to conditions that are about 10° C. or less from the thermal melting temperature T. as determined under the solution condition for a defined polynucleotide sequence. In some embodiments, a high stringency condition refers to conditions that permit hybridization of only those nucleic acid sequences that form stable hybrids in 0.018M NaCl at 65° C. (i.e., if a hybrid is not stable in 0.018M NaCl at 65° C., it will not be stable under high stringency conditions, as contemplated herein). High stringency conditions can be provided, for example, by hybridization in conditions equivalent to 50% formamide, 5×Denhart's solution, 5×SSPE, 0.2% SDS at 42° C., followed by washing in 0.1×SSPE, and 0.1% SDS at 65° C. Another high stringency condition is hybridizing in conditions equivalent to hybridizing in 5×SSC containing 0.1% (w/v) SDS at 65° C. and washing in 0.1×SSC containing 0.1% SDS at 65° C. Other high stringency hybridization conditions, as well as moderately stringent conditions, are described in the references cited above.

“Coding sequence” refers to that portion of a nucleic acid (e.g., a gene) that encodes an amino acid sequence of a protein.

“Codon optimized” refers to changes in the codons of the polynucleotide encoding a protein to those preferentially used in a particular organism such that the encoded protein is efficiently expressed in the organism of interest. Although the genetic code is degenerate in that most amino acids are represented by several codons, called “synonyms” or “synonymous” codons, it is well known that codon usage by particular organisms is nonrandom and biased towards particular codon triplets. This codon usage bias may be higher in reference to a given gene, genes of common function or ancestral origin, highly expressed proteins versus low copy number proteins, and the aggregate protein coding regions of an organism's genome. In some embodiments, the polynucleotides encoding the adenylate kinase enzymes may be codon optimized for optimal production in the host organism selected for expression.

“Preferred,” “optimal,” and “high codon usage bias” codons when used alone or in combination refer(s) interchangeably to codons that are used at higher frequency in the protein coding regions than other codons that code for the same amino acid. The preferred codons may be determined in relation to codon usage in a single gene, a set of genes of common function or origin, highly expressed genes, the codon frequency in the aggregate protein coding regions of the whole organism, codon frequency in the aggregate protein coding regions of related organisms, or combinations thereof. Codons whose frequency increases with the level of gene expression are typically optimal codons for expression. A variety of methods are known for determining the codon frequency (e.g., codon usage, relative synonymous codon usage) and codon preference in specific organisms, including multivariate analysis, for example, using cluster analysis or correspondence analysis, and the effective number of codons used in a gene (See e.g., GCG CodonPreference, Genetics Computer Group Wisconsin Package; CodonW, Peden, University of Nottingham; McInerney, Bioinform., 1998, 14:372-73; Stenico et al., Nucl. Acids Res., 1994, 222437-46; and Wright, Gene, 1990, 87:23-29). Codon usage tables are available for many different organisms (See e.g., Wada et al., Nucl. Acids Res., 1992, 20:2111-2118; Nakamura et al., Nucl. Acids Res., 2000, 28:292; Duret, et al., supra; Henaut and Danchin, in Escherichia coli and Salmonella, Neidhardt, et al. (eds.), ASM Press, Washington D.C., p. 2047-2066 (1996)). The data source for obtaining codon usage may rely on any available nucleotide sequence capable of coding for a protein. These data sets include nucleic acid sequences actually known to encode expressed proteins (e.g., complete protein coding sequences-CDS), expressed sequence tags (ESTS), or predicted coding regions of genomic sequences (See e.g., Mount, “Bioinformatics: Sequence and Genome Analysis,” Chapter 8, Cold Spring Harbor Laboratory Press, Cold Spring Harbor, N.Y. (2001); Uberbacher, Meth. Enzymol., 1996, 266:259-281; and Tiwari et al., Comput. Appl. Biosci., 1997, 13:263-270).

“Control sequence” refers herein to include all components, which are necessary or advantageous for the expression of a polynucleotide and/or polypeptide of the present application. Each control sequence may be native or foreign to the nucleic acid sequence encoding the polypeptide. Such control sequences include, but are not limited to, a leader, polyadenylation sequence, pro-peptide sequence, promoter sequence, signal peptide sequence, initiation sequence and transcription terminator. In some embodiments, the control sequences include a promoter, and transcriptional and translational stop signals.

“Operably linked” or “operatively linked” is defined herein as a configuration in which a control sequence is appropriately placed (i.e., in a functional relationship) at a position relative to a polynucleotide of interest such that the control sequence directs or regulates the expression of the polynucleotide, and where appropriate the encoded polypeptide of interest.

“Promoter sequence” refers to a nucleic acid sequence that defines and/or initiates expression of a polynucleotide of interest, such as a coding sequence. The promoter sequence contains transcriptional control sequences, which mediate the expression of a polynucleotide of interest. The promoter may be any nucleic acid sequence which shows transcriptional activity in the host cell of choice including mutant, truncated, and hybrid promoters, and may be obtained from genes encoding extracellular or intracellular polypeptides either homologous or heterologous to the host cell.

“Heterologous” refers to the relationship between two or more nucleic acid or protein sequences (e.g., a promoter sequence, signal peptide, terminator sequence, etc.) that are derived from different sources and are not associated in nature.

“Vector” refers to a polynucleotide construct for introducing a polynucleotide sequence into a cell. In some embodiments, the vector is an expression vector that is operably linked to a suitable control sequence capable of effecting the expression in a suitable host of the polynucleotide of interest and where appropriate the encoded polypeptide.

“Expression” includes any step involved in the production of the polypeptide including, but not limited to, transcription, post-transcriptional modification, translation, and post-translational modification. In some embodiments, the term also encompasses secretion of the polypeptide from a cell.

“Produces” refers to the production of proteins and/or other compounds by cells. It is intended that the term encompass any step involved in the production of polypeptides including, but not limited to, transcription, post-transcriptional modification, translation, and post-translational modification. In some embodiments, the term also encompasses secretion of the polypeptide from a cell.

“Culturing” refers to the growing of a population of microbial cells under any suitable conditions (e.g., using a liquid, gel or solid medium).

“Host cell” and “host strain” refer to suitable hosts for expression vectors comprising DNA provided herein (e.g., the polynucleotides encoding the adenylate kinase variants). In some embodiments, the host cells are prokaryotic or eukaryotic cells that have been transformed or transfected with vectors constructed using recombinant DNA techniques as known in the art.

“pH stable” refers to an adenylate kinase polypeptide that maintains similar activity (e.g., more than 60% to 80%) after exposure to high or low pH (e.g., 4.5-6 or 8 to 12) for a period of time (e.g., 0.5-24 hrs) compared to the untreated enzyme.

“Thermostable” refers to an adenylate kinase polypeptide that maintains similar activity (more than 60% to 80% for example) after exposure to elevated temperatures (e.g., 40-80° C.) for a period of time (e.g., 0.5-24 h) compared to the wild-type enzyme exposed to the same elevated temperature.

“Solvent stable” refers to an adenylate kinase polypeptide that maintains similar activity (more than e.g., 60% to 80%) after exposure to varying concentrations (e.g., 5-99%) of solvent (ethanol, isopropyl alcohol, dimethylsulfoxide [DMSO], tetrahydrofuran, 2-methyltetrahydrofuran, acetone, toluene, butyl adenylate, methyl tert-butyl ether, etc.) for a period of time (e.g., 0.5-24 h) compared to the wild-type enzyme exposed to the same concentration of the same solvent.

“Thermo- and solvent stable” refers to an adenylate kinase polypeptide that is both thermostable and solvent stable.

“Stereoselectivity” refers to the preferential formation in a chemical or enzymatic reaction of one stereoisomer over another. Stereoselectivity can be partial, where the formation of one stereoisomer is favored over the other, or it may be complete where only one stereoisomer is formed. When the stereoisomers are enantiomers, the stereoselectivity is referred to as enantioselectivity, the fraction (typically reported as a percentage) of one enantiomer in the sum of both. It is commonly alternatively reported in the art (typically as a percentage) as the enantiomeric excess (“e.e.”) calculated therefrom according to the formula [major enantiomer−minor enantiomer]/[major enantiomer+minor enantiomer]. Where the stereoisomers are diastereoisomers, the stereoselectivity is referred to as diastereoselectivity, the fraction (typically reported as a percentage) of one diastereomer in a mixture of two diastereomers, commonly alternatively reported as the diastereomeric excess (“d.e.”). Enantiomeric excess and diastereomeric excess are types of stereomeric excess.

“Regioselectivity” and “regioselective reaction” refer to a reaction in which one direction of bond making or breaking occurs preferentially over all other possible directions. Reactions can completely (100%) regioselective if the discrimination is complete, substantially regioselective (at least 75%), or partially regioselective (x %, wherein the percentage is set dependent upon the reaction of interest), if the product of reaction at one site predominates over the product of reaction at other sites.

“Chemoselectivity” refers to the preferential formation in a chemical or enzymatic reaction of one product over another.

“Suitable reaction conditions” refers to those conditions in the enzymatic conversion reaction solution (e.g., ranges of enzyme loading, substrate loading, temperature, pH, buffers, co-solvents, etc.) under which an adenylate kinase polypeptide of the present invention is capable of converting a substrate to the desired product compound. Some exemplary “suitable reaction conditions” are provided herein.

“Loading,” such as in “compound loading” or “enzyme loading” refers to the concentration or amount of a component in a reaction mixture at the start of the reaction.

“Substrate” in the context of an enzymatic conversion reaction process refers to the compound or molecule acted on by the engineered enzymes provided herein (e.g., engineered adenylate kinase polypeptides).

“Product” in the context of an enzymatic conversion process refers to the compound or molecule resulting from the action of an enzymatic polypeptide on a substrate.

“Increasing” yield of a product (e.g., a nucleoside triphosphate or analogue) from a reaction occurs when a particular component present during the reaction (e.g., an adenylate kinase enzyme) causes more product to be produced, compared with a reaction conducted under the same conditions with the same substrate and other substituents, but in the absence of the component of interest.

“Substantially free” of a particular enzyme if the amount of that enzyme compared with other enzymes that participate in catalyzing the reaction is less than about 2%, about 1%, or about 0.1% (wt/wt).

“Fractionating” a liquid (e.g., a culture broth) means applying a separation process (e.g., salt precipitation, column chromatography, size exclusion, and filtration) or a combination of such processes to provide a solution in which a desired protein comprises a greater percentage of total protein in the solution than in the initial liquid product.

“Alkyl” refers to saturated hydrocarbon groups of from 1 to 18 carbon atoms inclusively, either straight chained or branched, more preferably from 1 to 8 carbon atoms inclusively, and most preferably 1 to 6 carbon atoms inclusively. An alkyl with a specified number of carbon atoms is denoted in parenthesis (e.g., (C₁-C₄)alkyl refers to an alkyl of 1 to 4 carbon atoms).

“Alkenyl” refers to groups of from 2 to 12 carbon atoms inclusively, either straight or branched containing at least one double bond but optionally containing more than one double bond.

“Alkynyl” refers to groups of from 2 to 12 carbon atoms inclusively, either straight or branched containing at least one triple bond but optionally containing more than one triple bond, and additionally optionally containing one or more double bonded moieties.

“Heteroalkyl, “heteroalkenyl,” and heteroalkynyl,” refer to alkyl, alkenyl and alkynyl as defined herein in which one or more of the carbon atoms are each independently replaced with the same or different heteroatoms or heteroatomic groups. Heteroatoms and/or heteroatomic groups which can replace the carbon atoms include, but are not limited to, —O—, —S—, —S—O—, —NR′—, —PH—, —S(O)—, —S(O)2-, —S(O)NR′—, —S(O)2NR′—, and the like, including combinations thereof, where each Rα is independently selected from hydrogen, alkyl, heteroalkyl, cycloalkyl, heterocycloalkyl, aryl, and heteroaryl.

“Alkoxy” refers to the group —OR′ wherein R′ is an alkyl group is as defined above including optionally substituted alkyl groups as also defined herein.

“Amino” refers to the group —NH₁. Substituted amino refers to the group —NHR′, NR′R′, and NR′R′R′, where each R′ is independently selected from substituted or unsubstituted alkyl, cycloalkyl, cycloheteroalkyl, alkoxy, aryl, heteroaryl, heteroarylalkyl, acyl, alkoxycarbonyl, sulfanyl, sulfinyl, sulfonyl, and the like. Typical amino groups include, but are limited to, dimethylamino, diethylamino, trimethylammonium, triethylammonium, methylysulfonylamino, furanyl-oxy-sulfamino, and the like.

“Oxo” refers to ═O.

“Oxy” refers to a divalent group —O—, which may have various substituents to form different oxy groups, including ethers and esters.

“Carboxy” refers to —COOH.

“Carbonyl” refers to —C(O)—, which may have a variety of substituents to form different carbonyl groups including acids, acid halides, aldehydes, amides, esters, and ketones.

“Alkyloxycarbonyl” refers to —C(O)OR′, where R′ is an alkyl group as defined herein, which can be optionally substituted.

As used herein, “aminocarbonyl” refers to —C(O)NH₂. Substituted aminocarbonyl refers to —C(O)NR′R′, where the amino group NR′R′ is as defined herein.

“Halogen” and “halo” refer to fluoro, chloro, bromo and iodo.

“Hydroxy” refers to —OH.

“Cyano” refers to —CN.

“Aryl” refers to an unsaturated aromatic carbocyclic group of from 6 to 12 carbon atoms inclusively having a single ring (e.g., phenyl) or multiple condensed rings (e.g., naphthyl or anthryl). Exemplary aryls include phenyl, pyridyl, naphthyl and the like.

“Heterocycle,” “heterocyclic,” and interchangeably “heterocycloalkyl,” refer to a saturated or unsaturated group having a single ring or multiple condensed rings, from 2 to 10 carbon ring atoms inclusively and from 1 to 4 hetero ring atoms inclusively selected from nitrogen, sulfur or oxygen within the ring. Such heterocyclic groups can have a single ring (e.g., piperidinyl or tetrahydrofuryl) or multiple condensed rings (e.g., indolinyl, dihydrobenzofuran or quinuclidinyl). Examples of heterocycles include, but are not limited to, furan, thiophene, thiazole, oxazole, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, pyrrolidine, indoline and the like.

“Heteroaryl” refers to an aromatic heterocyclic group of from 1 to 10 carbon atoms inclusively and 1 to 4 heteroatoms inclusively selected from oxygen, nitrogen and sulfur within the ring. Such heteroaryl groups can have a single ring (e.g., pyridyl or furyl) or multiple condensed rings (e.g., indolizinyl or benzothienyl).

“Heteroarylalkyl” refers to an alkyl substituted with a heteroaryl (i.e., heteroaryl-alkyl-groups), preferably having from 1 to 6 carbon atoms inclusively in the alkyl moiety and from 5 to 12 ring atoms inclusively in the heteroaryl moiety. Such heteroarylalkyl groups are exemplified by pyridylmethyl and the like.

“Heteroarylalkenyl” refers to an alkenyl substituted with a heteroaryl (i.e., heteroaryl-alkenyl-groups), preferably having from 2 to 6 carbon atoms inclusively in the alkenyl moiety and from 5 to 12 ring atoms inclusively in the heteroaryl moiety.

“Heteroarylalkynyl” refers to an alkynyl substituted with a heteroaryl (i.e., heteroaryl-alkynyl-groups), preferably having from 2 to 6 carbon atoms inclusively in the alkynyl moiety and from 5 to 12 ring atoms inclusively in the heteroaryl moiety.

“Membered ring” is meant to embrace any cyclic structure. The number preceding the term “membered” denotes the number of skeletal atoms that constitute the ring. Thus, for example, cyclohexyl, pyridine, pyran and thiopyran are 6-membered rings and cyclopentyl, pyrrole, furan, and thiophene are 5-membered rings.

“Phosphate” as used herein refers to a functional group comprised of an orthophosphate ion (phosphorous atom covalently linked to four oxygen atoms). The orthophosphate ion is commonly found with one or more hydrogen atoms or organic groups. A phosphate group or chain may be modified, as further described herein.

“Phosphorylated” as used herein refers to the addition or presence of one of more phosphoryl groups (phosphorous atom covalently linked to the three oxygen atoms).

“Thiophosphate” refers to an instance where a non-bridging oxygen in a phosphate group of a phosphodiester bond, NMP, NDP, NTP or NQP is replaced with a sulfur. In some embodiments, nucleoside 5′-thiomonophosphate is referred to as NMP-S or NMPαS. In some embodiments, nucleoside-5′-1-thio(diphosphate) and nucleoside-5′-1-thio(triphosphate) are referred to as NDPαS or αS-NDP, and NTPaS or αS-NTP, respectively. In some embodiments, nucleoside-5′-2-thio(diphosphate) and nucleoside-5′-2-thio(triphosphate) are referred to as NDPβS and NTPβS, respectively.

“Dithiophosphate” refers to an instance where two non-bridging oxygens in a phosphate group of a phosphodiester bond, NMP, NDP, NTP or NQP are replaced with two sulfurs.

Unless otherwise specified, positions occupied by hydrogen in the foregoing groups can be further substituted with substituents exemplified by, but not limited to, hydroxy, oxo, nitro, methoxy, ethoxy, alkoxy, substituted alkoxy, trifluoromethoxy, haloalkoxy, fluoro, chloro, bromo, iodo, halo, methyl, ethyl, propyl, butyl, alkyl, alkenyl, alkynyl, substituted alkyl, trifluoromethyl, haloalkyl, hydroxyalkyl, alkoxyalkyl, thio, alkylthio, acyl, carboxy, alkoxycarbonyl, carboxamido, substituted carboxamido, alkylsulfonyl, alkylsulfinyl, alkylsulfonylamino, sulfonamido, substituted sulfonamido, cyano, amino, substituted amino, alkylamino, dialkylamino, aminoalkyl, acylamino, amidino, amidoximo, hydroxamoyl, phenyl, aryl, substituted aryl, aryloxy, arylalkyl, arylalkenyl, arylalkynyl, pyridyl, imidazolyl, heteroaryl, substituted heteroaryl, heteroaryloxy, heteroarylalkyl, heteroarylalkenyl, heteroarylalkynyl, cyclopropyl, cyclobutyl, cyclopentyl, cyclohexyl, cycloalkyl, cycloalkenyl, cycloalkylalkyl, substituted cycloalkyl, cycloalkyloxy, pyrrolidinyl, piperidinyl, morpholino, heterocycle, (heterocycle)oxy, and (heterocycle)alkyl; and preferred heteroatoms are oxygen, nitrogen, and sulfur. It is understood that where open valences exist on these substituents they can be further substituted with alkyl, cycloalkyl, aryl, heteroaryl, and/or heterocycle groups, that where these open valences exist on carbon they can be further substituted by halogen and by oxygen-, nitrogen-, or sulfur-bonded substituents, and where multiple such open valences exist, these groups can be joined to form a ring, either by direct formation of a bond or by formation of bonds to a new heteroatom, preferably oxygen, nitrogen, or sulfur. It is further understood that the above substitutions can be made provided that replacing the hydrogen with the substituent does not introduce unacceptable instability to the molecules of the present invention, and is otherwise chemically reasonable.

“Optional” and “optionally” mean that the subsequently described event or circumstance may or may not occur, and that the description includes instances where the event or circumstance occurs and instances in which it does not. One of ordinary skill in the art would understand that with respect to any molecule described as containing one or more optional substituents, only sterically practical and/or synthetically feasible compounds are meant to be included.

“Optionally substituted” refers to all subsequent modifiers in a term or series of chemical groups. For example, in the term “optionally substituted arylalkyl, the “alkyl” portion and the “aryl” portion of the molecule may or may not be substituted, and for the series “optionally substituted alkyl, cycloalkyl, aryl and heteroaryl,” the alkyl, cycloalkyl, aryl, and heteroaryl groups, independently of the others, may or may not be substituted.

### Engineered Adenylate Kinase Polypeptides

In one aspect, the present disclosure provides adenylate kinase enzymes engineered to have improved properties compared to the parent adenylate kinase enzyme. In some embodiments, the engineered adenylate kinase enzymes exhibit, among others, increased activity on nucleoside monophosphate (NMP) substrates, increased thermostability, and/or increased activity on modified NMP substrates. The engineered adenylate kinases are useful in the conversion of nucleoside monophosphate substrates to their corresponding nucleoside diphosphate products, particularly in enzyme cascade systems.

In some embodiments, an engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12-231 of an even-numbered SEQ ID NO. of SEQ ID NOs: 2-1302, 1356-2652, and 2676-3192, or to a reference sequence corresponding to an even-numbered SEQ ID NO. of SEQ ID NOs: 2-1302, 1356-2652, and 2676-3192, wherein the amino acid sequence comprises one or more substitutions relative to a reference sequence corresponding to amino acid residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or relative to a reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or to a reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, wherein the amino acid sequence comprises one or more substitutions relative to the reference sequence corresponding to amino acid residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12-231 of SEQ ID NO: 2, or to the reference sequence corresponding to SEQ ID NO: 2, wherein the amino acid sequence comprises one or more substitutions relative to the reference sequence corresponding to amino acid residues 12-231 of SEQ ID NO: 2, or relative to the reference sequence corresponding to SEQ ID NO: 2.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, wherein the amino acid sequence comprises one or more substitutions relative to the reference sequence corresponding to amino acid residues 12-231 of SEQ ID NO: 2, or relative to the reference sequence corresponding to SEQ ID NO: 2.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12-231 of an even-numbered SEQ ID NO. of SEQ ID NOs: 4-1302, 1356-2652, and 2676-3192, or to a reference sequence corresponding to an even-numbered SEQ ID NO. of SEQ ID NOs: 4-1302, 1356-2652, and 2676-3192, wherein the amino acid sequence comprises one or more substitutions relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 11, 13, 14, 15, 16, 18, 20, 21, 22, 26, 27, 29, 30, 31, 32, 34, 35, 36, 37, 38, 39, 40, 42, 43, 44, 46, 48, 50, 51, 52, 53, 54, 55, 56, 57, 58, 59, 60, 61, 62, 63, 64, 65, 66, 67, 68, 69, 70, 71, 73, 74, 75, 76, 77, 78, 79, 80, 81, 82, 83, 84, 87, 88, 89, 90, 91, 92, 93, 94, 97, 98, 99, 100, 101, 102, 104, 105, 108, 109, 110, 111, 112, 113, 115, 116, 117, 118, 119, 120, 122, 123, 124, 125, 126, 127, 128, 129, 131, 132, 133, 134, 135, 136, 137, 138, 139, 140, 141, 142, 143, 146, 148, 149, 150, 151, 152, 153, 154, 155, 156, 157, 161, 162, 163, 166, 168, 169, 170, 172, 173, 175, 178, 179, 180, 181, 182, 183, 184, 186, 187, 188, 190, 191, 192, 194, 195, 196, 197, 198, 199, 200, 201, 202, 203, 204, 205, 206, 207, 208, 210, 211, 212, 213, 214, 215, 216, 217, 218, 219, 220, 221, 222, 223, 224, 225, 226, 227, 228, 229, 230, or 231, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution, or amino acid residue 11D, 13G/S, 14C/E, 15F, 16M, 18C/E/L, 20A/P/R/T/V, 21L/N/R/S/T/V, 22A/S, 26L, 27C/S, 29Q/R, 30G/L/S/Y, 31R, 32F/I/L/N, 34A/E/S/V, 35A/E/F/L/S, 36A/E/I/L/M/Q/R/S/V/Y, 37F/G/I/L/R/Y, 38C/F/L/R, 39A/M/Q/T, 40C/F/L/S/T, 42T, 43A/C/G/H/N, 44S, 46E/F/L/M/Y, 48F/L, 50A/C/L/N/Q/R/S/V, 51F/G/K/L/M/R/T, 52A/H, 53A/M/S, 54G/H/I/M/Q/R/T, 55G/L/Q/S, 56E/Q/T, 57H/L/P/S, 58C/G/Y, 59E/G/H/M/P/R/T/V/W/Y, 60A/C/P/S/W, 61A/E/N/P/S/V, 62A/E/G/I/L/S, 63S, 64R, 65A/D/F/K/P/T, 66A/C/D/E/F/G/H/N/P/Q/R/S/T/V/W, 67R, 68A/D/E/G/I/L/Q/R/S/T/V/W/Y, 69F/L/Q/R/Y, 70R, 71T, 73I/R, 74C/G/Q/S, 75L, 76G/L/P, 77A/I/M/Q/S, 78C/G/N/T, 79A/K/L/P/W, 80A/D/G/L/N/P/R/S/V/W, 81F/I/L/Q/S, 82I/L/P/Q/T, 83G/H/L/S/T, 84A/E/H/M/S, 87A/E/I/K/L/M/R/V/Y, 88N/R/T, 89A/H/I/L/P/Q/T/V, 90C/E/F/K/S/T/V, 91A/F/G/L/S/V, 92L/S, 93A/E/G/P/S/T/V/Y, 94A/C/F/M/Q/S/T/V/Y, 97I, 98A/G/Q, 99A/C, 100F/S/V, 101-, 102A/C/N, 104A/F/H/I/L/Q/R/S/W, 105G/K/L/M/R/S, 108M/R/S/V, 109E/Y, 110C/F, 111E/P/R, 112A/C/E/K/M/N/Q/R/T, 113E/W/Y, 115G/K/R, 116A/E/F/L, 117L/N/S, 118A/G/L/R/S, 119F/K/L/P/R/S, 120G/K/L/R/S/T, 122A/H/S, 123S, 124V, 125I, 126A/E/S/V, 127I/L/P/S, 128C/E/I/K/N/R/S, 129I/L/P/S, 131A/G/V, 132G/K/L/T/V, 133A/E/F/L/Q/S/V/W, 134L, 135I/K/M/P/R/S, 136A/I/L/S/V/Y, 137L, 138C/I/M/V, 139A/H/L/R, 140G, 141G/V, 142F/L/M/W, 143A/C/G/P/R/S/T/V, 146D/H/N/R/V, 148F/H/M/P/Q/R/S/T, 149L, 150C/G/L/P/S/V, 151A/F/R/Y, 152F/H, 153C/K/S/V, 154Q/R, 155A/F/T/W, 156A/C/N/T/V, 157P/V, 161G/L, 162G, 163G/Q/S, 166C/F/L/P/S, 168G/L/N/Q/S, 169A/D/I/Y, 170A/G/H/P/R/S, 172A/H/M/S/T, 173F/K/R/S/T/V, 175F/L/S, 178E/G/N/R, 179A/C/G/I/L/P/V, 180G/H/P, 181C/I/V, 182A/G/I/L/Q/S, 183A/L/Q, 184I/K/M/N/R/S/T/V, 186L, 187G/Y, 188G/L, 190A/C/E/G/H/N/Q/R, 191D/K, 192A/H/I/W, 194F/L/R/V/Y, 195G/I/M/R, 196I, 197A/L/Q/V, 198G, 199R, 200A, 201A/F/K/L/S, 202E/G/M/S, 203A/E/L/R, 204R/S, 205L/P, 206W, 207A/L/T, 208E, 210V, 211A, 212C/H/I/L/M/N/P/Q/R/S/W, 213L/P/Q/S, 214A/E/L/M/P/R/T/W, 215A/P/V, 216A/D/E/G/H/L/M/N/P/R/S/T/V, 217E/G/H/P/T, 218A/I/L, 219A/F/G/L/P/S/T/W, 220E/V, 221D, 222I/T, 223T, 224G/S/T, 225A/L/Q/R/T/V, 226K/L/P/R/S/T, 227P, 228D/F/G/I/L/P/Q/R/S/T/V, 229C/I, 230A/M/P/Q, or 231A/Q/R/T, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution G11D, M13G/S, A14C/E, Yl5F, L16M, F18C/E/L, G20A/P/R/T/V, P21L/N/R/S/T/V, P22A/S, K26L, G27C/S, Y29Q/R, A30G/L/S/Y, K31R, I32F/I/L/N, Q34A/E/S/V, E35A/F/L/S, K36A/E/I/L/M/Q/R/S/V/Y, T37F/G/I/L/R/Y, G38C/F/L/R, I39A/M/Q/T, P40C/F/L/S/T, I42T, S43A/C/G/H/N, T44S, D46E/F/L/M/Y, F48L, D50A/C/L/N/Q/R/S/V, I51F/G/K/L/M/R/T, V52A/H, K53A/M/S, K54G/H/I/M/Q/R/T, E55G/L/Q/S, N56E/Q/T, D57H/L/P/S, E58C/G/Y, L59E/G/H/M/P/R/T/V/W/Y, G60A/C/P/S/W, K61A/E/N/P/S/V, K62A/E/G/I/L/S, I63S, K64R, E65A/D/F/K/P/T, I66A/C/D/E/F/G/H/N/P/Q/R/S/T/V/W, M67R, E68A/D/G/I/L/Q/R/S/T/V/W/Y, K69F/L/Q/R/Y, G70R, E71T, V731/R, P74C/G/Q/S, D75L, E76G/L/P, L77A/I/M/Q/S, V78C/G/N/T, N79A/K/L/P/W, E80A/D/G/L/N/P/R/S/V/W, V81F/I/L/Q/S, V82I/L/P/Q/T, K83G/H/L/S/T, R84A/E/H/M/S, S87A/E/I/K/L/M/R/V/Y, E88N/R/T, K89A/H/I/L/P/Q/T/V, D90C/E/F/K/S/T/V, C91A/F/G/L/S/V, E92L/S, K93A/E/G/P/S/T/V/Y, G94A/C/F/M/Q/S/T/V/Y, L97I, D98A/G/Q, G99A/C, Y100F/S/V, P101-, R102A/C/N, V104A/F/H/I/L/Q/R/S/W, A105G/K/L/M/R/S, E108M/R/S/V, F109E/Y, L110C/F, D111E/P/R, S112A/C/E/K/M/N/Q/R/T, F113E/W/Y, E115G/K/R, S116A/E/F/L, Q117L/N/S, N118A/G/L/R/S, K119F/K/L/P/R/S, Q120G/K/L/R/S/T, T122A/H/S, A123S, A124V, V125I, L126A/E/S/V, F127I/L/P/S, D128C/E/I/K/N/R/S, V129I/L/P/S, E131A/G/V, D132G/K/L/T/V, V133A/E/F/L/Q/S/W, V134L, V135I/K/M/P/R/S, Q136A/I/L/S/V/Y, R137L, L138C/I/M/V, T139A/H/L/R, S140G, R141G/V, R142F/L/M/W, I143A/C/G/P/R/S/T/V, K146D/H/N/R/V, G148F/H/M/P/Q/R/S/T, R149L, I150C/G/L/P/SN, Y151A/F/R, N152F/H, M153C/K/S/V, 1154Q/R, S155A/F/T/W, L156A/C/N/T/V, P157V, D161G/L, E162G, L163G/Q/S, D166C/F/L/P/S, K168G/L/N/Q/S, V169A/D/I/Y, K170A/G/H/P/R/S, V172A/H/M/S/T, Q173F/K/R/S/T/V, D175F/L/S, K178E/G/N/R, E179A/C/G/I/L/P/V, E180G/H/P, T181C/I/V, V182A/G/I/L/Q/S, R183A/L/Q, H184I/K/M/N/R/S/T/V, Y186L, K187G/Y, V188G/L, L190A/C/E/G/H/N/Q/R, E191D/K, K192A/H/I/W, Q194F/L/R/V/Y, P195G/I/M/R, V196I, I197A/L/Q/V, D198G, Y199R, Y200A, G201A/F/K/L/S, K202E/G/M/S, K203A/E/L/R, G204R/S, 1205L/P, L206W, K207A/L/T, R208E, D210V, G211A, T212C/H/I/L/M/N/P/Q/R/S/W, I213L/P/Q/S, G214A/E/L/M/P/R/T/W, I215A/P/V, D216A/D/E/G/H/L/M/N/P/R/S/T/V, N217E/G/H/P/T, V218A/I/L, V219A/F/G/L/P/S/T/W, A220E/V, E221D, V222I/T, L223T, K224G/S/T, I225A/L/Q/R/T/V, I226K/L/P/R/S/T, G227P, W228D/F/G/I/L/P/Q/R/S/T/V, S229C/I, D230A/M/P/Q, or K231A/Q/R/T, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 13, 18, 29, 30, 31, 32, 34, 35, 48, 50, 53, 55, 59, 60, 61, 62, 66, 68, 77, 78, 80, 83, 87, 100, 104, 105, 109, 118, 119, 127, 128, 133, 136, 139, 142, 143, 150, 151, 155, 157, 168, 170, 175, 184, 186, 190, 201, 207, 212, 213, 214, 216, 217, 218, 224, or 226, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution or amino acid residue 13G/S, 18C/E/L, 29R/Q, 30G/L/S/Y, 31R, 32F/I/L/N, 34A/E/S/V, 35A/E/F/L/S, 48F, 50A/C/L/N/Q/R/S/V, 53A/M/S, 55G/L/Q/S, 59E/G/H/L/M/P/R/T/V/W/Y, 60A/C/P/S/W, 61A/E/N/P/S/V, 62A/E/G/I/L/S, 66A/C/D/E/F/G/H/N/P/Q/R/S/T/V/W, 68A/D/E/G/I/L/Q/R/S/T/V/W/Y, 77A/I/M/Q/S, 78C/G/N/T, 80A/D/G/L/N/P/R/S/V/W, 83G/H/L/S/T, 87A/E/I/K/L/M/R/V/Y, 100F/S/V, 104A/F/H/I/L/Q/R/S/W, 105G/K/L/M/R/S, 109E/Y, 118A/G/L/R/S, 119F/K/L/P/R/S, 127I/L/P/S, 128C/E/I/K/N/R/S, 133A/E/F/L/Q/S/V/W, 136A/I/L/S/V/Y, 139A/H/L/R, 142F/L/M/W, 143A/C/G/P/R/S/T/V, 150C/G/L/P/S/V, 151A/F/R/Y, 155A/F/T/W, 157P/V, 168G/L/N/Q/S, 170A/G/H/P/R/S, 175F/L/S, 184I/K/M/N/R/S/T/V, 186L, 190A/C/E/G/H/N/Q/R, 201A/F/K/L/S, 207A/L/T, 212C/H/I/L/M/N/P/Q/R/S/W, 213L/P/Q/S, 214A/E/L/M/P/R/T/W, 216A/E/G/H/L/M/N/P/R/S/T/V/W, 217E/G/H/P/T, 218A/I/L, 224G/S/T, or 226K/L/P/R/S/T, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution or amino acid residue 13G, 18L, 29Q, 30G/S, 31R, 32I, 34S, 35A, 48L, 50Q, 53A, 55Q, 59V, 60A, 61S, 62S, 66W, 68L, 77I, 78G, 80A/P, 83L, 87K, 100F, 104A, 105R, 109Y, 118G, 119R, 127I/L, 128E/K, 133E, 136A/V, 139L, 142L/R, 143V, 150S, 151F, 155F, 157V, 168G, 170G/S, 175F/L, 184M, 186L, 190Q/R, 201A/S, 207A, 212S, 213Q, 214E, 216E/M/P/R, 217P, 218I, 224G, or 226L, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution M13G, F18L, Y29Q, A30G/S, K31R, R32I, Q34S, E35A, F48L, D50Q, K53A, E55Q, L59V, G60A, K61S, K62S, I66W, E68L, L77I, V78G, E80A/P, K83L, S87K, Y100F, V104A, A105R, F109Y, N118G, K119R, F127I/L, D128E/K, V133E, Q136A/V, T139L, R142L/R, I143V, I150S, Y151F, S155F, P157V, K168G, K170G/S, D175F/L, H184M, Y186L, L190Q/R, G201A/S, K207A, T212S, I213Q, G214E, D216E/M/P/R, N217P, V218I, K224G, or I226L, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 29, 30, 128, 142, 186, 212, or 213, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution, or amino acid residue 29Q, 30S, 128K, 142L, 186L, 212S, or 213Q, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution Y29Q, A30S, D128K, R142L, Y186L, T212S, or I213Q, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 29/30/128/142/186/212/213, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 29Q/30S/128K/142L/186L/212S/213Q, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set Y29Q/A30S/D128K/R142L/Y186L/T212S/I213Q, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 155 or 226, or combination thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution or amino acid residue 155F or 226L, or combination thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution S155F or I226L, or combination thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 155/226, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 155F/226L, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set S155F/I226L, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 78, 80, or 224, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution or amino acid residue 78G, 80A, or 224G, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution V78G, E80A, or K224G, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 78/80/224, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 78G/80A/224G, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set V78G/E80A/K224G, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 60 or 170, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution, or amino acid residue 60A or 170G, or combination thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution G60A or K170G, or combination thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 60/170, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 60A/170G, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set G60A/K170G, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 55 or 133, or combination thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution, or amino acid residue 55Q or 133E, or combination thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution E55Q or V133E, or combination thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 55/133, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 55Q/133E, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set E55Q/V133E, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 61 or 201, or combination thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution, or amino acid residue 61S or 201S, or combination thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution K61S or G201S, or combination thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 61/201, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 61S/201S, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set K61S/G201S, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 13, 30, 31, 53, 109, 119, or 168, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution, or amino acid residue 13G, 30G, 31R, 53A, 109Y, 119R, or 168G, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution M13G, S30G, K31R, K53A, F109Y, K119R, or K168G, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 13/30/31/53/109/119/168, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 13G/30G/31R/53A/109Y/119R/168G, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set M13G/S30G/K31R/K53A/F109Y/K119R/K168G, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 34, 35, or 105, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution or amino acid residue 34S, 35A, or 105R, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution Q34S, E35A, or A105R, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 34/35/105, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 34S/35A/105R, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set Q34S/E35A/A105R, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 59 or 139, or combination thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution or amino acid residue 59V or 139L, or combination thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution L59V or T139L, or combination thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 59/139, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 59V/139L, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set L59V/T139L, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 48 or 100, or combination thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution or amino acid residue 548L or 100F, or combination thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution F48L or Y100F, or combination thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 48/100, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 48L/100F, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set F48L/Y100F, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 32, 127, or 175, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution or amino acid residue 32I, 127I, or 175L, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution R32I, F127I, or D175L, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 32/127/175, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 32I/127I/175L, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set R32I/F127I/D175L, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 62, 128, or 190, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution or amino acid residue 62S, 128E, or 190R, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution K62S, K128E, or L190R, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 62/128/190, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 62S/128E/190R, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set K62S/K128E/L190R, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 136, 150, or 217, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution or amino acid residue 136A, 150S, or 217P, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution Q136A, I150S, or N217P, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 136/150/217, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 136A/150S/217P, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set Q136A/I150S/N217P, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 68, 142, 207, 216, or 218, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution, or amino acid residue 68L, 142R, 207A, 216P, or 218I, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution E68L, L142R, K207A, D216P, or V218I, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 68/142/207/216/218, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 68L/142R/207A/216P/218I, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set E68L/L142R/K207A/D216P/V218I, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 18, 118, or 170, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution or amino acid residue 18L, 118G, or 170S, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution F18L, N118G, or G170S, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 18/118/170, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 18L/118G/170S, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set F18L/N118G/G170S, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 77, 143, 214, or 216, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution, or amino acid residue 77I, 143V, 214E, or 216R, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution L77I, I143V, G214E, or P216R, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 77/143/214/216, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 77I/143V/214E/216R, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set L77I/I143V/G214E/P216R, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 50, 184, or 216, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution, or amino acid residue 50Q, 184M, or 216M, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution D50Q, H184M, or R216M, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 50/184/216, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 50Q/184M/216M, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set D50Q/H184M/R216M, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 104, 127, 136, or 175, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution, or amino acid residue 104A, 127L, 136V, or 175F, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution V104A, I127L, A136V, or L175F, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 104/127/136/175, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 104A/127L/136V/175F, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set V104A/I127L/A136V/L175F, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 151 or 157, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution, or amino acid residue 151F or 157V, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution Y151F or P157V, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 151/157, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 151F/157V, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set Y151F/P157V, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at amino acid position 83, 190, 201, or 216, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution, or amino acid residue 83L, 190Q, 201A, or 216E, or combinations thereof, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution K83L, R190Q, S201A, or M216E, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution set at amino acid positions 83/190/201/216, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set, or amino acid residues 83L/190Q/201A/216E, wherein the amino acid positions are relative to the reference sequence corresponding to amino acid residues 12-231 of SEQ ID NO: 2, or relative to the reference sequence corresponding to SEQ ID NO: 2. In some embodiments, the amino acid sequence of the engineered adenylate kinase comprises at least a substitution set K83L/R190Q/S201A/M216E, wherein the amino acid positions are relative to the reference sequence corresponding to amino acid residues 12-231 of SEQ ID NO: 2, or relative to the reference sequence corresponding to SEQ ID NO: 2. 151/157

In some embodiments, the amino acid sequence of the engineered adenylate kinase comprises at least a substitution or substitution set at amino acid position(s) 29/30/128/142/186/212/213, 18/142/212/213, 29/30/128/142/186, 40/135/214, 18/29/30/127/150, 82, 69, 118, 29/30, 206, 32, 16, 104, 111/135/136/214, 29/128/175, 178, 18/128/142, 32/108/111/156/191, 18/29/128/129/142/186, 32/133, 18/29/30/212/213/222, 29/65/127, 32/40/43/52/155/156/217, 18/29/128/129, 80, 32/43/138/152/191/214/215, 183, 18/175/186/212/213, 129, 18/29/30/65/129/142/150/186, 127/142/212/213, 30/65/142/222, 40/43/215, 136, 29/150/211/212/213/221, 43/111/136/219, 226, 88, 140/215/216, 32/133/134, or 18/29/128/186, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution or substitution set, or amino acid residue(s) 29Q/30S/128K/142L/186L/212S/213Q, 18L/142L/212S/213Q, 29Q/30S/128K/142L/186L, 40C/135P/214P, 18L/29Q/30S/127L/150S, 82I, 69Q, 118G, 29Q/305, 206W, 32N, 16M, 104I, 111E/135P/136A/214P, 29Q/128K/1755, 178N, 18L/128K/142L, 32N/108M/111E/156N/191K, 18L/29Q/128K/129I/142L/186L, 32N/133L, 18L/29Q/30S/212S/213Q/222I, 29Q/65K/127L, 32N/40C/43G/52H/155F/156N/217T, 18L/29Q/128K/129I, 80N, 32N/43G/138I/152H/191K/214P/215V, 183Q, 18L/1755/186L/212S/213Q, 129I, 18L/29Q/30S/65K/129I/142L/150S/186L, 127L/142L/212S/213Q, 30S/65K/142L/222I, 40C/43G/215V, 136A, 29Q/150S/211A/212S/213Q/221D, 43G/111E/136A/219W, 226L, 88N, 140G/215V/216A, 32N/133L/134L, or 18L/29Q/128K/186L, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution or substitution set Y29Q/A30S/D128K/R142L/Y186L/T212S/I213Q, F18L/R142L/T212S/I213Q, Y29Q/A30S/D128K/R142L/Y186L, P40C/V135P/G214P, F18L/Y29Q/A30S/F127L/I150S, V82I, K69Q, N118G, Y29Q/A30S, L206W, R32N, L16M, V104I, D111E/V135P/Q136A/G214P, Y29Q/D128K/D175S, K178N, F18L/D128K/R142L, R32N/E108M/D111E/L156N/E191K, F18L/Y29Q/D128K/V129I/R142L/Y186L, R32N/V133L, F18L/Y29Q/A30S/T212S/I213Q/V222I, Y29Q/E65K/F127L, R32N/P40C/S43G/V52H/S155F/L156N/N217T, F18L/Y29Q/D128K/V129I, E80N, R32N/S43G/L138I/N152H/E191K/G214P/I215V, R183Q, F18L/D175S/Y186L/T212S/I213Q, V129I, F18L/Y29Q/A30S/E65K/V129I/R142L/I150S/Y186L, F127L/R142L/T212S/I213Q, A30S/E65K/R142L/V222I, P40C/S43G/I215V, Q136A, Y29Q/I150S/G211A/T212S/I213Q/E221D, S43G/D111E/Q136A/V219W, I226L, E88N, S140G/I215V/D216A, R32N/V133L/V134L, or F18L/Y29Q/D128K/Y186L, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution at an amino acid position set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least one substitution set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution or substitution set at amino acid position(s) set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to amino acid residues 12-231 of SEQ ID NO: 2, or relative to the reference sequence corresponding to SEQ ID NO: 2.

In some embodiments, the amino acid sequence of the engineered adenylate kinase comprises at least a substitution or substitution set of an engineered adenylate kinase variant set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to amino acid residues 12-231 of SEQ ID NO: 2, or relative to the reference sequence corresponding to SEQ ID NO: 2.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference amino acid sequence comprising a substitution or substitution set of an engineered adenylate kinase variant set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to amino acid residues 12-231 of SEQ ID NO: 2, or relative to the reference sequence corresponding to SEQ ID NO: 2.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12-231 of an even-numbered SEQ ID NO. of SEQ ID NOs: 4-1302, 1356-2652, and 2676-3192, or to a reference sequence corresponding to an even-numbered SEQ ID NO. of SEQ ID NOs: 4-1302, 1356-2652, and 2676-3192.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, wherein the amino acid sequence comprises one or more substitutions relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 4-1302, 1356-2652, and 2676-3192, or to the reference sequence corresponding to an even-numbered SEQ ID NO. of SEQ ID NOs: 4-1302, 1356-2652, and 2676-3192, wherein the amino acid sequence comprises one or more substitutions relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the amino acid sequence of the engineered adenylate kinase comprises at least a substitution at amino acid position 11, 13, 14, 15, 16, 18, 20, 21, 22, 26, 27, 29, 30, 31, 32, 34, 35, 36, 37, 38, 39, 40, 42, 43, 44, 46, 48, 50, 51, 52, 53, 54, 55, 56, 57, 58, 59, 60, 61, 62, 63, 64, 65, 66, 67, 68, 69, 70, 71, 73, 74, 75, 76, 77, 78, 79, 80, 81, 82, 83, 84, 87, 88, 89, 90, 91, 92, 93, 94, 97, 98, 99, 100, 101, 102, 104, 105, 108, 109, 110, 111, 112, 113, 115, 116, 117, 118, 119, 120, 122, 123, 124, 125, 126, 127, 128, 129, 131, 132, 133, 134, 135, 136, 137, 138, 139, 140, 141, 142, 143, 146, 148, 149, 150, 151, 152, 153, 154, 155, 156, 157, 161, 162, 163, 166, 168, 169, 170, 172, 173, 175, 178, 179, 180, 181, 182, 183, 184, 186, 187, 188, 190, 191, 192, 194, 195, 196, 197, 198, 199, 200, 201, 202, 203, 204, 205, 206, 207, 208, 210, 211, 212, 213, 214, 215, 216, 217, 218, 219, 220, 221, 222, 223, 224, 225, 226, 227, 228, 229, 230, or 231, or combinations thereof, wherein the amino acid positions are relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the amino acid sequence of the engineered adenylate kinase comprises at least a substitution, or amino acid residue 1 ID, 13G/S, 14C/E, 15F, 16M, 18C/E/L, 20A/P/R/T/V, 21L/N/R/S/T/V, 22A/S, 26L, 27C/S, 29Q/R, 30G/L/S/Y, 31R, 32F/I/L/N, 34A/E/S/V, 35A/E/F/L/S, 36A/E/I/L/M/Q/R/S/V/Y, 37F/G/I/L/R/Y, 38C/F/L/R, 39A/M/Q/T, 40C/F/L/S/T, 42T, 43A/C/G/H/N, 44S, 46E/F/L/M/Y, 48F/L, 50A/C/L/N/Q/R/S/V, 51F/G/K/L/M/R/T, 52A/H, 53A/M/S, 54G/H/I/M/Q/R/T, 55G/L/Q/S, 56E/Q/T, 57H/L/P/S, 58C/G/Y, 59E/G/H/M/P/R/T/V/W/Y, 60A/C/P/S/W, 61A/E/N/P/S/V, 62A/E/G/I/L/S, 635, 64R, 65A/D/F/K/P/T, 66A/C/D/E/F/G/H/N/P/Q/R/S/T/V/W, 67R, 68A/D/E/G/I/L/Q/R/S/T/V/W/Y, 69F/L/Q/R/Y, 70R, 71T, 73I/R, 74C/G/Q/S, 75L, 76G/L/P, 77A/I/M/Q/S, 78C/G/N/T, 79A/K/L/P/W, 80A/D/G/L/N/P/R/S/V/W, 81F/I/L/Q/S, 82I/L/P/Q/T, 83G/H/L/S/T, 84A/E/H/M/S, 87A/E/I/K/L/M/R/V/Y, 88N/R/T, 89A/H/I/L/P/Q/T/V, 90C/E/F/K/S/T/V, 91A/F/G/L/S/V, 92L/S, 93A/E/G/P/S/T/V/Y, 94A/C/F/M/Q/S/T/V/Y, 97I, 98A/G/Q, 99A/C, 100F/S/V, 101-, 102A/C/N, 104A/F/H/I/L/Q/R/S/W, 105G/K/L/M/R/S, 108M/R/S/V, 109E/Y, 110C/F, 111E/P/R, 112A/C/E/K/M/N/Q/R/T, 113E/W/Y, 115G/K/R, 116A/E/F/L, 117L/N/S, 118A/G/L/R/S, 119F/K/L/P/R/S, 120G/K/L/R/S/T, 122A/H/S, 123S, 124V, 125I, 126A/E/S/V, 127I/L/P/S, 128C/E/I/K/N/R/S, 129I/L/P/S, 131A/G/V, 132G/K/L/T/V, 133F/Q/S/V/W, 133A/E/F/L/Q/S/V/W, 134L, 135I/K/M/P/R/S, 136A/I/L/S/V/Y, 137L, 138C/I/M/V, 139A/H/L/R, 140G, 141G/V, 142F/L/M/W, 143A/C/G/P/R/S/T/V, 146D/H/N/R/V, 148F/H/M/P/Q/R/S/T, 149L, 150C/G/L/P/S/V, 151A/F/R/Y, 152F/H, 153C/K/S/V, 154Q/R, 155A/F/T/W, 156A/C/N/T/V, 157P/V, 161G/L, 162G, 163G/Q/S, 166C/F/L/P/S, 168G/L/N/Q/S, 169A/D/I/Y, 170A/G/H/P/R/S, 172A/H/M/S/T, 173F/K/R/S/T/V, 175F/L/S, 178E/G/N/R, 179A/C/G/I/L/P/V, 180G/H/P, 181C/I/V, 182A/G/I/L/Q/S, 183A/L/Q, 184I/K/M/N/R/S/T/V, 186L, 187G/Y, 188G/L, 190A/C/E/G/H/N/Q/R, 191D/K, 192A/H/I/W, 194F/L/R/V/Y, 195G/I/M/R, 196I, 197A/L/Q/V, 198G, 199R, 200A, 201A/F/K/L/S, 202E/G/M/S, 203A/E/L/R, 204R/S, 205L/P, 206W, 207A/L/T, 208E, 210V, 211A, 212C/H/I/L/M/N/P/Q/R/S/W, 213L/P/Q/S, 214A/E/L/M/P/R/T/W, 215A/P/V, 216A/D/E/G/H/L/M/N/P/R/S/T/V, 217E/G/H/P/T, 218A/I/L, 219A/F/G/L/P/S/T/W, 220E/V, 221D, 222I/T, 223T, 224G/S/T, 225A/L/Q/R/T/V, 226K/L/P/R/S/T, 227P, 228D/F/G/I/L/P/Q/R/S/T/V, 229C/I, 230A/M/P/Q, or 231A/Q/R/T, or combinations thereof, wherein the amino acid positions are relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the amino acid sequence of the engineered adenylate kinase comprises at least a substitution at amino acid position 13, 18, 29, 30, 31, 32, 34, 35, 48, 50, 53, 55, 59, 60, 61, 62, 66, 68, 77, 78, 80, 83, 87, 100, 104, 105, 109, 118, 119, 127, 128, 133, 136, 139, 142, 143, 150, 151, 155, 157, 168, 170, 175, 184, 186, 190, 201, 207, 212, 213, 214, 216, 217, 218, 224, or 226, or combinations thereof, wherein the amino acid positions are relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the amino acid sequence of the engineered adenylate kinase comprises at least a substitution or amino acid residue 13G/S, 18C/E/L, 29R/Q, 30G/L/S/Y, 31R, 32F/I/L/N, 34A/E/S/V, 35A/E/F/L/S, 48F, 50A/C/L/N/Q/R/S/V, 53A/M/S, 55G/L/Q/S, 59E/G/H/L/M/P/R/T/V/W/Y, 60A/C/P/S/W, 61A/E/N/P/S/V, 62A/E/G/I/L/S, 66A/C/D/E/F/G/H/N/P/Q/R/S/T/V/W, 68A/D/E/G/I/L/Q/R/S/T/V/W/Y, 77A/I/M/Q/S, 78C/G/N/T, 80A/D/G/L/N/P/R/S/V/W, 83G/H/L/S/T, 87A/E/I/K/L/M/R/V/Y, 100F/S/V, 104A/F/H/I/L/Q/R/S/W, 105G/K/L/M/R/S, 109E/Y, 118A/G/L/R/S, 119F/K/L/P/R/S, 127I/L/P/S, 128C/E/I/K/N/R/S, 133A/E/F/L/Q/S/V/W, 136A/I/L/S/V/Y, 139A/H/L/R, 142F/L/M/W, 143A/C/G/P/R/S/T/V, 150C/G/L/P/S/V, 151A/F/R/Y, 155A/F/T/W, 157P/V, 168G/L/N/Q/S, 170A/G/H/P/R/S, 175F/L/S, 184I/K/M/N/R/S/T/V, 186L, 190A/C/E/G/H/N/Q/R, 201A/F/K/L/S, 207A/L/T, 212C/H/I/L/M/N/P/Q/R/S/W, 213L/P/Q/S, 214A/E/L/M/P/R/T/W, 216A/E/G/H/L/M/N/P/R/S/T/V/W, 217E/G/H/P/T, 218A/I/L, 224G/S/T, or 226K/L/P/R/S/T, or combinations thereof, wherein the amino acid positions are relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the amino acid sequence of the engineered adenylate kinase comprises at least a substitution or amino acid residue(s) 13G, 18L, 29Q, 30G/S, 31R, 32I, 34S, 35A, 48L, 50Q, 53A, 55Q, 59V, 60A, 61S, 62S, 66W, 68L, 77I, 78G, 80A/P, 83L, 87K, 100F, 104A, 105R, 109Y, 118G, 119R, 127I/L, 128E/K, 133E, 136A/V, 139L, 142L/R, 143V, 150S, 151F, 155F, 157V, 168G, 170G/S, 170S, 175F/L, 184M, 186L, 190Q/R, 201A/S, 207A, 212S, 213Q, 214E, 216E/M/P/R, 217P, 218I, 224G, or 226L, or combinations thereof, wherein the amino acid positions are relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 4, or to the reference sequence corresponding to SEQ ID NO: 4, wherein the amino acid sequence comprises one or more substitutions relative to the reference sequence corresponding to residues 12-231 of SEQ ID NO: 4, or relative to the reference sequence corresponding to SEQ ID NO: 4.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 86-372, or to the reference sequence corresponding to an even-numbered SEQ ID NO. of SEQ ID NOs: 86-372, wherein the amino acid sequence comprises one or more substitutions relative to the reference sequence corresponding to residues 12-231 of SEQ ID NO: 4, or relative to the reference sequence corresponding to SEQ ID NO: 4.

In some embodiments, the amino acid sequence of the engineered adenylate kinase comprises at least a substitution or substitution set at amino acid position(s) 100/101, 94, 179, 198, 57, 78, 68, 93, 77, 62, 14, 51, 118, 91, 163, 65, 173, 224, 66, 109, 60, 40/88, 191, 63, 68/170, 100, 108, 58, 180, 178, 199, 40, 80/203, 40/43, 39, 55, 110, 51/59, 155/226, 80, 219, 166, 202, 59, 38, 90, 80/184/203, 35, 222, 54, 111/153/155, 112, 175, 74, 105, 170, 117, 128, 61, 119, 155, 79, 220, 226, 108/155, 108/111, 111, 69/88, 43, 43/69/138, 40/43/88/134/178, 214, 80/82/184/219, 88/136, 16/40/43/88, 40/134, 88, 111/155, 223, 115, 190, 201, 126, 56, 216, 76, 69, 120, 116, 203, 194, or 138, wherein the amino acid positions are relative to the reference sequence corresponding to residues 12-231 of SEQ ID NO: 4, or relative to the reference sequence corresponding to SEQ ID NO: 4.

In some embodiments, the amino acid sequence of the engineered adenylate kinase comprises at least a substitution or substitution set, or amino acid residue(s) 100V/101-, 94S, 179L, 198G, 57P, 78G, 68R, 93V, 77S, 62L, 14C, 51R, 118R, 91F, 163G, 65P, 173R, 224G, 66E, 109E, 60S, 40C/88N, 191D, 63S, 68D/170P, 100S, 51M, 108S, 58G, 57H, 180G, 178R, 199R, 40C, 94Q, 80N/203A, 40C/43G, 39T, 62S, 40L, 55Q, 110C, 51R/59R, 155F/226L, 80N, 219T, 166P, 60P, 202S, 59T, 80A, 38L, 94C, 62A, 51K, 90E, 59M, 80N/184K/203A, 35S, 222T, 178G, 40T, 54M, 111E/153K/155F, 62I, 80G, 112M, 175S, 74S, 14E, 57L, 51G, 94F, 68A, 105R, 170A, 117N, 128S, 40F, 112C, 94A, 61A, 68W, 202G, 180P, 119R, 119R, I66G, 179G, 155F, 59V, 79L, 220E, 51F, 93A, 226L, 108M/155F, 108M/111E, 111E, 69Q/88N, 43G, 43G/69Q/138I, 40C/43G/88N/134L/178N, 214P, 80N/82I/184K/219W, 88N/136A, 16M/40C/43G/88N, 40C/134L, 88N, 111E/155F, 223T, 74G, 115R, 39Q, 93E, 119L, 190N, 202M, 55S, 51L, 112Q, 94T, 66A, 201A, 201K, 201L, 66R, 128N, 214L, 126S, 56E, 1791, 216S, 76P, 69Y, 115G, 166C, 120L, 116L, 108V, 203E, 194F, 138M, 178E, or 38C, wherein the amino acid positions are relative to the reference sequence corresponding to residues 12-231 of SEQ ID NO: 4, or relative to the reference sequence corresponding to SEQ ID NO: 4.

In some embodiments, the amino acid sequence of the engineered adenylate kinase comprises at least a substitution or substitution set Y100V/P101-, G94S, E179L, D198G, D57P, V78G, E68R, K93V, L77S, K62L, A14C, 151R, N118R, C91F, L163G, E65P, Q173R, K224G, 166E, F109E, G60S, P40C/E88N, E191D, 163S, E68D/K170P, Y100S, 151M, E108S, E58G, D57H, E180G, K178R, Y199R, P40C, G94Q, E80N/K203A, P40C/S43G, I39T, K62S, P40L, E55Q, L110C, I51R/L59R, S155F/I226L, E80N, V219T, D166P, G60P, K202S, L59T, E80A, G38L, G94C, K62A, I51K, D90E, L59M, E80N/H184K/K203A, E35S, V222T, K178G, P40T, K54M, D111E/M153K/S155F, K62I, E80G, S112M, D175S, P74S, A14E, D57L, I51G, G94F, E68A, A105R, K170A, Q117N, K128S, P40F, S112C, G94A, K61A, E68W, K202G, E180P, K119R, K119R, I66G, E179G, S155F, L59V, N79L, A220E, 151F, K93A, 1226L, E108M/S155F, E108M/D111E, D111E, K69Q/E88N, S43G, S43G/K69Q/L138I, P40C/S43G/E88N/V134L/K178N, G214P, E80N/V82I/H184K/V219W, E88N/Q136A, L16M/P40C/S43G/E88N, P40C/V134L, E88N, D111E/S155F, L223T, P74G, E115R, 139Q, K93E, K119L, L190N, K202M, E55S, I51L, S112Q, G94T, I66A, G201A, G201K, G201L, I66R, K128N, G214L, L126S, N56E, E1791, D216S, E76P, K69Y, E115G, D166C, Q120L, S116L, E108V, K203E, Q194F, L138M, K178E, or G38C, wherein the amino acid positions are relative to the reference sequence corresponding to residues 12-231 of SEQ ID NO: 4, or relative to the reference sequence corresponding to SEQ ID NO: 4.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 172, or to the reference sequence corresponding to SEQ ID NO: 172, wherein the amino acid sequence comprises one or more substitutions relative to the reference sequence corresponding to residues 12-231 of SEQ ID NO: 172, or relative to the reference sequence corresponding to SEQ ID NO: 172.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 374-444, or to the reference sequence corresponding to an even-numbered SEQ ID NO. of SEQ ID NOs: 374-444, wherein the amino acid sequence comprises one or more substitutions relative to the reference sequence corresponding to residues 12-231 of SEQ ID NO: 172, or relative to the reference sequence corresponding to SEQ ID NO: 172.

In some embodiments, the amino acid sequence of the engineered adenylate kinase comprises at least a substitution or substitution set at amino acid position(s) 66/78, 78/80/224, 78, 65/66, 78/224, 78/80/109, 40/60/94/226, 66/68, 78/80, 94/117/118/226, 68/109, 68, 94/179, 51/66/68/224, 94/117, 51/66/68/78/224, 66, 118, 93/94/180, 109, 68/224, 40/62/93, 93, 51/66, 94/117/118, 198/220, 93/117, 117/118, 93/198, 68/173, 40/117, 224, 40/118/226, 93/94, or 40/62/118, wherein the amino acid positions are relative to the reference sequence corresponding to residues 12-231 of SEQ ID NO: 172, or relative to the reference sequence corresponding to SEQ ID NO: 172.

In some embodiments, the amino acid sequence of the engineered adenylate kinase comprises at least a substitution or substitution set, or amino acid residue(s) 66E/78G, 78G/80A/224G, 78G, 65P/66E, 78G/224G, 78G/80A/109E, 40F/60S/94S/226I, 66A/68R, 66E/68R, 78G/80A, 94S/117N/118R/226I, 68R/109E, 68R, 94S/179L, 51R/66E/68R/224G, 94S/117N, 51R/66E/68R/78G/224G, 66E, 118R, 93V/94S/180P, 109E, 68R/224G, 40F/62L/93V, 93V, 51R/66E, 94S/117N/118R, 198G/220E, 93V/117N, 117N/118R, 93V/198G, 68R/173R, 40F/117N, 224G, 40F/118R/226I, 93V/94S, or 40F/62L/118R, wherein the amino acid positions are relative to the reference sequence corresponding to residues 12-231 of SEQ ID NO: 172, or relative to the reference sequence corresponding to SEQ ID NO: 172.

In some embodiments, the amino acid sequence of the engineered adenylate kinase comprises at least a substitution or substitution set I66E/V78G, V78G/E80A/K224G, V78G, E65P/I66E, V78G/K224G, V78G/E80A/F109E, P40F/G60S/G94S/L226I, I66A/E68R, I66E/E68R, V78G/E80A, G94S/Q117N/N118R/L226I, E68R/F109E, E68R, G94S/E179L, I51R/I66E/E68R/K224G, G94S/Q117N, I51R/I66E/E68R/V78G/K224G, I66E, N118R, K93V/G94S/E180P, F109E, E68R/K224G, P40F/K62L/K93V, K93V, I51R/I66E, G94S/Q117N/N118R, D198G/A220E, K93V/Q117N, Q117N/N118R, K93V/D198G, E68R/Q173R, P40F/Q117N, K224G, P40F/N118R/L226I, K93V/G94S, or P40F/K62L/N118R, wherein the amino acid positions are relative to the reference sequence corresponding to residues 12-231 of SEQ ID NO: 172, or relative to the reference sequence corresponding to SEQ ID NO: 172.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 376, or to the reference sequence corresponding to SEQ ID NO: 376, wherein the amino acid sequence comprises one or more substitutions relative to the reference sequence corresponding to residues 12-231 of SEQ ID NO: 376, or relative to the reference sequence corresponding to SEQ ID NO: 376.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 446-510, or to the reference sequence corresponding to an even-numbered SEQ ID NO. of SEQ ID NOs: 446-510, wherein the amino acid sequence comprises one or more substitutions relative to the reference sequence corresponding to residues 12-231 of SEQ ID NO: 376, or relative to the reference sequence corresponding to SEQ ID NO: 376.

In some embodiments, the amino acid sequence of the engineered adenylate kinase comprises at least a substitution or substitution set at amino acid position(s) 13, 60, 126, 104, 201, 59, 55, 133, 61, 53, 190, 57, 62, 181, 168, 54, 173, 170, 34, 183, 14, 60/62/124/170, 60/170, or 60/62, wherein the amino acid positions are relative to the reference sequence corresponding to residues 12-231 of SEQ ID NO: 376, or relative to the reference sequence corresponding to SEQ ID NO: 376.

In some embodiments, the amino acid sequence of the engineered adenylate kinase comprises at least a substitution or substitution set, or amino acid residue(s) 13S, 60A, 126E, 104L, 201S, 59E, 55Q, 133E, 61P, 59M, 13G, 53A, 190A, 57S, 62A, 181V, 55G, 168G, 54R, 54Q, 173R, 168Q, 168N, 170P, 34V, 59H, 183L, 173K, 183A, 14E, 60A/62A/124V/170G, 60A/170G, or 60A/62A, wherein the amino acid positions are relative to the reference sequence corresponding to residues 12-231 of SEQ ID NO: 376, or relative to the reference sequence corresponding to SEQ ID NO: 376.

In some embodiments, the amino acid sequence of the engineered adenylate kinase comprises at least a substitution or substitution set M13S, G60A, L126E, V104L, G201S, L59E, E55Q, V133E, K61P, L59M, M13G, K53A, L190A, D57S, K62A, T181V, E55G, K168G, K54R, K54Q, Q173R, K168Q, K168N, K170P, Q34V, L59H, R183L, Q173K, R183A, A14E, G60A/K62A/A124V/K170G, G60A/K170G, or G60A/K62A, wherein the amino acid positions are relative to the reference sequence corresponding to residues 12-231 of SEQ ID NO: 376, or relative to the reference sequence corresponding to SEQ ID NO: 376.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 508, or to the reference sequence corresponding to SEQ ID NO: 508, wherein the amino acid sequence comprises one or more substitutions relative to the reference sequence corresponding to residues 12-231 of SEQ ID NO: 508, or relative to the reference sequence corresponding to SEQ ID NO: 508.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 512-580, or to the reference sequence corresponding to an even-numbered SEQ ID NO. of SEQ ID NOs: 512-580, wherein the amino acid sequence comprises one or more substitutions relative to the reference sequence corresponding to residues 12-231 of SEQ ID NO: 508, or relative to the reference sequence corresponding to SEQ ID NO: 508.

In some embodiments, the amino acid sequence of the engineered adenylate kinase comprises at least a substitution or substitution set at amino acid position(s) 15, 15/68, 15/181, 13/15, 13/15/201, 54/55, 55/133, 55/133/197, 54/55/133/197, 68, 54/133/197, 54/197, 13/15/68, 34, 54, 133, 120, 34/133, 13/15/181, 55, 15/59, 68/181, 201, 181, 66, 15/59/181, 197, 13/15/59, 61, 34/61, 13, 34/69, or 163, wherein the amino acid positions are relative to the reference sequence corresponding to residues 12-231 of SEQ ID NO: 508, or relative to the reference sequence corresponding to SEQ ID NO: 508.

In some embodiments, the amino acid sequence of the engineered adenylate kinase comprises at least a substitution or substitution set, or amino acid residue(s) 15F, 15F/68A, 15F/181V, 13S/15F, 13S/15F/2015, 54Q/55Q, 55Q/133E, 55Q/133E/197A, 54Q/55Q/133E/197A, 68A, 54Q/133E/197A, 54Q/197A, 13S/15F/68Y, 34V, 54Q, 133E, 120K, 34V/133A, 13S/15F/181V, 55Q, 15F/59M, 68A/181V, 201S, 68Y, 181V, 66S, 15F/59M/181V, 197A, 13S/15F/59M, 61S, 34V/615, 13S, 61A, 34V/69R, or 163S, wherein the amino acid positions are relative to the reference sequence corresponding to residues 12-231 of SEQ ID NO: 508, or relative to the reference sequence corresponding to SEQ ID NO: 508.

In some embodiments, the amino acid sequence of the engineered adenylate kinase comprises at least a substitution or substitution set Y15F, Y15F/E68A, Y15F/T181V, M13S/Y15F, M13S/Y15F/G201S, K54Q/E55Q, E55Q/V133E, E55Q/V133E/I197A, K54Q/E55Q/V133E/I197A, E68A, K54Q/V133E/I197A, K54Q/I197A, M13S/Y15F/E68Y, Q34V, K54Q, V133E, Q120K, Q34V/V133A, M13S/Y15F/T181V, E55Q, Y15F/L59M, E68A/T181V, G201S, E68Y, T181V, I66S, Y15F/L59M/T181V, I197A, M13S/Y15F/L59M, K61S, Q34V/K61S, M13S, K61A, Q34V/K69R, or L163S, wherein the amino acid positions are relative to the reference sequence corresponding to residues 12-231 of SEQ ID NO: 508, or relative to the reference sequence corresponding to SEQ ID NO: 508.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 524, or to the reference sequence corresponding to SEQ ID NO: 524, wherein the amino acid sequence comprises one or more substitutions relative to the reference sequence corresponding to residues 12-231 of SEQ ID NO: 524, or relative to the reference sequence corresponding to SEQ ID NO: 524.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 582-608, or to the reference sequence corresponding to an even-numbered SEQ ID NO. of SEQ ID NOs: 582-608, wherein the amino acid sequence comprises one or more substitutions relative to the reference sequence corresponding to residues 12-231 of SEQ ID NO: 524, or relative to the reference sequence corresponding to SEQ ID NO: 524.

In some embodiments, the amino acid sequence of the engineered adenylate kinase comprises at least a substitution or substitution set at amino acid position(s) 120, 61/201, 34, 36, 105, 112, 31, or 146, wherein the amino acid positions are relative to the reference sequence corresponding to residues 12-231 of SEQ ID NO: 524, or relative to the reference sequence corresponding to SEQ ID NO: 524.

In some embodiments, the amino acid sequence of the engineered adenylate kinase comprises at least a substitution or substitution set, or amino acid residue(s) 120R, 61S/201S, 34S, 36I, 36V, 36A, 36L, 36M, 105S, 34A, 112E, 31R, 146D, or 112R, wherein the amino acid positions are relative to the reference sequence corresponding to residues 12-231 of SEQ ID NO: 524, or relative to the reference sequence corresponding to SEQ ID NO: 524.

In some embodiments, the amino acid sequence of the engineered adenylate kinase comprises at least a substitution or substitution set Q120R, K61S/G201S, Q34S, K36I, K36V, K36A, K36L, K36M, A105S, Q34A, S112E, K31R, K146D, or S112R, wherein the amino acid positions are relative to the reference sequence corresponding to residues 12-231 of SEQ ID NO: 524, or relative to the reference sequence corresponding to SEQ ID NO: 524.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 584, or to the reference sequence corresponding to SEQ ID NO: 584, wherein the amino acid sequence comprises one or more substitutions relative to the reference sequence corresponding to residues 12-231 of SEQ ID NO: 584, or relative to the reference sequence corresponding to SEQ ID NO: 584.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 610-666, or to the reference sequence corresponding to an even-numbered SEQ ID NO. of SEQ ID NOs: 610-666, wherein the amino acid sequence comprises one or more substitutions relative to the reference sequence corresponding to residues 12-231 of SEQ ID NO: 584, or relative to the reference sequence corresponding to SEQ ID NO: 584.

In some embodiments, the amino acid sequence of the engineered adenylate kinase comprises at least a substitution or substitution set at amino acid position(s) 13/30/31/53/109/119/168, 36/126/190, 126, 36/39/126, 126/190, 36/126, 36/190, 39/126, 13/109/119, 109, 36/120/190, 126/148, 190, 108/126, 30/31, 13/118/182, 31/53, 39, 119, 13/119/168, 53/168, 34, 34/65/146, 36/39, 119/168, 34/112, or 173, wherein the amino acid positions are relative to the reference sequence corresponding to residues 12-231 of SEQ ID NO: 584, or relative to the reference sequence corresponding to SEQ ID NO: 584.

In some embodiments, the amino acid sequence of the engineered adenylate kinase comprises at least a substitution or substitution set, or amino acid residue(s) 13G/30G/31R/53A/109Y/119R/168G, 36Y/126E/190A, 126E, 36Q/39M/126E, 126E/190A, 36Y/39M/126E, 36Q/126E, 36Y/190A, 39M/126E, 13G/109Y/119R, 109Y, 36Y/120G/190A, 126E/148T, 190A, 108R/126E, 30G/31R, 13G/118A/182I, 31R/53A, 39M, 119R, 13G/119R/168G, 53A/168G, 34S, 34S/65A/146D, 36Q/39M, 119R/168G, 34S/112R, 36Y/39M, or 173K, wherein the amino acid positions are relative to the reference sequence corresponding to residues 12-231 of SEQ ID NO: 584, or relative to the reference sequence corresponding to SEQ ID NO: 584.

In some embodiments, the amino acid sequence of the engineered adenylate kinase comprises at least a substitution or substitution set M13G/S30G/K31R/K53A/F109Y/K119R/K168G, K36Y/L126E/L190A, L126E, K36Q/I39M/L126E, L126E/L190A, K36Y/I39M/L126E, K36Q/L126E, K36Y/L190A, I39M/L126E, M13G/F109Y/K119R, F109Y, K36Y/Q120G/L190A, L126E/G148T, L190A, E108R/L126E, S30G/K31R, M13G/N118A/V182I, K31R/K53A, I39M, K119R, M13G/K119R/K168G, K53A/K168G, Q34S, Q34S/E65A/K146D, K36Q/I39M, K119R/K168G, Q34S/S112R, K36Y/I39M, or Q173K, wherein the amino acid positions are relative to the reference sequence corresponding to residues 12-231 of SEQ ID NO: 584, or relative to the reference sequence corresponding to SEQ ID NO: 584.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 610, or to the reference sequence corresponding to SEQ ID NO: 610, wherein the amino acid sequence comprises one or more substitutions relative to the reference sequence corresponding to residues 12-231 of SEQ ID NO: 610, or relative to the reference sequence corresponding to SEQ ID NO: 610.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 668-780, or to the reference sequence corresponding to an even-numbered SEQ ID NO. of SEQ ID NOs: 668-780, wherein the amino acid sequence comprises one or more substitutions relative to the reference sequence corresponding to residues 12-231 of SEQ ID NO: 610, or relative to the reference sequence corresponding to SEQ ID NO: 610.

In some embodiments, the amino acid sequence of the engineered adenylate kinase comprises at least a substitution or substitution set at amino acid position(s) 190, 115/126/190, 126/190, 34/35/105, 126, 59/126, 34/105/116/146, 48, 66, 59, 148, 82, 100, 27, 135, 77, 138, 52, 68, 139, 184, 81, 65, 153, 50, 181, 214, 172, 69, or 154, wherein the amino acid positions are relative to the reference sequence corresponding to residues 12-231 of SEQ ID NO: 610, or relative to the reference sequence corresponding to SEQ ID NO: 610.

In some embodiments, the amino acid sequence of the engineered adenylate kinase comprises at least a substitution or substitution set, or amino acid residue(s) 190A, 115K/126E/190A, 126E/190A, 34S/35A/105R, 126E, 59E/126E, 34S/105R/116E/146D, 48L, 66F, 59T, 148H, 82T, 59V, 59Y, 59P, 100F, 148F, 27S, 135R, 148T, 77M, 138C, 148Q, 126A, 52A, 66Q, 59W, 59G, 148M, 59M, 135S, 68L, 139A, 184I, 68R, 184V, 66S, 68Q, 81I, 68V, 65F, 153S, 126V, 68A, 66T, 50C, 138V, 181I, 214T, 139L, 68T, 148S, 172H, 138I, 69L, 81Q, or 154R, wherein the amino acid positions are relative to the reference sequence corresponding to residues 12-231 of SEQ ID NO: 610, or relative to the reference sequence corresponding to SEQ ID NO: 610.

In some embodiments, the amino acid sequence of the engineered adenylate kinase comprises at least a substitution or substitution set L190A, E115K/L126E/L190A, L126E/L190A, Q34S/E35A/A105R, L126E, L59E/L126E, Q34S/A105R/S116E/K146D, F48L, I66F, L59T, G148H, V82T, L59V, L59Y, L59P, Y100F, G148F, G27S, V135R, G148T, L77M, L138C, G148Q, L126A, V52A, I66Q, L59W, L59G, G148M, L59M, V135S, E68L, T139A, H184I, E68R, H184V, I66S, E68Q, V81I, E68V, E65F, M153S, L126V, E68A, I66T, D50C, L138V, T181I, G214T, T139L, E68T, G148S, V172H, L138I, K69L, V81Q, or I154R, wherein the amino acid positions are relative to the reference sequence corresponding to residues 12-231 of SEQ ID NO: 610, or relative to the reference sequence corresponding to SEQ ID NO: 610.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 674, or to the reference sequence corresponding to SEQ ID NO: 674, wherein the amino acid sequence comprises one or more substitutions relative to the reference sequence corresponding to residues 12-231 of SEQ ID NO: 674, or relative to the reference sequence corresponding to SEQ ID NO: 674.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 782-868, or to the reference sequence corresponding to an even-numbered SEQ ID NO. of SEQ ID NOs: 782-868, wherein the amino acid sequence comprises one or more substitutions relative to the reference sequence corresponding to residues 12-231 of SEQ ID NO: 674, or relative to the reference sequence corresponding to SEQ ID NO: 674.

In some embodiments, the amino acid sequence of the engineered adenylate kinase comprises at least a substitution or substitution set at amino acid position(s) 81/148/219, 68/148/219, 68/81/219, 148/219, 81/219, 136/138/139, 68/219, 66/68/219, 66/148/219, 66/68/148, 59, 59/135, 219, 59/139, 138, 126/148/219, 59/181, 81, 66/81/219, 126/219, 66/68/81/148/219, 181, 27/148, 135/136/139, 148, 59/135/138/181, 59/135/136/138/139, 59/135/136/138/181, 27, 27/126/148, 135/136/138, 68/81/126/148/219, 136, 68, 126/148, or 138/139, wherein the amino acid positions are relative to the reference sequence corresponding to residues 12-231 of SEQ ID NO: 674, or relative to the reference sequence corresponding to SEQ ID NO: 674.

In some embodiments, the amino acid sequence of the engineered adenylate kinase comprises at least a substitution or substitution set, or amino acid residue(s) 81I/148T/219L, 68A/148H/219L, 68A/81I/219L, 148H/219F, 68A/148T/219L, 81I/219L, 136L/138V/139L, 148T/219F, 148H/219L, E68A/V219L, 66T/68A/219L, 66Q/148H/219F, 148T/219L, 66T/68A/148H, 59V, 59V/135R, 219F, 59V/139L, 138V, 126A/148T/219L, 59V/181I, 81I, 66Q/81I/219L, 219L, 126A/219L, 66Q/68A/81I/148T/219L, 181I, 27S/148H, 135R/136L/139R, 136L/138V/139R, 148T, 59V/135R/138V/181I, 59V/135R/136L/138V/139R, 59V/135R/136L/138V/181I, 66Q/68G/81I/148H/219L, 27S, 27S/126A/148T, 135R/136L/138V, 68G/81I/126A/148H/219L, 136L, 27S/126A/148H, 68G, 126A/148T, or 138V/139R, wherein the amino acid positions are relative to the reference sequence corresponding to residues 12-231 of SEQ ID NO: 674, or relative to the reference sequence corresponding to SEQ ID NO: 674.

In some embodiments, the amino acid sequence of the engineered adenylate kinase comprises at least a substitution or substitution set V81I/G148T/V219L, E68A/G148H/V219L, E68A/V81I/V219L, G148H/V219F, E68A/G148T/V219L, V81I/V219L, Q136L/L138V/T139L, G148T/V219F, G148H/V219L, E68A/V219L, I66T/E68A/V219L, I66Q/G148H/V219F, G148T/V219L, I66T/E68A/G148H, L59V, L59V/V135R, V219F, L59V/T139L, L138V, L126A/G148T/V219L, L59V/T181I, V81I, I66Q/V81I/V219L, V219L, L126A/V219L, I66Q/E68A/V81I/G148T/V219L, T181I, G27S/G148H, V135R/Q136L/T139R, Q136L/L138V/T139R, G148T, L59V/V135R/L138V/T181I, L59V/V135R/Q136L/L138V/T139R, L59V/V135R/Q136L/L138V/T181I, I66Q/E68G/V81I/G148H/V219L, G27S, G27S/L126A/G148T, V135R/Q136L/L138V, E68G/V81I/L126A/G148H/V219L, Q136L, G27S/L126A/G148H, E68G, L126A/G148T, or L138V/T139R, wherein the amino acid positions are relative to the reference sequence corresponding to residues 12-231 of SEQ ID NO: 674, or relative to the reference sequence corresponding to SEQ ID NO: 674.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 816, or to the reference sequence corresponding to SEQ ID NO: 816, wherein the amino acid sequence comprises one or more substitutions relative to the reference sequence corresponding to residues 12-231 of SEQ ID NO: 816, or relative to the reference sequence corresponding to SEQ ID NO: 816.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 870-1014, or to the reference sequence corresponding to an even-numbered SEQ ID NO. of SEQ ID NOs: 870-1014, wherein the amino acid sequence comprises one or more substitutions relative to the reference sequence corresponding to residues 12-231 of SEQ ID NO: 816, or relative to the reference sequence corresponding to SEQ ID NO: 816.

In some embodiments, the amino acid sequence of the engineered adenylate kinase comprises at least a substitution or substitution set at amino acid position(s) 48/100/148, 48/66/148/219, 48/100, 48/100/135/136, 48/66/77/100/135/136/148, 48/100/136, 48/135/148, 48/148, 48/136/148, 48/135, 48/135/136, 66/100, 48/66/219, 48/135/219, 100/148, 48/136/219, 48/219, 48/135/148/219, 100/135/136, 100/136, 66/77/100/136/219, 100/135, 48/77/100/135/136, 100, 77/100/135, 48/66/100/135/136/148/219, 52/184, 66, 52/68, 136/148, 126/184, 148, 184, 52, 135/136/148, 126/138/184, 27/184, 27/126, 27/126/148, 27, 27/126/184, 66/100/135/219, 27/68, 52/68/81/126, 48/66/136/148, 66/136/148, 66/135, 66/136, 66/135/219, 136, 135/148/219, 68/126, 66/148/219, 27/68/126, 27/68/184, 126, 68/81/126, 77/135, 135/136, 77/136, 77/148, 82, 138/184, 77/135/136, 27/138, 81, 81/126, 27/126/138, 68, or 81/138, wherein the amino acid positions are relative to the reference sequence corresponding to residues 12-231 of SEQ ID NO: 816, or relative to the reference sequence corresponding to SEQ ID NO: 816.

In some embodiments, the amino acid sequence of the engineered adenylate kinase comprises at least a substitution or substitution set, or amino acid residue(s) 48L/100F/148H, 48L/66F/148F/219L, 48L/100F, 48L/100F/135R/136L, 48L/66F/77M/100F/135R/136L/148H, 48L/100F/136L, 48L/135R/148F, 48L/148F, 48L/136L/148F, 48L/135R, 48L/135R/136L, 66F/100F, 48L/66Q/219L, 48L/135R/219L, 100F/148H, 48L/136L/219L, 48L/219L, 48L/135R/148F/219L, 100F/135R/136L, 100F/136L, 66F/77M/100F/136L/219L, 100F/135R, 48L/77M/100F/135R/136L, 100F, 77M/100F/135R, 48L/66F/100F/135R/136L/148F/219L, 52A/184I, 66F, 52A/68L, 136L/148F, 126A/184I, 148F, 126A/184V, 184V, 52A, 135R/136L/148F, 126A/138V/184V, 184I, 27S/184V, 27S/126A, 27S/126A/148R, 27S, 27S/126A/184V, 66Q/100F/135R/219L, 27S/68A, 52A/68A/81L/126A, 66Q, 48L/66Q/136L/148F, 66F/136L/148H, 66Q/135R, 66Q/136L, 66F/135R/219L, 136L, 135R/148F/219L, 68A/126A, 66F/148H/219L, 27S/68L/126A, 27S/68L/184V, 126A, 68L/81L/126A, 77M/135R, 135R/136L, 77M/136L, 77M/148F, 82T, 138V/184V, 77M/135R/136L, 27S/138V, 81L, 81L/126A, 27S/126A/138V, 68A, or 81L/138V, wherein the amino acid positions are relative to the reference sequence corresponding to residues 12-231 of SEQ ID NO: 816, or relative to the reference sequence corresponding to SEQ ID NO: 816.

In some embodiments, the amino acid sequence of the engineered adenylate kinase comprises at least a substitution or substitution set F48L/Y100F/G148H, F48L/I66F/G148F/V219L, F48L/Y100F, F48L/Y100F/V135R/Q136L, F48L/I66F/L77M/Y100F/V135R/Q136L/G148H, F48L/Y100F/Q136L, F48L/V135R/G148F, F48L/G148F, F48L/Q136L/G148F, F48L/V135R, F48L/V135R/Q136L, I66F/Y100F, F48L/I66Q/V219L, F48L/V135R/V219L, Y100F/G148H, F48L/Q136L/V219L, F48L/V219L, F48L/V135R/G148F/V219L, Y100F/V135R/Q136L, Y100F/Q136L, I66F/L77M/Y100F/Q136L/V219L, Y100F/V135R, F48L/L77M/Y100F/V135R/Q136L, Y100F, L77M/Y100F/V135R, F48L/I66F/Y100F/V135R/Q136L/G148F/V219L, V52A/H184I, I66F, V52A/E68L, Q136L/G148F, L126A/H184I, G148F, L126A/H184V, H184V, V52A, V135R/Q136L/G148F, L126A/L138V/H184V, H184I, G27S/H184V, G27S/L126A, G27S/L126A/G148R, G27S, G27S/L126A/H184V, I66Q/Y100F/V135R/V219L, G27S/E68A, V52A/E68A/V81L/L126A, I66Q, F48L/I66Q/Q136L/G148F, I66F/Q136L/G148H, I66Q/V135R, I66Q/Q136L, I66F/V135R/V219L, Q136L, V135R/G148F/V219L, E68A/L126A, I66F/G148H/V219L, G27S/E68L/L126A, G27S/E68L/H184V, L126A, E68L/V81L/L126A, L77M/V135R, V135R/Q136L, L77M/Q136L, L77M/G148F, V82T, L138V/H184V, L77M/V135R/Q136L, G27S/L138V, V81L, V81L/L126A, G27S/L126A/L138V, E68A, or V81L/L138V, wherein the amino acid positions are relative to the reference sequence corresponding to residues 12-231 of SEQ ID NO: 816, or relative to the reference sequence corresponding to SEQ ID NO: 816.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 874, or to the reference sequence corresponding to SEQ ID NO: 874, wherein the amino acid sequence comprises one or more substitutions relative to the reference sequence corresponding to residues 12-231 of SEQ ID NO: 874, or relative to the reference sequence corresponding to SEQ ID NO: 874.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1016-1302, or to the reference sequence corresponding to an even-numbered SEQ ID NO. of SEQ ID NOs: 1016-1302, wherein the amino acid sequence comprises one or more substitutions relative to the reference sequence corresponding to residues 12-231 of SEQ ID NO: 874, or relative to the reference sequence corresponding to SEQ ID NO: 874.

In some embodiments, the amino acid sequence of the engineered adenylate kinase comprises at least a substitution or substitution set at amino acid position(s) 32/60/135/175/225, 32/81/127/128/135/175/225, 83/175/225, 135/175/225, 54/84/190, 55/190/212, 32/60/81/83/127/128/135/225, 175/225, 32/127/175, 84/190/212, 32/128/175, 81/175, 21/54, 54/55/212, 32/175/225, 128/175/225, 62/84/190, 81/135/225, 21/54/62/190/212, 60/81/83/175, 55/84/190, 32/81/83/135/225, 175, 62/212, 32/81/83, 32/81/225, 54/55/84/212, 55/190, 62/179, 54/84/179/212, 54/55/62/84, 81/135, 32/81/135, 179/190/212, 55/62, 100, 21/54/62/190, 190/212, 128/175, 21/62/190, 54/179/190, 54/190, 21/55/190, 21/54/62, 21/84, 21/55, 21/62, 21/179, 55/62/190/212, 54/62/84, 127/128/175/225, 54/62, 100/104, 54/55, 21/55/212, 62, 55/84, 55/62/212, 21/84/190, 225, 21/54/179/190, 83/135, 32/175, 21/54/78/190, 55/62/179/190/212, 21/54/190, 21/55/84/190, 62/84, 21/55/62, 21/62/84, 135, 32/60/81/127, 21/54/55/62/78, 54/84, 55/62/179/190, 54/55/62/84/179/190/212, 190, 83, 78, 54/62/190, 21/55/62/179/190, 32/60/127, 54/62/84/190/212, 21/62/179, 127/128/225, 32/225, 32/60/81, 81, 54, 21, 60/81/128/175, 84, 21/190/212, 60/128/225, 179, 21/55/62/190, 128, 60, 212, 55, 127, 32, 80, 220, 62/78/179, 55/62/84, 21/55/190/212, 32/127/128/225, 21/54/55/84/179, 21/190, 32/60/81/83/128/135, 127/128, or 21/55/179, wherein the amino acid positions are relative to the reference sequence corresponding to residues 12-231 of SEQ ID NO: 874, or relative to the reference sequence corresponding to SEQ ID NO: 874.

In some embodiments, the amino acid sequence of the engineered adenylate kinase comprises at least a substitution or substitution set, or amino acid residue(s) 32I/60S/135K/175L/225A, 32I/81L/127I/128N/135K/175L/225A, 83S/175F/225A, 135K/175L/225A, 54T/84H/190R, 55S/190H/212N, 32I/60S/81L/83S/127I/128N/135K/225A, 175F/225A, 32I/127I/175L, 84A/190H/212N, 32I/128N/175F, 81L/175L, 81L/175F, 21R/54T, 54T/55S/212N, 32I/175L/225A, 128N/175F/225A, 62S/84A/190R, 81L/135M/225A, 21R/54T/62S/190R/212N, 60S/81L/83G/175L, 55S/84A/190R, 32I/81L/83G/135K/225A, 175F, 62S/212N, 32I/81L/83S, 32I/81L/225A, 54T/55S/84A/212N, 55S/190R, 62S/179P, 62E/84A/190H, 54T/84A/179P/212N, 54T/55S/62S/84A, 81L/135M, 32I/81L/135K, 179P/190H/212N, 55S/62E, 100Y, 21R/54T/62E/190H, 190H/212N, 128N/175L, 21R/62S/190H, 54T/179P/190H, 54T/190R, 21R/55S/190H, 21R/54T/62E, 21R/84A, 21R/55S, 21R/62S, 21R/179P, 175L, 55S/62E/190H/212N, 54T/62E/84A, 127I/128N/175L/225A, 54T/62E, 100Y/104F, 55S/62E/190R/212N, 54T/555, 21R/55S/212N, 62S, 55S/84A, 55S/62S/212N, 21R/84A/190H, 225A, 21R/54T/179P/190R, 83S/135K, 32I/175L, 21R/54T/78T/190R, 62E, 55S/62E/179P/190H/212N, 81L/135K, 21R/54T/190H, 21R/55S/84A/190H, 62S/84A, 21R/55S/62E, 21R/62S/84A, 135M, 32I/60S/81L/127I, 21R/54T/55S/62E/78T, 100Y/104H, 54T/84A, 55S/62S/179P/190R, 54T/55S/62S/84A/179P/190H/212N, 190R, 190H, 83S, 100Y/104R, 78T, 54T/62E/190R, 21R/55S/62E/179P/190H, 78C, 32I/60S/127I, 54T/62E/84A/190H/212N, 21R/62S/179P, 100Y/104Q, 127I/128N/225A, 32I/225A, 32I/60S/81L, 225V, 81L, 54T, 21R, 60S/81L/128N/175L, 190Q, 84A, 21R/190R/212N, 190C, 135K, 60S/128N/225A, 225T, 62G, 225Q, 179P, 225R, 100Y/1045, 83H, 21R/55S/62E/190R, 100Y/104W, 190G, 128N, 84M, 60S, 78N, 84S, 62L, 128E, 212N, 55S, 127I, 21S, 32I, 80G, 84E, 220V, 62S/78T/179P, 190E, 55S/62E/84A, 21R/55S/190H/212N, 32I/127I/128N/225A, 21R/54T/55S/84A/179P, 21R/190R, 32I/60S/81L/83S/128N/135M, 127I/128N, or 21R/55S/179P, wherein the amino acid positions are relative to the reference sequence corresponding to residues 12-231 of SEQ ID NO: 874, or relative to the reference sequence corresponding to SEQ ID NO: 874.

In some embodiments, the amino acid sequence of the engineered adenylate kinase comprises at least a substitution or substitution set R32I/A60S/V135K/D175L/I225A, R32I/V81L/F127I/K128N/V135K/D175L/I225A, K83S/D175F/I225A, V135K/D175L/I225A, K54T/R84H/L190R, Q55S/L190H/S212N, R32I/A60S/V81L/K83S/F127I/K128N/V135K/I225A, D175F/I225A, R32I/F127I/D175L, R84A/L190H/S212N, R32I/K128N/D175F, V81L/D175L, V81L/D175F, P21R/K54T, K54T/Q55S/S212N, R32I/D175L/I225A, K128N/D175F/I225A, K62S/R84A/L190R, V81L/V135M/I225A, P21R/K54T/K62S/L190R/S212N, A60S/V81L/K83G/D175L, Q55S/R84A/L190R, R32I/V81L/K83G/V135K/I225A, D175F, K62S/S212N, R32I/V81L/K83S, R32I/V81L/I225A, K54T/Q55S/R84A/S212N, Q55S/L190R, K62S/E179P, K62E/R84A/L190H, K54T/R84A/E179P/S212N, K54T/Q55S/K62S/R84A, V81L/V135M, R32I/V81L/V135K, E179P/L190H/S212N, Q55S/K62E, F100Y, P21R/K54T/K62E/L190H, L190H/S212N, K128N/D175L, P21R/K62S/L190H, K54T/E179P/L190H, K54T/L190R, P21R/Q55S/L190H, P21R/K54T/K62E, P21R/R84A, P21R/Q55S, P21R/K62S, P21R/E179P, D175L, Q55S/K62E/L190H/S212N, K54T/K62E/R84A, F127I/K128N/D175L/I225A, K54T/K62E, F100Y/V104F, Q55S/K62E/L190R/S212N, K54T/Q55S, P21R/Q55S/S212N, K62S, Q55S/R84A, Q55S/K62S/S212N, P21R/R84A/L190H, I225A, P21R/K54T/E179P/L190R, K83S/V135K, R32I/D175L, P21R/K54T/G78T/L190R, K62E, Q55S/K62E/E179P/L190H/S212N, V81L/V135K, P21R/K54T/L190H, P21R/Q55S/R84A/L190H, K62S/R84A, P21R/Q55S/K62E, P21R/K62S/R84A, V135M, R32I/A60S/V81L/F127I, P21R/K54T/Q55S/K62E/G78T, F100Y/V104H, K54T/R84A, Q55S/K62S/E179P/L190R, K54T/Q55S/K62S/R84A/E179P/L190H/S212N, L190R, L190H, K83S, F100Y/V104R, G78T, K54T/K62E/L190R, P21R/Q55S/K62E/E179P/L190H, G78C, R32I/A60S/F127I, K54T/K62E/R84A/L190H/S212N, P21R/K62S/E179P, F100Y/V104Q, F127I/K128N/I225A, R32I/I225A, R32I/A60S/V81L, I225V, V81L, K54T, P21R, A60S/V81L/K128N/D175L, L190Q, R84A, P21R/L190R/S212N, L190C, V135K, A60S/K128N/I225A, I225T, K62G, I225Q, E179P, I225R, F100Y/V104S, K83H, P21R/Q55S/K62E/L190R, F100Y/V104W, L190G, K128N, R84M, A60S, G78N, R84S, K62L, K128E, S212N, Q55S, F127I, P21S, R32I, A80G, R84E, A220V, K62S/G78T/E179P, L190E, Q55S/K62E/R84A, P21R/Q55S/L190H/S212N, R32I/F127I/K128N/I225A, P21R/K54T/Q55S/R84A/E179P, P21R/L190R, R32I/A60S/V81L/K83S/K128N/V135M, F127I/K128N, or P21R/Q55S/E179P, wherein the amino acid positions are relative to the reference sequence corresponding to residues 12-231 of SEQ ID NO: 874, or relative to the reference sequence corresponding to SEQ ID NO: 874.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1032, or to the reference sequence corresponding to SEQ ID NO: 1032, wherein the amino acid sequence comprises one or more substitutions relative to the reference sequence corresponding to residues 12-231 of SEQ ID NO: 1032, or relative to the reference sequence corresponding to SEQ ID NO: 1032.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1356-1552, or to the reference sequence corresponding to an even-numbered SEQ ID NO. of SEQ ID NOs: 1356-1552, wherein the amino acid sequence comprises one or more substitutions relative to the reference sequence corresponding to residues 12-231 of SEQ ID NO: 1032, or relative to the reference sequence corresponding to SEQ ID NO: 1032.

In some embodiments, the amino acid sequence of the engineered adenylate kinase comprises at least a substitution or substitution set at amino acid position(s) 54/80/225, 55/80/180, 54/55/80/208/225, 84/128/155/190/212, 62/155/190, 83/84/155/190, 62/128/135/190, 225, 84/155/190, 128/190, 190/212, 83/84/155, 54/208/225, 84/155/190, 84/212, 84/123/155/212, 62/128/190, 55/80/208/225, 155/190, 83/84/190/212, 83/128/155/190/212, 55/180/225, 84/190/212, 54/80/180/208/225, 80/208, 135/155/190, 212, 80/180/208, 128/135/190/212, 128/155/212, 80/208/225, 83/212, 62/84/155/190, 54/179/225, 84, 83/84/190, 128/212, 54/55/80/208, 55, 62/128, 54/80/208/225, 62/83/84/190, 179/225, 190, 80/169/208, 62/128/212, 80, 62, 54/55/80/225, 54/80/180/208, 83/84/128/190/212, 62/83/190, 62/190, 80/179, 80/180, 54/80, 62/83/84/128/190/212, 83/84/135/212, 54/80/179/208, 62/84/128, 11/62/84/128/190/212, 80/179/208, 80/225, 62/84/128/190/212, 62/84/212, 80/179/180/208/225, 62/135, 55/208, 62/84, 128/135/190, 55/208/225, 84/128/190, 62/84/128/190, 62/135/212, 54/179/180/225, 62/84/135/212, 135, 54/55/225, 55/80/179/180, 84/135/190, 84/128, 54, 84/128/135, 54/179/208/225, 55/179/225, 128, 62/83/84/155/190, 84/135, 84/128/155, 55/179/180, 83/84/128/190, or 54/55/179/225, wherein the amino acid positions are relative to the reference sequence corresponding to residues 12-231 of SEQ ID NO: 1032, or relative to the reference sequence corresponding to SEQ ID NO: 1032.

In some embodiments, the amino acid sequence of the engineered adenylate kinase comprises at least a substitution or substitution set, or amino acid residue(s) 54T/80D/225A, 55S/80D/180H, 54T/55S/80D/208E/225L, 84M/128E/155T/190R/212N, 62S/155T/190H, 83T/84M/155T/190H, 62S/128E/135K/190H, 225A, 84M/155T/190R, 128E/190R, 190R/212N, 83T/84M/155T, 54T/208E/225A, 84M/155T/190H, 84H/212N, 84M/123S/155T/212N, 62S/128E/190R, 55S/80D/208E/225A, 155T/190R, 83T/84M/190R/212N, 83T/128E/155T/190H/212N, 55S/180H/225A, 84M/190R/212N, 54T/80D/180H/208E/225A, 80D/208E, 135K/155T/190R, 212N, 80D/180H/208E, 128E/135K/190R/212N, 128E/155T/212N, 80D/208E/225L, 83T/212N, 62S/84M/155T/190R, 54T/179V/225A, 84H, 83T/84M/190R, 128E/212N, 54T/55S/80D/208E, 190H/212N, 55S, 62S/128E, 54T/80D/208E/225A, 62S/83T/84M/190R, 179A/225A, 190R, 80D/169D/208E, 62S/128E/212N, 80D, 62S, 54T/55S/80D/225L, 54T/80D/180H/208E, 83T/84M/128E/190H/212N, 62S/83T/190R, 179V/225A, 62S/190R, 80D/179V, 80D/180H, 54T/80D, 62S/83T/84H/128E/190H/212N, 83T/84M/135K/212N, 54T/80D/179A/208E, 62S/84H/128E, 11D/62S/84M/128E/190H/212N, 80D/179V/208E, 80D/225A, 62S/84M/128E/190H/212N, 62S/84H/212N, 80D/179V/180H/208E/225L, 62S/135K, 55S/208E, 62S/84H, 128E/135K/190H, 55S/208E/225A, 190H, 84H/128E/190H, 62S/84H/128E/190R, 62S/135K/212N, 54T/179V/180H/225A, 128E/190H, 62S/84M/135K/212N, 135K, 54T/55S/225L, 55S/80D/179V/180H, 62S/84M/212N, 84M/135K/190R, 84M/128E, 54T, 84M/128E/135K, 54T/179A/208E/225A, 55S/179V/225L, 128E, 62S/83T/84M/155T/190R, 84M/135K, 84H/128E/155T, 55S/179V/180H, 83T/84M/128E/190R, 83T/84M/190H, 84M, or 54T/55S/179V/225A, wherein the amino acid positions are relative to the reference sequence corresponding to residues 12-231 of SEQ ID NO: 1032, or relative to the reference sequence corresponding to SEQ ID NO: 1032.

In some embodiments, the amino acid sequence of the engineered adenylate kinase comprises at least a substitution or substitution set K54T/A80D/I225A, Q55S/A80D/E180H, K54T/Q55S/A80D/R208E/I225L, R84M/K128E/F155T/L190R/S212N, K62S/F155T/L190H, K83T/R84M/F155T/L190H, K62S/K128E/V135K/L190H, I225A, R84M/F155T/L190R, K128E/L190R, L190R/S212N, K83T/R84M/F155T, K54T/R208E/I225A, R84M/F155T/L190H, R84H/S212N, R84M/A123S/F155T/S212N, K62S/K128E/L190R, Q55S/A80D/R208E/I225A, F155T/L190R, K83T/R84M/L190R/S212N, K83T/K128E/F155T/L190H/S212N, Q55S/E180H/I225A, R84M/L190R/S212N, K54T/A80D/E180H/R208E/I225A, A80D/R208E, V135K/F155T/L190R, S212N, A80D/E180H/R208E, K128E/V135K/L190R/S212N, K128E/F155T/S212N, A80D/R208E/I225L, K83T/S212N, K62S/R84M/F155T/L190R, K54T/E179V/I225A, R84H, K83T/R84M/L190R, K128E/S212N, K54T/Q55S/A80D/R208E, L190H/S212N, Q55S, K62S/K128E, K54T/A80D/R208E/I225A, K62S/K83T/R84M/L190R, E179A/I225A, L190R, A80D/V169D/R208E, K62S/K128E/S212N, A80D, K62S, K54T/Q55S/A80D/I225L, K54T/A80D/E180H/R208E, K83T/R84M/K128E/L190H/S212N, K62S/K83T/L190R, E179V/I225A, K62S/L190R, A80D/E179V, A80D/E180H, K54T/A80D, K62S/K83T/R84H/K128E/L190H/S212N, K83T/R84M/V135K/S212N, K54T/A80D/E179A/R208E, K62S/R84H/K128E, G11D/K62S/R84M/K128E/L190H/S212N, A80D/E179V/R208E, A80D/I225A, K62S/R84M/K128E/L190H/S212N, K62S/R84H/S212N, A80D/E179V/E180H/R208E/I225L, K62S/V135K, Q55S/R208E, K62S/R84H, K128E/V135K/L190H, Q55S/R208E/I225A, L190H, R84H/K128E/L190H, K62S/R84H/K128E/L190R, K62S/V135K/S212N, K54T/E179V/E180H/I225A, K128E/L190H, K62S/R84M/V135K/S212N, V135K, K54T/Q55S/I225L, Q55S/A80D/E179V/E180H, K62S/R84M/S212N, R84M/V135K/L190R, R84M/K128E, K54T, R84M/K128E/V135K, K54T/E179A/R208E/I225A, Q55S/E179V/I225L, K128E, K62S/K83T/R84M/F155T/L190R, R84M/V135K, R84H/K128E/F155T, Q55S/E179V/E180H, K83T/R84M/K128E/L190R, K83T/R84M/L190H, R84M, or K54T/Q55S/E179V/J225A, wherein the amino acid positions are relative to the reference sequence corresponding to residues 12-231 of SEQ ID NO: 1032, or relative to the reference sequence corresponding to SEQ ID NO: 1032.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1388, or to the reference sequence corresponding to SEQ ID NO: 1388, wherein the amino acid sequence comprises one or more substitutions relative to the reference sequence corresponding to residues 12-231 of SEQ ID NO: 1388, or relative to the reference sequence corresponding to SEQ ID NO: 1388.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1554-1786, or to the reference sequence corresponding to an even-numbered SEQ ID NO. of SEQ ID NOs: 1554-1786, wherein the amino acid sequence comprises one or more substitutions relative to the reference sequence corresponding to residues 12-231 of SEQ ID NO: 1388, or relative to the reference sequence corresponding to SEQ ID NO: 1388.

In some embodiments, the amino acid sequence of the engineered adenylate kinase comprises at least a substitution or substitution set at amino acid position(s) 143, 228, 136, 82, 156, 205, 226, 150, 219, 217, 218, 68, 142, 212, 213, 79, 230, 207, 155, 227, 129, 210, 133, 151, 43, 204, 182, 82/136/150/217, 136/150, 79/81/143/156/212/228, 82/136, 79/81/143/212, 79/133/228, 81/143/156/212, 136/219, 82/136/226, 136/150/217, 133/156, 133/143/228, 150/217, 82/136/150, 81, 81/143, 81/133/228, 143/228, 212/228, 79/81, 133/228, 133/212, 136/217, 216, 215, or 127, wherein the amino acid positions are relative to the reference sequence corresponding to residues 12-231 of SEQ ID NO: 1388, or relative to the reference sequence corresponding to SEQ ID NO: 1388.

In some embodiments, the amino acid sequence of the engineered adenylate kinase comprises at least a substitution or substitution set, or amino acid residue(s) 143S, 228G, 136A, 143T, 82Q, 143A, 156C, 205P, 82P, 226P, 228L, 143C, 150G, 150S, 219P, 150P, 217H, 226T, 143G, 218L, 68S, 142R, 212I, 212L, 228P, 213S, 226S, 156T, 79P, 230A, 218A, 143R, 228S, 68L, 207T, 228R, 155A, 136S, 150L, 150V, 227P, 129L, 230Q, 210V, 205L, 68G, 133W, 155W, 212R, 207A, 151F, 212P, 218I, 129P, 43A, 230P, 129S, 68A, 142F, 204S, 136Y, 182S, 133S, 228I, 43C, 217G, 129I, 82Q/136L/150G/217P, 136A/150S, 79P/81S/143A/156C/212I/228G, 82Q/136A, 79P/81S/143A/212I, 217P, 79P/133F/228L, 81S/143A/156T/212I, 136A/219A, 82Q/136A/226P, 136A/150S/217P, 136A/150G, 133F, 133F/156C, 133F/143A/228L, 136L, 150S/217P, 82Q/136A/150S, 81S, 81S/143T, 81S/133F/228L, 143A/228L, 212I/228G, 79P/81S, 133F/228L, 133F/212M, 136L/217H, 82P/136L, 216H, 215P, 143P, 151R, 212M, 213P, 127S, 133Q, 212W, 219G, or 219S, wherein the amino acid positions are relative to the reference sequence corresponding to residues 12-231 of SEQ ID NO: 1388, or relative to the reference sequence corresponding to SEQ ID NO: 1388.

In some embodiments, the amino acid sequence of the engineered adenylate kinase comprises at least a substitution or substitution set I143S, W228G, Q136A, I143T, V82Q, I143A, L156C, 1205P, V82P, L226P, W228L, I143C, I150G, I150S, V219P, I150P, N217H, L226T, I143G, V218L, E68S, L142R, S212I, S212L, W228P, Q213S, L226S, L156T, N79P, D230A, V218A, I143R, W228S, E68L, K207T, W228R, F155A, Q136S, I150L, I150V, G227P, V129L, D230Q, D210V, I205L, E68G, E133W, F155W, S212R, K207A, Y151F, S212P, V218I, V129P, S43A, D230P, V129S, E68A, L142F, G204S, Q136Y, V182S, E133S, W228I, S43C, N217G, V129I, V82Q/Q136L/I150G/N217P, Q136A/I150S, N79P/V81S/I143A/L156C/S212I/W228G, V82Q/Q136A, N79P/V81S/I143A/S212I, N217P, N79P/E133F/W228L, V81S/I143A/L156T/S212I, Q136A/V219A, V82Q/Q136A/L226P, Q136A/I1505/N217P, Q136A/I150G, E133F, E133F/L156C, E133F/I143A/W228L, Q136L, I150S/N217P, V82Q/Q136A/I150S, V81S, V81S/I143T, V81S/E133F/W228L, I143A/W228L, S212I/W228G, N79P/V81S, E133F/W228L, E133F/S212M, Q136L/N217H, V82P/Q136L, D216H, I215P, I143P, Y151R, S212M, Q213P, I127S, E133Q, S212W, V219G, or V219S, wherein the amino acid positions are relative to the reference sequence corresponding to residues 12-231 of SEQ ID NO: 1388, or relative to the reference sequence corresponding to SEQ ID NO: 1388.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1708, or to the reference sequence corresponding to SEQ ID NO: 1708, wherein the amino acid sequence comprises one or more substitutions relative to the reference sequence corresponding to residues 12-231 of SEQ ID NO: 1708, or relative to the reference sequence corresponding to SEQ ID NO: 1708.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1788-1968, or to the reference sequence corresponding to an even-numbered SEQ ID NO. of SEQ ID NOs: 1788-1968, wherein the amino acid sequence comprises one or more substitutions relative to the reference sequence corresponding to residues 12-231 of SEQ ID NO: 1708, or relative to the reference sequence corresponding to SEQ ID NO: 1708.

In some embodiments, the amino acid sequence of the engineered adenylate kinase comprises at least a substitution or substitution set at amino acid position(s) 127, 143/155/219/228, 219, 127/142, 142/151/207/218, 143/213, 151/218, 127/142/218, 142/151, 143/155/205/213/228, 127/142/207, 127/216/218, 142/151/207, 143/219, 127/207, 127/218, 143/219/228, 127/216, 68/127/207, 143/155/212/228, 151, 143/213/228, 143/155/205, 151/207/216, 143/228, 68/127/207/218, 143/155, 143, 205/213/219, 155/212/219/228, 205/219/228, 143/205, 142/207, 143/205/219, 143/212/213, 127/207/218, 142, 127/142/151/207/218, 143/205/219/228, 205/213/228, 68/151/207, 68/127/142/207, 68/127, 218, 127/142/196, 142/207/218, 68/142/216, 68/127/216/218, 68/127/218, 151/216/218, 143/212/228, 127/142/151, 151/207, 127/142/216, 129/207/218, 68/142/151/207, 68/142/151, 151/207/218, 228, 155/213, 127/142/151/218, 127/151, 155/228, 68/142/207, 80/151/207, 205/228, 68/142/207/216/218, 68/218, 68/207, 216/218, 68/142, 212, 205, or 142/207/216/218, wherein the amino acid positions are relative to the reference sequence corresponding to residues 12-231 of SEQ ID NO: 1708, or relative to the reference sequence corresponding to SEQ ID NO: 1708.

In some embodiments, the amino acid sequence of the engineered adenylate kinase comprises at least a substitution or substitution set, or amino acid residue(s) 127P, 143S/155A/219P/228R, 219P, 127P/142R, 142R/151A/207A/218A, 143G/213L, 151A/218A, 127P/142R/218I, 142R/151A, 143S/155A/205L/213L/228R, 127P/142R/207A, 127P/216P/218A, 142R/151A/207A, 143S/219P, 127P/207A, 127P/218I, 143G/219P/228R, 127P/216P, 68L/127P/207L, 143G/155A/212R/228R, 151A, 143S/219P/228R, 143S/213L/228R, 143G/155A/205L, 151A/207A/216P, 143G/228R, 68L/127P/207A, 68L/127P/207A/218A, 143G/155A, 143G, 205L/213L/219P, 155A/212R/219P/228R, 143S/228R, 205L/219P/228R, 143G/205L, 142R/207A, 143G/205L/219P, 143G/212R/213L, 127P/207L/218I, 142R, 127P/216P/218I, 127P/142R/151A/207A/218I, 143S/205L/219P/228R, 205L/213L/228R, 143G/219P, 142R/207L, 68L/151A/207L, 143S/213L, 142R/151A/207L, 127P/142R/207L, 68L/127P/142R/207L, 143S/205L, 68L/127P, 218A, 127P/142R/196I, 142R/207L/218A, 1435/155A, 143S, 68L/142R/216P, 68L/127P/216P/218I, 68L/127P/218I, 151A/216P/218I, 143S/212R/228R, 127P/142R/151A, 151A/207L, 127P/142R/216P, 129I/207A/218A, 142R/207L/218I, 68L/142R/151A/207A, 151A/207A, 68L/142R/151A, 151A/207L/218I, 228R, 155A/213L, 127P/142R/151A/218I, 127P/151A, 155A/228R, 143S/155A/205L, 151A/216P/218A, 68L/142R/207L, 80V/151A/207L, 205L/228R, 68L/142R/207A/216P/218I, 68L/218I, 68L/207A, 216P/218I, 68L/142R, 212R, 68L/207L, 205L, or 142R/207A/216P/218I, wherein the amino acid positions are relative to the reference sequence corresponding to residues 12-231 of SEQ ID NO: 1708, or relative to the reference sequence corresponding to SEQ ID NO: 1708.

In some embodiments, the amino acid sequence of the engineered adenylate kinase comprises at least a substitution or substitution set I127P, I143S/F155A/V219P/W228R, V219P, I127P/L142R, L142R/Y151A/K207A/V218A, I143G/Q213L, Y151A/V218A, I127P/L142R/V218I, L142R/Y151A, I143S/F155A/I205L/Q213L/W228R, I127P/L142R/K207A, I127P/D216P/V218A, L142R/Y151A/K207A, I143S/V219P, I127P/K207A, I127P/V218I, I143G/V219P/W228R, I127P/D216P, E68L/I127P/K207L, I143G/F155A/S212R/W228R, Y151A, I143S/V219P/W228R, I143S/Q213L/W228R, I143G/F155A/I205L, Y151A/K207A/D216P, I143G/W228R, E68L/I127P/K207A, E68L/I127P/K207A/V218A, I143G/F155A, I143G, I205L/Q213L/V219P, F155A/S212R/V219P/W228R, I143S/W228R, I205L/V219P/W228R, I143G/I205L, L142R/K207A, I143G/I205L/V219P, I143G/S212R/Q213L, I127P/K207L/V218I, L142R, I127P/D216P/V218I, I127P/L142R/Y151A/K207A/V218I, I143S/I205L/V219P/W228R, I205L/Q213L/W228R, I143G/V219P, L142R/K207L, E68L/Y151A/K207L, I143S/Q213L, L142R/Y151A/K207L, I127P/L142R/K207L, E68L/I127P/L142R/K207L, I143S/I205L, E68L/I127P, V218A, I127P/L142R/V196I, L142R/K207L/V218A, I143S/F155A, I143S, E68L/L142R/D216P, E68L/I127P/D216P/V218I, E68L/I127P/V218I, Y151A/D216P/V218I, I143S/S212R/W228R, I127P/L142R/Y151A, Y151A/K207L, I127P/L142R/D216P, V129I/K207A/V218A, L142R/K207L/V218I, E68L/L142R/Y151A/K207A, Y151A/K207A, E68L/L142R/Y151A, Y151A/K207L/V218I, W228R, F155A/Q213L, I127P/L142R/Y151A/V218I, I127P/Y151A, F155A/W228R, I143S/F155A/I205L, Y151A/D216P/V218A, E68L/L142R/K207L, A80V/Y151A/K207L, I205L/W228R, E68L/L142R/K207A/D216P/V218I, E68L/V218I, E68L/K207A, D216P/V218I, E68L/L142R, S212R, E68L/K207L, I205L, or L142R/K207A/D216P/V218I, wherein the amino acid positions are relative to the reference sequence corresponding to residues 12-231 of SEQ ID NO: 1708, or relative to the reference sequence corresponding to SEQ ID NO: 1708.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1952, or to the reference sequence corresponding to SEQ ID NO: 1952, wherein the amino acid sequence comprises one or more substitutions relative to the reference sequence corresponding to residues 12-231 of SEQ ID NO: 1952, or relative to the reference sequence corresponding to SEQ ID NO: 1952.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1970-2032, or to the reference sequence corresponding to an even-numbered SEQ ID NO. of SEQ ID NOs: 1970-2032, wherein the amino acid sequence comprises one or more substitutions relative to the reference sequence corresponding to residues 12-231 of SEQ ID NO: 1952, or relative to the reference sequence corresponding to SEQ ID NO: 1952.

In some embodiments, the amino acid sequence of the engineered adenylate kinase comprises at least a substitution or substitution set at amino acid position(s) 18/118, 87/119/192, 119/173/192, 118, 87/119, 18/118/170, 18, 119, 192, 87, 89, 59, 170, 64, 112, 93, 184, 146, 51, 125, 192, 191, 117, 172, or 163, wherein the amino acid positions are relative to the reference sequence corresponding to residues 12-231 of SEQ ID NO: 1952, or relative to the reference sequence corresponding to SEQ ID NO: 1952.

In some embodiments, the amino acid sequence of the engineered adenylate kinase comprises at least a substitution or substitution set, or amino acid residue(s) 18L/118G, 87M/119K/192A, 119K/173R/192A, 118G, 87M/119K, 18L/118G/1705, 18L, 119K, 192A, 87E, 87M, 89Q, 192I, 59L, 170S, 64R, 112N, 93E, 184S, 146N, 112K, 51T, 125I, 93S, 192W, 112A, 191D, 51M, 117L, 93Y, 172T, or 163Q, wherein the amino acid positions are relative to the reference sequence corresponding to residues 12-231 of SEQ ID NO: 1952, or relative to the reference sequence corresponding to SEQ ID NO: 1952.

In some embodiments, the amino acid sequence of the engineered adenylate kinase comprises at least a substitution or substitution set F18L/N118G, S87M/R119K/K192A, R119K/Q173R/K192A, N118G, S87M/R119K, F18L/N118G/G170S, F18L, RI 19K, K192A, S87E, S87M, K89Q, K192I, V59L, G170S, K64R, S112N, K93E, H184S, K146N, S112K, I51T, V125I, K93S, K192W, S112A, E191D, I51M, Q117L, K93Y, V172T, or L163Q, wherein the amino acid positions are relative to the reference sequence corresponding to residues 12-231 of SEQ ID NO: 1952, or relative to the reference sequence corresponding to SEQ ID NO: 1952.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1980, or to the reference sequence corresponding to SEQ ID NO: 1980, wherein the amino acid sequence comprises one or more substitutions relative to the reference sequence corresponding to residues 12-231 of SEQ ID NO: 1980, or relative to the reference sequence corresponding to SEQ ID NO: 1980.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2034-2134, or to the reference sequence corresponding to an even-numbered SEQ ID NO. of SEQ ID NOs: 2034-2134, wherein the amino acid sequence comprises one or more substitutions relative to the reference sequence corresponding to residues 12-231 of SEQ ID NO: 1980, or relative to the reference sequence corresponding to SEQ ID NO: 1980.

In some embodiments, the amino acid sequence of the engineered adenylate kinase comprises at least a substitution or substitution set at amino acid position(s) 87/119/192, 87/192, 87/119, 119/192, 192, 119, 146/192, 87, 59/87, 59/87/112/146, 146, 87/112/119, 74, 75, 66, 181, 81, 73, 77, 188, 216, 82, 136, 214, 21, 217, 154, 133, 71, 212, or 228, wherein the amino acid positions are relative to the reference sequence corresponding to residues 12-231 of SEQ ID NO: 1980, or relative to the reference sequence corresponding to SEQ ID NO: 1980.

In some embodiments, the amino acid sequence of the engineered adenylate kinase comprises at least a substitution or substitution set, or amino acid residue(s) 87E/119K/192A, 87E/192A, 87E/119K, 119K/192A, 87M/119K, 192A, 119K, 146N/192A, 87E, 87M/192A, 87M, 59L/87E, 59L/87E/112A/146N, 146N, 87E/112A/119K, 74G, 75L, 66A, 181V, 66W, 66G, 66Q, 81F, 66H, 73R, 66N, 77S, 77A, 66D, 66V, 188L, 77Q, 73I, 216E, 82L, 136L, 214L, 188G, 214T, 21S, 66T, 217E, 154R, 216T, 133V, 71T, 214R, 136I, 212H, 214P, or 228Q, wherein the amino acid positions are relative to the reference sequence corresponding to residues 12-231 of SEQ ID NO: 1980, or relative to the reference sequence corresponding to SEQ ID NO: 1980.

In some embodiments, the amino acid sequence of the engineered adenylate kinase comprises at least a substitution or substitution set S87E/R119K/K192A, S87E/K192A, S87E/R119K, R119K/K192A, S87M/R119K, K192A, RI 19K, K146N/K192A, S87E, S87M/K192A, S87M, V59L/S87E, V59L/S87E/S112A/K146N, K146N, S87E/S112A/R119K, P74G, D75L, I66A, T181V, I66W, I66G, I66Q, V81F, I66H, V73R, I66N, L77S, L77A, I66D, I66V, V188L, L77Q, V73I, P216E, V82L, A136L, G214L, V188G, G214T, P21S, I66T, P217E, I154R, P216T, E133V, E71T, G214R, A136I, S212H, G214P, or W228Q, wherein the amino acid positions are relative to the reference sequence corresponding to residues 12-231 of SEQ ID NO: 1980, or relative to the reference sequence corresponding to SEQ ID NO: 1980.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2072, or to the reference sequence corresponding to SEQ ID NO: 2072, wherein the amino acid sequence comprises one or more substitutions relative to the reference sequence corresponding to residues 12-231 of SEQ ID NO: 2072, or relative to the reference sequence corresponding to SEQ ID NO: 2072.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2136-2264, or to the reference sequence corresponding to an even-numbered SEQ ID NO. of SEQ ID NOs: 2136-2264, wherein the amino acid sequence comprises one or more substitutions relative to the reference sequence corresponding to residues 12-231 of SEQ ID NO: 2072, or relative to the reference sequence corresponding to SEQ ID NO: 2072.

In some embodiments, the amino acid sequence of the engineered adenylate kinase comprises at least a substitution or substitution set at amino acid position(s) 87, 194, 197, 93, 202, 38, 91, 183, 39, 146, 203, 37, 92, 36, 89, 131, 132, 90, 148, 35/197, 231, or 56, wherein the amino acid positions are relative to the reference sequence corresponding to residues 12-231 of SEQ ID NO: 2072, or relative to the reference sequence corresponding to SEQ ID NO: 2072.

In some embodiments, the amino acid sequence of the engineered adenylate kinase comprises at least a substitution or substitution set, or amino acid residue(s) 87R, 87K, 87L, 194R, 197L, 87Y, 93G, 202E, 38C, 91V, 194L, 183L, 93T, 39T, 91L, 146H, 203E, 203L, 146N, 37G, 194Y, 37R, 92L, 36R, 36S, 89T, 131A, 132T, 90F, 197Q, 90V, 148S, 91S, 35E/197V, 132V, 197A, 197V, 93P, 92S, 36M, 39A, 194V, 87V, 132G, 38F, 90T, 231R, 148F, 91G, 91A, 36I, 87E, 37L, 231T, 231A, 89P, 202S, 36E, 231Q, 87A, 146V, 146R, 56T, 93A, or 203R, wherein the amino acid positions are relative to the reference sequence corresponding to residues 12-231 of SEQ ID NO: 2072, or relative to the reference sequence corresponding to SEQ ID NO: 2072.

In some embodiments, the amino acid sequence of the engineered adenylate kinase comprises at least a substitution or substitution set S87R, S87K, S87L, Q194R, I197L, S87Y, K93G, K202E, G38C, C91V, Q194L, R183L, K93T, I39T, C91L, K146H, K203E, K203L, K146N, T37G, Q194Y, T37R, E92L, K36R, K36S, K89T, E131A, D132T, D90F, I197Q, D90V, G148S, C91S, A35E/I197V, D132V, I197A, I197V, K93P, E92S, K36M, I39A, Q194V, S87V, D132G, G38F, D90T, K231R, G148F, C91G, C91A, K36I, S87E, T37L, K231T, K231A, K89P, K202S, K36E, K231Q, S87A, K146V, K146R, N56T, K93A, or K203R, wherein the amino acid positions are relative to the reference sequence corresponding to residues 12-231 of SEQ ID NO: 2072, or relative to the reference sequence corresponding to SEQ ID NO: 2072.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2138, or to the reference sequence corresponding to SEQ ID NO: 2138, wherein the amino acid sequence comprises one or more substitutions relative to the reference sequence corresponding to residues 12-231 of SEQ ID NO: 2138, or relative to the reference sequence corresponding to SEQ ID NO: 2138.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2266-2366, or to the reference sequence corresponding to an even-numbered SEQ ID NO. of SEQ ID NOs: 2266-2366, wherein the amino acid sequence comprises one or more substitutions relative to the reference sequence corresponding to residues 12-231 of SEQ ID NO: 2138, or relative to the reference sequence corresponding to SEQ ID NO: 2138.

In some embodiments, the amino acid sequence of the engineered adenylate kinase comprises at least a substitution or substitution set at amino acid position(s) 161, 162, 153, 216, 212, 156, 214, 229, 77/143/214/216, 143/156/216, 77/143, 77/143/216, 77/214/216, 77/216, 143/156, 156/216, 143, 77, 77/214, 77/143/161/162/212/214, 77/162/214/216, 77/143/156/214/216, 77/143/156/162/212/216, 77/143/156/161/214/216, 77/156/162/216, 156/161/214/216, 77/143/162/214/216, 143/216, 214/216, 143/156/214/216, 143/214/216, 77/143/212/216, 143/156/161/162/216, 156/162/214, 77/143/156/161/162, 143/161/214/216, 77/161/216, or 143/212/214/216, wherein the amino acid positions are relative to the reference sequence corresponding to residues 12-231 of SEQ ID NO: 2138, or relative to the reference sequence corresponding to SEQ ID NO: 2138.

In some embodiments, the amino acid sequence of the engineered adenylate kinase comprises at least a substitution or substitution set, or amino acid residue(s) 161G, 162G, 153C, 216L, 212Q, 216H, 156V, 161L, 214W, 229C, 216M, 216V, 214A, 214M, 77I/143V/214E/216R, 143V/156V/216R, 77I/143V, 77I/143V/216R, 77I/214E/216R, 77I/216L, 143V/156V, 156V/216R, 143V, 77I/214E/216L, 77I, 77I/214E, 77I/143V/161L/162G/212Q/214E, 77I/162G/214E/216L, 77I/143V/156V/214E/216L, 77I/143V/156V/162G/212Q/216R, 77I/143V/156V/161L/214E/216R, 77I/216R, 77I/156V/162G/216R, 156V/161L/214E/216L, 77I/143V/162G/214W/216L, 143V/216L, 214E/216L, 143V/156V/214E/216R, 143V/214E/216L, 143V/214E/216R, 214E, 77I/143V/212Q/216R, 143V/156V/161L/162G/216R, 156V/162G/214E, 77I/143V/156V/161L/162G, 143V/161L/214E/216R, 77I/161L/216R, 143V/212Q/214E/216R, 77I/143V/214W/216R, 143V/216R, or 216R, wherein the amino acid positions are relative to the reference sequence corresponding to residues 12-231 of SEQ ID NO: 2138, or relative to the reference sequence corresponding to SEQ ID NO: 2138.

In some embodiments, the amino acid sequence of the engineered adenylate kinase comprises at least a substitution or substitution set D161G, E162G, M153C, P216L, S212Q, P216H, L156V, D161L, G214W, S229C, P216M, P216V, G214A, G214M, L77I/I143V/G214E/P216R, I143V/L156V/P216R, L77I/I143V, L77I/I143V/P216R, L77I/G214E/P216R, L77I/P216L, I143V/L156V, L156V/P216R, I143V, L77I/G214E/P216L, L77I, L77I/G214E, L77I/I143V/D161L/E162G/S212Q/G214E, L77I/E162G/G214E/P216L, L77I/I143V/L156V/G214E/P216L, L77I/I143V/L156V/E162G/S212Q/P216R, L77I/I143V/L156V/D161L/G214E/P216R, L77I/P216R, L77I/L156V/E162G/P216R, L156V/D161L/G214E/P216L, L77I/I143V/E162G/G214W/P216L, I143V/P216L, G214E/P216L, I143V/L156V/G214E/P216R, I143V/G214E/P216L, I143V/G214E/P216R, G214E, L77I/I143V/S212Q/P216R, I143V/L156V/D161L/E162G/P216R, L156V/E162G/G214E, L77I/I143V/L156V/D161L/E162G, I143V/D161L/G214E/P216R, L77I/D161L/P216R, I143V/S212Q/G214E/P216R, L77I/I143V/G214W/P216R, I143V/P216R, or P216R, wherein the amino acid positions are relative to the reference sequence corresponding to residues 12-231 of SEQ ID NO: 2138, or relative to the reference sequence corresponding to SEQ ID NO: 2138.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2294, or to the reference sequence corresponding to SEQ ID NO: 2294, wherein the amino acid sequence comprises one or more substitutions relative to the reference sequence corresponding to residues 12-231 of SEQ ID NO: 2294, or relative to the reference sequence corresponding to SEQ ID NO: 2294.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2368-2594, or to the reference sequence corresponding to an even-numbered SEQ ID NO. of SEQ ID NOs: 2368-2594, wherein the amino acid sequence comprises one or more substitutions relative to the reference sequence corresponding to residues 12-231 of SEQ ID NO: 2294, or relative to the reference sequence corresponding to SEQ ID NO: 2294.

In some embodiments, the amino acid sequence of the engineered adenylate kinase comprises at least a substitution at amino acid position 80, 105, 228, 50, 175, 170, 82, 173, 65, 53, 184, 122, 195, 68, 89, 34, 118, 119, 87, 179, 117, 113, 54, 190, 216, 88, 36, 166, 38, 169, 66, 142, 136, 131, 187, 40, 74, 127, 55, 148, 215, 64, 155, 116, 90, 132, 60, 94, 112, 120, 37, 108, 35, 51, 61, 212, 172, 59, or 56, or combinations thereof, wherein the amino acid positions are relative to the reference sequence corresponding to residues 12-231 of SEQ ID NO: 2294, or relative to the reference sequence corresponding to SEQ ID NO: 2294.

In some embodiments, the amino acid sequence of the engineered adenylate kinase comprises at least a substitution, or amino acid residue 80P, 105K, 228S, 50Q, 175F, 170R, 82L, 173S, 65T, 53M, 184V, 122S, 195M, 228L, 68S, 89A, 34E, 118S, 53S, 119F, 87A, 179C, 117S, 113E, 50C, 80G, 122H, 89V, 80R, 54I, 190Q, 216M, 88R, 80D, 36Q, 105S, 195G, 68V, 105L, 175D, 184T, 166L, 89H, 38R, 54G, 38F, 89I, 169I, 66C, 142W, 117L, 136Y, 131V, 187Y, 40L, 173T, 74C, 228V, 127L, 80L, 55G, 184M, 136V, 228T, 148R, 88T, 228Q, 66S, 215V, 195R, 190G, 64R, 184R, 122A, 505, 142M, 155W, 80S, 216G, 116A, 90C, 132K, 105G, 60W, 94Y, 112T, 87S, 187G, 120T, 87I, 68R, 37Y, 173R, 105M, 108V, 35L, 68I, 166F, 35F, 120S, 184S, 169A, 51L, 61V, 116F, 65D, 54H, 212C, 172S, 55L, 59L, 60C, 228F, or 56Q, or combinations thereof, wherein the amino acid positions are relative to the reference sequence corresponding to residues 12-231 of SEQ ID NO: 2294, or relative to the reference sequence corresponding to SEQ ID NO: 2294.

In some embodiments, the amino acid sequence of the engineered adenylate kinase comprises at least a substitution A80P, R105K, W228S, D50Q, L175F, S170R, V82L, Q173S, E65T, A53M, H184V, T122S, P195M, W228L, L68S, K89A, S34E, G118S, A53S, R119F, K87A, E179C, Q117S, F113E, D50C, A80G, T122H, K89V, A80R, K54I, R190Q, R216M, E88R, A80D, K36Q, R105S, P195G, L68V, R105L, L175D, H184T, D166L, K89H, G38R, K54G, G38F, K89I, V169I, W66C, R142W, Q117L, A136Y, E131V, K187Y, P40L, Q173T, P74C, W228V, I127L, A80L, Q55G, H184M, A136V, W228T, G148R, E88T, W228Q, W66S, I215V, P195R, R190G, K64R, H184R, T122A, D50S, R142M, F155W, A80S, R216G, S116A, D90C, D132K, R105G, A60W, G94Y, S112T, K87S, K187G, Q120T, K87I, L68R, T37Y, Q173R, R105M, E108V, A35L, L68I, D166F, A35F, Q120S, H184S, V169A, I51L, S61V, S116F, E65D, K54H, S212C, V172S, Q55L, V59L, A60C, W228F, or N56Q, or combinations thereof, wherein the amino acid positions are relative to the reference sequence corresponding to residues 12-231 of SEQ ID NO: 2294, or relative to the reference sequence corresponding to SEQ ID NO: 2294.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2368, or to the reference sequence corresponding to SEQ ID NO: 2368, wherein the amino acid sequence comprises one or more substitutions relative to the reference sequence corresponding to residues 12-231 of SEQ ID NO: 2368, or relative to the reference sequence corresponding to SEQ ID NO: 2368.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2596-2652 or 2676-2714, or to the reference sequence corresponding to an even-numbered SEQ ID NO. of SEQ ID NOs: 2596-2652 or 2676-2714, wherein the amino acid sequence comprises one or more substitutions relative to the reference sequence corresponding to residues 12-231 of SEQ ID NO: 2368, or relative to the reference sequence corresponding to SEQ ID NO: 2368.

In some embodiments, the amino acid sequence of the engineered adenylate kinase comprises at least a substitution or substitution set at amino acid position(s) 20, 21, 22, 43, 44, 46, 48, 138, 181, 182, 136/228, 82/142/173/184/216, 184/216, 50/184/216, 127/173/184, 105/136/170/175/228, 105/175, 50/184, 105/170/175/228, 50/127/173/184, 50/142/184/216, 50/82/127/216, 50, 50/82/173/216, 50/127/142/184/216, 50/82/127, 105/136, 105/228, 105/136/175/228, 173/216, 50/173, 228, 127/173, 105/136/170/195, 50/82/216, 175/228, or 50/82/127/142/184/216, wherein the amino acid positions are relative to the reference sequence corresponding to residues 12-231 of SEQ ID NO: 2368, or relative to the reference sequence corresponding to SEQ ID NO: 2368.

In some embodiments, the amino acid sequence of the engineered adenylate kinase comprises at least a substitution or substitution set, or amino acid residue(s) 20A, 21L, 21N, 21V, 22A, 43A, 43G, 43H, 44S, 46E, 46F, 46L, 46M, 46Y, 48F, 138M, 138V, 181C, 181V, 182A, 136Y/228V, 82L/142M/173T/184T/216M, 184T/216M, 50Q/184M/216M, 127L/173R/184M, 105K/136V/170R/175F/228T, 105K/175F, 127L/173T/184M, 50Q/184T, 105K/170R/175F/228V, 50Q/127L/173R/184M, 50Q/142M/184M/216M, 50Q/82L/127L/216M, 50Q, 50Q/82L/173T/216M, 50Q/127L/142W/184M/216M, 50Q/82L/127L, 136V/228V, 105K/136V, 105K/228V, 105K/136V/175F/228V, 173T/216M, 50Q/173T, 228V, 127L/173R, 105K/136V/170R/195G, 50Q/82L/216M, 175F/228V, or 50Q/82L/127L/142M/184M/216M, wherein the amino acid positions are relative to the reference sequence corresponding to residues 12-231 of SEQ ID NO: 2368, or relative to the reference sequence corresponding to SEQ ID NO: 2368.

In some embodiments, the amino acid sequence of the engineered adenylate kinase comprises at least a substitution or substitution set G20A, P21L, P21N, P21V, P22A, S43A, S43G, S43H, T44S, D46E, D46F, D46L, D46M, D46Y, L48F, L138M, L138V, T181C, T181V, V182A A136Y/W228V, V82L/R142M/Q173T/H184T/R216M, H184T/R216M, D50Q/H184M/R216M, I127L/Q173R/H184M, R105K/A136V/S170R/L175F/W228T, R105K/L175F, I127L/Q173T/H184M, D50Q/H184T, R105K/S170R/L175F/W228V, D50Q/I127L/Q173R/H184M, D50Q/R142M/H184M/R216M, D50Q/V82L/I127L/R216M, D50Q, D50Q/V82L/Q173T/R216M, D50Q/I127L/R142W/H184M/R216M, D50Q/V82L/I127L, A136V/W228V, R105K/A136V, R105K/W228V, R105K/A136V/L175F/W228V, Q173T/R216M, D50Q/Q173T, W228V, I127L/Q173R, R105K/A136V/S170R/P195G, D50Q/V82L/R216M, L175F/W228V, or D50Q/V82L/I127L/R142M/H184M/R216M, wherein the amino acid positions are relative to the reference sequence corresponding to residues 12-231 of SEQ ID NO: 2368, or relative to the reference sequence corresponding to SEQ ID NO: 2368.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2602, or to the reference sequence corresponding to SEQ ID NO: 2602, wherein the amino acid sequence comprises one or more substitutions relative to the reference sequence corresponding to residues 12-231 of SEQ ID NO: 2602, or relative to the reference sequence corresponding to SEQ ID NO: 2602.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2716-2988, or to the reference sequence corresponding to an even-numbered SEQ ID NO. of SEQ ID NOs: 2716-2988, wherein the amino acid sequence comprises one or more substitutions relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution or substitution set at amino acid position(s) 18, 18/56, 20, 21, 26, 27, 29, 30, 32, 42, 43, 50, 66, 67, 69, 70, 80, 98, 99, 102, 135, 137, 139, 141, 142, 143, 148, 149, 152, 153, 156, 173, 184, or 216, wherein the amino acid positions are relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution or substitution set, or amino acid residue(s) 18C/56T, 18E, 20P, 20R, 20T, 20V, 21L, 21R, 21T, 26L, 27C, 29R, 30L, 30S, 30Y, 32L, 42T, 43N, 50R, 66P, 67R, 69F, 70R, 80G, 80S, 98A, 98G, 98Q, 99A, 99C, 102A, 102C, 102N, 135I, 137L, 139H, 141G, 141V, 142L, 143T, 148P, 148S, 149L, 152F, 153V, 156A, 173F, 184N, 216D, 216E, 216G, 216L, 216N, 216P, or 216W, wherein the amino acid positions are relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution or substitution set L18C/N56T, L18E, G20P, G20R, G20T, G20V, P21L, P21R, P21T, K26L, G27C, Q29R, G30L, G30S, G30Y, I32L, I42T, S43N, Q50R, W66P, M67R, K69F, G70R, P80G, P80S, D98A, D98G, D98Q, G99A, G99C, R102A, R102C, R102N, V135I, R137L, L139H, R141G, R141V, R142L, V143T, G148P, G148S, R149L, N152F, M153V, L156A, Q173F, M184N, M216D, M216E, M216G, M216L, M216N, M216P, or M216W, wherein the amino acid positions are relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution or substitution set at amino acid position(s) 65, 65/104, 65/104/105/116/127, 65/104/105/127/136, 65/104/136, 65/104/136/170, 65/105/116/136/170, 65/105/127/136, 65/105/127/136/175, 65/105/127/170, 65/105/127/175, 65/105/136, 65/105/136/170, 65/105/170, 65/116, 65/127/136, 65/127/136/170, 65/127/170, 94/173, 94/187, 104/105, 104/105/127, 104/105/127/136/175/195, 104/105/136/170, 104/127, 104/127/136/170, 104/127/136/175, 105, 105/116/127/136/170/175, 105/127/136, 105/127/136/195, 105/136, 105/136/195, 116/127/136/175, 127/195, 132, 136, or 170, wherein the amino acid positions are relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution or substitution set 65T, 65T/104A, 65T/104A/105K/116A/127L, 65T/104A/105K/127L/136V, 65T/104A/136V, 65T/104A/136V/170R, 65T/105K/116A/136V/170R, 65T/105K/127L/136V, 65T/105K/127L/136V/175F, 65T/105K/127L/170R, 65T/105K/127L/175F, 65T/105K/136V, 65T/105K/136V/170R, 65T/105K/170R, 65T/116A, 65T/127L/136V/170R, 65T/127L/136Y, 65T/127L/170R, 94Y/173R, 94Y/187G, 104A/105K, 104A/105K/127L, 104A/105K/127L/136V/175F/195G, 104A/105K/136V/170R, 104A/127L, 104A/127L/136V/170R, 104A/127L/136V/175F, 105K, 105K/116A/127L/136Y/170R/175F, 105K/127L/136V, 105K/127L/136V/195G, 105K/136V, 105K/136V/195G, 116A/127L/136Y/175F, 127L/195G, 132K, 136V, and 170R, wherein the amino acid positions are relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution or substitution set E65T, E65T/V104A, E65T/V104A/R105K/S116A/I127L, E65T/V104A/R105K/I127L/A136V, E65T/V104A/A136V, E65T/V104A/A136V/S170R, E65T/R105K/S116A/A136V/S170R, E65T/R105K/I127L/A136V, E65T/R105K/I127L/A136V/L175F, E65T/R105K/I127L/S170R, E65T/R105K/I127L/L175F, E65T/R105K/A136V, E65T/R105K/A136V/S170R, E65T/R105K/S170R, E65T/S116A, E65T/I127L/A136V/S170R, E65T/I127L/A136Y, E65T/I127L/S170R, G94Y/Q173R, G94Y/K187G, V104A/R105K, V104A/R105K/I127L, V104A/R105K/I127L/A136V/L175F/P195G, V104A/R105K/A136V/S170R, V104A/I127L, V104A/I127L/A136V/S170R, V104A/I127L/A136V/L175F, R105K, R105K/S116A/I127L/A136Y/S170R/L175F, R105K/I127L/A136V, R105K/I127L/A136V/P195G, R105K/A136V, R105K/A136V/P195G, S116A/I127L/A136Y/L175F, I127L/P195G, D132K, A136V, or S170R, wherein the amino acid positions are relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution at amino acid position 36, 37, 40, 50, 59, 89, 117, 120, 128, 168, or 203, or combinations thereof, wherein the amino acid positions are relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution, or amino acid residue 36V, 37I, 37L, 40S, 50A, 59T, 59Y, 89P, 117L, 120L, 1281, 168L, or 203L, or combinations thereof, wherein the amino acid positions are relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution K36V, T37I, T37L, P40S, Q50A, V59T, V59Y, K89P, Q117L, Q120L, E1281, G168L, or K203L, or combinations thereof, wherein the amino acid positions are relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution at amino acid position 32, 36, 37, 50, 58, 61, 89, 90, 94, 97, 104, 110, 111, 118, 119, 128, 131, 132, 166, 169, 170, 172, 192, 195, or 200, or combinations thereof, wherein the amino acid positions are relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution, or amino acid residue 32F, 36S, 37F, 50N, 50V, 58Y, 61E, 61N, 89L, 90S, 94M, 94V, 97I, 104R, 110F, 111P, 111R, 118L, 119P, 128C, 128R, 131G, 132L, 166L, 166S, 169Y, 170G, 172A, 192H, 195I, or 200A, or combinations thereof, wherein the amino acid positions are relative to the reference sequence corresponding to residues 12-231 of SEQ ID NO: 2602, or relative to the reference sequence corresponding to SEQ ID NO: 2602.

In some embodiments, the amino acid sequence of the engineered adenylate kinase comprises at least a substitution I32F, K36S, T37F, Q50N, Q50V, E58Y, S61E, S61N, K89L, D90S, G94M, G94V, L97I, V104R, L110F, D111P, D111R, G118L, R119P, E128C, E128R, E131G, D132L, D166L, D166S, V169Y, S170G, V172A, K192H, P195I, or Y200A, or combinations thereof, wherein the amino acid positions are relative to the reference sequence corresponding to residues 12-231 of SEQ ID NO: 2602, or relative to the reference sequence corresponding to SEQ ID NO: 2602.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2832, or to the reference sequence corresponding to SEQ ID NO: 2832, wherein the amino acid sequence comprises one or more substitutions relative to the reference sequence corresponding to residues 12-231 of SEQ ID NO: 2832, or relative to the reference sequence corresponding to SEQ ID NO: 2832.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2990-3032, or to the reference sequence corresponding to an even-numbered SEQ ID NO. of SEQ ID NOs: 2990-3032, wherein the amino acid sequence comprises one or more substitutions relative to the reference sequence corresponding to residues 12-231 of SEQ ID NO: 2832, or relative to the reference sequence corresponding to SEQ ID NO: 2832.

In some embodiments, the amino acid sequence of the engineered adenylate kinase comprises at least a substitution or substitution set at amino acid position(s) 36, 36/50, 36/50/89/93/110, 36/50/89/93/139, 36/50/89/139/170, 36/50/89/172, 36/89, 36/170, 50, 50/89/93, 50/89/93/139, 50/93, 50/170, 89/170/172, 90/104, 90/151/157, 104/151/154, 104/151/154/157, 104/154/157/216, 151/157, 151/216, or 216, wherein the amino acid positions are relative to the reference sequence corresponding to residues 12-231 of SEQ ID NO: 2832, or relative to the reference sequence corresponding to SEQ ID NO: 2832.

In some embodiments, the amino acid sequence of the engineered adenylate kinase comprises at least a substitution or substitution set 36S, 36S/50N, 36S/50N/89L/93V/110F, 36S/50N/89L/93V/139R, 36S/50N/89L/139R/170G, 36S/50N/89L/172A, 36S/89L, 36S/170G, 50N, 50N/89L/93V, 50N/89L/93V/139R, 50N/93V, 50N/170G, 89L/170G/172A, 90S/104R, 90S/151F/157V, 104R/151F/154Q, 104R/151F/154Q/157V, 104R/154Q/157V/216E, 151F/157V, 151F/216E, or 216D, wherein the amino acid positions are relative to the reference sequence corresponding to residues 12-231 of SEQ ID NO: 2832, or relative to the reference sequence corresponding to SEQ ID NO: 2832.

In some embodiments, the amino acid sequence of the engineered adenylate kinase comprises at least a substitution or substitution set K36S, K36S/Q50N, K36S/Q50N/K89L/K93V/L1101F, K36S/Q50N/K89L/K93V/L139R, K36S/Q50N/K89L/L139R/S170G, K36S/Q50N/K89L/V172A, K36S/K89L, K36S/S170G, Q50N, Q50N/K89L/K93V, Q50N/K89L/K93V/L139R, Q50N/K93V, Q50N/S170G, K89L/S170G/V172A, D90S/A104R, D90S/Y151F/P157V, A104R/Y151F/I154Q, A104R/Y151F/I154Q/P157V, A104R/I154Q/P157V/M216E, Y151F/P157V, Y151F/M216E, or M216D, wherein the amino acid positions are relative to the reference sequence corresponding to residues 12-231 of SEQ ID NO: 2832, or relative to the reference sequence corresponding to SEQ ID NO: 2832.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2994, or to the reference sequence corresponding to SEQ ID NO: 2994, wherein the amino acid sequence comprises one or more substitutions relative to the reference sequence corresponding to residues 12-231 of SEQ ID NO: 2994, or relative to the reference sequence corresponding to SEQ ID NO: 2994.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 3034-3150, or to the reference sequence corresponding to an even-numbered SEQ ID NO. of SEQ ID NOs: 3034-3150, wherein the amino acid sequence comprises one or more substitutions relative to the reference sequence corresponding to residues 12-231 of SEQ ID NO: 2994, or relative to the reference sequence corresponding to SEQ ID NO: 2994.

In some embodiments, the amino acid sequence of the engineered adenylate kinase comprises at least a substitution at amino acid position 50, 58, 59, 68, 74, 76, 79, 80, 83, 90, 112, 113, 119, 157, 170, 172, 182, 184, 217, 224, 226, 228, 229, 230, or 231, or combinations thereof, wherein the amino acid positions are relative to the reference sequence corresponding to residues 12-231 of SEQ ID NO: 2994, or relative to the reference sequence corresponding to SEQ ID NO: 2994.

In some embodiments, the amino acid sequence of the engineered adenylate kinase comprises at least a substitution, or amino acid residue 50L, 58C, 59W, 68A, 68E, 68V, 74Q, 76G, 76L, 79A, 79K, 79L, 79P, 79W, 80W, 90K, 112C, 113W, 119S, 170H, 170P, 170R, 172M, 182G, 182L, 182Q, 184R, 217T, 224S, 224T, 226K, 226R, 228D, 230M, or 231R, or combinations thereof, wherein the amino acid positions are relative to the reference sequence corresponding to residues 12-231 of SEQ ID NO: 2994, or relative to the reference sequence corresponding to SEQ ID NO: 2994.

In some embodiments, the amino acid sequence of the engineered adenylate kinase comprises at least a substitution Q50L, E58C, V59W, L68A, L68E, L68V, P74Q, E76G, E76L, N79A, N79K, N79L, N79P, N79W, P80W, D90K, S112C, F113W, R119S, S170H, S170P, S170R, V172M, V182G, V182L, V182Q, M184R, P217T, G224S, G224T, L226K, L226R, W228D, D230M, or K231R, or combinations thereof, wherein the amino acid positions are relative to the reference sequence corresponding to residues 12-231 of SEQ ID NO: 2994, or relative to the reference sequence corresponding to SEQ ID NO: 2994.

In some embodiments, the amino acid sequence of the engineered adenylate kinase comprises at least a substitution or substitution set at amino acid position(s) 79/104/157/228, 79/157/228/229, 79/157/229, 79/228, 83/104, 83/104/151/168/173/190, 83/113, 83/173/190, 83/173/190/201, 83/173/201, 83/190, 83/190/201/216, 83/216, 104/157, 157/173/190/216, 157/183, 157/190, 157/190/216, 157/228, 173/216, or 201/216, wherein the amino acid positions are relative to the reference sequence corresponding to residues 12-231 of SEQ ID NO: 2994, or relative to the reference sequence corresponding to SEQ ID NO: 2994.

In some embodiments, the amino acid sequence of the engineered adenylate kinase comprises at least a substitution or substitution set, or amino acid residue(s) 79W/104R/157P/228V, 79W/157P/228V/229I, 79W/157P/229I, 79W/228V, 83L, 83L/104R, 83L/104R/151Y/168S/173T/190Q, 83L/113Y, 83L/173T/201A, 83L/173V/190Q, 83L/173V/190Q/201F, 83L/190Q, 83L/190Q/201A/216E, 83L/216E, 104R/157P, 157P, 157P/173T/190Q/216E, 157P/183L, 157P/190Q, 157P/190Q/216E, 157P/228V, 173T/216E, 201F/216E, or 229I, wherein the amino acid positions are relative to the reference sequence corresponding to residues 12-231 of SEQ ID NO: 2994, or relative to the reference sequence corresponding to SEQ ID NO: 2994.

In some embodiments, the amino acid sequence of the engineered adenylate kinase comprises at least a substitution or substitution set N79W/A104R/V157P/W228V, N79W/V157P/W228V/S229I, N79W/V157P/S229I, N79W/W228V, K83L, K83L/A104R, K83L/A104R/F151Y/G168S/Q173T/R190Q, K83L/F113Y, K83L/Q173T/S201A, K83L/Q173V/R190Q, K83L/Q173V/R190Q/S201F, K83L/R190Q, K83L/R190Q/S201A/M216E, K83L/M216E, A104R/V157P, V157P, V157P/Q173T/R190Q/M216E, V157P/R183L, V157P/R190Q, V157P/R190Q/M216E, V157P/W228V, Q173T/M216E, S201F/M216E, or S229I, wherein the amino acid positions are relative to the reference sequence corresponding to residues 12-231 of SEQ ID NO: 2994, or relative to the reference sequence corresponding to SEQ ID NO: 2994.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 3104, or to the reference sequence corresponding to SEQ ID NO: 3104, wherein the amino acid sequence comprises one or more substitutions relative to the reference sequence corresponding to residues 12-231 of SEQ ID NO: 3104, or relative to the reference sequence corresponding to SEQ ID NO: 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 3152-3192, or to the reference sequence corresponding to an even-numbered SEQ ID NO. of SEQ ID NOs: 3152-3192, wherein the amino acid sequence comprises one or more substitutions relative to the reference sequence corresponding to residues 12-231 of SEQ ID NO: 3104, or relative to the reference sequence corresponding to SEQ ID NO: 3104.

In some embodiments, the amino acid sequence of the engineered adenylate kinase comprises at least a substitution or substitution set at amino acid position(s) 104, 104/157, 104/157/228/229, 104/157/229, 104/170/190/228, 104/201/228, 157/228, 157/228/229, 170/190/228, 170/190/228/229, 190, 190/229, or 201/228, wherein the amino acid positions are relative to the reference sequence corresponding to residues 12-231 of SEQ ID NO: 3104, or relative to the reference sequence corresponding to SEQ ID NO: 3104.

In some embodiments, the amino acid sequence of the engineered adenylate kinase comprises at least a substitution or substitution set, or amino acid residue(s) 104R, 104R/157P, 104R/157P/228V/229I, 104R/157P/229I, 104R/170P/190R/228V, 104R/201S/228V, 157P/228V, 157P/228V/229I, 170P/190R/228V, 170P/190R/228V/229I, 190R, 190R/229I, or 2015/228V, wherein the amino acid positions are relative to the reference sequence corresponding to residues 12-231 of SEQ ID NO: 3104, or relative to the reference sequence corresponding to SEQ ID NO: 3104.

In some embodiments, the amino acid sequence of the engineered adenylate kinase comprises at least a substitution or substitution set A104R, A104R/V157P, A104R/V157P/W228V/S229I, A104R/V157P/S229I, A104R/S170P/Q190R/W228V, A104R/A201S/W228V, V157P/W228V, V157P/W228V/5229I, S170P/Q190R/W228V, S170P/Q190R/W228V/S229I, Q190R, Q190R/S229I, or A201S/W228V, wherein the amino acid positions are relative to the reference sequence corresponding to residues 12-231 of SEQ ID NO: 3104, or relative to the reference sequence corresponding to SEQ ID NO: 3104.

In some embodiments, the amino acid sequence of the engineered adenylate kinase comprises at least a substitution or substitution set at amino acid position(s) 104, 104/157/190/228, 104/170/190, 104/170/228, 104/190, 104/190/201, 104/190/228, 104/228/229, or 228, wherein the amino acid positions are relative to the reference sequence corresponding to residues 12-231 of SEQ ID NO: 3104, or relative to the reference sequence corresponding to SEQ ID NO: 3104.

In some embodiments, the amino acid sequence of the engineered adenylate kinase comprises at least a substitution or substitution set, or amino acid residue(s) 104R, 104R/157P/190R/228V, 104R/170P/190R, 104R/170P/228V, 104R/190R, 104R/190R/201S, 104R/190R/228V, 104R/228V/229I, or 228V, wherein the amino acid positions are relative to the reference sequence corresponding to residues 12-231 of SEQ ID NO: 3104, or relative to the reference sequence corresponding to SEQ ID NO: 3104.

In some embodiments, the amino acid sequence of the engineered adenylate kinase comprises at least a substitution or substitution set A104R, A104R/V157P/Q190R/W228V, A104R/S170P/Q190R, A104R/S170P/W228V, A104R/Q190R, A104R/Q190R/A201S, A104R/Q190R/W228V, A104R/W228V/S229I, or W228V, wherein the amino acid positions are relative to the reference sequence corresponding to residues 12-231 of SEQ ID NO: 3104, or relative to the reference sequence corresponding to SEQ ID NO: 3104.

In some embodiments, the amino acid sequence of the engineered adenylate kinase comprises at least a substitution at an amino acid position set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the amino acid sequence of the engineered adenylate kinase comprises at least one substitution set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the amino acid sequence of the engineered adenylate kinase comprises at least a substitution or substitution set at amino acid position(s) set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the amino acid sequence of the engineered adenylate kinase comprises at least a substitution or substitution set of an engineered adenylate kinase variant set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference amino acid sequence comprising a substitution or substitution set of an engineered adenylate kinase variant set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the engineered adenylate kinase comprises an amino acid sequence comprising residues 12-231 of an even-numbered SEQ ID NO. of SEQ ID NOs: 4-1302, 1356-2652, and 2676-3192, or comprising an even-numbered SEQ ID NO. of SEQ ID NOs: 4-1302, 1356-2652, and 2676-3192. In some embodiments, the amino acid sequence of the engineered adenylate kinase optionally includes 1, 2, 3, 4, 5, 6, 7, 8, 9, or up to 10 substitutions, insertions, and/or deletions. In some embodiments, the amino acid sequence of the engineered adenylate kinase includes 1, 2, 3, 4, 5, 6, 7, 8, 9, or up to 10 substitutions. In some embodiments, the amino acid sequence of the engineered adenylate kinase optionally includes 1, 2, 3, 4, or 5 substitutions, insertions, and/or deletions. In some embodiments, the amino acid sequence of the engineered adenylate kinase optionally includes 1, 2, 3, 4, or 5 substitutions.

In some embodiments, the engineered adenylate kinase comprises an amino acid sequence comprising residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or comprising SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104. In some embodiments, the amino acid sequence of the engineered adenylate kinase optionally includes 1, 2, 3, 4, 5, 6, 7, 8, 9, or up to 10 substitutions, insertions, and/or deletions. In some embodiments, the amino acid sequence of the engineered adenylate kinase includes 1, 2, 3, 4, 5, 6, 7, 8, 9, or up to 10 substitutions. In some embodiments, the amino acid sequence of the engineered adenylate kinase optionally includes 1, 2, 3, 4, or 5 substitutions, insertions, and/or deletions. In some embodiments, the amino acid sequence of the engineered adenylate kinase optionally includes 1, 2, 3, 4, or 5 substitutions.

In some embodiments, the engineered adenylate kinase polypeptide has 1, 2, 3, 4, or up to 5 substitutions in the amino acid sequence. In some embodiments, the engineered adenylate kinase polypeptide has 1, 2, 3, or 4 substitutions in the amino acid sequence.

In some embodiments, the engineered adenylate kinase described herein has adenylate kinase activity and at least one improved property as compared to a reference adenylate kinase.

In some embodiments, the engineered adenylate kinase has increased activity on NMP substrate as compared to the reference adenylate kinase. In some embodiments, the NMP substrate is AMP, CMP, GMP, UMP, or TMP. In some embodiments, the engineered adenylate kinase has at least 1.1, 1.2, 1.3, 1.4, 1.5, 2, 3, 4, 5, 10, or more fold activity compared to the reference adenylate kinase.

In some embodiments, the engineered adenylate kinase is capable of converting NMP substrate to NDP product at greater than 30%, 40%, 50%, 60%, 70%, 80%, 85%, 90%, 91%, 92%, 93%, 94%, 95%, 96%, 97%, 98%, or 99%. In some embodiments, the engineered adenylate kinase is capable of converting substrate AMP, GMP, CMP, UMP, or TMP to product ADP, GDP, CDP, UDP, or TDP, respectively, at greater than 30%, 40%, 50%, 60%, 70%, 80%, 85%, 90%, 91%, 92%, 93%, 94%, 95%, 96%, 97%, 98%, or 99%.

In some embodiments, the engineered adenylate kinase has increased activity on 2′-fluoro modified nucleoside monophosphate and/or increased activity on 2′-O-methyl modified nucleoside monophosphate. In some embodiments, the engineered adenylate kinase has at least 1.1, 1.2, 1.3, 1.4, 1.5, 2, 3, 4, 5, 10, or more fold activity with 2′-fluoro modified nucleoside monophosphate and/or 2′-O-methyl modified nucleoside monophosphate as compared to the reference adenylate kinase. In some embodiments, the 2′-fluoro modified nucleoside monophosphate substrate is 2′-F-AMP, 2′-F-GMP, 2′-F-CMP, 2′-F-UMP, or 2′-F-TMP. In some embodiments, the 2′-O-methyl modified nucleoside monophosphate substrate is 2′-O-methyl-AMP, 2′-O-methyl-GMP, 2′-O-methyl-CMP, 2′-O-methyl-UMP, or 2′-O-methyl-TMP. Exemplary increases in activity with 2′-modified nucleoside monophosphate substrates are provided in the Examples.

In some embodiments, the engineered adenylate kinase is capable of converting substrate 2′-fluoro-NMP to product 2′-fluoro-NDP at greater than 30%, 40%, 50%, 60%, 70%, 80%, 85%, 90%, 91%, 92%, 93%, 94%, 95%, 96%, 97%, 98%, or 99%. In some embodiments, the engineered adenylate kinase is capable of converting substrate 2′-fluoro-AMP, 2′-fluoro-GMP, 2′-fluoro-CMP, 2′-fluoro-UMP, or 2′-fluoro-TMP to product 2′-fluoro-ADP, 2′-fluoro-GDP, 2′-fluoro-CDP, 2′-fluoro-UDP, or 2′-fluoro-TDP, respectively, at greater than 30%, 40%, 50%, 60%, 70%, 80%, 85%, 90%, 91%, 92%, 93%, 94%, 95%, 96%, 97%, 98%, or 99%.

In some embodiments, the engineered adenylate kinase is capable of converting substrate 2′-O-methyl-NMP substrate to product 2′-O-methyl-NDP at greater than 30%, 40%, 50%, 60%, 70%, 80%, 85%, 90%, 91%, 92%, 93%, 94%, 95%, 96%, 97%, 98%, or 99%. In some embodiments, the engineered adenylate kinase is capable of converting substrate 2′-O-methyl-AMP, 2′-O-methyl-GMP, 2′-O-methyl-CMP, 2′-O-methyl-UMP, or 2′-O-methyl-TMP to product 2′-O-methyl-ADP, 2′-O-methyl-GDP, 2′-O-methyl-CDP, 2′-O-methyl-UDP, or 2′-O-methyl-TDP, respectively, at greater than 30%, 40%, 50%, 60%, 70%, 80%, 85%, 90%, 91%, 92%, 93%, 94%, 95%, 96%, 97%, 98%, or 99%.

Exemplary conditions for the conversions are provided in the Examples, including for NMP substrates, 2′-fluoro NDP substrates, and 2′-O-methyl NDP substrates.

In some embodiments, the engineered adenylate kinase exhibits increased stability as compared to the reference adenylate kinase. In some embodiments, the engineered adenylate kinase exhibis increased thermostability, e.g., at 50° C. to 70° C. Exemplary stability and thermostability conditions are provided in the Examples.

In some embodiments, the engineered adenylate kinase exhibits an improved property selected from i) increased activity on unmodified nucleoside monophosphate (NMP), ii) increased stability, iii) increased thermostability, iv) increased activity on 2′-fluoro modified nucleoside monophosphate, and v) increased activity on 2′-O-methyl modified nucleoside monophosphate, or any combinations of i), ii), iii), iv), and v), as compared to a reference adenylate kinase.

In some embodiments, the reference adenylate kinase for comparison of an improved property has an amino acid sequence corresponding to residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or an amino acid sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104. In some embodiments, the reference adenylate kinase has an amino acid sequence corresponding to residues 12-231 of SEQ ID NO: 2, or an amino acid sequence corresponding to SEQ ID NO: 2.

In some embodiments, the present disclosure further provides an adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to
(a) a sequence corresponding to residues 12 to 228 of SEQ ID NO: 1336,
   a sequence corresponding to residues 12 to 228 of SEQ ID NO: 1338,
   a sequence corresponding to residues 12 to 225 of SEQ ID NO: 1340, or
   a sequence corresponding to residues 12 to 228 of SEQ ID NO: 1342; or
(b) a sequence corresponding to SEQ ID NO: 1336;
   a sequence corresponding to SEQ ID NO: 1338;
   a sequence corresponding to SEQ ID NO: 1340; or
   a sequence corresponding to SEQ ID NO: 1342.

In some embodiments, the present disclosure provides an adenylate kinase comprising an amino acid sequence comprising:
(a) residues 12 to 228 of SEQ ID NO: 1336,
   residues 12 to 228 of SEQ ID NO: 1338,
   residues 12 to 225 of SEQ ID NO: 1340,
   residues 12 to 228 of SEQ ID NO: 1342; or
(b) SEQ ID NO: 1336;
   SEQ ID NO: 1338;
   SEQ ID NO: 1340; or
   SEQ ID NO: 1342.

In some embodiments, the engineered adenylate kinase is provided in the form of a fusion polypeptide. In some embodiments, the engineered adenylate kinase is fused to variety of polypeptide sequences, such as, by way of example and not limitation, polypeptide tags that can be used for detection and/or purification. In some embodiments, the fusion protein of the engineered adenylate kinase comprises a glycine-histidine or histidine-tag (His-tag). In some embodiments, the fusion protein of the engineered adenylate kinase comprises a polylysine, e.g., 2-12 units of lysine, such as for conjugation to a support medium. In some embodiments, the fusion protein of the engineered adenylate kinase comprises an epitope tag, such as c-myc, FLAG, V5, or hemagglutinin (HA). In some embodiments, the fusion protein of the engineered adenylate kinase comprises a GST, SUMO, Strep, MBP, or GFP tag. In some embodiments, the fusion is to the amino (N—) terminus of engineered adenylate kinase polypeptide. In some embodiments, the fusion is to the carboxy (C—) terminus of the adenylate kinase polypeptide.

In some embodiments, the present disclosure further provides functional fragments or biologically active fragments of engineered adenylate kinase polypeptides described herein. Thus, for each and every embodiment herein of an engineered adenylate kinase, a functional fragment or biologically active fragment of the engineered adenylate kinase is provided herewith. In some embodiments, a functional fragment or biologically active fragments of an engineered adenylate kinase comprises at least about 90%, 91%, 92%, 93%, 94%, 95%, 96%, 97%, 98%, or 99% of the activity of the adenylate kinase polypeptide from which it was derived (i.e., the parent adenylate kinase). In some embodiments, functional fragments or biologically active fragments comprise at least about 90%, 91%, 92%, 93%, 94%, 95%, 96%, 97%, 98%, or 99% of the parent sequence of the adenylate kinase. In some embodiments the functional fragment will be truncated by less than 5, less than 10, less than 15, less than 10, less than 25, less than 30, less than 35, less than 40, less than 45, and less than 50 amino acids.

In some embodiments, a functional fragment of an engineered adenylate kinase herein comprises at least 70%, 75%, 80%, 81%, 82%, 83%, 84%, 85%, 90%, 91%, 92%, 93%, 94%, 95%, 96%, 97%, 98%, or 99% of the parent sequence of the engineered adenylate kinase. In some embodiments, the functional fragment will be truncated by less than 5, less than 10, less than 15, less than 10, less than 25, less than 30, less than 35, less than 40, less than 45, less than 50, less than 55, less than 60, less than 65, or less than 70 amino acids.

In some embodiments, the functional fragments or biologically active fragments of the engineered adenylate kinase polypeptide described herein include at least a mutation or mutation set in the amino acid sequence of an engineered adenylate kinase described herein. Accordingly, in some embodiments, the functional fragments or biologically active fragments of the engineered adenylate kinase displays the enhanced or improved property associated with the mutation or mutation set in the parent adenylate kinase.

In some embodiments, the engineered adenylate kinase is purified, as described herein. In some embodiments, the purified preparation has the engineered adenylate kinase at least 60%, 70%, 80%, 85%, 90%, or 95% greater of the protein content of the preparation.

In some embodiments, an engineered adenylate kinase described herein is provided immobilized on a substrate or support medium, such as a solid substrate, a porous substrate, a membrane, or particles. The polypeptide can be entrapped in matrixes or membranes. In some embodiments, matrices include polymeric materials such as calcium-alginate, agar, k-carrageenin, polyacrylamide, and collagen. In some embodiments, the solid matrices, includes, among others, activated carbon, porous ceramic, and diatomaceous earth. In some embodiments, the matrix is a particle, a membrane, or a fiber. Types of membranes include, among others, nylon, cellulose, polysulfone, or polyacrylate.

In some embodiments, the engineered adenylate kinase is immobilized on the surface of a support material. In some embodiments, the polypeptide is adsorbed on the support material. In some embodiments, the polypeptide is immobilized on the support material by covalent attachment. Support materials include, among others, inorganic materials, such as alumina, silica, porous glass, ceramics, diatomaceous earth, clay, and bentonite, or organic materials, such as cellulose (CMC, DEAE-cellulose), starch, activated carbon, polyacrylamide, polystyrene, polymethacrylate, polyacrylate, and ion-exchange resins, such as Amberlite, Sephadex, and Dowex. Various functional groups known in the art may be used to conjugate the engineered adenylate kinase to the substrate or support medium, e.g., epoxide, imide, etc.

### Polynucleotides Encoding Engineered Polypeptides, Expression Vectors and Host Cells

In another aspect, the present disclosure provides recombinant polynucleotides encoding the engineered adenylate kinases described herein. In some embodiments, the recombinant polynucleotides are operably linked to one or more heterologous regulatory sequences that control gene expression to create a recombinant polynucleotide construct capable of expressing the engineered adenylate kinase. In some embodiments, an expression construct containing at least one heterologous polynucleotide encoding the engineered adenylate kinase polypeptide(s) is introduced into appropriate host cells to express the corresponding adenylate kinase polypeptide(s).

As will be apparent to the skilled artisan, availability of a protein sequence and the knowledge of the codons corresponding to the various amino acids provide a description of all the polynucleotides capable of encoding the subject polypeptides. The degeneracy of the genetic code, where the same amino acids are encoded by alternative or synonymous codons, allows an extremely large number of nucleic acids to be made, all of which encode an engineered adenylate kinase of the present disclosure. Thus, the present disclosure provides methods and compositions for the production of each and every possible variation of polynucleotides that could be made that encode the engineered adenylate kinase polypeptides described herein by selecting combinations based on the possible codon choices, and all such polynucleotide variants are to be considered specifically disclosed for any polypeptide described herein, including the amino acid sequences presented in the Examples (e.g., Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2) and in the Sequence Listing.

In some embodiments, the codons are preferably optimized for utilization by the chosen host cell for protein production. In some embodiments, preferred codons in bacteria are used for expression in bacteria. In some embodiments, preferred codons in fungal cells are used for expression in fungal cells. In some embodiments, preferred codons in insect cells are used for expression in insect cells. In some embodiments, preferred codons in mammalian cells are used for expression in mammalian cells. In some embodiments, codon optimized polynucleotides encoding an engineered adenylate kinase polypeptide described herein contain preferred codons at about 40%, 50%, 60%, 70%, 80%, 90%, or greater than 90% of the codon positions in the full-length coding region.

Accordingly, in some embodiments, a recombinant polynucleotide of the present disclosure comprises a polynucleotide sequence encoding an engineered adenylate kinase polypeptide described herein. In some embodiments, the polynucleotide sequence of the recombinant polynucleotide has preferred codons for expression (e.g., codon optimized). In some embodiments, the polynucleotide sequence of the recombinant polynucleotide is codon optimized for expression in eukaryotic or prokaryotic cells, as further discussed herein.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12-231 of an even-numbered SEQ ID NO. of SEQ ID NOs: 2-1302, 1356-2652, and 2676-3192, or to a reference sequence corresponding to an even-numbered SEQ ID NO. of SEQ ID NOs: 2-1302, 1356-2652, and 2676-3192, wherein the amino acid sequence comprises one or more substitutions relative to a reference sequence corresponding to amino acid residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or relative to a reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or to a reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, wherein the amino acid sequence comprises one or more substitutions relative to the reference sequence corresponding to amino acid residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12-231 of SEQ ID NO: 2, or to the reference sequence corresponding to SEQ ID NO: 2, wherein the amino acid sequence comprises one or more substitutions relative to the reference sequence corresponding to amino acid residues 12-231 of SEQ ID NO: 2, or relative to the reference sequence corresponding to SEQ ID NO: 2.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, wherein the amino acid sequence comprises one or more substitutions relative to the reference sequence corresponding to amino acid residues 12-231 of SEQ ID NO: 2, or relative to the reference sequence corresponding to SEQ ID NO: 2.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12-231 of an even-numbered SEQ ID NO. of SEQ ID NOs: 4-1302, 1356-2652, and 2676-3192, or to a reference sequence corresponding to an even-numbered SEQ ID NO. of SEQ ID NOs: 4-1302, 1356-2652, and 2676-3192, wherein the amino acid sequence comprises one or more substitutions relative to the reference sequence corresponding to amino acid residues 12-231 of SEQ ID NO: 2, or relative to the reference sequence corresponding to SEQ ID NO: 2.

In some embodiments, the recombinant polynucleotide comprises a polynucleotide sequence encoding a engineered adenylate kinase comprising an amino acid sequence comprising at least a substitution at amino acid position 11, 13, 14, 15, 16, 18, 20, 21, 22, 26, 27, 29, 30, 31, 32, 34, 35, 36, 37, 38, 39, 40, 42, 43, 44, 46, 48, 50, 51, 52, 53, 54, 55, 56, 57, 58, 59, 60, 61, 62, 63, 64, 65, 66, 67, 68, 69, 70, 71, 73, 74, 75, 76, 77, 78, 79, 80, 81, 82, 83, 84, 87, 88, 89, 90, 91, 92, 93, 94, 97, 98, 99, 100, 101, 102, 104, 105, 108, 109, 110, 111, 112, 113, 115, 116, 117, 118, 119, 120, 122, 123, 124, 125, 126, 127, 128, 129, 131, 132, 133, 134, 135, 136, 137, 138, 139, 140, 141, 142, 143, 146, 148, 149, 150, 151, 152, 153, 154, 155, 156, 157, 161, 162, 163, 166, 168, 169, 170, 172, 173, 175, 178, 179, 180, 181, 182, 183, 184, 186, 187, 188, 190, 191, 192, 194, 195, 196, 197, 198, 199, 200, 201, 202, 203, 204, 205, 206, 207, 208, 210, 211, 212, 213, 214, 215, 216, 217, 218, 219, 220, 221, 222, 223, 224, 225, 226, 227, 228, 229, 230, or 231, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the recombinant polynucleotide comprises a polynucleotide sequence encoding an engineered adenylate kinase comprising an amino acid sequence comprising at least a substitution at amino acid position 13, 18, 29, 30, 31, 32, 34, 35, 48, 50, 53, 55, 59, 60, 61, 62, 66, 68, 77, 78, 80, 83, 87, 100, 104, 105, 109, 118, 119, 127, 128, 133, 136, 139, 142, 143, 150, 151, 155, 157, 168, 170, 175, 184, 186, 190, 201, 207, 212, 213, 214, 216, 217, 218, 224, or 226, or combinations thereof, wherein the amino acid positions are relative to the reference sequence corresponding to amino acid residues 12-231 of SEQ ID NO: 2, or relative to the reference sequence corresponding to SEQ ID NO: 2.

In some embodiments, the recombinant polynucleotide comprises a polynucleotide sequence encoding an engineered adenylate kinase comprising an amino acid sequence comprising at least a substitution or substitution set at amino acid position(s) 29/30/128/142/186/212/213, 18/142/212/213, 29/30/128/142/186, 40/135/214, 18/29/30/127/150, 82, 69, 118, 29/30, 206, 32, 16, 104, 111/135/136/214, 29/128/175, 178, 18/128/142, 32/108/111/156/191, 18/29/128/129/142/186, 32/133, 18/29/30/212/213/222, 29/65/127, 32/40/43/52/155/156/217, 18/29/128/129, 80, 32/43/138/152/191/214/215, 183, 18/175/186/212/213, 129, 18/29/30/65/129/142/150/186, 127/142/212/213, 30/65/142/222, 40/43/215, 136, 29/150/211/212/213/221, 43/111/136/219, 226, 88, 140/215/216, 32/133/134, or 18/29/128/186, wherein the amino acid positions are relative to the reference sequence corresponding to amino acid residues 12-231 of SEQ ID NO: 2, or relative to the reference sequence corresponding to SEQ ID NO: 2.

In some embodiments, the recombinant polynucleotide comprises a polynucleotide sequence encoding an engineered adenylate kinase comprising an amino acid sequence comprising at least a substitution at an amino acid position set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to amino acid residues 12-231 of SEQ ID NO: 2, or relative to the reference sequence corresponding to SEQ ID NO: 2.

In some embodiments, the recombinant polynucleotide comprises a polynucleotide sequence encoding an engineered adenylate kinase comprising an amino acid sequence comprising at least one substitution set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to amino acid residues 12-231 of SEQ ID NO: 2, or relative to the reference sequence corresponding to SEQ ID NO: 2.

In some embodiments, the recombinant polynucleotide comprises a polynucleotide sequence encoding an engineered adenylate kinase comprising an amino acid sequence comprising at least a substitution or substitution set at amino acid position(s) set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to amino acid residues 12-231 of SEQ ID NO: 2, or relative to the reference sequence corresponding to SEQ ID NO: 2.

In some embodiments, the recombinant polynucleotide comprises a polynucleotide sequence encoding an engineered adenylate kinase comprising an amino acid sequence comprising at least a substitution or substitution set of an engineered adenylate kinase variant set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to amino acid residues 12-231 of SEQ ID NO: 2, or relative to the reference sequence corresponding to SEQ ID NO: 2.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference amino acid sequence comprising a substitution or substitution set of an engineered adenylate kinase variant set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to amino acid residues 12-231 of SEQ ID NO: 2, or relative to the reference sequence corresponding to SEQ ID NO: 2.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to residues 12-231 of an even-numbered SEQ ID NO. of SEQ ID NOs: 4-1302, 1356-2652, and 2676-3192, or to a reference sequence corresponding to an even-numbered SEQ ID NO. of SEQ ID NOs: 4-1302, 1356-2652, and 2676-3192.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, wherein the amino acid sequence comprises one or more substitutions relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 4-1302, 1356-2652, and 2676-3192, or to the reference sequence corresponding to an even-numbered SEQ ID NO. of SEQ ID NOs: 4-1302, 1356-2652, and 2676-3192, wherein the amino acid sequence comprises one or more substitutions relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide comprises a polynucleotide sequence encoding an engineered adenylate kinase comprising an amino acid sequence comprising at least a substitution at amino acid position 11, 13, 14, 15, 16, 18, 20, 21, 22, 26, 27, 29, 30, 31, 32, 34, 35, 36, 37, 38, 39, 40, 42, 43, 44, 46, 48, 50, 51, 52, 53, 54, 55, 56, 57, 58, 59, 60, 61, 62, 63, 64, 65, 66, 67, 68, 69, 70, 71, 73, 74, 75, 76, 77, 78, 79, 80, 81, 82, 83, 84, 87, 88, 89, 90, 91, 92, 93, 94, 97, 98, 99, 100, 101, 102, 104, 105, 108, 109, 110, 111, 112, 113, 115, 116, 117, 118, 119, 120, 122, 123, 124, 125, 126, 127, 128, 129, 131, 132, 133, 134, 135, 136, 137, 138, 139, 140, 141, 142, 143, 146, 148, 149, 150, 151, 152, 153, 154, 155, 156, 157, 161, 162, 163, 166, 168, 169, 170, 172, 173, 175, 178, 179, 180, 181, 182, 183, 184, 186, 187, 188, 190, 191, 192, 194, 195, 196, 197, 198, 199, 200, 201, 202, 203, 204, 205, 206, 207, 208, 210, 211, 212, 213, 214, 215, 216, 217, 218, 219, 220, 221, 222, 223, 224, 225, 226, 227, 228, 229, 230, or 231, or combinations thereof, wherein the amino acid positions are relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide comprises a polynucleotide sequence encoding an engineered adenylate kinase comprising an amino acid sequence comprising at least a substitution at amino acid position 13, 18, 29, 30, 31, 32, 34, 35, 48, 50, 53, 55, 59, 60, 61, 62, 66, 68, 77, 78, 80, 83, 87, 100, 104, 105, 109, 118, 119, 127, 128, 133, 136, 139, 142, 143, 150, 151, 155, 157, 168, 170, 175, 184, 186, 190, 201, 207, 212, 213, 214, 216, 217, 218, 224, or 226, or combinations thereof, wherein the amino acid positions are relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 4, or to the reference sequence corresponding to SEQ ID NO: 4, wherein the amino acid sequence comprises one or more substitutions relative to the reference sequence corresponding to residues 12-231 of SEQ ID NO: 4, or relative to the reference sequence corresponding to SEQ ID NO: 4.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 86-372, or to the reference sequence corresponding to an even-numbered SEQ ID NO. of SEQ ID NOs: 86-372, wherein the amino acid sequence comprises one or more substitutions relative to the reference sequence corresponding to residues 12-231 of SEQ ID NO: 4, or relative to the reference sequence corresponding to SEQ ID NO: 4.

In some embodiments, the recombinant polynucleotide comprises a polynucleotide sequence encoding an engineered adenylate kinase comprising an amino acid sequence comprising at least a substitution or substitution set at amino acid position(s) 100/101, 94, 179, 198, 57, 78, 68, 93, 77, 62, 14, 51, 118, 91, 163, 65, 173, 224, 66, 109, 60, 40/88, 191, 63, 68/170, 100, 108, 58, 180, 178, 199, 40, 80/203, 40/43, 39, 55, 110, 51/59, 155/226, 80, 219, 166, 202, 59, 38, 90, 80/184/203, 35, 222, 54, 111/153/155, 112, 175, 74, 105, 170, 117, 128, 61, 119, 155, 79, 220, 226, 108/155, 108/111, 111, 69/88, 43, 43/69/138, 40/43/88/134/178, 214, 80/82/184/219, 88/136, 16/40/43/88, 40/134, 88, 111/155, 223, 115, 190, 201, 126, 56, 216, 76, 69, 120, 116, 203, 194, or 138, wherein the amino acid sequence comprises one or more substitutions relative to the reference sequence corresponding to residues 12-231 of SEQ ID NO: 4, or relative to the reference sequence corresponding to SEQ ID NO: 4.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 172, or to the reference sequence corresponding to SEQ ID NO: 172, wherein the amino acid sequence comprises one or more substitutions relative to the reference sequence corresponding to residues 12-231 of SEQ ID NO: 172, or relative to the reference sequence corresponding to SEQ ID NO: 172.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 374-444, or to the reference sequence corresponding to an even-numbered SEQ ID NO. of SEQ ID NOs: 374-444, wherein the amino acid sequence comprises one or more substitutions relative to the reference sequence corresponding to residues 12-231 of SEQ ID NO: 172, or relative to the reference sequence corresponding to SEQ ID NO: 172.

In some embodiments, the recombinant polynucleotide comprises a polynucleotide sequence encoding an engineered adenylate kinase comprising an amino acid sequence comprising at least a substitution or substitution set at amino acid position(s) 66/78, 78/80/224, 78, 65/66, 78/224, 78/80/109, 40/60/94/226, 66/68, 78/80, 94/117/118/226, 68/109, 68, 94/179, 51/66/68/224, 94/117, 51/66/68/78/224, 66, 118, 93/94/180, 109, 68/224, 40/62/93, 93, 51/66, 94/117/118, 198/220, 93/117, 117/118, 93/198, 68/173, 40/117, 224, 40/118/226, 93/94, or 40/62/118, wherein the amino acid sequence comprises one or more substitutions relative to the reference sequence corresponding to residues 12-231 of SEQ ID NO: 172, or relative to the reference sequence corresponding to SEQ ID NO: 172.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 376, or to the reference sequence corresponding to SEQ ID NO: 376, wherein the amino acid sequence comprises one or more substitutions relative to the reference sequence corresponding to residues 12-231 of SEQ ID NO: 376, or relative to the reference sequence corresponding to SEQ ID NO: 376.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 446-510, or to the reference sequence corresponding to an even-numbered SEQ ID NO. of SEQ ID NOs: 446-510, wherein the amino acid sequence comprises one or more substitutions relative to the reference sequence corresponding to residues 12-231 of SEQ ID NO: 376, or relative to the reference sequence corresponding to SEQ ID NO: 376.

In some embodiments, the recombinant polynucleotide comprises a polynucleotide sequence encoding an engineered adenylate kinase comprising an amino acid sequence comprising at least a substitution or substitution set at amino acid position(s) 13, 60, 126, 104, 201, 59, 55, 133, 61, 53, 190, 57, 62, 181, 168, 54, 173, 170, 34, 183, 14, 60/62/124/170, 60/170, or 60/62, wherein the amino acid positions are relative to the reference sequence corresponding to residues 12-231 of SEQ ID NO: 376, or relative to the reference sequence corresponding to SEQ ID NO: 376.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 508, or to the reference sequence corresponding to SEQ ID NO: 508, wherein the amino acid sequence comprises one or more substitutions relative to the reference sequence corresponding to residues 12-231 of SEQ ID NO: 508, or relative to the reference sequence corresponding to SEQ ID NO: 508.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 512-580, or to the reference sequence corresponding to an even-numbered SEQ ID NO. of SEQ ID NOs: 512-580, wherein the amino acid sequence comprises one or more substitutions relative to the reference sequence corresponding to residues 12-231 of SEQ ID NO: 508, or relative to the reference sequence corresponding to SEQ ID NO: 508.

In some embodiments, the recombinant polynucleotide comprises a polynucleotide sequence encoding an engineered adenylate kinase comprising an amino acid sequence comprising at least a substitution or substitution set at amino acid position(s) 15, 15/68, 15/181, 13/15, 13/15/201, 54/55, 55/133, 55/133/197, 54/55/133/197, 68, 54/133/197, 54/197, 13/15/68, 34, 54, 133, 120, 34/133, 13/15/181, 55, 15/59, 68/181, 201, 181, 66, 15/59/181, 197, 13/15/59, 61, 34/61, 13, 34/69, or 163, wherein the amino acid positions are relative to the reference sequence corresponding to residues 12-231 of SEQ ID NO: 508, or relative to the reference sequence corresponding to SEQ ID NO: 508.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 524, or to the reference sequence corresponding to SEQ ID NO: 524, wherein the amino acid sequence comprises one or more substitutions relative to the reference sequence corresponding to residues 12-231 of SEQ ID NO: 524, or relative to the reference sequence corresponding to SEQ ID NO: 524.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 582-608, or to the reference sequence corresponding to an even-numbered SEQ ID NO. of SEQ ID NOs: 582-608, wherein the amino acid sequence comprises one or more substitutions relative to the reference sequence corresponding to residues 12-231 of SEQ ID NO: 524, or relative to the reference sequence corresponding to SEQ ID NO: 524.

In some embodiments, the recombinant polynucleotide comprises a polynucleotide sequence encoding an engineered adenylate kinase comprising an amino acid sequence comprising at least a substitution or substitution set at amino acid position(s) 120, 61/201, 34, 36, 105, 112, 31, or 146, wherein the amino acid positions are relative to the reference sequence corresponding to residues 12-231 of SEQ ID NO: 524, or relative to the reference sequence corresponding to SEQ ID NO: 524.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 584, or to the reference sequence corresponding to SEQ ID NO: 584, wherein the amino acid sequence comprises one or more substitutions relative to the reference sequence corresponding to residues 12-231 of SEQ ID NO: 584, or relative to the reference sequence corresponding to SEQ ID NO: 584.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 610-666, or to the reference sequence corresponding to an even-numbered SEQ ID NO. of SEQ ID NOs: 610-666, wherein the amino acid sequence comprises one or more substitutions relative to the reference sequence corresponding to residues 12-231 of SEQ ID NO: 584, or relative to the reference sequence corresponding to SEQ ID NO: 584.

In some embodiments, the recombinant polynucleotide comprises a polynucleotide sequence encoding an engineered adenylate kinase comprising an amino acid sequence comprising at least a substitution or substitution set at amino acid position(s) 13/30/31/53/109/119/168, 36/126/190, 126, 36/39/126, 126/190, 36/126, 36/190, 39/126, 13/109/119, 109, 36/120/190, 126/148, 190, 108/126, 30/31, 13/118/182, 31/53, 39, 119, 13/119/168, 53/168, 34, 34/65/146, 36/39, 119/168, 34/112, or 173, wherein the amino acid positions are relative to the reference sequence corresponding to residues 12-231 of SEQ ID NO: 584, or relative to the reference sequence corresponding to SEQ ID NO: 584.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 610, or to the reference sequence corresponding to SEQ ID NO: 610, wherein the amino acid sequence comprises one or more substitutions relative to the reference sequence corresponding to residues 12-231 of SEQ ID NO: 610, or relative to the reference sequence corresponding to SEQ ID NO: 610.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 668-780, or to the reference sequence corresponding to an even-numbered SEQ ID NO. of SEQ ID NOs: 668-780, wherein the amino acid sequence comprises one or more substitutions relative to the reference sequence corresponding to residues 12-231 of SEQ ID NO: 610, or relative to the reference sequence corresponding to SEQ ID NO: 610.

In some embodiments, the recombinant polynucleotide comprises a polynucleotide sequence encoding an engineered adenylate kinase comprising an amino acid sequence comprising at least a substitution or substitution set at amino acid position(s) 190, 115/126/190, 126/190, 34/35/105, 126, 59/126, 34/105/116/146, 48, 66, 59, 148, 82, 100, 27, 135, 77, 138, 52, 68, 139, 184, 81, 65, 153, 50, 181, 214, 172, 69, or 154, wherein the amino acid positions are relative to the reference sequence corresponding to residues 12-231 of SEQ ID NO: 610, or relative to the reference sequence corresponding to SEQ ID NO: 610.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 674, or to the reference sequence corresponding to SEQ ID NO: 674, wherein the amino acid sequence comprises one or more substitutions relative to the reference sequence corresponding to residues 12-231 of SEQ ID NO: 674, or relative to the reference sequence corresponding to SEQ ID NO: 674.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 782-868, or to the reference sequence corresponding to an even-numbered SEQ ID NO. of SEQ ID NOs: 782-868, wherein the amino acid sequence comprises one or more substitutions relative to the reference sequence corresponding to residues 12-231 of SEQ ID NO: 674, or relative to the reference sequence corresponding to SEQ ID NO: 674.

In some embodiments, the recombinant polynucleotide comprises a polynucleotide sequence encoding an engineered adenylate kinase comprising an amino acid sequence comprising at least a substitution or substitution set at amino acid position(s) 81/148/219, 68/148/219, 68/81/219, 148/219, 81/219, 136/138/139, 68/219, 66/68/219, 66/148/219, 66/68/148, 59, 59/135, 219, 59/139, 138, 126/148/219, 59/181, 81, 66/81/219, 126/219, 66/68/81/148/219, 181, 27/148, 135/136/139, 148, 59/135/138/181, 59/135/136/138/139, 59/135/136/138/181, 27, 27/126/148, 135/136/138, 68/81/126/148/219, 136, 68, 126/148, or 138/139, wherein the amino acid positions are relative to the reference sequence corresponding to residues 12-231 of SEQ ID NO: 674, or relative to the reference sequence corresponding to SEQ ID NO: 674.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 816, or to the reference sequence corresponding to SEQ ID NO: 816, wherein the amino acid sequence comprises one or more substitutions relative to the reference sequence corresponding to residues 12-231 of SEQ ID NO: 816, or relative to the reference sequence corresponding to SEQ ID NO: 816.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 870-1014, or to the reference sequence corresponding to an even-numbered SEQ ID NO. of SEQ ID NOs: 870-1014, wherein the amino acid sequence comprises one or more substitutions relative to the reference sequence corresponding to residues 12-231 of SEQ ID NO: 816, or relative to the reference sequence corresponding to SEQ ID NO: 816.

In some embodiments, the recombinant polynucleotide comprises a polynucleotide sequence encoding an engineered adenylate kinase comprising an amino acid sequence comprising at least a substitution or substitution set at amino acid position(s) 48/100/148, 48/66/148/219, 48/100, 48/100/135/136, 48/66/77/100/135/136/148, 48/100/136, 48/135/148, 48/148, 48/136/148, 48/135, 48/135/136, 66/100, 48/66/219, 48/135/219, 100/148, 48/136/219, 48/219, 48/135/148/219, 100/135/136, 100/136, 66/77/100/136/219, 100/135, 48/77/100/135/136, 100, 77/100/135, 48/66/100/135/136/148/219, 52/184, 66, 52/68, 136/148, 126/184, 148, 184, 52, 135/136/148, 126/138/184, 27/184, 27/126, 27/126/148, 27, 27/126/184, 66/100/135/219, 27/68, 52/68/81/126, 48/66/136/148, 66/136/148, 66/135, 66/136, 66/135/219, 136, 135/148/219, 68/126, 66/148/219, 27/68/126, 27/68/184, 126, 68/81/126, 77/135, 135/136, 77/136, 77/148, 82, 138/184, 77/135/136, 27/138, 81, 81/126, 27/126/138, 68, or 81/138, wherein the amino acid positions are relative to the reference sequence corresponding to residues 12-231 of SEQ ID NO: 816, or relative to the reference sequence corresponding to SEQ ID NO: 816.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 874, or to the reference sequence corresponding to SEQ ID NO: 874, wherein the amino acid sequence comprises one or more substitutions relative to the reference sequence corresponding to residues 12-231 of SEQ ID NO: 874, or relative to the reference sequence corresponding to SEQ ID NO: 874.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1016-1302, or to the reference sequence corresponding to an even-numbered SEQ ID NO. of SEQ ID NOs: 1016-1302, wherein the amino acid sequence comprises one or more substitutions relative to the reference sequence corresponding to residues 12-231 of SEQ ID NO: 874, or relative to the reference sequence corresponding to SEQ ID NO: 874.

In some embodiments, the recombinant polynucleotide comprises a polynucleotide sequence encoding an engineered adenylate kinase comprising an amino acid sequence comprising at least a substitution or substitution set at amino acid position(s) 32/60/135/175/225, 32/81/127/128/135/175/225, 83/175/225, 135/175/225, 54/84/190, 55/190/212, 32/60/81/83/127/128/135/225, 175/225, 32/127/175, 84/190/212, 32/128/175, 81/175, 21/54, 54/55/212, 32/175/225, 128/175/225, 62/84/190, 81/135/225, 21/54/62/190/212, 60/81/83/175, 55/84/190, 32/81/83/135/225, 175, 62/212, 32/81/83, 32/81/225, 54/55/84/212, 55/190, 62/179, 54/84/179/212, 54/55/62/84, 81/135, 32/81/135, 179/190/212, 55/62, 100, 21/54/62/190, 190/212, 128/175, 21/62/190, 54/179/190, 54/190, 21/55/190, 21/54/62, 21/84, 21/55, 21/62, 21/179, 55/62/190/212, 54/62/84, 127/128/175/225, 54/62, 100/104, 54/55, 21/55/212, 62, 55/84, 55/62/212, 21/84/190, 225, 21/54/179/190, 83/135, 32/175, 21/54/78/190, 55/62/179/190/212, 21/54/190, 21/55/84/190, 62/84, 21/55/62, 21/62/84, 135, 32/60/81/127, 21/54/55/62/78, 54/84, 55/62/179/190, 54/55/62/84/179/190/212, 190, 83, 78, 54/62/190, 21/55/62/179/190, 32/60/127, 54/62/84/190/212, 21/62/179, 127/128/225, 32/225, 32/60/81, 81, 54, 21, 60/81/128/175, 84, 21/190/212, 60/128/225, 179, 21/55/62/190, 128, 60, 212, 55, 127, 32, 80, 220, 62/78/179, 55/62/84, 21/55/190/212, 32/127/128/225, 21/54/55/84/179, 21/190, 32/60/81/83/128/135, 127/128, or 21/55/179, wherein the amino acid positions are relative to the reference sequence corresponding to residues 12-231 of SEQ ID NO: 874, or relative to the reference sequence corresponding to SEQ ID NO: 874.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1032, or to the reference sequence corresponding to SEQ ID NO: 1032, wherein the amino acid sequence comprises one or more substitutions relative to the reference sequence corresponding to residues 12-231 of SEQ ID NO: 1032, or relative to the reference sequence corresponding to SEQ ID NO: 1032.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1356-1552, or to the reference sequence corresponding to an even-numbered SEQ ID NO. of SEQ ID NOs: 1356-1552, wherein the amino acid sequence comprises one or more substitutions relative to the reference sequence corresponding to residues 12-231 of SEQ ID NO: 1032, or relative to the reference sequence corresponding to SEQ ID NO: 1032.

In some embodiments, the recombinant polynucleotide comprises a polynucleotide sequence encoding an engineered adenylate kinase comprising an amino acid sequence comprising at least a substitution or substitution set at amino acid position(s) 54/80/225, 55/80/180, 54/55/80/208/225, 84/128/155/190/212, 62/155/190, 83/84/155/190, 62/128/135/190, 225, 84/155/190, 128/190, 190/212, 83/84/155, 54/208/225, 84/155/190, 84/212, 84/123/155/212, 62/128/190, 55/80/208/225, 155/190, 83/84/190/212, 83/128/155/190/212, 55/180/225, 84/190/212, 54/80/180/208/225, 80/208, 135/155/190, 212, 80/180/208, 128/135/190/212, 128/155/212, 80/208/225, 83/212, 62/84/155/190, 54/179/225, 84, 83/84/190, 128/212, 54/55/80/208, 55, 62/128, 54/80/208/225, 62/83/84/190, 179/225, 190, 80/169/208, 62/128/212, 80, 62, 54/55/80/225, 54/80/180/208, 83/84/128/190/212, 62/83/190, 62/190, 80/179, 80/180, 54/80, 62/83/84/128/190/212, 83/84/135/212, 54/80/179/208, 62/84/128, 11/62/84/128/190/212, 80/179/208, 80/225, 62/84/128/190/212, 62/84/212, 80/179/180/208/225, 62/135, 55/208, 62/84, 128/135/190, 55/208/225, 84/128/190, 62/84/128/190, 62/135/212, 54/179/180/225, 62/84/135/212, 135, 54/55/225, 55/80/179/180, 84/135/190, 84/128, 54, 84/128/135, 54/179/208/225, 55/179/225, 128, 62/83/84/155/190, 84/135, 84/128/155, 55/179/180, 83/84/128/190, or 54/55/179/225, wherein the amino acid positions are relative to the reference sequence corresponding to residues 12-231 of SEQ ID NO: 1032, or relative to the reference sequence corresponding to SEQ ID NO: 1032.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1388, or to the reference sequence corresponding to SEQ ID NO: 1388, wherein the amino acid sequence comprises one or more substitutions relative to the reference sequence corresponding to residues 12-231 of SEQ ID NO: 1388, or relative to the reference sequence corresponding to SEQ ID NO: 1388.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1554-1786, or to the reference sequence corresponding to an even-numbered SEQ ID NO. of SEQ ID NOs: 1554-1786, wherein the amino acid sequence comprises one or more substitutions relative to the reference sequence corresponding to residues 12-231 of SEQ ID NO: 1388, or relative to the reference sequence corresponding to SEQ ID NO: 1388.

In some embodiments, the recombinant polynucleotide comprises a polynucleotide sequence encoding an engineered adenylate kinase comprising an amino acid sequence comprising at least a substitution or substitution set at amino acid position(s) 143, 228, 136, 82, 156, 205, 226, 150, 219, 217, 218, 68, 142, 212, 213, 79, 230, 207, 155, 227, 129, 210, 133, 151, 43, 204, 182, 82/136/150/217, 136/150, 79/81/143/156/212/228, 82/136, 79/81/143/212, 79/133/228, 81/143/156/212, 136/219, 82/136/226, 136/150/217, 133/156, 133/143/228, 150/217, 82/136/150, 81, 81/143, 81/133/228, 143/228, 212/228, 79/81, 133/228, 133/212, 136/217, 216, 215, or 127, wherein the amino acid positions are relative to the reference sequence corresponding to residues 12-231 of SEQ ID NO: 1388, or relative to the reference sequence corresponding to SEQ ID NO: 1388.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1708, or to the reference sequence corresponding to SEQ ID NO: 1708, wherein the amino acid sequence comprises one or more substitutions relative to the reference sequence corresponding to residues 12-231 of SEQ ID NO: 1708, or relative to the reference sequence corresponding to SEQ ID NO: 1708.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1788-1968, or to the reference sequence corresponding to an even-numbered SEQ ID NO. of SEQ ID NOs: 1788-1968, wherein the amino acid sequence comprises one or more substitutions relative to the reference sequence corresponding to residues 12-231 of SEQ ID NO: 1708, or relative to the reference sequence corresponding to SEQ ID NO: 1708.

In some embodiments, the recombinant polynucleotide comprises a polynucleotide sequence encoding an engineered adenylate kinase comprising an amino acid sequence comprising at least a substitution or substitution set at amino acid position(s) 127, 143/155/219/228, 219, 127/142, 142/151/207/218, 143/213, 151/218, 127/142/218, 142/151, 143/155/205/213/228, 127/142/207, 127/216/218, 142/151/207, 143/219, 127/207, 127/218, 143/219/228, 127/216, 68/127/207, 143/155/212/228, 151, 143/213/228, 143/155/205, 151/207/216, 143/228, 68/127/207/218, 143/155, 143, 205/213/219, 155/212/219/228, 205/219/228, 143/205, 142/207, 143/205/219, 143/212/213, 127/207/218, 142, 127/142/151/207/218, 143/205/219/228, 205/213/228, 68/151/207, 68/127/142/207, 68/127, 218, 127/142/196, 142/207/218, 68/142/216, 68/127/216/218, 68/127/218, 151/216/218, 143/212/228, 127/142/151, 151/207, 127/142/216, 129/207/218, 68/142/151/207, 68/142/151, 151/207/218, 228, 155/213, 127/142/151/218, 127/151, 155/228, 68/142/207, 80/151/207, 205/228, 68/142/207/216/218, 68/218, 68/207, 216/218, 68/142, 212, 205, or 142/207/216/218, wherein the amino acid positions are relative to the reference sequence corresponding to residues 12-231 of SEQ ID NO: 1708, or relative to the reference sequence corresponding to SEQ ID NO: 1708.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1952, or to the reference sequence corresponding to SEQ ID NO: 1952, wherein the amino acid sequence comprises one or more substitutions relative to the reference sequence corresponding to residues 12-231 of SEQ ID NO: 1952, or relative to the reference sequence corresponding to SEQ ID NO: 1952.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 1970-2032, or to the reference sequence corresponding to an even-numbered SEQ ID NO. of SEQ ID NOs: 1970-2032, wherein the amino acid sequence comprises one or more substitutions relative to the reference sequence corresponding to residues 12-231 of SEQ ID NO: 1952, or relative to the reference sequence corresponding to SEQ ID NO: 1952.

In some embodiments, the recombinant polynucleotide comprises a polynucleotide sequence encoding an engineered adenylate kinase comprising an amino acid sequence comprising at least a substitution or substitution set at amino acid position(s) 18/118, 87/119/192, 119/173/192, 118, 87/119, 18/118/170, 18, 119, 192, 87, 89, 59, 170, 64, 112, 93, 184, 146, 51, 125, 192, 191, 117, 172, or 163, wherein the amino acid positions are relative to the reference sequence corresponding to residues 12-231 of SEQ ID NO: 1952, or relative to the reference sequence corresponding to SEQ ID NO: 1952.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 1980, or to the reference sequence corresponding to SEQ ID NO: 1980, wherein the amino acid sequence comprises one or more substitutions relative to the reference sequence corresponding to residues 12-231 of SEQ ID NO: 1980, or relative to the reference sequence corresponding to SEQ ID NO: 1980.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2034-2134, or to the reference sequence corresponding to an even-numbered SEQ ID NO. of SEQ ID NOs: 2034-2134, wherein the amino acid sequence comprises one or more substitutions relative to the reference sequence corresponding to residues 12-231 of SEQ ID NO: 1980, or relative to the reference sequence corresponding to SEQ ID NO: 1980.

In some embodiments, the recombinant polynucleotide comprises a polynucleotide sequence encoding an engineered adenylate kinase comprising an amino acid sequence comprising at least a substitution or substitution set at amino acid position(s) 87/119/192, 87/192, 87/119, 119/192, 192, 119, 146/192, 87, 59/87, 59/87/112/146, 146, 87/112/119, 74, 75, 66, 181, 81, 73, 77, 188, 216, 82, 136, 214, 21, 217, 154, 133, 71, 212, or 228, wherein the amino acid positions are relative to the reference sequence corresponding to residues 12-231 of SEQ ID NO: 1980, or relative to the reference sequence corresponding to SEQ ID NO: 1980.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2072, or to the reference sequence corresponding to SEQ ID NO: 2072, wherein the amino acid sequence comprises one or more substitutions relative to the reference sequence corresponding to residues 12-231 of SEQ ID NO: 2072, or relative to the reference sequence corresponding to SEQ ID NO: 2072.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2136-2264, or to the reference sequence corresponding to an even-numbered SEQ ID NO. of SEQ ID NOs: 2136-2264, wherein the amino acid sequence comprises one or more substitutions relative to the reference sequence corresponding to residues 12-231 of SEQ ID NO: 2072, or relative to the reference sequence corresponding to SEQ ID NO: 2072.

In some embodiments, the recombinant polynucleotide comprises a polynucleotide sequence encoding an engineered adenylate kinase comprising an amino acid sequence comprising at least a substitution or substitution set at amino acid position(s) 87, 194, 197, 93, 202, 38, 91, 183, 39, 146, 203, 37, 92, 36, 89, 131, 132, 90, 148, 35/197, 231, or 56, wherein the amino acid positions are relative to the reference sequence corresponding to residues 12-231 of SEQ ID NO: 2072, or relative to the reference sequence corresponding to SEQ ID NO: 2072.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2138, or to the reference sequence corresponding to SEQ ID NO: 2138, wherein the amino acid sequence comprises one or more substitutions relative to the reference sequence corresponding to residues 12-231 of SEQ ID NO: 2138, or relative to the reference sequence corresponding to SEQ ID NO: 2138.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2266-2366, or to the reference sequence corresponding to an even-numbered SEQ ID NO. of SEQ ID NOs: 2266-2366, wherein the amino acid sequence comprises one or more substitutions relative to the reference sequence corresponding to residues 12-231 of SEQ ID NO: 2138, or relative to the reference sequence corresponding to SEQ ID NO: 2138.

In some embodiments, the recombinant polynucleotide comprises a polynucleotide sequence encoding an engineered adenylate kinase comprising an amino acid sequence comprising at least a substitution or substitution set at amino acid position(s) 161, 162, 153, 216, 212, 156, 214, 229, 77/143/214/216, 143/156/216, 77/143, 77/143/216, 77/214/216, 77/216, 143/156, 156/216, 143, 77, 77/214, 77/143/161/162/212/214, 77/162/214/216, 77/143/156/214/216, 77/143/156/162/212/216, 77/143/156/161/214/216, 77/156/162/216, 156/161/214/216, 77/143/162/214/216, 143/216, 214/216, 143/156/214/216, 143/214/216, 77/143/212/216, 143/156/161/162/216, 156/162/214, 77/143/156/161/162, 143/161/214/216, 77/161/216, or 143/212/214/216, wherein the amino acid positions are relative to the reference sequence corresponding to residues 12-231 of SEQ ID NO: 2138, or relative to the reference sequence corresponding to SEQ ID NO: 2138.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2294, or to the reference sequence corresponding to SEQ ID NO: 2294, wherein the amino acid sequence comprises one or more substitutions relative to the reference sequence corresponding to residues 12-231 of SEQ ID NO: 2294, or relative to the reference sequence corresponding to SEQ ID NO: 2294.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2368-2594, or to the reference sequence corresponding to an even-numbered SEQ ID NO. of SEQ ID NOs: 2368-2594, wherein the amino acid sequence comprises one or more substitutions relative to the reference sequence corresponding to residues 12-231 of SEQ ID NO: 2294, or relative to the reference sequence corresponding to SEQ ID NO: 2294.

In some embodiments, the recombinant polynucleotide comprises a polynucleotide sequence encoding an engineered adenylate kinase comprising an amino acid sequence comprising at least a substitution at amino acid position 80, 105, 228, 50, 175, 170, 82, 173, 65, 53, 184, 122, 195, 68, 89, 34, 118, 119, 87, 179, 117, 113, 54, 190, 216, 88, 36, 166, 38, 169, 66, 142, 136, 131, 187, 40, 74, 127, 55, 148, 215, 64, 155, 116, 90, 132, 60, 94, 112, 120, 37, 108, 35, 51, 61, 212, 172, 59, or 56, or combinations thereof, wherein the amino acid positions are relative to the reference sequence corresponding to residues 12-231 of SEQ ID NO: 2294, or relative to the reference sequence corresponding to SEQ ID NO: 2294.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2368, or to the reference sequence corresponding to SEQ ID NO: 2368, wherein the amino acid sequence comprises one or more substitutions relative to the reference sequence corresponding to residues 12-231 of SEQ ID NO: 2368, or relative to the reference sequence corresponding to SEQ ID NO: 2368.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2596-2652 or 2676-2714, or to the reference sequence corresponding to an even-numbered SEQ ID NO. of SEQ ID NOs: 2596-2652 or 2676-2714, wherein the amino acid sequence comprises one or more substitutions relative to the reference sequence corresponding to residues 12-231 of SEQ ID NO: 2368, or relative to the reference sequence corresponding to SEQ ID NO: 2368.

In some embodiments, the recombinant polynucleotide comprises a polynucleotide sequence encoding an engineered adenylate kinase comprising an amino acid sequence comprising at least a substitution or substitution set at amino acid position(s) 20, 21, 22, 43, 44, 46, 48, 138, 181, 182, 136/228, 82/142/173/184/216, 184/216, 50/184/216, 127/173/184, 105/136/170/175/228, 105/175, 50/184, 105/170/175/228, 50/127/173/184, 50/142/184/216, 50/82/127/216, 50, 50/82/173/216, 50/127/142/184/216, 50/82/127, 105/136, 105/228, 105/136/175/228, 173/216, 50/173, 228, 127/173, 105/136/170/195, 50/82/216, 175/228, or 50/82/127/142/184/216, wherein the amino acid positions are relative to the reference sequence corresponding to residues 12-231 of SEQ ID NO: 2368, or relative to the reference sequence corresponding to SEQ ID NO: 2368.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2602, or to the reference sequence corresponding to SEQ ID NO: 2602, wherein the amino acid sequence comprises one or more substitutions relative to the reference sequence corresponding to residues 12-231 of SEQ ID NO: 2602, or relative to the reference sequence corresponding to SEQ ID NO: 2602.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2716-2988, or to the reference sequence corresponding to an even-numbered SEQ ID NO. of SEQ ID NOs: 2716-2988, wherein the amino acid sequence comprises one or more substitutions relative to the reference sequence corresponding to residues 12-231 of SEQ ID NO: 2602, or relative to the reference sequence corresponding to SEQ ID NO: 2602.

In some embodiments, the recombinant polynucleotide comprises a polynucleotide sequence encoding an engineered adenylate kinase comprising at least a substitution or substitution set at amino acid position(s) 18, 18/56, 20, 21, 26, 27, 29, 30, 32, 42, 43, 50, 66, 67, 69, 70, 80, 98, 99, 102, 135, 137, 139, 141, 142, 143, 148, 149, 152, 153, 156, 173, 184, or 216, wherein the amino acid positions are relative to the reference sequence corresponding to residues 12-231 of SEQ ID NO: 2602, or relative to the reference sequence corresponding to SEQ ID NO: 2602.

In some embodiments, the recombinant polynucleotide comprises a polynucleotide sequence encoding an engineered adenylate kinase comprising at least a substitution or substitution set at amino acid position(s) 65, 65/104, 65/104/105/116/127, 65/104/105/127/136, 65/104/136, 65/104/136/170, 65/105/116/136/170, 65/105/127/136, 65/105/127/136/175, 65/105/127/170, 65/105/127/175, 65/105/136, 65/105/136/170, 65/105/170, 65/116, 65/127/136, 65/127/136/170, 65/127/170, 94/173, 94/187, 104/105, 104/105/127, 104/105/127/136/175/195, 104/105/136/170, 104/127, 104/127/136/170, 104/127/136/175, 105, 105/116/127/136/170/175, 105/127/136, 105/127/136/195, 105/136, 105/136/195, 116/127/136/175, 127/195, 132, 136, or 170, wherein the amino acid positions are relative to the reference sequence corresponding to residues 12-231 of SEQ ID NO: 2602, or relative to the reference sequence corresponding to SEQ ID NO: 2602.

In some embodiments, the recombinant polynucleotide comprises a polynucleotide sequence encoding an engineered adenylate kinase comprising at least a substitution at amino acid position 36, 37, 40, 50, 59, 89, 117, 120, 128, 168, or 203, or combinations thereof, wherein the amino acid positions are relative to the reference sequence corresponding to residues 12-231 of SEQ ID NO: 2602, or relative to the reference sequence corresponding to SEQ ID NO: 2602.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2832, or to the reference sequence corresponding to SEQ ID NO: 2832, wherein the amino acid sequence comprises one or more substitutions relative to the reference sequence corresponding to residues 12-231 of SEQ ID NO: 2832, or relative to the reference sequence corresponding to SEQ ID NO: 2832.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 2990-3032, or to the reference sequence corresponding to an even-numbered SEQ ID NO. of SEQ ID NOs: 2990-3032, wherein the amino acid sequence comprises one or more substitutions relative to the reference sequence corresponding to residues 12-231 of SEQ ID NO: 2832, or relative to the reference sequence corresponding to SEQ ID NO: 2832.

In some embodiments, the recombinant polynucleotide comprises a polynucleotide sequence encoding an engineered adenylate kinase comprising at least a substitution or substitution set at amino acid position(s) 36, 36/50, 36/50/89/93/110, 36/50/89/93/139, 36/50/89/139/170, 36/50/89/172, 36/89, 36/170, 50, 50/89/93, 50/89/93/139, 50/93, 50/170, 89/170/172, 90/104, 90/151/157, 104/151/154, 104/151/154/157, 104/154/157/216, 151/157, 151/216, or 216, wherein the amino acid positions are relative to the reference sequence corresponding to residues 12-231 of SEQ ID NO: 2832, or relative to the reference sequence corresponding to SEQ ID NO: 2832.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 2994, or to the reference sequence corresponding to SEQ ID NO: 2994, wherein the amino acid sequence comprises one or more substitutions relative to the reference sequence corresponding to residues 12-231 of SEQ ID NO: 2994, or relative to the reference sequence corresponding to SEQ ID NO: 2994.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 3034-3150, or to the reference sequence corresponding to an even-numbered SEQ ID NO. of SEQ ID NOs: 3034-3150, wherein the amino acid sequence comprises one or more substitutions relative to the reference sequence corresponding to residues 12-231 of SEQ ID NO: 2994, or relative to the reference sequence corresponding to SEQ ID NO: 2994.

In some embodiments, the recombinant polynucleotide comprises a polynucleotide sequence encoding an engineered adenylate kinase comprising at least a substitution or substitution set at amino acid position(s) 50, 58, 59, 68, 74, 76, 79, 80, 83, 90, 112, 113, 119, 157, 170, 172, 182, 184, 217, 224, 226, 228, 229, 230, or 231, wherein the amino acid positions are relative to the reference sequence corresponding to residues 12-231 of SEQ ID NO: 2994, or relative to the reference sequence corresponding to SEQ ID NO: 2994.

In some embodiments, the recombinant polynucleotide comprises a polynucleotide sequence encoding an engineered adenylate kinase comprising at least a substitution or substitution set at amino acid position(s) 79/104/157/228, 79/157/228/229, 79/157/229, 79/228, 83/104, 83/104/151/168/173/190, 83/113, 83/173/190, 83/173/190/201, 83/173/201, 83/190, 83/190/201/216, 83/216, 104/157, 157/173/190/216, 157/183, 157/190, 157/190/216, 157/228, 173/216, or 201/216, wherein the amino acid positions are relative to the reference sequence corresponding to residues 12-231 of SEQ ID NO: 2994, or relative to the reference sequence corresponding to SEQ ID NO: 2994.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 3104, or to the reference sequence corresponding to SEQ ID NO: 3104, wherein the amino acid sequence comprises one or more substitutions relative to the reference sequence corresponding to residues 12-231 of SEQ ID NO: 3104, or relative to the reference sequence corresponding to SEQ ID NO: 3104.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 3152-3192, or to the reference sequence corresponding to an even-numbered SEQ ID NO. of SEQ ID NOs: 3152-3192, wherein the amino acid sequence comprises one or more substitutions relative to the reference sequence corresponding to residues 12-231 of SEQ ID NO: 3104, or relative to the reference sequence corresponding to SEQ ID NO: 3104.

In some embodiments, the recombinant polynucleotide comprises a polynucleotide sequence encoding an engineered adenylate kinase comprising an amino acid sequence of the engineered adenylate kinase comprises at least a substitution or substitution set at amino acid position(s) 104, 104/157, 104/157/190/228, 104/170/228, 104/157/228/229, 104/157/229, 104/170/190, 104/170/190/228, 104/190, 104/190/201, 104/190/228, 104/201/228, 104/228/229, 157/228, 157/228/229, 170/190/228, 170/190/228/229, 190, 190/229, 201/228, or 228, wherein the amino acid positions are relative to the reference sequence corresponding to residues 12-231 of SEQ ID NO: 3104, or relative to the reference sequence corresponding to SEQ ID NO: 3104.

In some embodiments, the recombinant polynucleotide comprises a polynucleotide sequence encoding an engineered adenylate kinase comprising an amino acid sequence comprising at least a substitution at an amino acid position set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide comprises a polynucleotide sequence encoding an engineered adenylate kinase comprising an amino acid sequence comprising at least one substitution set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide comprises a polynucleotide sequence encoding an engineered adenylate kinase comprising an amino acid sequence comprising at least a substitution or substitution set at amino acid position(s) set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide comprises a polynucleotide sequence encoding an engineered adenylate kinase comprising an amino acid sequence comprising at least a substitution or substitution set of an engineered adenylate kinase variant set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide comprises a polynucleotide sequence encoding an engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference amino acid sequence comprising a substitution or substitution set of an engineered adenylate kinase variant set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, wherein the amino acid positions are relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide comprises a polynucleotide sequence encoding an engineered adenylate kinase comprising an amino acid sequence comprising residues 12-231 of an even-numbered SEQ ID NO. of SEQ ID NOs: 4-1302, 1356-2652, and 2676-3192, or comprising an even-numbered SEQ ID NO. of SEQ ID NOs: 4-1302, 1356-2652, and 2676-3192.

In some embodiments, the recombinant polynucleotide comprises a polynucleotide sequence encoding an engineered adenylate kinase comprising an amino acid sequence comprising residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or wherein the amino acid sequence comprises SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to nucleotide residues 34-693 of SEQ ID NO: 1, 3, 171, 375, 507, 523, 583, 609, 673, 815, 873, 1031, 1387, 1707, 1951, 1979, 2071, 2137, 2293, 2367, 2601, 2831, 2993, or3103, or to a reference polynucleotide sequence corresponding to SEQ ID NO: 1, 3, 171, 375, 507, 523, 583, 609, 673, 815, 873, 1031, 1387, 1707, 1951, 1979, 2071, 2137, 2293, 2367, 2601, 2831, 2993, or 3103, wherein the recombinant polynucleotide encodes an adenylate kinase.

In some embodiments, the recombinant polynucleotide comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference polynucleotide sequence corresponding to nucleotide residues 34-693 of an odd-numbered SEQ ID NO. of SEQ ID NOs: 3-1301, 1355-2651, and 2675-3191, or to a reference polynucleotide sequence corresponding to an odd-numbered SEQ ID NO. of SEQ ID NOs: 3-1301, 1355-2651, and 2675-3191, wherein the recombinant polynucleotide encodes an engineered adenylate kinase.

In some embodiments, the recombinant polynucleotide comprises a polynucleotide sequence comprising nucleotide residues 34-693 of SEQ ID NO: 1, 3, 171, 375, 507, 523, 583, 609, 673, 815, 873, 1031, 1387, 1707, 1951, 1979, 2071, 2137, 2293, 2367, 2601, 2831, 2993, or 3103, or comprising SEQ ID NO: 1, 3, 171, 375, 507, 523, 583, 609, 673, 815, 873, 1031, 1387, 1707, 1951, 1979, 2071, 2137, 2293, 2367, 2601, 2831, 2993, or 3103.

In some embodiments, the recombinant polynucleotide comprises a polynucleotide sequence comprising nucleotide residues 34-693 of an odd-numbered SEQ ID NO. of SEQ ID NOs: 3-1301, 1355-2651, and 2675-3191, or comprising an odd-numbered SEQ ID NO. of SEQ ID NOs: 3-1301, 1355-2651, and 2675-3191.

In some embodiments, the present disclosure provides a recombinant polynucleotide capable of hybridizing under highly stringent conditions to a reference polynucleotide encoding an engineered adenylate kinase polypeptide described herein, e.g., a recombinant polynucleotide provided in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, or a reverse complement thereof. In some embodiments, the recombinant polynucleotide hybridizes under highly stringent conditions to a reference polynucleotide corresponding to nucleotide residues 34-693 of SEQ ID NO: 3, 171, 375, 507, 523, 583, 609, 673, 815, or 873, 1031, 1387, 1707, 1951, 1979, 2071, 2137, 2293, 2367, 2601, 2831, 2993, or 3103, or to a reference polynucleotide sequence corresponding to SEQ ID NO: 3, 171, 375, 507, 523, 583, 609, 673, 815, or 873, 1031, 1387, 1707, 1951, 1979, 2071, 2137, 2293, 2367, 2601, 2831, 2993, or 3103, orareverse complement thereof. In some embodiments, the recombinant polynucleotide hybridizes under highly stringent conditions to a reference polynucleotide corresponding to nucleotide residues 34-693 of an odd-numbered SEQ ID NO. of SEQ ID NOs: 3-1301, 1355-2651, and 2675-3191, or corresponding to an odd-numbered SEQ ID NO. of SEQ ID NOs: 3-1301, 1355-2651, and 2675-3191, or a reverse complement thereof.

In some embodiments, the present disclosure provides a recombinant polynucleotide capable of hybridizing under highly stringent conditions to a reverse complement of a reference polynucleotide encoding an engineered adenylate kinase polypeptide described herein, wherein the recombinant polynucleotide hybridizing under stringent conditions encodes an adenylate kinase polypeptide comprising an amino acid sequence having one or more amino acid differences as compared to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, at residue positions selected from any positions as set forth in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2. In some embodiments, the recombinant polynucleotide that hybridizes under highly stringent conditions to a reverse complement of a reference polynucleotide encoding an engineered adenylate kinase polypeptide described herein encodes an adenylate kinase polypeptide having one or more amino acid differences present in an engineered adenylate kinase having an amino acid sequence corresponding to residues 12-231 of an even-numbered SEQ ID NO. of SEQ ID NOs: 3-1301, 1355-2651, and 2675-3191, or an amino acid sequence corresponding to an even-numbered SEQ ID NO. of SEQ ID NOs: 3-1301, 1355-2651, and 2675-3191, wherein the amino acid differences are relative to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

In some embodiments, the recombinant polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 34-693 of SEQ ID NO: 3, 171, 375, 507, 523, 583, 609, 673, 815, or 873, 1031, 1387, 1707, 1951, 1979, 2071, 2137, 2293, 2367, 2601, 2831, 2993, or 3103, or to a reference polynucleotide sequence corresponding to SEQ ID NO: 3, 171, 375, 507, 523, 583, 609, 673, 815, or 873, 1031, 1387, 1707, 1951, 1979, 2071, 2137, 2293, 2367, 2601, 2831, 2993, or 3103, or a reverse complement thereof. In some embodiments, the recombinant polynucleotide that hybridizes under highly stringent conditions comprises a polynucleotide sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reference polynucleotide sequence corresponding to nucleotide residues 34-693 of an odd-numbered SEQ ID NO. of SEQ ID NOs: 3-1301, 1355-2651, and 2675-3191, or an odd-numbered SEQ ID NO. of SEQ ID NOs: 3-1301, 1355-2651, and 2675-3191, or a reverse complement thereof.

In some additional embodiments, the polynucleotide hybridizing under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reverse complement of a polynucleotide reference sequence corresponding to nucleotide residues 34-693 of SEQ ID NO: 3, 171, 375, 507, 523, 583, 609, 673, 815, or 873, 1031, 1387, 1707, 1951, 1979, 2071, 2137, 2293, 2367, 2601, 2831, 2993, or 3103, or to a reference polynucleotide sequence corresponding to SEQ ID NO: 3, 171, 375, 507, 523, 583, 609, 673, 815, or 873, 1031, 1387, 1707, 1951, 1979, 2071, 2137, 2293, 2367, 2601, 2831, 2993, or 3103 encodes an engineered adenylate kinase polypeptide. In some additional embodiments, the polynucleotide hybridizing under highly stringent conditions comprises a polynucleotide sequence having at least 75%, 80%, 81%, 82%, 83%, 84%, 85%, 86%, 87%, 88%, 89%, 90%, 91%, 92%, 93%, 94%, 95%, 96%, 97%, 98%, 99% or more sequence identity to a reverse complement of a polynucleotide reference sequence corresponding to nucleotide residues 34-693 of an odd-numbered SEQ ID NO. of SEQ ID NOs: 3-1301, 1355-2651, and 2675-3191, or an odd-numbered SEQ ID NO. of SEQ ID NOs: 3-1301, 1355-2651, and 2675-3191 encodes an engineered adenylate kinase polypeptide.

In some embodiments, the recombinant polynucleotide comprises a polynucleotide sequence encoding an adenylate kinase comprising an amino acid sequence comprising
(a) residues 12 to 228 of SEQ ID NO: 1336,
   residues 12 to 228 of SEQ ID NO: 1338,
   residues 12 to 225 of SEQ ID NO: 1340, or
   residues 12 to 228 of SEQ ID NO: 1342; or
(b) SEQ ID NO: 1336;
   SEQ ID NO: 1338;
   SEQ ID NO: 1340; or
   SEQ ID NO: 1342.

In some embodiments, the recombinant polynucleotide comprises a polynucleotide sequence comprising
(a) nucleotide residues 34 to 684 of SEQ ID NO: 1335;
   nucleotide residues 34 to 684 of SEQ ID NO: 1337;
   nucleotide residues 34 to 675 of SEQ ID NO: 1339; or
   nucleotide residues 34 to 684 of SEQ ID NO: 1341; or
(b) SEQ ID NO: 1335;
   SEQ ID NO: 1337;
   SEQ ID NO: 1339; or
   SEQ ID NO: 1341.

In some embodiments, a recombinant polynucleotide encoding any of the adenylate kinase provided herein is manipulated in a variety of ways to provide for expression of the polypeptide. In some embodiments, the recombinant polynucleotide encoding the polypeptides are provided as expression vectors where one or more control sequences is operably linked to the recombinant polynucleotide to regulate the expression of the polynucleotide and/or encoded polypeptide. The techniques for modifying polynucleotides and nucleic acid sequences utilizing recombinant DNA methods are known in the art.

In some embodiments, the control sequences include, among others, promoter sequences, Kozak sequence, leader sequences, polyadenylation sequences, pro-peptide sequences, signal peptide sequences, regulatory elements, and transcription terminators. As known in the art, suitable promoters can be selected based on the host cells used. For bacterial host cells, suitable promoters for directing transcription of the nucleic acid constructs of the present disclosure, include, but are not limited to promoters obtained from the E. coli lac operon, Streptomyces coelicolor agarase gene (dagA), Bacillus subtilis levansucrase gene (sacB), Bacillus licheniformis alpha-amylase gene (amyL), Bacillus stearothermophilus maltogenic amylase gene (amyM), Bacillus amyloliquefaciens alpha-amylase gene (amyQ), Bacillus licheniformis penicillinase gene (penP), Bacillus subtilis xylA and xylB genes, and prokaryotic beta-lactamase gene (See e.g., Villa-Kamaroff et al., Proc. Natl Acad. Sci. USA, 1978, 75:3727-3731), as well as the tac promoter (See e.g., DeBoer et al., Proc. Natl Acad. Sci. USA, 1983, 80: 21-25). Exemplary promoters for filamentous fungal host cells, include, but are not limited to promoters obtained from the genes for Aspergillus oryzae TAKA amylase, Rhizomucor miehei aspartic proteinase, Aspergillus niger neutral alpha-amylase, Aspergillus niger acid stable alpha-amylase, Aspergillus niger or Aspergillus awamori glucoamylase (glaA), Rhizomucor miehei lipase, Aspergillus oryzae alkaline protease, Aspergillus oryzae triose phosphate isomerase, Aspergillus nidulans acetamidase, and Fusarium oxysporum trypsin-like protease (See e.g., WO 96/00787), as well as the NA2-tpi promoter (a hybrid of the promoters from the genes for Aspergillus niger neutral alpha-amylase and Aspergillus oryzae triose phosphate isomerase), and mutant, truncated, and hybrid promoters thereof. Exemplary yeast cell promoters can be from the genes can be from the genes for Saccharomyces cerevisiae enolase (ENO-1), Saccharomyces cerevisiae galactokinase (GAL1), Saccharomyces cerevisiae alcohol dehydrogenase/glyceraldehyde-3-phosphate dehydrogenase (ADH2/GAP), and Saccharomyces cerevisiae 3-phosphoglycerate kinase. In some embodiments, promoters effective in Pichia cells are used. Other useful promoters for yeast host cells are known in the art (See e.g., Romanos et al., Yeast, 1992, 8:423-488). In some embodiments, for insect host cells, suitable promoters include, among others, baculovirus promoters (e.g., P10 and polyhedron promoters), OpIE2 promoter, and Nephotettix cincticeps actin promoters. In some embodiments, promoters effective in Pichia cells are used. In some embodiments, for insect host cells, suitable promoters include, among others, baculovirus promoters (e.g., P10 and polyhedron promoters), OpIE2 promoter, and Nephotettix cincticeps actin promoters. In some embodiments, for mammalian host cells, suitable promoters include, among others, promoters of cytomegalovirus (CMV), chicken β-actin promoter fused with the CMV enhancer, simian virus 40 (SV40), human phosphoglycerate kinase, beta actin, elongation factor-la or glyceraldehyde-3-phosphate dehydrogenase, or Gallus β-actin.

In some embodiments, the control sequence is also a suitable transcription terminator sequence (i.e., a sequence recognized by a host cell to terminate transcription). In some embodiments, the terminator sequence is operably linked to the 3′ terminus of the nucleic acid sequence encoding the leucine decarboxylase polypeptide. Any suitable terminator which is functional in the host cell of choice finds use in the present invention. For bacterial expression, the transcription terminators can be a Rho-dependent terminators that rely on a Rho transcription factor, or a Rho-independent, or intrinsic terminators, which do not require a transcription factor. Exemplary bacterial transcription terminators are described in Peters et al., J Mol Biol., 2011, 412(5):793-813. Exemplary transcription terminators for filamentous fungal host cells can be obtained from the genes for Aspergillus oryzae TAKA amylase, Aspergillus niger glucoamylase, Aspergillus nidulans anthranilate synthase, Aspergillus niger alpha-glucosidase, and Fusarium oxysporum trypsin-like protease. Exemplary terminators for yeast host cells can be obtained from the genes for Saccharomyces cerevisiae enolase, Saccharomyces cerevisiae cytochrome C (CYC1), and Saccharomyces cerevisiae glyceraldehyde-3-phosphate dehydrogenase. Other useful terminators for yeast host cells are known in the art (See e.g., Romanos et al., supra). Exemplary terminators for mammalian cells include, but are not limited to those from cytomegalovirus (CMV), Simian virus 40 (SV40), from Homo sapiens growth hormone hGH, from bovine growth hormone BGH, and from human or rabbit beta globulin.

In some embodiments, the control sequence is also a suitable leader sequence (i.e., a non-translated region of an mRNA that is important for translation by the host cell). In some embodiments, the leader sequence is operably linked to the 5′ terminus of the nucleic acid sequence encoding the adenylate kinase polypeptide. Any suitable leader sequence that is functional in the host cell of choice find use in the present invention. Exemplary leaders for filamentous fungal host cells are obtained from the genes for Aspergillus oryzae TAKA amylase, and Aspergillus nidulans triose phosphate isomerase. Suitable leaders for yeast host cells are obtained from the genes for Saccharomyces cerevisiae enolase (ENO-1), Saccharomyces cerevisiae 3-phosphoglycerate kinase, Saccharomyces cerevisiae alpha-factor, and Saccharomyces cerevisiae alcohol dehydrogenase/glyceraldehyde-3-phosphate dehydrogenase (ADH2/GAP). Suitable leaders for mammalian host cells include but are not limited to the 5′-UTR element present in orthopoxvirus mRNA.

In some embodiments, the control sequence comprises a 3′ untranslated nucleic acid region and polyadenylation tail nucleic acid sequence, sequences operably linked to the 3′ terminus of the protein coding nucleic acid sequence which mediate binding to proteins involved in mRNA trafficking and translation and mRNA half-life. Any polyadenylation sequence and 3′ UTR which is functional in the host cell of choice may be used in the present invention. Exemplary polyadenylation sequences for filamentous fungal host cells include, but are not limited to those from the genes for Aspergillus oryzae TAKA amylase, Aspergillus niger glucoamylase, Aspergillus nidulans anthranilate synthase, Fusarium oxysporum trypsin-like protease, and Aspergillus niger alpha-glucosidase. Useful polyadenylation sequences for yeast host cells are also known in the art (See e.g., Guo and Sherman, Mol. Cell. Biol., 1995, 15:5983-5990). Useful polyadenylation and 3′ UTR sequences for mammalian host cells include, but are not limited to, the 3′-UTRs of α- and β-globin mRNAs that harbor several sequence elements that increase the stability and translation of mRNA.

In some embodiments, the control sequence is a signal peptide (i.e., a coding region that codes for an amino acid sequence linked to the amino terminus of a polypeptide and directs the encoded polypeptide into the cell's secretory pathway). In some embodiments, the 5′ end of the coding sequence of the nucleic acid sequence inherently contains a signal peptide coding region naturally linked in translation reading frame with the segment of the coding region that encodes the secreted polypeptide. Alternatively, in some embodiments, the 5′ end of the coding sequence contains a signal peptide coding region that is foreign to the coding sequence. Any suitable signal peptide coding region which directs the expressed polypeptide into the secretory pathway of a host cell of choice finds use for expression of the engineered polypeptide(s). Effective signal peptide coding regions for bacterial host cells are the signal peptide coding regions include, but are not limited to those obtained from the genes for Bacillus NCIB 11837 maltogenic amylase, Bacillus stearothermophilus alpha-amylase, Bacillus licheniformis subtilisin, Bacillus licheniformis beta-lactamase, Bacillus stearothermophilus neutral proteases (nprT, nprS, nprM), and Bacillus subtilis prsA. Further signal peptides are known in the art (See e.g., Simonen and Palva, Microbiol. Rev., 1993, 57:109-137). In some embodiments, effective signal peptide coding regions for filamentous fungal host cells include, but are not limited to the signal peptide coding regions obtained from the genes for Aspergillus oryzae TAKA amylase, Aspergillus niger neutral amylase, Aspergillus niger glucoamylase, Rhizomucor miehei aspartic proteinase, Humicola insolens cellulase, and Humicola lanuginosa lipase. Useful signal peptides for yeast host cells include, but are not limited to those from the genes for Saccharomyces cerevisiae alpha-factor and Saccharomyces cerevisiae invertase. Useful signal peptides for mammalian host cells include but are not limited to, those from the genes for immunoglobulin gamma (IgG) and the signal peptide in a human secreted protein, such as human beta-galactosidase polypeptide.

In some embodiments, the control sequence is a regulatory sequence that facilitates the regulation of the expression of the recombinant polynucleotide and/or encoded polypeptide. Examples of regulatory systems are those that cause the expression of the gene to be turned on or off in response to a chemical or physical stimulus, including the presence of a regulatory compound. In prokaryotic host cells, suitable regulatory sequences include, but are not limited to the lac, tac, and trp operator systems. In yeast host cells, suitable regulatory systems include, but are not limited to the ADH2 system or GAL1 system. In filamentous fungi, suitable regulatory sequences include, but are not limited to the TAKA alpha-amylase promoter, Aspergillus niger glucoamylase promoter, and Aspergillus oryzae glucoamylase promoter. In mammalian cells, suitable regulatory systems include, among others, zinc-inducible sheep metallothionine (MT) promoter, dexamethasone (Dex)-inducible promoter, mouse mammary tumor virus (MMTV) promoter; ecdysone insect promoter, tetracycline-inducible promoter system, RU486-inducible promoter system, and the rapamycin-inducible promoter system.

In another aspect, the present disclosure provides an expression vector comprising a recombinant polynucleotide encoding an adenylate kinase polypeptide, where the recombinant polynucleotide is operably or operatively linked to a control sequence, such as a promoter and a terminator, a replication origin, etc., depending on the type of hosts into which they are to be introduced. The recombinant expression vector may be any suitable vector (e.g., a plasmid or virus), that can be conveniently subjected to recombinant DNA procedures and bring about the expression of the adenylate kinase polynucleotide sequence. The choice of the vector typically depends on the compatibility of the vector with the host cell into which the vector is to be introduced. The vectors may be linear or closed circular plasmids.

In some embodiments, the expression vector is an autonomously replicating vector (i.e., a vector that exists as an extra-chromosomal entity, the replication of which is independent of chromosomal replication, such as a plasmid, an extra-chromosomal element, a minichromosome, or an artificial chromosome). The vector may contain any means for assuring self-replication. In some alternative embodiments, the vector may be one which, when introduced into the host cell, is integrated into the genome and replicated together with the chromosome(s) into which it has been integrated. Furthermore, a single vector or plasmid or two or more vectors or plasmids which together contain the total DNA to be introduced into the genome of the host cell, or a transposon may be used.

In some embodiment, recombinant polynucleotides may be provided on a non-replicating expression vector or plasmid. In some embodiments, the non-replicating expression vector or plasmid can be based on viral vectors defective in replication (see, e.g., Travieso et al., npj Vaccines, 2022, Vol. 7, Article 75).

In some embodiments, the expression vector contains one or more selectable markers, which permit easy selection of transformed cells. A “selectable marker” is a gene, the product of which provides for biocide or viral resistance, resistance to heavy metals, prototrophy to auxotrophs, and the like. Examples of bacterial selectable markers include, but are not limited to the dal genes from Bacillus subtilis or Bacillus licheniformis, or markers, which confer antibiotic resistance such as ampicillin, kanamycin, chloramphenicol or tetracycline resistance. Suitable markers for yeast host cells include, but are not limited to ADE2, HIS3, LEU2, LYS2, MET3, TRP1, and URA3. Selectable markers for use in filamentous fungal host cells include, but are not limited to, amdS (acetamidase; e.g., from A. nidulans or A. orzyae), argB (ornithine carbamoyltransferases), bar (phosphinothricin acetyltransferase; e.g., from S. hygroscopicus), hph (hygromycin phosphotransferase), niaD (nitrate reductase), pyrG (orotidine-5′-phosphate decarboxylase; e.g., from A. nidulans or A. orzyae), sC (sulfate adenyltransferase), and trpC (anthranilate synthase), as well as equivalents thereof. Selectable marker for mammalian cells include, among others, chloramphenicol acetyl transferase (CAT), nourseothricin N-acetyl transferase, blasticidin-S deaminase, blastcidin S acetyltransferase, Sh ble (Zeocin® resistance), aminoglycoside 3′-phosphotransferase (neomycin resistance), hph (hygromycin resistance), thymidine kinase, and puromycin N-acetyl-transferase.

In another aspect, the present disclosure provides a host cell comprising at least one recombinant polynucleotide encoding an adenylate kinase polypeptide of the present disclosure, the recombinant polynucleotide(s) being operatively linked to one or more control sequences for expression of the adenylate kinase polypeptide. In some embodiments, the host cells suitable for use in expressing the polypeptides encoded by the expression vectors is a prokaryotic cell or eukaryotic cell known in the art. In some embodiments, the host cell is a bacterial cell, including, among others, E. coli, B. subtilis, Vibrio fluvialis, Streptomyces and Salmonella typhimurium cell. Exemplary bacterial host cells also include various Escherichia coli strains (e.g., W3110 (AfhuA) and BL21). In some embodiments, the host cell is a fungal cell, such as filamentous fungal cell or yeast cell. n some embodiments, suitable fungal host cells include, among others, Pichia, Saccharomyces, Yarrowia, Kluyveromyces, Aspergillus, Trichoderma, Neurospora, Mucor, Penicillium T. Trichoderma, or Myceliophthora fungal cell. Exemplary fungal host cell includes, among others, Pichia pastoris, Yarrowia lipolytica, Kluyveromyces marxianus, Kluyveromyces lactis, Aspergillus niger, Aspergillus oryzae, Aspergillus fumigatus Trichoderma reesei. Neurospora crassa, Mucor circinelloides, Penicillium chrysogenum T. reesei, Trichoderma harzianum, Saccharomyces cerevisiae, or Myceliophthora thermophile. In some embodiments, the host cell is an insect cell. In some embodiments, a suitable insect host cell is a lepidopteran or dipteran insect cell. Exemplary insect host cell includes, among others, Sf9 cell, Sf21 cell, Schneider 2 cell, and BTI-TN-5B1-4 (High Five) cell. In some embodiments, the host cell is a mammalian cell. In some embodiments, the host cell is a human cell or rodent cell. Exemplary mammalian cells include, among others, Expi293, HeLa, U2OS, A549, HT1080, CAD, P19, NIH 3T3, L929, Hek 293, 293F, 293E, 293T, COS, Vero, NS0, Sp2/0 cell, DUKX-X11, MCF-7, Y79, SO—Rb50, Hep G2, J558L, and CHO cell.

In another aspect, the present disclosure provides a method of producing the adenylate kinase polypeptides, where the method comprises culturing a host cell comprising an expression vector capable of expressing or producing the adenylate kinase polypeptide under suitable culture conditions such that the adenylate kinase polypeptide is expressed or produced. In some embodiments, the method comprises a step of isolating the adenylate kinase from the culture medium and/or host cell, as described herein. In some further embodiments, the method further comprises a step of purifying the expressed adenylate kinase polypeptide.

In some embodiments, the adenylate kinase polypeptide expressed in a host cell is recovered from the cells and/or the culture medium using any one or more of the well-known techniques for protein purification, including, among others, lysozyme or detergent treatment, sonication, filtration, salting-out, ultra-centrifugation, and chromatography, such as described herein.

Chromatographic techniques for isolation/purification of the adenylate kinase polypeptides include, among others, reverse phase chromatography, high-performance liquid chromatography, ion-exchange chromatography, hydrophobic-interaction chromatography, size-exclusion chromatography, gel electrophoresis, and affinity chromatography. Conditions for purifying the adenylate kinase depends, in part, on factors such as net charge, hydrophobicity, hydrophilicity, molecular weight, molecular shape, etc., and will be apparent to those having skill in the art. In some embodiments, affinity techniques may be used to isolate the adenylate kinase. For affinity chromatography purification, an antibody that specifically binds adenylate kinase polypeptide may be used. In some embodiments, an affinity tag, e.g., His-tag, can be introduced into the adenylate kinase polypeptide for purposes of isolation/purification.

Appropriate culture media and growth conditions for the above-described host cells are well known in the art. Polynucleotides for expression of the adenylate kinases may be introduced into cells by various methods known in the art. Techniques include, among others, electroporation, biolistic particle bombardment, liposome mediated transfection, calcium chloride transfection, and protoplast fusion.

In some embodiments, the polynucleotides encoding the adenylate kinase polypeptide can be prepared by standard solid-phase methods, according to known synthetic methods. In some embodiments, polynucleotide fragments can be individually synthesized, then joined (e.g., by enzymatic or chemical litigation methods, or polymerase mediated methods) to form any desired continuous sequence. For example, polynucleotides and oligonucleotides disclosed herein can be prepared by chemical synthesis using the classical phosphoramidite method (See e.g., Beaucage et al., Tetra. Lett., 1981, 22:1859-69; and Matthes et al., EMBO J., 1984, 3:801-05), as it is typically practiced in automated synthetic methods.

In some embodiments, a method for preparing the adenylate kinase can comprise: (a) synthesizing a polynucleotide encoding a polypeptide comprising an amino acid sequence selected from the amino acid sequence of an adenylate kinase, such as described in the Tables of the Examples, and (b) expressing the engineered adenylate kinase encoded by the polynucleotide. In some embodiments of the method, the amino acid sequence encoded by the polynucleotide can optionally have one or several (e.g., up to 3, 4, 5, or up to 10) amino acid residue deletions, insertions and/or substitutions. In some embodiments, the amino acid sequence has optionally 1-2, 1-3, 1-4, 1-5, 1-6, 1-7, 1-8, 1-9, 1-10, 1-15, 1-20, 1-21, 1-22, 1-23, 1-24, 1-25, 1-30, 1-35, 1-40, 1-45, or 1-50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 17, 18, 19, 20, 21, 22, 23, 24, 25, 30, 30, 35, 40, 45, or 50 amino acid residue deletions, insertions and/or substitutions. In some embodiments, the amino acid sequence has optionally 1, 2, 3, 4, 5, 6, 7, 8, 9, 10, 11, 12, 13, 14, 15, 16, 18, 20, 21, 22, 23, 24, or 25 amino acid residue deletions, insertions and/or substitutions. In some embodiments, the substitutions can be conservative or non-conservative substitutions. The expressed polypeptide can be assessed for the desired property, e.g., adenylate kinase activity on one or more NMPs.

### Compositions

In a further aspect, the present disclosure provides compositions of the engineered adenylate kinases disclosed herein. In some embodiments, the engineered adenylate kinase polypeptide in the composition is isolated or purified. In some embodiments, the adenylate kinase is combined with other components and compounds to provide compositions and formulations comprising the engineered adenylate kinase polypeptide as appropriate for different applications and uses.

In some embodiments, the composition comprises at least one adenylate kinase described herein. For example, a composition comprises at least one engineered adenylate kinase exemplified in Tables 7.2, 8.2, 9.2, 10.2, 11.2, 12.1, 13.2, 14.1, 15.2, 16.2, 17.2, 18.2, 19.2, 20.2, 21.2, 23.2, 24.2, 25.2, 26.2, 27.2, 28.2, 29.2, 30.2, 31.2, 32.2, 33.2, 34.2, 35.2, 36.2, 37.2, 38.2, 40.2, 41.2, 42.2, 43.2, 44.2, 45.2, 46.2, 47.2, 48.2, and 49.2, and the Sequence Listing. In some embodiments, the composition comprising an engineered adenylate kinase is an aqueous solution. In some embodiments, the composition comprising an engineered adenylate kinase is a lyophilizate.

In some embodiments, the composition further comprises at least a buffer, including the suitable buffers described herein, e.g., MOPS, triethanolamine, etc. In some embodiments, the composition further comprises an additional enzyme, including, among others, an adenosine kinase, acetate kinase, pyruvate oxidase, or 3′-O-kinase, or combinations thereof.

In some embodiments, the composition further comprises an NMP substrate. In some embodiments, the NMP substrate is a modified NMP. In some embodiments, the modified NMP substrate is modified on the 2′- and/or 3′-position of the sugar moiety (e.g., 2′-halo, 2′-O—CH₃), the nucleobase, or the phosphate group (e.g., NMP-S, NMPαS), as further described herein.

In some embodiments, the NMP substrate is an unmodified NMP. In some embodiments, the NMP substrate is AMP, GMP, CMP, UMP, or TMP. In some embodiments, the concentration of NMP in the composition is greater than that found in cells, e.g., bacterial cells or mammalian cells. In some embodiments, the concentration of NMP in the composition is at least 1 mM, 2 mM, 3 mM, 4 mM, 5 mM, 7 mM, or 10 mM, or greater. In some embodiments, the NMP substrate concentration is about 0.1 mM to 200 mM, 0.5 mM to 180 mM, 1 mM to 150 mM, 2 mM to 120 mM, 5 mM to 100 mM, 10 mM to 80 mM, or 20 mM to 60 mM, as further described herein. In some embodiments, the NMP substrate concentration is about 100 mM-200 mM. In some embodiments, the NMP substrate concentration is about 0.1 mM, 0.5 mM, 1 mM, 2 mM, 5 mM, 10 mM, 20 mM, 60 mM, 80 mM, 100 mM, 120 mM, 150 mM, 180 mM, or 200 mM.

In some embodiments, the concentration of AMP in the composition is at least 1 mM, 2 mM, 3 mM, 4 mM, 5 mM, 7 mM, or 10 mM, or greater. In some embodiments, the concentration of AMP in the composition is about 0.1 mM to 200 mM, 0.5 mM to 180 mM, 1 mM to 150 mM, 2 mM to 120 mM, 5 mM to 100 mM, 10 mM to 80 mM, or 20 mM to 60 mM. In some embodiments, the AMP substrate concentration is about 100 mM-200 mM. In some embodiments, the concentration of AMP in the composition is at least 0.1 mM, 0.5 mM, 1 mM, 2 mM, 3 mM, 4 mM, 5 mM, 7 mM, 10 mM, 20 mM, 60 mM, 80 mM, 100 mM, 120 mM, 150 mM, 180 mM, or 200 mM, or greater.

In some embodiments, the concentration of GMP in the composition is greater than 1 mM, 2 mM, 3 mM, 4 mM, 5 mM, 7 mM, or 10 mM, or greater. In some embodiments, the concentration of GMP in the composition is about 0.1 mM to 200 mM, 0.5 mM to 180 mM, 1 mM to 150 mM, 2 mM to 120 mM, 5 mM to 100 mM, 10 mM to 80 mM, or 20 mM to 60 mM. In some embodiments, the GMP substrate concentration is about 100 mM-200 mM. In some embodiments, the concentration of GMP in the composition is at least 0.1 mM, 0.5 mM, 1 mM, 2 mM, 3 mM, 4 mM, 5 mM, 7 mM, 10 mM, 20 mM, 60 mM, 80 mM, 100 mM, 120 mM, 150 mM, 180 mM, or 200 mM, or greater.

In some embodiments, the concentration of CMP in the composition is at least 0.5 mM, 1 mM, 2 mM, 3 mM, 4 mM, 5 mM, 7 mM, or 10 mM, or greater. In some embodiments, the concentration of CMP in the composition is about 0.1 mM to 200 mM, 0.5 mM to 180 mM, 1 mM to 150 mM, 2 mM to 120 mM, 5 mM to 100 mM, 10 mM to 80 mM, or 20 mM to 60 mM. In some embodiments, the CMP substrate concentration is about 100 mM-200 mM. In some embodiments, the concentration of CMP in the composition is at least 0.1 mM, 0.5 mM, 1 mM, 2 mM, 3 mM, 4 mM, 5 mM, 7 mM, 10 mM, 20 mM, 60 mM, 80 mM, 100 mM, 120 mM, 150 mM, 180 mM, or 200 mM, or greater.

In some embodiments, the concentration of UMP in the composition is at least 1 mM, 2 mM, 3 mM, 4 mM, 5 mM, 7 mM, or 10 mM or greater. In some embodiments, the concentration of UMP in the composition is about 0.1 mM to 200 mM, 0.5 mM to 180 mM, 1 mM to 150 mM, 2 mM to 120 mM, 5 mM to 100 mM, 10 mM to 80 mM, or 20 mM to 60 mM. In some embodiments, the UMP substrate concentration is about 100 mM-200 mM. In some embodiments, the concentration of UMP in the composition is at least 0.1 mM, 0.5 mM, 1 mM, 2 mM, 3 mM, 4 mM, 5 mM, 7 mM, 10 mM, 20 mM, 60 mM, 80 mM, 100 mM, 120 mM, 150 mM, 180 mM, or 200 mM, or greater.

In some embodiments, the concentration of TMP in the composition is at least 0.1 mM, 0.5 mM, 1 mM, 2 mM, 3 mM, 4 mM, 5 mM, 7 mM, or 10 mM, or greater. In some embodiments, the concentration of TMP in the composition is about 0.1 mM to 200 mM, 0.5 mM to 180 mM, 1 mM to 150 mM, 2 mM to 120 mM, 5 mM to 100 mM, 10 mM to 80 mM, or 20 mM to 60 mM. In some embodiments, the TMP substrate concentration is about 100 mM-200 mM. In some embodiments, the concentration of TMP in the composition is at least 0.1 mM, 0.5 mM, 1 mM, 2 mM, 3 mM, 4 mM, 5 mM, 7 mM, 10 mM, 20 mM, 60 mM, 80 mM, 100 mM, 120 mM, 150 mM, 180 mM, or 200 mM, or greater.

In some embodiments, the NMP substrate is a modified NMP. In some embodiments, the modified NMP substrate is modified on the 2′- and/or 3′-position of the sugar moiety (e.g., 2′-halo, 2′-O-methyl, 3′-O-phosphate, etc.), the nucleobase, the phosphate group (e.g., NMPαS), or any combinations thereof. In some embodiments, the modified NMP substrate concentration is about 0.1 mM to 200 mM, 0.5 mM to 180 mM, 1 mM to 150 mM, 2 mM to 120 mM, 5 mM to 100 mM, 10 mM to 80 mM, or 20 mM to 60 mM, as further described herein. In some embodiments, the modified NMP substrate concentration is about 100 mM-200 mM. In some embodiments, the modified NMP substrate concentration is about 0.1 mM, 0.5 mM, 1 mM, 2 mM, 5 mM, 10 mM, 20 mM, 60 mM, 80 mM, 100 mM, 120 mM, 150 mM, 180 mM, or 200 mM.

In some embodiments, the modified NMP substrate comprises a 2′-fluoro modified NMP. In some embodiments, the 2′-F modified nucleoside is 2′-F-AMP, 2′-F-GMP, 2′-F-CMP, 2′-F-2′-UMP, and/or 2-F-TMP. In some embodiments, the modified nucleoside comprises a 2′-O—CH₃ modified NMP. In some embodiments, the 2′-O—CH₃ modified nucleoside is 2′-O—CH₃ AMP, 2′-O—CH₃ GMP, 2′-O—CH₃ CMP, 2′-O—CH₃ UMP, and/or 2′-O—CH₃ TMP. Other 2′-modifications are described below.

In some embodiments, the modified NMP is modified at the 3′-position of the sugar moiety. In some embodiments, the 3′-position of the sugar moiety is modified with a blocking group, preferably a reversible blocking group. In some embodiments, the blocking group is a formate, benzoylformate, acetate, propionate, isobutyrate, aminoxy (—ONH₂), O-methyl, O-methoxymethyl, O-methylthiomethyl, O-benzyloxymethyl, O-allyl, O-(2-nitrobenzyl), O-azidomethyl (O—CH₂N₃), O-tert-butyldithiomethyl, phosphate, diphosphate, or triphosphate. Reversible 3′-blocked nucleoside/nucleotides are described in Chen et al., Genomics, Proteomics & Bioinformatics, 2013, 11(1):34-40, Metzker et al., Nucleic Acids Res., 1994, 22 (20): 4259-4267; Sabat et al., Front Chem. 2023; 11: 1161462; and patent publications U.S. Pat. Nos. 5,763,594, 9,650,406, US20200216891; WO2004/018497; and WO 2014/139596; all of which are incorporated by reference) In some embodiments, the modified sugar moiety is a 3′-O-phosphate.

In some embodiments, the modified NMP substrate comprises modified 2′- and 3′-positions of the sugar moiety, such as described herein. Exemplary modified NNP substrates with modified 2′- and 3′-positions include, among others, 2′-fluoro or 2′-O-methyl, and 3′-O-allyl, 3′-O-(2-nitrobenzyl), 3′-O-azidomethyl (O—CH₂N₃), 3′-O-tert-butyldithiomethyl, or 3′-phosphate. Other such modifications are described herein.

In some embodiments, the modified sugar moiety of the NMP substrate in the compositions is a “locked” nucleotide (e.g., locked NMP). In some embodiments, the locked NMP is a locked AMP, locked GMP, locked CMP, locked TMP, or locked UMP.

In some embodiments of the composition, the modified NMP substrate comprises a modified phosphate. In some embodiments, the modified nucleoside diphosphate comprises an alpha-thiodiphosphate (NMPαS). In some embodiments, the modified NMP substrate with a modified phosphate also has a modified sugar moiety and/or modified nucleobase.

In some embodiments, the modified NMP substrate comprises a modified nucleobase. In some embodiments, the modified nucleobases is 5-bromo-uracil, 5-iodo-uracil, 6-mCEPh-purine, 6-phenylpyrrolocytidine, N2-alkyl 8-oxoguanosine, difluorotoluene, difluorobenzene, dichlorobenzene, imidazole, or benzimidazole. Other nucleobases are described herein.

In some embodiments, the composition further comprises a phosphate donor for the adenylate kinase, for example, NTP. In some embodiments, the phosphate donor NTP has the same nucleoside structure as the NMP substrate. By way of example and not limitation, if the NMP substrate is modified at the 2′-position of the sugar moiety, the donor NTP has the same modified 2′-position of the sugar moiety. Additionally, the phosphate donor NTP has the same nucleobase as the NMP substrate.

In some embodiments, the composition comprises an engineered adenylate kinase and one or more components of a NTP regenerating system. In some embodiments, the components of the NTP regeneration system includes, among others, pyruvate kinase, creatine kinase, adenylate kinase, and/or polyphosphate kinase. In some embodiments, the components of the NTP regeneration system includes a substrate (i.e., phosphate donor) for the enzyme in the NTP regenerating system, for example, phosphoenolpyruvate, creatine phosphate, and polyphosphate, respectively.

In some embodiments, where the NTP regenerating system uses an acetate kinase, the NTP regenerating system further comprises a pyruvate oxidase, pyruvate, and phosphate, for converting the acetate to acetyl phosphate.

In some embodiments, the composition comprises an immobilized engineered adenylate kinase, as described herein. In some embodiments, the immobilized adenylate kinase is immobilized with other enzymes, e.g., an adenosine kinase, acetate kinase, and/or 3′-O-kinase.

### Uses and Methods

In another aspect, the present disclosure provides uses of the engineered adenylate kinase enzymes, either alone or in combination with other enzymes. In some embodiments, the engineered adenylate kinase is used in the production of nucleoside diphosphate (NDP) from nucleoside monophosphate (NMP). In some embodiments, a method of converting nucleoside monophosphate to nucleoside diphosphate comprises contacting a nucleoside monophosphate with an engineered adenylate kinase described herein in the presence of phosphate donor under suitable reaction conditions to convert the nucleoside monophosphate to the corresponding product nucleoside diphosphate.

In some embodiments, the nucleoside monophosphate substrate is a naturally occurring or unmodified nucleoside monophosphate. In some embodiments, unmodified nucleoside diphosphate refers to nucleosides present in naturally occurring DNA or mRNA, where the nucleosides have a 5′-phosphate.

In some embodiments, the nucleoside monophosphate has at the 2′-position of the sugar moiety a H or OH.

In some embodiments, the nucleoside monophosphate comprises a nucleobase selected from adenine, cytosine, guanine, thymine, uracil, xanthine, hypoxanthine, 2,6-diaminopurine, purine, 6,8-diaminopurine, 5-methylcytosine (m⁵C), 2-thiouridine, pseudouridine, dihydrouridine, inosine, and 7-methylguanosine (m⁷G).

In some embodiments, the unmodified nucleoside monophosphate is AMP, GMP, UMP, CMP, or TMP, and wherein the nucleoside has at the 2′-position of the sugar moiety an OH, thereby resulting in corresponding product rADP, rGDP, rUDP, rCDP, or rTDP, respectively.

In some embodiments, the unmodified nucleoside monophosphate is AMP, GMP, UMP, CMP, or TMP, and wherein the nucleoside has at the 2′-position of the sugar moiety an H, thereby by resulting in corresponding product dADP, dGDP, dUDP, dCDP, or dTDP, respectively.

In some embodiments of the method, the nucleoside monophosphate substrate is a modified nucleoside monophosphate, thereby resulting in production of the corresponding modified nucleotide diphosphate. In some embodiments, the modified nucleotide is modified in the sugar moiety, the nucleobase, or phosphate, or any combination of modified sugar moiety, nucleobase, and phosphate.

In some embodiments of the method, the modified nucleoside monophosphate comprises a modified sugar moiety. In some embodiments, the modified sugar moiety is modified at the 2′-position and/or 3′-position of the sugar moiety.

In some embodiments, the modified sugar moiety is a modified at the 2′-position of the sugar moiety. In some embodiments, the 2′-position of the sugar moiety is halo, 2′-O—R′, or 2′-O—COR′, where R′ is an alkyl, alkyloxyalkyl, cycloalkyl, heterocyclyl, aryl, heteroaryl, cycloalkylalkyl, heterocyclylalkyl, arylalkyl, or heteroarylalkyl. In some embodiments, R′ is a C₁-C₄alkyl. In some embodiments, the modified 2′-position is a 2′-O—R′, wherein in R′ is alkyloxyalkyl, alkylamine, cyanoalkyl, or —C(O)-alkyl. In some embodiments, the 2′-position of the sugar moiety of the nucleoside substrate is —O—R′, wherein R′ is —CH₃ or —CH₂CH₃ or —CH₂CH₂OCH₃. In some embodiments, the modified 2′-position is 2′-O-(2-methoxyethyl), 2′-O-allyl, 2′-O-propargyl, 2′-O-ethylamine, 2′-O-cyanoethyl, or 2′-O-acetalester.

In some embodiments, the 2′-position of the sugar moiety is halo. In some embodiments, the halo is F (2′-F) or Br (2′-Br).

In some embodiments, the modified nucleoside monophosphate substrate is a locked nucleoside. In some embodiments, the locked nucleoside is a locked C, locked GMP, locked CMP, locked TMP, or locked UMP. In some embodiments, the ribose moiety of the locked nucleoside is in the C3′-endo (beta-D) or C2′-endo (alpha-L) conformation. In some embodiments, the locked nucleoside has a methylene bridge. In some embodiments, the locked nucleoside has an ethylene bridge. Various locked nucleotides are described in, for example, WO 99/014226, WO 00/66604, WO 98/039352, WO 2004/046160, WO 00/047599, WO 2007/134181, WO 2010/077578, WO 2010/036698, WO 2007/090071, WO 2009/006478, WO 2011/156202, WO 2008/154401, WO 2009/067647, WO 2008/150729, Morita et al., Bioorganic & Med. Chem. Lett. 2002, 12, 73-76, Seth et al. J. Org. Chem. 2010, Vol 75(5) pp. 1569-81, and Mitsuoka et al., Nucleic Acids Research 2009, 37(4), 1225-1238, and Wan and Seth, J. Medical Chemistry 2016, 59, 9645-9667.

In some embodiments of the method, the modified sugar moiety is modified at the 3′-position of the sugar moiety. In some embodiments, the 3′-position of the sugar residue is modified with a blocking group, preferably a reversible blocking group. In some embodiments, the blocking group is a formate, benzoylformate, acetate, propionate, isobutyrate, aminoxy (—ONH₂), O-methyl, O-methoxymethyl, O-methylthiomethyl, O-benzyloxymethyl, O-allyl, O-(2-nitrobenzyl), O-azidomethyl (O—CH₂N₃), O-tert-butyldithiomethyl, sulfate, phosphate, diphosphate, or triphosphate. Reversible 3′-blocked nucleoside/nucleotides are described in Chen et al., Genomics, Proteomics & Bioinformatics, 2013, 11(1):34-40, Metzker et al., Nucleic Acids Res., 1994, 22 (20): 4259-4267; and patent publications U.S. Pat. Nos. 5,763,594, 9,650,406, US20200216891; WO2004/018497; and WO 2014/139596; all of which are incorporated by reference) In some embodiments, the modified sugar moiety is a 3′-O-phosphate. For example, a modified NMP substrate with a 3′-O-phosphate has the structure pNp, where the 3′-OH of the sugar moiety is modified with a phosphate.

In some embodiments, the modified NMP substrate comprises a modified nucleobase with a modification at the 2′- and the 3′-position of the sugar moiety. In some embodiments, any modification at the 2′-position of the sugar moiety is combined with any compatible modification at the 3′-position of the sugar moiety. In some embodiments, the modified NMP substrate a 2′-modification, including the 2′-modifications described herein, and a 3′-modification comprising a 3′-blocking group, particularly a 3′-reversible blocking group. In some embodiments, the 2′-modification is -2′-O-(2-methoxyethyl), 2′-O-allyl, 2′-O-propargyl, 2′-O-ethylamine, 2′-O-cyanoethyl, 2′-O-acetalester, 2′-O-methyl, 2′-O-ethyl, or 2′-fluoro, and the 3′-modification is 3′-O-methyl, 3′-O-methoxymethyl, 3′-O-methylthiomethyl, 3′-O-benzyloxymethyl, 3′-O-allyl, 3′-O-(2-nitrobenzyl), 3′-O-azidomethyl (O—CH₂N3), 3′-O-tert-butyldithiomethyl, phosphate, or triphosphate. Exemplary modified NMP substrates with a 2′- and 3′-modified sugar moiety, include among others, 2′-fluoro-AMP-3′-phosphate, 2′-fluoro-GMP-3′-phosphate, 2′-fluoro-UMP-3′-phosphate, 2′-fluoro-CMP-3′-phosphate, 2′-fluoro-TMP-3′-phosphate, 2′-O-methyl-AMP-3′-phosphate, 2′-O-methyl-GMP-3′-phosphate, 2′-O-methyl-UMP-3′-phosphate, 2′-O-methyl-CMP-3′-phosphate, and 2′-O-methyl-TMP-3′-phosphate.

In some embodiments of the method, the modified nucleoside monophosphate comprises a modified nucleobase. In some embodiments, the modified nucleobase is 5-bromo-uracil, 5-iodo-uracil, 6-mCEPh-purine, 6-phenylpyrrolocytidine, N2-alkyl 8-oxoguanosine, difluorotoluene, difluorobenzene, dichlorobenzene, imidazole, or benzimidazole.

In some embodiments, the nucleobase of the nucleoside monophosphate substrate is, among others, 5-methylcytosine, pyridin-4-one, pyridin-2-one, phenyl, pseudouracil, 3-methyl uracil, dihydrouridine, naphthyl, aminophenyl, 5-alkylcytidines, 5-alkyluridines, 5-halouridines, 6-azapyrimidines, 6-alkylpyrimidines, 5-(1-propynyl)-uridine, 5-(1-propynyl)-cytidine, quesosine, 2-thiouridine, 4-thiouridine, 4-acetyltidine, 5-(carboxyhydroxymethyl)uridine, 5-carboxymethylaminomethyl-2-thiouridine, 5-carboxymethylaminomethyluridine, -D-galactosylqueosine, 1-methyladenosine, 1-methylinosine, 2,2-dimethylguanosine, 3-methylcytidine, 2-methyladenosine, 2-methylguanosine, N6-methyladenosine, 7-methylguanosine, 5-methoxyaminomethyl-2-thiouridine, 5-methylaminomethyluridine, 5-methylcarbonylmethyluridine, 5-methyloxyuridine, 5-methyl-2-thiouridine, 2-methylthio-N6-isopentenyladenosine, -D-mannosylqueosine, uridine-5-oxyacetic acid, 2-thiocytidine, N1-methyl-adenine, N6-methyl-adenine, 8′-azido-adenine, N,N-dimethyl-adenosine, aminoallyl-adenosine, 5′-methyl-urdine, pseudouridine, N1-methyl-pseudouridine, 5′-hydroxy-methyl-uridine, 2′-thio-uridine, 4′-thio-uridine, hypoxanthine, xanthine, 5′-methyl-cytidine, 5′-hydroxy-methyl-cytidine, 6′-thio-guanine, or N7-methyl-guanine.

In some embodiments of the method, the modified nucleoside monophosphate comprises a modified phosphate. In some embodiments, the modified nucleoside monophosphate comprises a 5′-thiophosphate (NMP-S), thereby resulting in product nucleoside alpha-thiodiphosphate (NDPαS). In some embodiments, the NDPαS product is (Rp)-NDPαS, (Sp)-NDPαS, or a mixture of (Rp)-NDPαS and (Sp)-NDPαS diastereomers. In some embodiments, the method further comprises the step of separating the (Rp)-NDPαS and (Sp)-NDPαS diastereomeric products.

It is to be understood that the NMPαS can have a modified nucleoside, e.g., modification of the sugar residue at the 2′- and/or 3′-positions, a modified nucleobase, or any combinations thereof as described herein. Exemplary modified NMPαS substrates with a 2′-modified sugar moiety, include among others, 2′-fluoro-AMPαS, 2′-fluoro-GMPαS, 2′-fluoro-UMPαS, 2′-fluoro-CMPαS, 2′-fluoro-TMPαS, 2′-O-methyl-AMPαS, 2′-O-methyl-GMPαS, 2′-O-methyl-UMPαS, 2′-O-methyl-CMPαS, and 2′-O-methyl-TMPαS. Exemplary modified NMPαS substrates with a 3′-modified sugar moiety, include among others, AMPαS-3′-phosphate, GMPαS-3′-phosphate, UMPαS-3′-phosphate, CMPαS-3′-phosphate, and TMPαS-3′-phosphate. Exemplary modified NMPαS substrates with a 2′- and 3′-modified sugar moiety, include among others, 2′-fluoro-AMPαS-3′-phosphate, 2′-fluoro-GMPαS-3′-phosphate, 2′-fluoro-UMPαS-3′-phosphate, 2′-fluoro-CMPαS-3′-phosphate, 2′-fluoro-TMPαS-3′-phosphate, 2′-O-methyl-AMPαS-3′-phosphate, 2′-O-methyl-GMPαS-3′-phosphate, 2′-O-methyl-UMPαS-3′-phosphate, 2′-O-methyl-CMPαS-3′-phosphate, and 2′-O-methyl-TMPαS-3′-phosphate.

In some embodiments of the method, the phosphate donor is a nucleotide triphosphate (NTP). In some embodiments, in some embodiments, the phosphate donor NTP is rATP or dATP. In some embodiments, the phosphate donor NTP has the same nucleotide or nucleoside structure as the NMP substrate. For example, if the NMP substrate is modified at the 2′-position of the sugar moiety, the phosphate donor NTP has the same modification in the sugar moiety. Additionally, the nucleobase of the NTP donor has the same nucleobase as the NMP substrate.

In some embodiments, the phosphate donor NTP is present at a concentration of about 0.001 mM to 1 mM, 0.005 mM to 0.5 mM, 0.01 mM to 0.4 mM, 0.05 mM to 0.3 mM, or 0.1 mM to 0.2 mM. In some embodiments, the phosphate donor NTP is present at a concentration of about 0.001 mM, 0.005 mM, 0.01 mM, 0.05 mM, 0.1 mM, 0.2 mM, 0.5 mM, or 1 mM.

In some embodiments, the method further comprises a NTP regenerating or recycling system for regenerating NTP from the NDP produced by the adenylate kinase reaction. In some embodiments of the method, the NTP regenerating system comprises a creatine kinase, a pyruvate kinase, a polyphosphate kinase, and/or an acetate-kinase. In the description of enzymes herein, the database accession numbers (e.g., UniProt, GenBank, NCBI, etc.) disclosing the amino acid sequence are provided in parentheses.

In some embodiments, the NTP regenerating system is pyruvate kinase and substrate phosphoenolpyruvate. Various suitable pyruvate kinases are known in the art. In some embodiments, the pyruvate kinase for use in the NTP regenerating system is the pyruvate kinase of, among others, Escherichia coli str. K-12 substr. MG1655 (A0A3L1NNV5), Geobacillus stearothermophilus ATCC 7953 (Q02499), Schizosaccharomyces pombe (Q10208), Saccharomyces cerevisiae S288C (D6VPH8), Gallus gallus liver (F1NW43), Solanum tuberosum (P22200), Oryctolagus cuniculus M1/2 (O18919), Mus musculus muscle (P52480), Rattus norvegicus M1/2 (A0A8L2Q7B9), or Homo sapiens muscle (A0A804F729).

In some embodiments, the NTP regenerating system is creatine kinase and substrate creatine phosphate. Various suitable creatine kinases are known in the art. In some embodiments, the creatine kinase for use in the NTP regenerating system is the creatine kinase of, among others, Danio rerio (A8E5L0), Gallus gallus mitochondrial (P11009), Mus musculus (P07310), Bos taurus (Q5E9Y4), Oryctolagus cuniculus (P00563), or Homo sapiens M-type (P06732).

In some embodiments, the NTP regenerating system is polyphosphate kinase and substrate polyphosphate. Various suitable polyphosphate kinases are known in the art. In a preferred embodiment, the polyphosphate kinase is PPK1. In some embodiments, the PPK1 for use in the NTP regenerating system is the PPK1 of, among others, Pseudomonas putida DOT-TIE (AFO50238.1), Escherichia coli K-12 P0A7B1 (AAC75554.1), Clostridium acetobutylicum ATCC 824 (NP_347259.1), Thermosynechococcus elongatus (WP_011056068), Acidithiobacillus ferrooxidans (WP_064219446), Acidithiobacillus thiooxidans (WP_031572361), Bacillus acidicola (WP_066264350), Acetobacter aceti (GAN58028), Acetobacter aceti (WP_077811826.1), Thioalkalivibrio denitrificans (WP_077277945.1), or Psychromonas ingrahamii (WP_041766473.1).

In some embodiments, the NTP regenerating system is an acetate kinase and its substrate acetyl phosphate and/or propionyl phosphate. Various suitable acetates kinases are known in the art and also describe herein. In some embodiments, the acetate kinase for use in the NTP regeneration system is an acetate kinase of Escherichia coli str. K-12 substr. MG1655 (NP_416799.1), Corynebacterium jeikeium K411 (WP_011272972.1), Lactococcus cremoris subsp. cremoris KW2 (WP_011835968.1), Lactococcus lactis (WP_004254593.1), Marinitoga sp. 38H-ov (WP_165147355.1), Thermotoga sp. KOL6 (WP_101510533.1), Thermosipho melaniensis (WP_012057479.1), Thermotoga sp. RQ7 (WP_041844042.1), or Thermosipho africanus (WP_004102380.1).

In some embodiments, where the NTP regenerating system is an acetate kinase, the method further comprises a pyruvate oxidase and substrate pyruvate and inorganic phosphate. The pyruvate oxidase converts acetate to acetyl phosphate, which is then used as a phosphate donor by the acetate kinase in the NTP regenerating system. Various pyruvate oxidases are known in the art. In some embodiments, the pyruvate oxidases are homologs of pyruvate oxidases. In some embodiments, the pyruvate oxidase is a pyruvate oxidase of Bifidobacterium mongoliense (A0A087C4V4), Alkalibacterium subtropicum (A0A1I1KLE2), Pisciglobus halotolerans (A0A1I3CCM7), Jeotgalibaca sp PTS2502 (A0A1U7E9W7), Vagococcus fluvialis (A0A8I2AXT4), Candidatus Gracilibacteria bacterium (A0A2M7FGE0), Bavarilcoccus seileri (A0A3D4S346), Bifidobacterium aquikefiri (A0A261G4D1), Aerococcus urinae (F2I8Y6), or Aerococcus suis (A0A1W1YA59).

In some embodiments, the method further comprises a catalase, which converts H₂O₂ produced by the pyruvate oxidase to H₂O. Various suitable catalases are known in the art. In some embodiments, the catalase can be the catalase of Archaeoglobus fulgidus, Bacillus stearothermophilus, E. coli, Mycobacterium intracellulare, Synechococcus sp PCC7942, Arabidopsis thaliana, Pisum sativum, or Saccharomyces cerevisiae (see, e.g., Zamocky, M., Progress in Biophysics and Molecular Biology, 1999, 72(1):19-66); see also WO1992017571). In some embodiments, the catalase is a catalase of Bos taurus (P00432), Aspergillus niger (A0A254TZH3), Helicobacter pylori (JON6C6), Drosophila melanogaster (P17336) and Rattus norvegicus (P04762).

In the embodiments as provided herein and illustrated in the Examples, various ranges of suitable reaction conditions that can be used in the processes, including but not limited to, substrate loading, co-substrate loading, pH, temperature, buffer, solvent system, cofactor, polypeptide loading, and reaction time. Further suitable reaction conditions for carrying out the process for biocatalytic conversion of substrate compounds to product compounds using the enzymes described herein can be supplemented in view of the guidance provided herein to the skilled artisan, including, but not limited to, contacting the enzymes and one or more substrate compounds under experimental reaction conditions of concentration, pH, temperature, and solvent conditions, and detecting the product compound. The present disclosure contemplates any suitable reaction conditions that may find use in the methods described herein.

In some embodiments, the substrate compound in the reaction mixtures can be varied, taking into consideration, for example, the desired amount of product compound, the effect of each substrate concentration on enzyme activity, stability of enzyme under reaction conditions, and the percent conversion of each substrate to product. In some embodiments, the suitable reaction conditions comprise a substrate compound (e.g., NMP) loading of at least about 0.1 uM to 1 uM, 1 uM to 2 uM, 2 uM to 3 uM, 3 uM to 5 uM, 5 uM to 10 uM, or 10 uM to 100 uM or greater. In some embodiments, the suitable reaction conditions comprise a substrate compound (e.g., NMP) loading of about 0.1 mM to 200 mM, 0.5 mM to 180 mM, 1 mM to 150 mM, 2 mM to 120 mM, 5 mM to 100 mM, 10 mM to 80 mM, or 20 mM to 60 mM. In some embodiments, the suitable reactions conditions comprise a nucleoside substrate compound loading of about 0.1 mM, 0.5 mM, 1 mM, 2 mM, 5 mM, 10 mM, 20 mM, 60 mM, 80 mM, 100 mM, 120 mM, 150 mM, 180 mM, or 200 mM.

In some embodiments, the suitable reaction conditions comprise a substrate compound loading of at least about 0.5 to about 25 g/L, 1 to about 25 g/L, 5 to about 25 g/L, about 10 to about 25 g/L, about 20 to about 25 g/L, or about 30 to about 60 g/L. In some embodiments, the suitable reaction conditions comprise a substrate compound loading of at least about 0.5 g/L, at least about 1 g/L, at least about 5 g/L, at least about 10 g/L, at least about 15 g/L, at least about 20 g/L, at least about 30 g/L, at least about 40 g/L, at least about 50 g/L, or at least about 60 g/L, or even greater.

In carrying out the processes described herein, the enzymes may be added to the reaction mixture in the form of a purified enzyme, partially purified enzyme, whole cells transformed with gene(s) encoding the enzyme, as cell extracts and/or lysates of such cells, and/or as an enzyme immobilized on a solid support. Whole cells transformed with gene(s) encoding the enzyme(s) or cell extracts, lysates thereof, and isolated enzymes may be employed in a variety of different forms, including solid (e.g., lyophilized, spray-dried, and the like) or semisolid (e.g., a crude paste). The cell extracts or cell lysates may be partially purified by precipitation (ammonium sulfate, polyethyleneimine, heat treatment or the like, followed by a desalting procedure prior to lyophilization (e.g., ultrafiltration, dialysis, etc.). Any of the enzyme preparations (including whole cell preparations) may be stabilized by crosslinking using known crosslinking agents, such as, for example, glutaraldehyde or immobilization to a solid phase (e.g., Eupergit C, and the like).

In some embodiments, the gene(s) encoding the polypeptides can be transformed into host cell separately or together into the same host cell. For example, in some embodiments one set of host cells can be transformed with gene(s) encoding one polypeptide and another set can be transformed with gene(s) encoding another polypeptide. Both sets of transformed cells can be utilized together in the reaction mixture in the form of whole cells, or in the form of lysates or extracts derived therefrom. In other embodiments, a host cell can be transformed with gene(s) encoding multiple polypeptides. In some embodiments the polypeptides can be expressed in the form of secreted polypeptides and the culture medium containing the secreted polypeptides can be used for the synthesis reaction.

In some embodiments, the improved activity of the engineered adenylate kinase polypeptides disclosed herein provides for processes wherein higher percentage conversion can be achieved with lower concentrations of the engineered polypeptide. In some embodiments of the process, the suitable reaction conditions comprise an engineered polypeptide amount of about 0.1% w/w, 0.2% (w/w), 0.5% (w/w), 1% (w/w), 2% (w/w), 5% (w/w), 10% (w/w), 20% (w/w), 30% (w/w), 40% (w/w), 50% (w/w), 75% (w/w), 100% (w/w) or more of substrate compound loading.

In some embodiments, the engineered polypeptide is present at a molar ratio of engineered polypeptide to substrate of about 50 to 1, 25 to 1, 10 to 1, 5 to 1, 1 to 1, 1 to 5, 1 to 10, 1 to 25 or 1 to 50. In some embodiments, the engineered polypeptide is present at a molar ratio of engineered polypeptide to substrate from a range of about 50 to 1 to a range of about 1 to 50 or 1 to 100.

In some embodiments, the engineered polypeptide is present at about 0.01 g/L to about 50 g/L; about 0.01 to about 0.1 g/L; about 0.05 g/L to about 50 g/L; about 0.1 g/L to about 40 g/L; about 1 g/L to about 40 g/L; about 2 g/L to about 40 g/L; about 5 g/L to about 40 g/L; about 5 g/L to about 30 g/L; about 0.1 g/L to about 10 g/L; about 0.5 g/L to about 10 g/L; about 1 g/L to about 10 g/L; about 0.1 g/L to about 5 g/L; about 0.5 g/L to about 5 g/L; or about 0.1 g/L to about 2 g/L. In some embodiments, the adenylate kinase polypeptide is present at about 0.01 g/L, 0.05 g/L, 0.1 g/L, 0.2 g/L, 0.5 g/L, 1, 2 g/L, 5 g/L, 10 g/L, 15 g/L, 20 g/L, 25 g/L, 30 g/L, 35 g/L, 40 g/L, or 50 g/L.

In the embodiments of the process, the reaction conditions comprise a suitable pH. The desired pH or desired pH range can be maintained by use of an acid or base, an appropriate buffer, or a combination of buffering and acid or base addition. The pH of the reaction mixture can be controlled before and/or during the course of the reaction. In some embodiments, the suitable reaction conditions comprise a solution pH from about 4 to about 10, pH from about 5 to about 10, pH from about 5 to about 9, pH from about 6 to about 9, pH from about 6 to about 8. In some embodiments, the reaction conditions comprise a solution pH of about 4, 4.5, 5, 5.5, 6, 6.5, 7, 7.5, 8, 8.5, 9, 9.5, or 10.

In some embodiments, in the course of the reaction, the pH of the reaction mixture may change. The pH of the reaction mixture may be maintained at a desired pH or within a desired pH range, for example, among others, by the addition of an acid or a base, before and/or during the course of the reaction. Alternatively, the pH may be controlled by using a buffer. Accordingly, in some embodiments, the reaction condition comprises a buffer. Suitable buffers to maintain desired pH ranges are known in the art and include, by way of example and not limitation, borate, potassium phosphate, 2-(N-morpholino)ethanesulfonic acid (MES), 3-(N-morpholino)propanesulfonic acid (MOPS), acetate, triethanolamine, and 2-amino-2-hydroxymethyl-propane-1,3-diol (Tris), and the like. In some embodiments, the buffer is present in a concentration of 1 mM-500 mM, 5 mM to 450 mM, 10 mM to 400 mM, 20 mM to 350 mM, 30 mM to 300 mM, 40 mM to 200 mM, or 50 mM to 100 mM. In some embodiments, the buffer is present in the composition at about 1 mM, 5 mM, 10 mM, 20 mM, 30 mM, 40 mM, 50 mM, 100 mM, 150 mM, 200 mM, 250 mM, 300 mM, 350 mM, 400 mM, 450 mM or 500 mM. In some embodiments, the reaction conditions comprise water as a suitable solvent with no buffer present.

In the embodiments of the processes herein, a suitable temperature is used for the reaction conditions, for example, taking into consideration the increase in reaction rate at higher temperatures, and the activity of the enzyme during the reaction time period. In some embodiments, the suitable reaction conditions comprise a temperature of about 10° C. to about 95° C., about 10° C. to about 75° C., about 15° C. to about 95° C., about 20° C. to about 95° C., about 20° C. to about 65° C., about 25° C. to about 70° C., or about 50° C. to about 70° C. In some embodiments, the suitable reaction conditions comprise a temperature of about 10° C., 15° C., 20° C., 25° C., 30° C., 35° C., 40° C., 45° C., 50° C., 55° C., 60° C., 65° C., 70° C., 75° C., 80° C., 85° C., 90° C. or 95° C. In some embodiments, the temperature during the enzymatic reaction can be maintained at a specific temperature throughout the course of the reaction. In some embodiments, the temperature during the enzymatic reaction can be adjusted over a temperature profile during the course of the reaction.

In some embodiments, the processes are carried out in a solvent. Suitable solvents include water, aqueous buffer solutions, organic solvents, polymeric solvents, and/or co-solvent systems, which generally comprise aqueous solvents, organic solvents and/or polymeric solvents. The aqueous solvent (water or aqueous co-solvent system) may be pH-buffered or unbuffered. In some embodiments, the processes using the engineered adenylate kinase polypeptides can be carried out in an aqueous co-solvent system comprising an organic solvent (e.g., ethanol, isopropanol (IPA), dimethyl sulfoxide (DMSO), dimethylformamide (DMF) ethyl acetate, butyl acetate, 1-octanol, heptane, octane, methyl t butyl ether (MTBE), toluene, and the like), ionic or polar solvents (e.g., 1-ethyl 4 methylimidazolium tetrafluoroborate, 1-butyl-3-methylimidazolium tetrafluoroborate, 1-butyl 3 methylimidazolium hexafluorophosphate, glycerol, polyethylene glycol, and the like). In some embodiments, the co-solvent can be a polar solvent, such as a polyol, dimethylsulfoxide (DMSO), or lower alcohol. The non-aqueous co-solvent component of an aqueous co-solvent system may be miscible with the aqueous component, providing a single liquid phase, or may be partly miscible or immiscible with the aqueous component, providing two liquid phases. Exemplary aqueous co-solvent systems can comprise water and one or more co-solvents selected from an organic solvent, polar solvent, and polyol solvent. In general, the co-solvent component of an aqueous co-solvent system is chosen such that it does not adversely inactivate the enzymes under the reaction conditions. Appropriate co-solvent systems can be readily identified by measuring the enzymatic activity of the specified enzymes with a defined substrate of interest in the candidate solvent system, utilizing an enzyme activity assay, such as those described herein.

In some embodiments of the process, the suitable reaction conditions comprise an aqueous co-solvent, where the co-solvent comprises DMSO at about 1% to about 50% (v/v), about 1 to about 40% (v/v), about 2% to about 40% (v/v), about 5% to about 30% (v/v), about 10% to about 30% (v/v), or about 10% to about 20% (v/v). In some embodiments of the process, the suitable reaction conditions can comprise an aqueous co-solvent comprising ethanol at about 1% (v/v), about 5% (v/v), about 10% (v/v), about 15% (v/v), about 20% (v/v), about 25% (v/v), about 30% (v/v), about 35% (v/v), about 40% (v/v), about 45% (v/v), or about 50% (v/v).

In some embodiments, the reaction conditions comprise a surfactant for stabilizing or enhancing the reaction. Surfactants can comprise non-ionic, cationic, anionic and/or amphiphilic surfactants. Exemplary surfactants, include by way of example and not limitation, nonyl phenoxypolyethoxylethanol (NP40), TRITON™ X-100 polyethylene glycol tert-octylphenyl ether, polyoxyethylene-stearylamine, cetyltrimethylammonium bromide, sodium oleylamidosulfate, polyoxyethylene-sorbitanmonostearate, hexadecyldimethylamine, etc. Any surfactant that may stabilize or enhance the reaction may be employed. The concentration of the surfactant to be employed in the reaction may be generally from 0.1 to 50 mg/mL, particularly from 1 to 20 mg/mL.

In some embodiments, the reaction conditions include an antifoam agent, which aids in reducing or preventing formation of foam in the reaction solution, such as when the reaction solutions are mixed or sparged. Anti-foam agents include non-polar oils (e.g., minerals, silicones, etc.), polar oils (e.g., fatty acids, alkyl amines, alkyl amides, alkyl sulfates, etc.), and hydrophobic (e.g., treated silica, polypropylene, etc.), some of which also function as surfactants. Exemplary anti-foam agents include Y-30® (Dow Corning), poly-glycol copolymers, oxy/ethoxylated alcohols, and polydimethylsiloxanes. In some embodiments, the anti-foam can be present at about 0.001% (v/v) to about 5% (v/v), about 0.01% (v/v) to about 5% (v/v), about 0.1% (v/v) to about 5% (v/v), or about 0.1% (v/v) to about 2% (v/v). In some embodiments, the anti-foam agent can be present at about 0.001% (v/v), about 0.01% (v/v), about 0.1% (v/v), about 0.5% (v/v), about 1% (v/v), about 2% (v/v), about 3% (v/v), about 4% (v/v), or about 5% (v/v) or more as desirable to promote the reaction.

In some embodiments, the quantities of reactants used in the synthesis reaction will generally vary depending on the quantities of product desired, and concomitantly the amount of substrates employed. Those having ordinary skill in the art will readily understand how to vary these quantities to tailor them to the desired level of productivity and scale of production.

In some embodiments, the order of addition of reactants is not critical. The reactants may be added together at the same time to a solvent (e.g., monophasic solvent, biphasic aqueous co-solvent system, and the like), or alternatively, some of the reactants may be added separately, and some together at different time points. For example, where applicable, the cofactor, co-substrate and substrate may be added first to the solvent.

In some embodiments, the reaction components (e.g., enzyme, salts, etc.) may be provided to the reaction in a variety of different forms, including powder (e.g., lyophilized, spray dried, and the like), solution, emulsion, suspension, and the like. The reactants can be readily lyophilized or spray dried using methods and equipment that are known to those having ordinary skill in the art. For example, the protein solution can be frozen at −80° C. in small aliquots, then added to a pre-chilled lyophilization chamber, followed by the application of a vacuum.

For improved mixing efficiency when an aqueous co-solvent system is used, the polypeptide(s), and co-substrate may be added and mixed into the aqueous phase first. The substrate may be added and mixed in, followed by the organic phase or the substrate may be dissolved in the organic phase and mixed in. Alternatively, the substrate may be premixed in the organic phase, prior to addition to the aqueous phase.

The processes of the present invention are generally allowed to proceed until further conversion of substrate to product does not change significantly with reaction time (e.g., less than 10% of substrate being converted, or less than 5% of substrate being converted). In some embodiments, the reaction is allowed to proceed until there is complete or near complete conversion of substrate to product. Transformation of substrate to product can be monitored using known methods by detecting substrate and/or product, with or without derivatization. Suitable analytical methods include gas chromatography, HPLC, MS, and the like. In some embodiments, after suitable conversion to product, the reactants are separated from the product and additional reactants are added.

Any of the processes disclosed herein using the polypeptides for the preparation of products can be carried out under a range of suitable reaction conditions, including but not limited to ranges of substrates, temperature, pH, solvent system, substrate loading, polypeptide loading, cofactor loading, and reaction time. In one example, the suitable reaction conditions for the conversion of a NMP to a NDP comprise: (a) substrate loading of about 1-200 mM NMP; (b) about 0.01 g/L to 5 g/L engineered adenylate kinase polypeptide; (c) 1-100 mM MgCl₂; (e) 5 to 100 mM of buffer, e.g., Tris-HCl; (f) 0.01-100 mM NTP (e.g., ATP); (g) pH at 5-9; and (h) temperature of about 15° C. to 70° C. In some embodiments, additional reaction components or additional techniques carried out to supplement the reaction conditions. These can include taking measures to stabilize or prevent inactivation of the enzyme, reduce product inhibition, shift reaction equilibrium to formation of the desired product.

As described herein, the methods of using the polypeptides described herein can be carried out using the polypeptides bound or immobilized on a solid support. Methods of polypeptide immobilization are known in the art. In some embodiments, the engineered polypeptides can be bound non-covalently or covalently. Various methods for conjugation and immobilization of enzymes to solid supports (e.g., resins, membranes, beads, glass, etc.) are described in, for example, Yi et al., Proc. Biochem., 2007, 42(5):895-898; Martin et al., Appl. Microbiol. Biotechnol., 2007, 76(4): 843-851; Koszelewski et al., J. Mol. Cat. B: Enzymatic, 2010, 63: 39-44; Truppo et al., Org. Proc. Res. Dev., published online: dx.doi.org/10.1021/op200157c; Hermanson, “Bioconjugate Techniques,” 2nd Ed., Academic Press, Cambridge, MA (2008); Mateo et al., Biotechnol. Prog., 2002, 18(3):629-34; and “Bioconjugation Protocols: Strategies and Methods,” In “Methods in Molecular Biology,” Niemeyer (ed.), Humana Press, New York, NY (2004); the disclosures of each which are incorporated by reference herein). Solid supports useful for immobilizing the engineered polypeptides of the present disclosure include, but are not limited to, beads or resins comprising polymethacrylate with epoxide functional groups, polymethacrylate with amino epoxide functional groups, styrene/DVB copolymer or polymethacrylate with octadecyl functional groups. In some embodiments, exemplary solid supports useful for immobilizing the engineered polypeptides of the present invention include, but are not limited to, EnginZyme (including, EziG-1, EziG-1, and EziG-3), chitosan beads, Eupergit C, and SEPABEADs (Mitsubishi) (including EC-EP, EC-HFA/S, EXA252, EXE119 and EXE120).

In further embodiments, any of the above described processes for the conversion of one or more substrate compounds to product compound can further comprise one or more steps selected from: extraction; isolation; purification; and crystallization of product compound. Methods, techniques, and protocols for extracting, isolating, purifying, and/or crystallizing the product from biocatalytic reaction mixtures produced by the above disclosed processes are known to the ordinary artisan. Additionally, illustrative methods are provided in the Examples below.

### EXAMPLES

The following Examples, including experiments and results achieved, are provided for illustrative purposes only and are not to be construed as limiting the present invention.

In the experimental disclosure below, the following abbreviations apply where applicable: ppm (parts per million); M (molar); mM (millimolar), uM and μM (micromolar); nM (nanomolar); mol (moles); gm and g (gram); mg (milligrams); ug and μg (micrograms); L and l (liter); ml and mL (milliliter); ul, μl, mL, and μL (microliter); cm (centimeters); mm (millimeters); um and μm (micrometers); sec. (seconds); min(s) (minute(s)); h(s) and hr(s) (hour(s)); U (units); MW (molecular weight); D (daltons); rpm (rotations per minute); ° C. (degrees Celcius); LB (Luria Broth); IPTG (isopropyl-β-D-thiogalactoside); LiK (lithium-potassium); DNA (deoxyribonucleic acid); and RNA (ribonucleic acid).

### Nucleotide Modifications and Oligonucleotides

Description of modifications in oligonucleotide sequences

| Alias(es) | Description |
|---|---|
| A, dA | 2′-deoxyadenosine |
| C, dC | 2′-deoxycytidine |
| G, dG | 2′-deoxyguanosine |
| T, dT | 2′-deoxythymidine |
| rA | adenosine ribonucleotide |
| rC | cytidine ribonucleotide |
| rG | guanosine ribonucleotide |
| rU | uridine ribonucleotide |
| mA | 2′-methoxyadenosine |
| mC | 2′-methoxycytidine |
| mG | 2′-methoxyguanosine |
| mU | 2′-methoxyuridine |
| N{number}, e.g. T15 | Poly(N) of length {number} |
| * | phosphorothioate linkage |
| FAM-, /56-FAM/ | 5′-6-carboxyfluorescein |
| 5′P, /5Phos/ | 5′-phosphorate |
| 3′P, /3Phos/ | 3′-phosphorate |
| fA, /52FA/, /i2FA/, /32FA/ | 2′-fluoroadenosine |
| fG, /52FG/, /i2FG/, /32FG/ | 2′-fluoroguanosine |
| fU, /52FU/, /i2FU/, /32FU/ | 2′-fluorouridine |
| fC, /52FC/, /i2FC/, /32FC/ | 2′-fluorocytidine |

Oligonucleotide sequences are represented as DNA sequences. In this context, A, G, C and T refer to 2′-deoxyribonucleotides. In other contexts, A, G, C, and T can refer to nucleotides with modifications at the 2′ position.

| | SEQ ID | |
|---|---|---|
| Alias | NO: | Oligo sequence |
| T15AT*mG | 1321 | TTTTTTTTTTTTTTAT*mG |
| FAM-T15AT*mG | 1322 | /56-FAM/TTTTTTTTTTTTTTTAT*mG |
| T15mGmAmC | 1323 | TTTTTTTTTTTTTTmGmAmC |
| FAM-T15mGmAmC | 1324 | /56-FAM/TTTTTTTTTTTTTTTmGmAmC |
| T15AT*mGrG | 1325 | TTTTTTTTTTTTTTAT*mGrG |
| FAM-T15AT*mGrG | 1326 | /56-FAM/TTTTTTTTTTTTTTTAT*mGrG |
| T15AT*mGrA | 1327 | TTTTTTTTTTTTTTAT*mGrA |
| FAM-T15AT*mGrA | 1328 | /56-FAM/TTTTTTTTTTTTTTTAT*mGrA |
| T15mGmAmCrG | 1329 | TTTTTTTTTTTTTTmGmAmCrG |
| FAM-T15mGmAmCrG | 1330 | /56-FAM/TTTTTTTTTTTTTTTmGmAmCrG |
| T15mGmAmC/32FG/ | 1331 | TTTTTTTTTTTTTTmGmAmC/32FG/ |
| FAM-T15mGmAmC/32FG/ | 1332 | /56-FAM/TTTTTTTTTTTTTTTmGmAmC/32FG/ |
| T15mGmAmCmU | 1333 | TTTTTTTTTTTTTTmGmAmCmU |
| FAM-T15mGmAmCmU | 1334 | /56-FAM/TTTTTTTTTTTTTTTmGmAmCmU |
| T15mGmAmC | 2653 | TTTTTTTTTTTTTTTmGmAmC |
| T15mGmAmC/52FG/ | 2654 | TTTTTTTTTTTTTTTmGmAmC/52FG/ |
| FAM-T15mGmAmC/52FG/ | 2655 | /56-FAM/TTTTTTTTTTTTTTTmGmAmC/52FG/ |
| T15mGmAmCmA | 2656 | TTTTTTTTTTTTTTTmGmAmCmA |
| FAM-T15mGmAmCmA | 2657 | /56-FAM/TTTTTTTTTTTTTTTmGmAmCmA |
| T15mGmAmCmU | 2658 | TTTTTTTTTTTTTTTmGmAmCmU |
| T15mGmAmCmG | 2659 | TTTTTTTTTTTTTTTmGmAmCmG |
| FAM-T15mGmAmCmG | 2660 | /56-FAM/TTTTTTTTTTTTTTTmGmAmCmG |
| FAM-T15mGmAmC | 3193 | /56-FAM/TTTTTTTTTTTTTTTmGmAmC |
| T15mGmAmC | 3194 | TTTTTTTTTTTTTTTmGmAmC |
| FAM-T15mGmAmC/32FG/ | 3195 | /56-FAM/TTTTTTTTTTTTTTTmGmAmC/32FG/ |
| T15mGmAmC/32FG/ | 3196 | TTTTTTTTTTTTTTTmGmAmC/32FG/ |
| FAM-T15mGmAmCmU | 3197 | /56-FAM/TTTTTTTTTTTTTTTmGmAmCmU |
| T15mGmAmCmU | 3198 | TTTTTTTTTTTTTTTmGmAmCmU |
| FAM-T15mGmAmCmG | 3199 | /56-FAM/TTTTTTTTTTTTTTTmGmAmCmG |
| T15mGmAmCmG | 3200 | TTTTTTTTTTTTTTTmGmAmCmG |

### Example 1

### Gene Acquisition and Adenylate Kinase (AdyK) Selection

Synthetic genes encoding N-terminal 6-histidine tagged versions of wild-type (WT) and evolved adenylate kinase enzymes (AdK) were cloned into the pCK900 vector system (See e.g., U.S. Pat. No. 9,714,437, which is hereby incorporated by reference in its entirety) and subsequently expressed in an E. coli strain derived from W3110TKO.

Cells transformed with the adenylate kinase expression construct were grown at shake-flask scale, as described in Example 3. Cells were then lysed, purified, and dialyzed into storage buffer (20 mM Tris-HCl, pH 7.4, 100 mM KCl, 0.1 mM EDTA, and 50% glycerol). After overnight dialysis, protein samples were removed, and adenylate kinase enzyme concentrations were measured by absorption at 280 nm using a NanoDrop™ 1000 spectrophotometer. Soluble protein concentrations are summarized in Table 1.1 below.

TABLE 1.1
Soluble Enzyme Production of Variants
Relative to SEQ ID NO: 1342

| SEQ | | FIOP Soluble Enzyme |
|---|---|---|
| ID NO: | Source organism of | Production (Relative |
| (nt/aa) | AdK gene sequence | to SEQ ID NO: 1342) |
| 1335/1336 | Saccharomyces cerevisiae | +++ |
| 1337/1338 | Saccharomyces cerevisiae | +++ |
| 1/2 | Thermotoga neapolitana | ++ |
| 1339/1340 | Escherichia coli | ++ |
| 1341/1342 | Geobacillus | + |
| | stearothermophilus | |

Levels of increased soluble enzyme production were determined relative to the reference polypeptide of SEQ ID NO: 1342 and defined as follows:
“+” 1.00 to 1.10,
“++” >1.10,
“+++” >2.10

The wild-type (WT) adenylate kinase (AdyK) enzyme (SEQ ID NO: 2) encoded by the genome of Thermotoga neapolitana (UniProt ID: Q8GGL2) was selected for protein engineering. A synthetic gene (SEQ ID NO: 1) encoding an N-terminal 6-histidine tagged version of the WT AdyK was designed with codon optimization for E. coli expression, synthesized, and subcloned into the E. coli expression vector pCK100900i (See e.g., U.S. Pat. No. 7,629,157 and US Pat. Appln. Publn. 2016/0244787, all of which are hereby incorporated by reference). This plasmid construct was transformed into an E. coli strain derived from W3110. Directed evolution techniques generally known by those skilled in the art were used to generate libraries of gene variants from these plasmids. The substitutions in the enzyme variants described herein are indicated with reference to the N-terminal 6-histidine tagged version of the WT AdyK enzyme (i.e., SEQ ID NO: 2) or variants thereof, as indicated.

### Example 2

AdyK Expression and Lysate Processing for High throughput (HTP) Screening

### High Throughput (HTP) Growth of AdvK Enzyme and Variants

Transformed E. coli cells were selected by plating onto LB agar plates containing 1% glucose and 30 μg/mL chloramphenicol. After overnight incubation at 37° C., colonies were placed into the wells of 96-well shallow flat bottom NUNC™ (Thermo-Scientific) plates filled with 180 μl/well LB medium supplemented with 1% glucose and 30 μg/mL chloramphenicol. The cultures were allowed to grow overnight for 18-20 hours in a shaker (200 rpm, 30° C., and 85% relative humidity; Kuhner). Overnight growth samples (20 μL) were transferred into Costar 96-well deep plates filled with 380 μL of Terrific Broth supplemented with 30 μg/mL chloramphenicol. The plates were incubated for 120 min in a shaker (250 rpm, 30° C., and 85% relative humidity; Kuhner) until the OD₆₀₀ reached between 0.4-0.8. The cells were then induced with 40 μL of 10 mM IPTG in sterile water and incubated overnight for 18-20 hr in a shaker (250 rpm, 30° C., and 85% relative humidity; Kuhner). The cells were pelleted (4,000 rpm for 20 min), the supernatants were discarded, and the cells were frozen at −80° C. prior to analysis.

Thermal Lysis of HTP Cell Pellets with Lysozyme (Examples 13-25)

For lysis, 400 μL lysis buffer containing 50 mM triethanolamine buffer, pH 7.5, and 0.1 g/L lysozyme were added to the cell pellet in each well. The cells were shaken vigorously at room temperature for 5 min on a bench top shaker. An aliquot of the re-suspended cells (5-100 μL) was transferred to a 96-well format 200 μL BioRad PCR plate, diluting to 100 uL in lysis buffer if necessary, then briefly spun-down prior to 1 h heat treatment at the temperature indicated, typically 45-60° C. Following heat-treatment, the cell debris was pelleted by centrifugation (4,000 rpm at 4° C. for 10 min), and clear supernatants were then used in biocatalytic reactions to determine their activity levels.

### Example 3

### Shake Flask Expression and Purification of Adenylate Kinase (AdyK)

### Shake Flask Expression

Selected HTP cultures grown as described above were plated onto LB agar plates with 1% glucose and 30 μg/mL chloramphenicol and grown overnight at 37° C. A single colony from each culture was transferred to 5 mL of LB broth with 1% glucose and 30 μg/mL chloramphenicol. The cultures were grown for 20 h at 30° C., 250 rpm, and subcultured at a dilution of approximately 1:50 into 250 mL of Terrific Broth with 30 μg/mL of chloramphenicol, to a final OD₆₀₀ of about 0.05. The cultures were incubated for approximately 195 min at 30° C., 250 rpm, to an OD₆₀₀ of about 0.6, and then induced with the addition of IPTG at a final concentration of 1 mM. The induced cultures were incubated for 20 h at 30° C., 250 rpm. Following this incubation period, the cultures were centrifuged at 4,000 rpm for 10 min. The culture supernatant was discarded, and the pellets were resuspended in 35 mL of 20 mM triethanolamine, pH 7.5. This cell suspension was chilled in an ice bath and lysed using a Microfluidizer cell disruptor (Microfluidics M-110L). The crude lysate was pelleted by centrifugation (10,000 rpm for 60 min at 4° C.), and the supernatant was then filtered through a 0.2 μm PES membrane to further clarify the lysate.

Purification of AdyK from Shake Flask Lysates

AdyK lysates were supplemented with 1/50^{th} volume of SF elution buffer (50 mM Tris-HCl, 500 mM NaCl, 250 mM imidazole, 0.02% v/v Triton X-100 reagent) per well. Lysates were then purified using an AKTA Start purification system and a 5 mL HisTrap FF column (GE Healthcare). The SF wash buffer comprised 50 mM Tris-HCl, 300 mM NaCl, 20 mM imidazole, 0.02% v/v Triton X-100 reagent.

TABLE 3.1
Purification Parameters

| Parameter | Volume | |
|---|---|---|
| Column volume | 5 | mL |
| Flow rate | 8-12 | mL/min |
| Pressure limit | 0.3 | MPa |
| Sample volume | 50 | mL |
| Equilibration volume | 5 column volumes (CV) = 25 mL | |
| Wash Unbound volume | 20 CV = 100 mLs | |
| Elution | Isocratic (step) | |
| Elution volume | 5 CV = 25 mL | |
| Fraction volume | 1.5 | mL |

| | |
|---|---|
| RE-equilibration volume | 5 CV = 25 mL |

Elution fractions containing protein were identified by UV absorption (A280) and pooled, then dialyzed overnight in dialysis buffer (20 mM Tris-HCl, pH 7.4, 100 mM KCl, 0.1 mM EDTA, and 50% glycerol) in a 3.5K Slide-A-Lyzer™ dialysis cassette (Thermo Fisher) for buffer exchange. AdyK concentrations in the preparations were measured by absorption at 280 nm.

### Example 4

### Biosynthetic Cascade Reactions for Production of Nucleotide Triphosphates (NTPs)

### NTP Biosynthetic Reaction Setup

Reactions were performed in 384-well format 40 μL BioRad PCR plates. AdyK variants were assayed in the presence of adenosine kinase (AdoK/5′-O-kinase) and/or acetate kinase (AcK) variants to enable direct conversion of nucleosides to the corresponding triphosphate. The reactions were set up as follows: (i) all reaction components, except for the nucleoside substrate and the AdyK lysate, were premixed in a single solution and were aliquoted into each well of the 384-well plates, (ii) AdyK lysate solution was then added into the wells, and (iii) an aliquot of the substrate nucleoside in DMSO was added to initiate the reaction. The reaction plate was heat-sealed with a peelable aluminum seal and incubated in a thermocycler at the indicated temperature and reaction time, then held at 10° C. prior to analysis.

### Example 5

### High-Performance Liquid Chromatography (HPLC) Analysis of Phosphorylated Products

### Sample Preparation for Reaction Analysis Using HPLC

The nucleoside substrates, along with their respective 5′-monophosphate (NMP), 5′-diphosphate (NDP), and 5′-triphosphate (NTP) products produced using reactions set up as described in Example 4 were analyzed using HPLC. Mobile phases consisted of 50 mM potassium phosphate (pH 7) with 2 mM tetrabutylammonium hydrogen sulfate (Solvent A), acetonitrile (solvent B), and water (Solvent C). Products were detected by UV absorption at 254 nm. In some instances, a Zorbax RR StableBond Aq, 3.0×150 mm, 3.5 μm (Agilent, #863954-314) column was used. In other instances, a Zorbax RR StableBond Aq, 3.0×100 mm, 3.5 μm (Agilent, #861954-314) column was used, while in other instances, a Zorbax RR StableBond Aq, 2.1×50 mm, 3.5 μm (Agilent, #871700-914) column was used.

### Example 6

### Analysis of NTP Production in Biosynthetic Cascade Using Capillary Electrophoresis

### Coupling of NTP Biosynthetic Reactions to TdT for CE

For high-throughput (HTP) determination of NTP yield, NTP biosynthetic cascade reactions were terminated with either a heat killed at 95° C. for 2 min or by dilution with 75% methanol. Samples were then diluted into a coupling reaction. The reaction contained 20 mM triethanolamine (TEoA), 0.25 mM CoCl₂, 0.001 units of inorganic pyrophosphate (New England Biolabs), 4 μM SED ID NO: 1320 (TdT enzyme variant), 12.375 μM of unlabeled oligonucleotide, and 0.125 μM 5′-6-FAM-labeled oligonucleotide. Reactions were carried out at 50° C. for 60 min, followed by 2 min at 95° C.

### Sample Preparation for Reaction Analysis Using CE

For analysis of reaction samples, capillary electrophoresis was performed using either an ABI 3500XL Genetic Analyzer (ThermoFisher) or a SeqStudio™ Flex Genetic Analyzer (ThermoFisher). Reactions (1 μL) were quenched by the additions of 19 μL of 1 mM aqueous ethylenediaminetetraacetic acid (EDTA) Quenched reactions were diluted to 1.25 nM oligonucleotide, and a 2-μL aliquot of this solutions was transferred to a new 96-well MicroAmp Optical PCR plate or a 384-well MicroAmp Optical PCR plate containing 18 μL Hi-Di™ Formamide (ThermoFisher) containing the Alexa633 size standard. The ABI 3500XL and SeqStudio™ Flex were configured with POP6 polymer, 50 cm capillaries, and a 55° C. oven temperature. Pre-run settings were 18 kV for 50 sec. Injection was 10 kV for 2 sec, and the run settings were 19 kV for 620-640 sec. FAM-labeled oligo substrates and products were identified by their sizes relative to the sizing ladder.

### Example 7

### Improvements Over SEQ ID NO: 2 in Conversion of Nucleoside Monophosphates to Nucleotides

### HTP Screening for Improved AdyK Variants

Adenylate kinase of SEQ ID NO: 2 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 7.1.

Reactions were performed as described in Example 4 using conditions summarized in Table 7.1. Data were collected using the HPLC assay described in Example 5.

TABLE 7.1
Reaction conditions

Lysis Buffer - TEoA (pH 7.5), 0.1 g/L lysozyme; Lysis
Conditions - 100 μL, 60° C., 60 min; Reaction buffer -
50 mM Tris, 50 mM LiK(acetyl phosphate), 10 μM ATP,
10 mM magnesium chloride, pH 8.0; Lysate concentration
(vol %) - 50; Reaction Conditions - 50 μL, 30° C., 1 hr;
Nucleoside substrate - GMP; Substrate Concentration -
10 mM; Auxiliary Cascade Enzymes (AcK) - SEQ ID NO: 1314
(10 μM); Quench - 75% methanol; Analytic dilution - XU.

Activity relative to SEQ ID NO: 2 (Activity FIOP) was calculated based on the percentage of extension products observed for the variant compared with the percentage observed with SEQ ID NO: 2 (where the percent product may be set as the average of replicates or else the highest single sample as appropriate). The results are shown in Table 7.2.

TABLE 7.2
Adenylate kinase activity relative to SEQ ID NO: 2

| SEQ | | FIOP Percent |
|---|---|---|
| ID NO: | Amino Acid Differences | Product Relative |
| (nt/aa) | (Relative to SEQ ID NO: 2) | to SEQ ID NO: 2 |
| 3/4 | Y29Q/A30S/D128K/R142L/Y186L/ | +++ |
| | T212S/I213Q | |
| 5/6 | F18L/R142L/T212S/I213Q | +++ |
| 7/8 | Y29Q/A30S/D128K/R142L/Y186L | +++ |
| 9/10 | P40C/V135P/G214P | ++ |
| 11/12 | F18L/Y29Q/A30S/F127L/I150S | ++ |
| 13/14 | V82I | ++ |
| 15/16 | K69Q | ++ |
| 17/18 | N118G | + |
| 19/20 | Y29Q/A30S | + |
| 21/22 | L206W | + |
| 23/24 | R32N | + |
| 25/26 | L16M | + |
| 27/28 | V104I | + |
| 29/30 | D111E/V135P/Q136A/G214P | + |
| 31/32 | Y29Q/D128K/D175S | + |
| 33/34 | K178N | + |
| 35/36 | F18L/D128K/R142L | + |
| 37/38 | R32N/E108M/D111E/L156N/ | + |
| | E191K | |
| 39/40 | F18L/Y29Q/D128K/V129I/R142L/ | + |
| | Y186L | |
| 41/42 | R32N/V133L | + |
| 43/44 | F18L/Y29Q/A30S/T212S/I213Q/ | + |
| | V222I | |
| 45/46 | Y29Q/E65K/F127L | + |
| 47/48 | R32N/P40C/S43G/V52H/S155F/ | + |
| | L156N/N217T | |
| 49/50 | F18L/Y29Q/D128K/V129I | + |
| 51/52 | E80N | + |
| 53/54 | R32N/S43G/L138I/N152H/E191K/ | + |
| | G214P/I215V | |
| 55/56 | R183Q | + |
| 57/58 | F18L/D175S/Y186L/T212S/I213Q | + |
| 59/60 | V129I | + |
| 61/62 | F18L/Y29Q/A30S/E65K/V129I/ | + |
| | R142L/I150S/Y186L | |
| 63/64 | F127L/R142L/T212S/I213Q | + |
| 65/66 | A30S/E65K/R142L/V222I | + |
| 67/68 | P40C/S43G/I215V | + |
| 69/70 | Q136A | + |
| 71/72 | Y29Q/I150S/G211A/T212S/I213Q/ | + |
| | E221D | |
| 73/74 | S43G/D111E/Q136A/V219W | + |
| 75/76 | I226L | + |
| 77/78 | E88N | + |
| 79/80 | S140G/I215V/D216A | + |
| 81/82 | R32N/V133L/V134L | + |
| 83/84 | F18L/Y29Q/D128K/Y186L | + |

Levels of increased activity were determined relative to the reference polypeptide of SEQ ID NO: 2 and defined as follows:
“+” 1.20 to 1.52,
“++” >1.52,
“+++” >2.23.

### Example 8

### Improvements Over SEQ ID NO: 4 in Conversion of Nucleoside Monophosphates to Nucleotides

### HTP Screening for Improved AdyK Variants

Adenylate kinase of SEQ ID NO: 4 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 8.1.

Reactions were performed as described in Example 4 using conditions summarized in Table 8.1. Data were collected using the HPLC assay described in Example 5.

TABLE 8.1
Reaction conditions

Lysis Buffer - TEoA (pH 7.5), 0.1 g/L lysozyme; Lysis
Conditions - 100 μL, 60° C., 60 min; Reaction buffer -
50 mM Tris, 50 mM LiK(acetyl phosphate), 10 μM ATP,
10 mM magnesium chloride, pH 8.0; Lysate concentration
(vol %) - 50; Reaction Conditions - 1 μL, 30° C., 1 hr;
Nucleoside substrate - GMP; Substrate Concentration -
10 mM; Auxiliary Cascade Enzymes (AcK) - SEQ ID NO: 1314
(1 μM); Dilution into Coupling Reaction - 80X; Substrate
Oligonucleotides - SEQ ID NO: 1321, SEQ ID NO: 1322;
Product Oligonucleotides - SEQ ID NO: 1325, SEQ ID NO: 1326.

Activity relative to SEQ ID NO: 4 (Activity FIOP) was calculated based on the percentage of extension products observed for the variant compared with the percentage observed with SEQ ID NO: 4 (where the percent product may be set as the average of replicates or else the highest single sample as appropriate). The results are shown in Table 8.2.

TABLE 8.2
Adenylate kinase activity relative to SEQ ID NO: 4

| SEQ | | FIOP Percent |
|---|---|---|
| ID NO: | Amino Acid Differences | Product Relative |
| (nt/aa) | (Relative to SEQ ID NO: 4) | to SEQ ID NO: 4 |
| 85/86 | Y100V/P101- | +++ |
| 87/88 | G94S | +++ |
| 89/90 | E179L | +++ |
| 91/92 | D198G | +++ |
| 93/94 | D57P | ++ |
| 95/96 | V78G | ++ |
| 97/98 | E68R | ++ |
| 99/100 | K93V | ++ |
| 101/102 | L77S | ++ |
| 103/104 | K62L | ++ |
| 105/106 | A14C | ++ |
| 107/108 | I51R | ++ |
| 109/110 | N118R | ++ |
| 111/112 | C91F | ++ |
| 113/114 | L163G | ++ |
| 115/116 | E65P | ++ |
| 117/118 | Q173R | ++ |
| 119/120 | K224G | ++ |
| 121/122 | I66E | ++ |
| 123/124 | F109E | ++ |
| 125/126 | G60S | ++ |
| 127/128 | P40C/E88N | ++ |
| 129/130 | E191D | ++ |
| 131/132 | I63S | ++ |
| 133/134 | E68D/K170P | ++ |
| 135/136 | Y100S | ++ |
| 137/138 | I51M | ++ |
| 139/140 | E108S | ++ |
| 141/142 | E58G | ++ |
| 143/144 | D57H | + |
| 145/146 | E180G | + |
| 147/148 | K178R | + |
| 149/150 | Y199R | + |
| 151/152 | P40C | + |
| 153/154 | G94Q | + |
| 155/156 | E80N/K203A | + |
| 157/158 | P40C/S43G | + |
| 159/160 | I39T | + |
| 161/162 | K62S | + |
| 163/164 | P40L | + |
| 165/166 | E55Q | + |
| 167/168 | L110C | + |
| 169/170 | I51R/L59R | + |
| 171/172 | S155F/I226L | + |
| 173/174 | E80N | + |
| 175/176 | V219T | + |
| 177/178 | D166P | + |
| 179/180 | G60P | + |
| 181/182 | K202S | + |
| 183/184 | L59T | + |
| 185/186 | E80A | + |
| 187/188 | G38L | + |
| 189/190 | G94C | + |
| 191/192 | K62A | + |
| 193/194 | I51K | + |
| 195/196 | D90E | + |
| 197/198 | L59M | + |
| 199/200 | E80N/H184K/K203A | + |
| 201/202 | E35S | + |
| 203/204 | V222T | + |
| 205/206 | K178G | + |
| 207/208 | P40T | + |
| 209/210 | K54M | + |
| 211/212 | D111E/M153K/S155F | + |
| 213/214 | K62I | + |
| 215/216 | E80G | + |
| 217/218 | S112M | + |
| 219/220 | D175S | + |
| 221/222 | P74S | + |
| 223/224 | A14E | + |
| 225/226 | D57L | + |
| 227/228 | I51G | + |
| 229/230 | G94F | + |
| 231/232 | E68A | + |
| 233/234 | A105R | + |
| 235/236 | K170A | + |
| 237/238 | Q117N | + |
| 239/240 | K128S | + |
| 241/242 | P40F | + |
| 243/244 | S112C | + |
| 245/246 | G94A | + |
| 247/248 | K61A | + |
| 249/250 | E68W | + |
| 251/252 | K202G | + |
| 253/254 | E180P | + |
| 255/256 | K119R | + |
| 257/258 | I66G | + |
| 259/260 | E179G | + |
| 261/262 | S155F | + |
| 263/264 | L59V | + |
| 265/266 | N79L | + |
| 267/268 | A220E | + |
| 269/270 | I51F | + |
| 271/272 | K93A | + |
| 273/274 | I226L | + |
| 275/276 | E108M/S155F | + |
| 277/278 | E108M/D111E | + |
| 279/280 | D111E | + |
| 281/282 | K69Q/E88N | + |
| 283/284 | S43G | + |
| 285/286 | S43G/K69Q/L138I | + |
| 287/288 | P40C/S43G/E88N/V134L/K178N | + |
| 289/290 | G214P | + |
| 291/292 | E80N/V82I/H184K/V219W | + |
| 293/294 | E88N/Q136A | + |
| 295/296 | L16M/P40C/S43G/E88N | + |
| 297/298 | P40C/V134L | + |
| 299/300 | E88N | + |
| 301/302 | D111E/S155F | + |

Levels of increased activity were determined relative to the reference polypeptide of SEQ ID NO: 4 and defined as follows:
“+” 1.25 to 3.52,
“++” >3.52,
“+++” >8.02.

### Example 9

### Improvements Over SEQ ID NO: 4 in Conversion of Nucleoside Monophosphates to Nucleotides

### HTP Screening for Improved AdyK Variants

Adenylate kinase SEQ ID NO: 4 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 9.1.

Reactions were performed as described in Example 4 using conditions summarized in Table 9.1. Data were collected using the HPLC assay described in Example 5.

TABLE 9.1
Reaction conditions

Lysis Buffer - TEoA (pH 7.5), 0.1 g/L lysozyme; Lysis
Conditions - 100 μL, 60° C., 60 min; Reaction buffer -
50 mM Tris, 50 mM LiK(acetyl phosphate), 10 μM ATP,
10 mM magnesium chloride, pH 8.0; Lysate concentration
(vol %) - 20; Reaction Conditions - 1 μL, 30° C., 1 hr;
Nucleoside substrate - AMP; Substrate Concentration -
10 mM; Auxiliary Cascade Enzyme (Ack) - SEQ ID NO: 1314
(1 μM); Dilution into Coupling Reaction - 200X; Substrate
Oligonucleotides - SEQ ID NO: 1321, SEQ ID NO: 1322; Product
Oligonucleotides - SEQ ID NO: 1327, SEQ ID NO: 1328.

Activity relative to SEQ ID NO: 4 (Activity FIOP) was calculated based on the percentage of extension products observed for the variant compared with the percentage observed with SEQ ID NO: 4 (where the percent product may be set as the average of replicates or else the highest single sample as appropriate). The results are shown in Table 9.2.

TABLE 9.2
Adenylate kinase activity relative to SEQ ID NO: 4

| SEQ ID | Amino Acid Differences | FIOP Percent Product |
|---|---|---|
| NO: | (Relative to | Relative to |
| (nt/aa) | SEQ ID NO: 4) | SEQ ID NO: 4 |
| 87/88 | G94S | + |
| 93/94 | D57P | + |
| 95/96 | V78G | +++ |
| 123/124 | F109E | + |
| 135/136 | Y100S | ++ |
| 139/140 | E108S | + |
| 141/142 | E58G | + |
| 145/146 | E180G | ++ |
| 147/148 | K178R | + |
| 161/162 | K62S | + |
| 165/166 | E55Q | ++ |
| 201/202 | E35S | ++ |
| 207/208 | P40T | + |
| 213/214 | K62I | + |
| 231/232 | E68A | + |
| 239/240 | K128S | +++ |
| 243/244 | S112C | + |
| 247/248 | K61A | + |
| 251/252 | K202G | +++ |
| 255/256 | K119R | + |
| 257/258 | I66G | + |
| 259/260 | E179G | + |
| 265/266 | N79L | + |
| 271/272 | K93A | ++ |
| 303/304 | L223T | +++ |
| 305/306 | P74G | +++ |
| 307/308 | E115R | +++ |
| 309/310 | I39Q | +++ |
| 311/312 | K93E | +++ |
| 313/314 | K119L | ++ |
| 315/316 | L190N | ++ |
| 317/318 | K202M | ++ |
| 319/320 | E55S | ++ |
| 321/322 | I51L | ++ |
| 323/324 | S112Q | ++ |
| 325/326 | G94T | ++ |
| 327/328 | I66A | ++ |
| 329/330 | G201A | ++ |
| 331/332 | G201K | + |
| 333/334 | G201L | + |
| 335/336 | I66R | + |
| 337/338 | K128N | + |
| 339/340 | G214L | + |
| 341/342 | L126S | + |
| 343/344 | N56E | + |
| 345/346 | E179I | + |
| 347/348 | D216S | + |
| 349/350 | E76P | + |
| 351/352 | K69Y | + |
| 353/354 | E115G | + |
| 355/356 | D166C | + |
| 357/358 | Q120L | + |
| 359/360 | S116L | + |
| 361/362 | E108V | + |
| 363/364 | K203E | + |
| 365/366 | Q194F | + |
| 367/368 | L138M | + |
| 369/370 | K178E | + |
| 371/372 | G38C | + |

Levels of increased activity were determined relative to the reference polypeptide of SEQ ID NO: 4 and defined as follows: “+” 1.05 to 1.16, “++” > 1.16, “+++” > 1.27.

### Example 10

### Improvements Over SEQ ID NO: 172 in Conversion of Nucleoside Monophosphates to Nucleotides

### HTP Screening for Improved AdyK Variants

Adenylate kinase of SEQ ID NO: 172 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 10.1.

Reactions were performed as described in Example 4 using conditions summarized in Table 10.1. Data were collected using the HPLC assay described in Example 5.

TABLE 10.1
Reaction conditions

Lysis Buffer - TEoA (pH 7.5), 0.1 g/L lysozyme; Lysis Conditions - 100 μL, 60° C., 60 min;
Reaction buffer - 50 mM Tris, 50 mM LiK(acetyl phosphate), 10 μM ATP, 10 mM magnesium
chloride, pH 8.0; Lysate concentration (vol %) - 50; Reaction Conditions - 1 μL, 30° C., 1 hr;
Nucleoside substrate - GMP; Substrate Concentration - 10 mM; Auxiliary Cascade Enzymes (Ack) -
SEQ ID NO: 1314 (1 μM); Dilution into Coupling Reaction - 80X; Substrate Oligonucleotides -
SEQ ID NO: 1321, SEQ ID NO: 1322; Product Oligonucleotides - SEQ ID NO: 1325, SEQ ID NO:
1326.

Activity relative to SEQ ID NO: 172 (Activity FIOP) was calculated based on the percentage of extension products observed for the variant compared with the percentage observed with SEQ ID NO: 172 (where the percent product may be set as the average of replicates or else the highest single sample as appropriate). The results are shown in Table 10.2.

TABLE 10.2
Adenylate kinase activity relative to SEQ ID NO: 172

| SEQ ID | Amino Acid Differences | FIOP Percent Product |
|---|---|---|
| NO: | (Relative to | Relative to |
| (nt/aa) | SEQ ID NO: 172) | SEQ ID NO: 172 |
| 373/374 | I66E/V78G | +++ |
| 375/376 | V78G/E80A/K224G | +++ |
| 377/378 | V78G | +++ |
| 379/380 | E65P/I66E | +++ |
| 381/382 | V78G/K224G | ++ |
| 383/384 | V78G/E80A/F109E | ++ |
| 385/386 | P40F/G60S/G94S/L226I | ++ |
| 387/388 | I66A/E68R | ++ |
| 389/390 | I66E/E68R | ++ |
| 391/392 | V78G/E80A | ++ |
| 393/394 | G94S/Q117N/N118R/L226I | + |
| 395/396 | E68R/F109E | + |
| 397/398 | E68R | + |
| 399/400 | G94S/E179L | + |
| 401/402 | I51R/I66E/E68R/K224G | + |
| 403/404 | G94S/Q117N | + |
| 405/406 | I51R/I66E/E68R/V78G/K224G | + |
| 407/408 | I66E | + |
| 409/410 | N118R | + |
| 411/412 | K93V/G94S/E180P | + |
| 413/414 | F109E | + |
| 415/416 | E68R/K224G | + |
| 417/418 | P40F/K62L/K93V | + |
| 419/420 | K93V | + |
| 421/422 | I51R/I66E | + |
| 423/424 | G94S/Q117N/N118R | + |
| 425/426 | D198G/A220E | + |
| 427/428 | K93V/Q117N | + |
| 429/430 | Q117N/N118R | + |
| 431/432 | K93V/D198G | + |
| 433/434 | E68R/Q173R | + |
| 435/436 | P40F/Q117N | + |
| 437/438 | K224G | + |
| 439/440 | P40F/N118R/L226I | + |
| 441/442 | K93V/G94S | + |
| 443/444 | P40F/K62L/N118R | + |

Levels of increased activity were determined relative to the reference polypeptide of SEQ ID NO: 172 and defined as follows: “+” 1.54 to 2.95, “++” > 2.95, “+++” > 5.18.

### Example 11

### Improvements Over SEQ ID NO: 376 in Conversion of Nucleosides to Nucleotides

### HTP Screening for Improved AdyK Variants

Adenylate kinase of SEQ ID NO: 376 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 11.1.

Reactions were performed as described in Example 4 using conditions summarized in Table 11.1. Data were collected using the HPLC assay described in Example 5.

TABLE 11.1
Reaction conditions

Lysis Buffer - TEoA (pH 7.5), 0.1 g/L lysozyme; Lysis Conditions - 100 μL, 70° C., 60 min;
Reaction buffer - 50 mM Tris, 50 mM LiK(acetyl phosphate), 10 μM ATP, 10 mM magnesium
chloride, pH 8.0; Lysate concentration (vol %) - 50; Reaction Conditions - 1 μL, 30° C., 1 hr;
Nucleoside substrate - G; Substrate Concentration - 10 mM; Auxiliary Cascade Enzymes
(AdoK/AcK) - SEQ ID NO: 1304 (10 μM), SEQ ID NO: 1316 (10 μM); Dilution into Coupling
Reaction - 80X; Substrate Oligonucleotides - SEQ ID NO: 1321, SEQ ID NO: 1322; Product
Oligonucleotides - SEQ ID NO: 1325, SEQ ID NO: 1326.

Stability relative to SEQ ID NO: 376 (Stability FIOP) was calculated based on the percentage of extension products observed for the variant compared with the percentage observed with SEQ ID NO: 376 (where the percent product may be set as the average of replicates or else the highest single sample as appropriate). The results are shown in Table 11.2.

TABLE 11.2
Adenylate kinase activity relative to SEQ ID NO: 376

| SEQ ID | Amino Acid Differences | FIOP Percent Product |
|---|---|---|
| NO: | (Relative to | Relative to |
| (nt/aa) | SEQ ID NO: 376) | SEQ ID NO: 376 |
| 445/446 | M13S | +++ |
| 447/448 | G60A | +++ |
| 449/450 | L126E | +++ |
| 451/452 | V104L | +++ |
| 453/454 | G201S | ++ |
| 455/456 | L59E | ++ |
| 457/458 | E55Q | ++ |
| 459/460 | V133E | ++ |
| 461/462 | K61P | ++ |
| 463/464 | L59M | ++ |
| 465/466 | M13G | ++ |
| 467/468 | K53A | ++ |
| 469/470 | L190A | + |
| 471/472 | D57S | + |
| 473/474 | K62A | + |
| 475/476 | T181V | + |
| 477/478 | E55G | + |
| 479/480 | K168G | + |
| 481/482 | K54R | + |
| 483/484 | K54Q | + |
| 485/486 | Q173R | + |
| 487/488 | K168Q | + |
| 489/490 | K168N | + |
| 491/492 | K170P | + |
| 493/494 | Q34V | + |
| 495/496 | L59H | + |
| 497/498 | R183L | + |
| 499/500 | Q173K | + |
| 501/502 | R183A | + |
| 503/504 | A14E | + |

Levels of increased activity were determined relative to the reference polypeptide of SEQ ID NO: 376 and defined as follows: “+” 1.11 to 2.01, “++” > 2.01, “+++” > 3.01.

### Example 12

### Improvements Over SEQ ID NO: 376 in Conversion of Nucleosides to Nucleotides

### Shake Flask Characterization of AdyK Variants

AdyK variants SEQ ID NO: 376, SEQ ID NO: 506, SEQ ID NO: 508, and SEQ ID NO: 510 were expressed and purified as described in Example 3.

To assess activity, each variant was added to a 5 μL reaction at a final concentration of 5 μM. The reaction contained 50 mM Tris (pH 8.0), 50 mM lithium potassium acetyl phosphate, 10 μM ATP, 10 mM MgCl₂, 10 μM SEQ ID NO: 1304, 10 μM SEQ ID NO: 1314, and 10 mM guanosine. Reactions were incubated in a Multitron (Infors) shaker at 30° C. & 400 rpm for 60 min. Reactions were then quenched and diluted 40-fold with 75% methanol and analyzed by HPLC as described in Example 5. Activity relative to SEQ ID NO: 376 (Activity FIOP) was calculated based on the percent product observed for variants compared to the percent product observed in the reaction with SEQ ID NO: 376 (where the percent product may be set as the average of replicates or else the highest single sample as appropriate). The results are shown in Table 12.1.

TABLE 12.1
Adenylate kinase activity relative to SEQ ID NO: 376

| SEQ ID | Amino Acid Differences | FIOP Percent Product |
|---|---|---|
| NO: | (Relative to | Relative to |
| (nt/aa) | SEQ ID NO: 376 | SEQ ID NO: 376 |
| 505/506 | G60A/K62A/A124V/K170G | ++ |
| 507/508 | G60A/K170G | + |
| 509/510 | G60A/K62A | + |

Levels of increased activity were determined relative to the reference polypeptide of SEQ ID NO: 376 and defined as follows: “+” 2.81 to 2.99, “++” > 2.99.

### Example 13

Improvements Over SEQ ID NO: 508 in conversion of Nucleosides to Nucleotides

### HTP Screening for Improved AdyK Variants

Adenylate kinase of SEQ ID NO: 508 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 13.1.

Reactions were performed as described in Example 4 using conditions summarized in Table 13.1. Data were collected using the HPLC assay described in Example 5.

TABLE 13.1
Reaction conditions

Lysis Buffer - TEoA (pH 7.5), 0.1 g/L lysozyme; Lysis Conditions - 100 μL, 60° C., 60 min;
Reaction buffer - 50 mM Tris, 50 mM LiK(acetyl phosphate), 10 μM ATP, 10 mM magnesium
chloride, pH 8.0; Lysate concentration (vol %) - 50; Reaction Conditions - 1 μL, 30° C., 1 hr;
Nucleoside substrate - G; Substrate Concentration - 10 mM; Auxiliary Cascade Enzymes
(AdoK/AcK) - SEQ ID NO: 1304 (10 μM), SEQ ID NO: 1316 (10 μM); Dilution into Coupling
Reaction - 160X; Substrate Oligonucleotides - SEQ ID NO: 1321, SEQ ID NO: 1322; Product
Oligonucleotides - SEQ ID NO: 1325, SEQ ID NO: 1326.

Activity relative to SEQ ID NO: 508 (Activity FlOP) was calculated based on the percentage of extension products observed for the variant compared with the percentage observed with SEQ ID NO: 508 (where the percent product may be set as the average of replicates or else the highest single sample as appropriate). The results are shown in Table 13.2.

TABLE 13.2
Adenylate kinase activity relative to SEQ ID NO: 508

| SEQ ID | Amino Acid Differences | FIOP Percent Product |
|---|---|---|
| NO: | (Relative to | Relative to |
| (nt/aa) | SEQ ID NO: 508) | SEQ ID NO: 508 |
| 511/512 | Y15F | +++ |
| 513/514 | Y15F/E68A | +++ |
| 515/516 | Y15F/T181V | +++ |
| 517/518 | M13S/Y15F | +++ |
| 519/520 | M13S/Y15F/G201S | +++ |
| 521/522 | K54Q/E55Q | +++ |
| 523/524 | E55Q/V133E | +++ |
| 525/526 | E55Q/V133E/I197A | ++ |
| 527/528 | K54Q/E55Q/V133E/I197A | ++ |
| 529/530 | E68A | ++ |
| 531/532 | K54Q/V133E/I197A | ++ |
| 533/534 | K54Q/I197A | ++ |
| 535/536 | M13S/Y15F/E68Y | ++ |
| 537/538 | Q34V | ++ |
| 539/540 | K54Q | ++ |
| 541/542 | V133E | ++ |
| 543/544 | Q120K | + |
| 545/546 | Q34V/V133A | + |
| 547/548 | M13S/Y15F/T181V | + |
| 549/550 | E55Q | + |
| 551/552 | Y15F/L59M | + |
| 553/554 | E68A/T181V | + |
| 555/556 | G201S | + |
| 557/558 | E68Y | + |
| 559/560 | T181V | + |
| 561/562 | I66S | + |
| 563/564 | Y15F/L59M/T181V | + |
| 565/566 | I197A | + |
| 567/568 | M13S/Y15F/L59M | + |
| 569/570 | K61S | + |
| 571/572 | Q34V/K61S | + |
| 573/574 | M13S | + |
| 575/576 | K61A | + |
| 577/578 | Q34V/K69R | + |
| 579/580 | L163S | + |

Levels of increased activity were determined relative to the reference polypeptide of SEQ ID NO: 508 and defined as follows: “+” 1.06 to 1.39, “++” > 1.39, “+++” > 1.60.

### Example 14

### Improvements Over SEQ ID NO: 524 in Conversion of Nucleosides to Nucleotides

### Shake Flask Characterization of AdyK Variants

AdyK variants SEQ ID NO: 524, SEQ ID NO: 582, and SEQ ID NO: 584 were expressed and purified as described in Example 3.

To assess activity, each variant was added to a 5 μL reaction at a final concentration of 5 μM. The reaction contained 50 mM Tris (pH 8.0), 50 mM lithium potassium acetyl phosphate, 1 μM ATP, 10 mM MgCl₂, 1 μM SEQ ID NO: 1304, 1 μM SEQ ID NO: 1316, and 10 mM guanosine. Reactions were incubated in a Multitron (Infors) shaker at 30° C. & 400 rpm for 60 min. Reactions were then quenched and diluted 40-fold with 75% methanol and analyzed by HPLC as described in Example 5. Activity relative to SEQ ID NO: 524 (Activity FIOP) was calculated based on the percent product observed for variants compared to the percent product observed in the reaction with SEQ ID NO: 524 (where the percent product may be set as the average of replicates or else the highest single sample as appropriate). The results are shown in Table 14.1.

TABLE 14.1
Adenylate kinase activity relative to SEQ ID NO: 524

| SEQ ID | Amino Acid Differences | FIOP Percent Product |
|---|---|---|
| NO: | (Relative to | Relative to |
| (nt/aa) | SEQ ID NO: 524) | SEQ ID NO: 524 |
| 581/582 | Q120R | + |
| 583/584 | K61S/G201S | ++ |

Levels of increased activity were determined relative to the reference polypeptide of SEQ ID NO: 524 and defined as follows: “+” 1.48 to 1.67, “++” > 1.67.

### Example 15

### Improvements Over SEQ ID NO: 524 in Conversion of Nucleosides to Nucleotides

### HTP Screening for Improved AdyK Variants

Adenylate kinase of SEQ ID NO: 524 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 15.1.

Reactions were performed as described in Example 4 using conditions summarized in Table 15.1. Data were collected using the HPLC assay described in Example 5.

TABLE 15.1
Reaction conditions

Lysis Buffer - TEoA (pH 7.5), 0.1 g/L lysozyme; Lysis Conditions - 100 μL, 60° C., 60 min;
Reaction buffer - 50 mM Tris, 50 mM LiK(acetyl phosphate), 10 μM ATP, 10 mM magnesium
chloride, pH 8.0; Lysate concentration (vol %) - 5; Reaction Conditions - 1 μL, 30° C., 1 hr;
Nucleoside substrate - G; Substrate Concentration - 10 mM; Auxiliary Cascade Enzymes
(AdoK/AcK) - SEQ ID NO: 1308 (10 μM), SEQ ID NO: 1316 (10 μM); Dilution into Coupling
Reaction - 80X; Substrate Oligonucleotides - SEQ ID NO: 1323, SEQ ID NO: 1324; Product
Oligonucleotides - SEQ ID NO: 1329, SEQ ID NO: 1330.

Activity relative to SEQ ID NO: 524 (Activity FIOP) was calculated based on the percentage of extension products observed for the variant compared with the percentage observed with SEQ ID NO: 524 (where the percent product may be set as the average of replicates or else the highest single sample as appropriate). The results are shown in Table 15.2.

TABLE 15.2
Adenylate kinase activity relative to SEQ ID NO: 524

| SEQ ID | Amino Acid Differences | FIOP Percent Product |
|---|---|---|
| NO: | (Relative to | Relative to |
| (nt/aa) | SEQ ID NO: 524) | SEQ ID NO: 524 |
| 585/586 | Q34S | +++ |
| 587/588 | K36I | +++ |
| 589/590 | K36V | ++ |
| 591/592 | K36A | ++ |
| 593/594 | K36L | + |
| 595/596 | K36M | + |
| 597/598 | A105S | + |
| 599/600 | Q34A | + |
| 601/602 | S112E | + |
| 603/604 | K31R | + |
| 605/606 | K146D | + |
| 607/608 | S112R | + |

Levels of increased activity were determined relative to the reference polypeptide of SEQ ID NO: 524 and defined as follows: “+” 1.02 to 1.15, “++” > 1.15, “+++” > 1.30.

### Example 16

### Improvements Over SEQ ID NO: 584 in Conversion of Nucleosides to Nucleotides

### HTP Screening for Improved AdyK Variants

Adenylate kinase of SEQ ID NO: 584 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 16.1.

Reactions were performed as described in Example 4 using conditions summarized in Table 16.1. Data were collected using the HPLC assay described in Example 5.

TABLE 16.1
Reaction conditions

Lysis Buffer - TEoA (pH 7.5), 0.1 g/L lysozyme; Lysis Conditions - 100 μL, 60° C., 60 min; Reaction
buffer - 50 mM Tris, 50 mM LiK(acetyl phosphate), 10 μM ATP, 10 mM magnesium chloride, pH
8.0; Lysate concentration (vol %) - 2.5; Reaction Conditions - 1 μL, 30° C., 1 hr; Nucleoside substrate -
G; Substrate Concentration - 10 mM; Auxiliary Cascade Enzymes (AdoK/AcK) - SEQ ID NO: 1306
(10 μM), SEQ ID NO: 1316 (10 μM); Dilution into Coupling Reaction - 800X; Substrate
Oligonucleotides - SEQ ID NO: 1321, SEQ ID NO: 1322; Product Oligonucleotides - SEQ ID NO:
1325, SEQ ID NO: 1326.

Activity relative to SEQ ID NO: 584 (Activity FIOP) was calculated based on the percentage of extension products observed for the variant compared with the percentage observed with SEQ ID NO: 584 (where the percent product may be set as the average of replicates or else the highest single sample as appropriate). The results are shown in Table 16.2.

TABLE 16.2
Adenylate kinase activity relative to SEQ ID NO: 584

| SEQ ID | Amino Acid Differences | FIOP Percent Product |
|---|---|---|
| NO: | (Relative to | Relative to |
| (nt/aa) | SEQ ID NO: 584) | SEQ ID NO: 584 |
| 609/610 | M13G/S30G/K31R/K53A/ | +++ |
| | F109Y/K119R/K168G | |
| 611/612 | K36Y/L126E/L190A | +++ |
| 613/614 | L126E | +++ |
| 615/616 | K36Q/I39M/L126E | +++ |
| 617/618 | L126E/L190A | +++ |
| 619/620 | K36Y/I39M/L126E | +++ |
| 621/622 | K36Q/L126E | ++ |
| 623/624 | K36Y/L190A | ++ |
| 625/626 | I39M/L126E | ++ |
| 627/628 | M13G/F109Y/K119R | ++ |
| 629/630 | F109Y | ++ |
| 631/632 | K36Y/Q120G/L190A | ++ |
| 633/634 | L126E/G148T | + |
| 635/636 | L190A | + |
| 637/638 | E108R/L126E | + |
| 639/640 | S30G/K31R | + |
| 641/642 | M13G/N118A/V182I | + |
| 643/644 | K31R/K53A | + |
| 645/646 | I39M | + |
| 647/648 | K119R | + |
| 649/650 | M13G/K119R/K168G | + |
| 651/652 | K53A/K168G | + |
| 653/654 | Q34S | + |
| 655/656 | Q34S/E65A/K146D | + |
| 657/658 | K36Q/I39M | + |
| 659/660 | K119R/K168G | + |
| 661/662 | Q34S/S112R | + |
| 663/664 | K36Y/I39M | + |
| 665/666 | Q173K | + |

Levels of increased activity were determined relative to the reference polypeptide of SEQ ID NO: 584 and defined as follows: “+” 1.09 to 1.29, “++” > 1.29, “+++” > 1.46.

### Example 17

### Improvements Over SEQ ID NO: 610 in Conversion of Nucleosides to Nucleotides

### HTP Screening for Improved AdyK Variants

Adenylate kinase of SEQ ID NO: 610 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 17.1.

Reactions were performed as described in Example 4 using conditions summarized in Table 17.1. Data were collected using the HPLC assay described in Example 5.

TABLE 17.1
Reaction conditions

Lysis Buffer - TEoA (pH 7.5), 0.1 g/L lysozyme; Lysis Conditions - 100 μL, 60° C., 60 min; Reaction
buffer - 50 mM Tris, 50 mM LiK(acetyl phosphate), 10 μM ATP, 10 mM magnesium chloride, pH 8.0;
Lysate concentration (vol %) - 2.5; Reaction Conditions - 1 μL, 30° C., 1 hr; Nucleoside substrate - G;
Substrate Concentration - 10 mM; Auxiliary Cascade Enzymes (AdoK/AcK) - SEQ ID NO: 1308
(10 μM), SEQ ID NO: 1318 (10 μM); Dilution into Coupling Reaction - 800X; Substrate Oligonucleotides -
SEQ ID NO: 1323, SEQ ID NO: 1324; Product Oligonucleotides - SEQ ID NO: 1329, SEQ ID NO:
1330.

Activity relative to SEQ ID NO: 610 (Activity FIOP) was calculated based on the percentage of extension products observed for the variant compared with the percentage observed with SEQ ID NO: 610 (where the percent product may be set as the average of replicates or else the highest single sample as appropriate). The results are shown in Table 17.2.

TABLE 17.2
Adenylate kinase activity relative to SEQ ID NO: 610

| SEQ ID | Amino Acid Differences | FIOP Percent Product |
|---|---|---|
| NO: | (Relative to | Relative to |
| (nt/aa) | SEQ ID NO: 610) | SEQ ID NO: 610 |
| 667/668 | L190A | +++ |
| 669/670 | E115K/L126E/L190A | ++ |
| 671/672 | L126E/L190A | ++ |
| 673/674 | Q34S/E35A/A105R | + |
| 675/676 | L126E | + |
| 677/678 | L59E/L126E | + |
| 679/680 | Q34S/A105R/S116E/K146D | + |

Levels of increased activity were determined relative to the reference polypeptide of SEQ ID NO: 610 and defined as follows: “+” 1.42 to 2.72, “++” > 2.72, “+++” > 3.83.

### Example 18

### Improvements Over SEQ ID NO: 610 in Conversion of Nucleosides to Nucleotides

### HTP Screening for Improved AdyK Variants

Adenylate kinase of SEQ ID NO: 610 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 18.1.

Reactions were performed as described in Example 4 using conditions summarized in Table 18.1. Data were collected using the HPLC assay described in Example 5.

TABLE 18.1
Reaction conditions

Lysis Buffer - TEoA (pH 7.5), 0.1 g/L lysozyme; Lysis Conditions - 100 μL, 60° C., 60 min; Reaction
buffer - 50 mM Tris, 50 mM LiK(acetyl phosphate), 10 μM ATP, 10 mM magnesium chloride, pH 8.0;
Lysate concentration (vol %) - 25; Reaction Conditions - 1 μL, 30° C., 16 hr; Nucleoside substrate - fG;
Substrate Concentration - 10 mM; Auxiliary Cascade Enzymes (AdoK/AcK) - SEQ ID NO: 1310 (50
μM), SEQ ID NO: 1318 (10 μM); Dilution into Coupling Reaction - 80X; Substrate Oligonucleotides -
SEQ ID NO: 1323, SEQ ID NO: 1324; Product Oligonucleotides - SEQ ID NO: 1331, SEQ ID NO:
1332.

Activity relative to SEQ ID NO: 610 (Activity FIOP) was calculated based on the percentage of extension products observed for the variant compared with the percentage observed with SEQ ID NO: 610 (where the percent product may be set as the average of replicates or else the highest single sample as appropriate). The results are shown in Table 18.2.

TABLE 18.2
Adenylate kinase activity relative to SEQ ID NO: 610

| | | FIOP Percent |
|---|---|---|
| SEQ ID NO: | Amino Acid Differences | Product Relative to |
| (nt/aa) | (Relative to SEQ ID NO: 610) | SEQ ID NO: 610 |
| 681/682 | F48L | +++ |
| 683/684 | I66F | +++ |
| 685/686 | L59T | +++ |
| 687/688 | G148H | +++ |
| 689/690 | V82T | +++ |
| 691/692 | L59V | ++ |
| 693/694 | L59Y | ++ |
| 695/696 | L59P | ++ |
| 697/698 | Y100F | ++ |
| 699/700 | G148F | ++ |
| 701/702 | G27S | ++ |
| 703/704 | V135R | ++ |
| 705/706 | G148T | ++ |
| 707/708 | L77M | ++ |
| 709/710 | L138C | ++ |
| 711/712 | G148Q | ++ |
| 713/714 | L126A | ++ |
| 715/716 | V52A | ++ |
| 717/718 | I66Q | ++ |
| 719/720 | L59W | ++ |
| 721/722 | L59G | + |
| 723/724 | G148M | + |
| 725/726 | L59M | + |
| 727/728 | V135S | + |
| 729/730 | E68L | + |
| 731/732 | T139A | + |
| 733/734 | H184I | + |
| 735/736 | E68R | + |
| 737/738 | H184V | + |
| 739/740 | I66S | + |
| 741/742 | E68Q | + |
| 743/744 | V81I | + |
| 745/746 | E68V | + |
| 747/748 | E65F | + |
| 749/750 | M153S | + |
| 751/752 | L126V | + |
| 753/754 | E68A | + |
| 755/756 | I66T | + |
| 757/758 | D50C | + |
| 759/760 | L138V | + |
| 761/762 | T181I | + |
| 763/764 | G214T | + |
| 765/766 | T139L | + |
| 767/768 | E68T | + |
| 769/770 | G148S | + |
| 771/772 | V172H | + |
| 773/774 | L138I | + |
| 775/776 | K69L | + |
| 777/778 | V81Q | + |
| 779/780 | I154R | + |

Levels of increased activity were determined relative to the reference polypeptide of SEQ ID NO: 610 and defined as follows: “+” 1.11 to 1.52, “++” > 1.52, “+++” > 1.95.

### Example 19

### Improvements Over SEQ ID NO: 674 in Conversion of Nucleosides to Nucleotides

### HTP Screening for Improved AdyK Variants

SEQ ID NO: 674 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 19.1.

Reactions were performed as described in Example 4 using conditions summarized in Table 19.1. Data were collected using the HPLC assay described in Example 5.

TABLE 19.1
Reaction conditions

Lysis Buffer - TEoA (pH 7.5), 0.1 g/L lysozyme; Lysis Conditions - 100 μL, 60° C., 60 min; Reaction
buffer - 50 mM Tris, 50 mM LiK(acetyl phosphate), 10 μM ATP, 10 mM magnesium chloride, pH
8.0; Lysate concentration (vol %) - 1; Reaction Conditions - 1 μL, 30° C., 1 hr; Nucleoside substrate -
G; Substrate Concentration - 10 mM; Auxiliary Cascade Enzymes (AdoK/AcK) - SEQ ID NO: 1310
(10 μM), SEQ ID NO: 1318 (10 μM); Dilution into Coupling Reaction - 800X; Substrate
Oligonucleotides - SEQ ID NO: 1323, SEQ ID NO: 1324; Product Oligonucleotides - SEQ ID NO:
1329, SEQ ID NO: 1330.

Activity relative to SEQ ID NO: 674 (Activity FIOP) was calculated based on the percentage of extension products observed for the variant compared with the percentage observed with SEQ ID NO: 674 (where the percent product may be set as the average of replicates or else the highest single sample as appropriate). The results are shown in Table 19.2.

TABLE 19.2
Adenylate kinase activity relative to SEQ ID NO: 674

| | | FIOP Percent |
|---|---|---|
| SEQ ID NO: | Amino Acid Differences | Product Relative to |
| (nt/aa) | (Relative to SEQ ID NO: 674) | SEQ ID NO: 674 |
| 781/782 | V81I/G148T/V219L | +++ |
| 783/784 | E68A/G148H/V219L | +++ |
| 785/786 | E68A/V81I/V219L | +++ |
| 787/788 | G148H/V219F | +++ |
| 789/790 | E68A/G148T/V219L | +++ |
| 791/792 | V81I/V219L | +++ |
| 793/794 | Q136L/L138V/T139L | ++ |
| 795/796 | G148T/V219F | ++ |
| 797/798 | G148H/V219L | ++ |
| 799/800 | E68A/V219L | ++ |
| 801/802 | I66T/E68A/V219L | ++ |
| 803/804 | I66Q/G148H/V219F | ++ |
| 805/806 | G148T/V219L | ++ |
| 807/808 | I66T/E68A/G148H | ++ |
| 809/810 | L59V | ++ |
| 811/812 | L59V/V135R | ++ |
| 813/814 | V219F | ++ |
| 815/816 | L59V/T139L | + |
| 817/818 | L138V | + |
| 819/820 | L126A/G148T/V219L | + |
| 821/822 | L59V/T181I | + |
| 823/824 | V81I | + |
| 825/826 | I66Q/V81I/V219L | + |
| 827/828 | V219L | + |
| 829/830 | L126A/V219L | + |
| 831/832 | I66Q/E68A/V81I/G148T/V219L | + |
| 833/834 | T181I | + |
| 835/836 | G27S/G148H | + |
| 837/838 | V135R/Q136L/T139R | + |
| 839/840 | Q136L/L138V/T139R | + |
| 841/842 | G148T | + |
| 843/844 | L59V/V135R/L138V/T181I | + |
| 845/846 | L59V/V135R/Q136L/L138V/T139R | + |
| 847/848 | L59V/V135R/Q136L/L138V/T181I | + |
| 849/850 | I66Q/E68G/V81I/G148H/V219L | + |
| 851/852 | G27S | + |
| 853/854 | G27S/L126A/G148T | + |
| 855/856 | V135R/Q136L/L138V | + |
| 857/858 | E68G/V81I/L126A/G148H/V219L | + |
| 859/860 | Q136L | + |
| 861/862 | G27S/L126A/G148H | + |
| 863/864 | E68G | + |
| 865/866 | L126A/G148T | + |
| 867/868 | L138V/T139R | + |

Levels of increased activity were determined relative to the reference polypeptide of SEQ ID NO: 674 and defined as follows: “+” 1.20 to 3.48, “++” > 3.48, “+++” > 5.78.

### Example 20

### Improvements Over SEQ ID NO: 816 in Conversion of Nucleosides to Nucleotides

### HTP Screening for Improved AdyK Variants

Adenylate kinase of SEQ ID NO: 816 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 20.1.

Reactions were performed as described in Example 4 using conditions summarized in Table 20.1. Data were collected using the HPLC assay described in Example 5.

TABLE 20.1
Reaction conditions

Lysis Buffer - TEoA (pH 7.5), 0.1 g/L lysozyme; Lysis Conditions - 100 μL, 60° C., 60 min;
Reaction buffer - 50 mM Tris, 50 mM LiK(acetyl phosphate), 10 μM ATP, 10 mM magnesium
chloride, pH 8.0; Lysate concentration (vol %) - 25; Reaction Conditions - 1 μL, 30° C., 16 hr;
Nucleoside substrate - fG; Substrate Concentration - 10 mM; Auxiliary Cascade Enzymes
(AdoK/AcK) - SEQ ID NO: 1310 (10 μM), SEQ ID NO: 1318 (10 μM); Dilution into Coupling
Reaction - 800X; Substrate Oligonucleotides - SEQ ID NO: 1323, SEQ ID NO: 1324; Product
Oligonucleotides - SEQ ID NO: 1331, SEQ ID NO: 1332.

Activity relative to SEQ ID NO: 816 (Activity FIOP) was calculated based on the percentage of extension products observed for the variant compared with the percentage observed with SEQ ID NO: 816 (where the percent product may be set as the average of replicates or else the highest single sample as appropriate). The results are shown in Table 20.2.

TABLE 20.2
Adenylate kinase activity relative to SEQ ID NO: 816

| | | FIOP Percent |
|---|---|---|
| SEQ ID NO: | Amino Acid Differences | Product Relative to |
| (nt/aa) | (Relative to SEQ ID NO: 816) | SEQ ID NO: 816 |
| 869/870 | F48L/Y100F/G148H | +++ |
| 871/872 | F48L/I66F/G148F/V219L | +++ |
| 873/874 | F48L/Y100F | +++ |
| 875/876 | F48L/Y100F/V135R/Q136L | +++ |
| 877/878 | F48L/I66F/L77M/Y100F/V135R/ | +++ |
| | Q136L/G148H | |
| 879/880 | F48L/Y100F/Q136L | +++ |
| 881/882 | F48L/V135R/G148F | +++ |
| 883/884 | F48L/G148F | +++ |
| 885/886 | F48L/Q136L/G148F | +++ |
| 887/888 | F48L/V135R | +++ |
| 889/890 | F48L/V135R/Q136L | +++ |
| 891/892 | I66F/Y100F | +++ |
| 893/894 | F48L/I66Q/V219L | +++ |
| 895/896 | F48L/V135R/V219L | ++ |
| 897/898 | Y100F/G148H | ++ |
| 899/900 | F48L/Q136L/V219L | ++ |
| 901/902 | F48L/V219L | ++ |
| 903/904 | F48L/V135R/G148F/V219L | ++ |
| 905/906 | Y100F/V135R/Q136L | ++ |
| 907/908 | Y100F/Q136L | ++ |
| 909/910 | I66F/L77M/Y100F/Q136L/V219L | ++ |
| 911/912 | Y100F/V135R | ++ |
| 913/914 | F48L/L77M/Y100F/V135R/Q136L | ++ |
| 915/916 | Y100F | ++ |
| 917/918 | L77M/Y100F/V135R | + |
| 919/920 | F48L/I66F/Y100F/V135R/Q136L/ | + |
| | G148F/V219L | |
| 921/922 | V52A/H184I | + |
| 923/924 | I66F | + |
| 925/926 | V52A/E68L | + |
| 927/928 | Q136L/G148F | + |
| 929/930 | L126A/H184I | + |
| 931/932 | G148F | + |
| 933/934 | L126A/H184V | + |
| 935/936 | H184V | + |
| 937/938 | V52A | + |
| 939/940 | V135R/Q136L/G148F | + |
| 941/942 | L126A/L138V/H184V | + |
| 943/944 | H184I | + |
| 945/946 | G27S/H184V | + |
| 947/948 | G27S/L126A | + |
| 949/950 | G27S/L126A/G148R | + |
| 951/952 | G27S | + |
| 953/954 | G27S/L126A/H184V | + |
| 955/956 | I66Q/Y100F/V135R/V219L | + |
| 957/958 | G27S/E68A | + |
| 959/960 | V52A/E68A/V81L/L126A | + |
| 961/962 | I66Q | + |
| 963/964 | F48L/I66Q/Q136L/G148F | + |
| 965/966 | I66F/Q136L/G148H | + |
| 967/968 | I66Q/V135R | + |
| 969/970 | I66Q/Q136L | + |
| 971/972 | I66F/V135R/V219L | + |
| 973/974 | Q136L | + |
| 975/976 | V135R/G148F/V219L | + |
| 977/978 | E68A/L126A | + |
| 979/980 | I66F/G148H/V219L | + |
| 981/982 | G27S/E68L/L126A | + |
| 983/984 | G27S/E68L/H184V | + |
| 985/986 | L126A | + |
| 987/988 | E68L/V81L/L126A | + |
| 989/990 | L77M/V135R | + |
| 991/992 | V135R/Q136L | + |
| 993/994 | L77M/Q136L | + |
| 995/996 | L77M/G148F | + |
| 997/998 | V82T | + |
| 999/1000 | L138V/H184V | + |
| 1001/1002 | L77M/V135R/Q136L | + |
| 1003/1004 | G27S/L138V | + |
| 1005/1006 | V81L | + |
| 1007/1008 | V81L/L126A | + |
| 1009/1010 | G27S/L126A/L138V | + |
| 1011/1012 | E68A | + |
| 1013/1014 | V81L/L138V | + |

Levels of increased activity were determined relative to the reference polypeptide of SEQ ID NO: 816 and defined as follows: “+” 1.06 to 3.04, “++” > 3.04, “+++” > 5.27.

### Example 21

### Improvements Over SEQ ID NO: 874 in Conversion of Nucleoside Monophosphates to Nucleotides

### HTP Screening for Improved AdyK Variants

Adenylate kinase of SEQ ID NO: 874 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 21.1.

Reactions were performed as described in Example 4 using conditions summarized in Table 21.1. Data were collected using the HPLC assay described in Example 5.

TABLE 21.1
Reaction conditions

Lysis Buffer - TEoA (pH 7.5), 0.1 g/L lysozyme; Lysis Conditions - 100 μL, 50° C., 60 min; Reaction
buffer - 50 mM Tris, 50 mM LiK(acetyl phosphate), 10 μM ATP, 10 mM magnesium chloride, pH
8.0; Lysate concentration (vol %) - 25; Reaction Conditions - 1 μL, 30° C., 1 hr; Nucleoside substrate -
mUMP; Substrate Concentration - 10 mM; Auxiliary Cascade Enzyme (AcK) - SEQ ID NO: 1318 (10
μM); Dilution into Coupling Reaction - 80X; Substrate Oligonucleotides - SEQ ID NO: 1323, SEQ
ID NO: 1324; Product Oligonucleotides - SEQ ID NO: 1333, SEQ ID NO: 1334.

Activity relative to SEQ ID NO: 874 (Activity FIOP) was calculated based on the percentage of extension products observed for the variant compared with the percentage observed with SEQ ID NO: 874 (where the percent product may be set as the average of replicates or else the highest single sample as appropriate). The results are shown in Table 21.2.

TABLE 21.2
Adenylate kinase activity relative to SEQ ID NO: 874

| | | FIOP Percent |
|---|---|---|
| SEQ ID NO: | Amino Acid Differences | Product Relative to |
| (nt/aa) | (Relative to SEQ ID NO: 874) | SEQ ID NO: 874 |
| 1015/1016 | R32I/A60S/V135K/D175L/1225A | +++ |
| 1017/1018 | R32I/V81L/F127I/K128N/V135K/ | +++ |
| | D175L/I225A | |
| 1019/1020 | K83S/D175F/I225A | +++ |
| 1021/1022 | V135K/D175L/1225A | +++ |
| 1023/1024 | K54T/R84H/L190R | +++ |
| 1025/1026 | Q55S/L190H/S212N | +++ |
| 1027/1028 | R32I/A60S/V81L/K83S/F127I/ | +++ |
| | K128N/V135K/I225A | |
| 1029/1030 | D175F/I225A | +++ |
| 1031/1032 | R32I/F127I/D175L | +++ |
| 1033/1034 | R84A/L190H/S212N | +++ |
| 1035/1036 | R32I/K128N/D175F | +++ |
| 1037/1038 | V81L/D175L | +++ |
| 1039/1040 | V81L/D175F | +++ |
| 1041/1042 | P21R/K54T | +++ |
| 1043/1044 | K54T/Q55S/S212N | +++ |
| 1045/1046 | R32I/D175L/1225A | +++ |
| 1047/1048 | K128N/D175F/I225A | +++ |
| 1049/1050 | K62S/R84A/L190R | +++ |
| 1051/1052 | V81L/V135M/I225A | ++ |
| 1053/1054 | P21R/K54T/K62S/L190R/S212N | ++ |
| 1055/1056 | A60S/V81L/K83G/D175L | ++ |
| 1057/1058 | Q55S/R84A/L190R | ++ |
| 1059/1060 | R32I/V81L/K83G/V135K/I225A | ++ |
| 1061/1062 | D175F | ++ |
| 1063/1064 | K62S/S212N | ++ |
| 1065/1066 | R32I/V81L/K83S | ++ |
| 1067/1068 | R32I/V81L/I225A | ++ |
| 1069/1070 | K54T/Q55S/R84A/S212N | ++ |
| 1071/1072 | Q55S/L190R | ++ |
| 1073/1074 | K62S/E179P | ++ |
| 1075/1076 | K62E/R84A/L190H | ++ |
| 1077/1078 | K54T/R84A/E179P/S212N | ++ |
| 1079/1080 | K54T/Q55S/K62S/R84A | ++ |
| 1081/1082 | V81L/V135M | ++ |
| 1083/1084 | R32I/V81L/V135K | ++ |
| 1085/1086 | E179P/L190H/S212N | ++ |
| 1087/1088 | Q55S/K62E | ++ |
| 1089/1090 | F100Y | ++ |
| 1091/1092 | P21R/K54T/K62E/L190H | ++ |
| 1093/1094 | L190H/S212N | ++ |
| 1095/1096 | K128N/D175L | ++ |
| 1097/1098 | P21R/K62S/L190H | ++ |
| 1099/1100 | K54T/E179P/L190H | ++ |
| 1101/1102 | K54T/L190R | ++ |
| 1103/1104 | P21R/Q55S/L190H | ++ |
| 1105/1106 | P21R/K54T/K62E | ++ |
| 1107/1108 | P21R/R84A | ++ |
| 1109/1110 | P21R/Q55S | ++ |
| 1111/1112 | P21R/K62S | ++ |
| 1113/1114 | P21R/E179P | ++ |
| 1115/1116 | D175L | + |
| 1117/1118 | Q55S/K62E/L190H/S212N | + |
| 1119/1120 | K54T/K62E/R84A | + |
| 1121/1122 | F127I/K128N/D175L/I225A | + |
| 1123/1124 | K54T/K62E | + |
| 1125/1126 | F100Y/V104F | + |
| 1127/1128 | Q55S/K62E/L190R/S212N | + |
| 1129/1130 | K54T/Q55S | + |
| 1131/1132 | P21R/Q55S/S212N | + |
| 1133/1134 | K62S | + |
| 1135/1136 | Q55S/R84A | + |
| 1137/1138 | Q55S/K62S/S212N | + |
| 1139/1140 | P21R/R84A/L190H | + |
| 1141/1142 | I225A | + |
| 1143/1144 | P21R/K54T/E179P/L190R | + |
| 1145/1146 | K83S/V135K | + |
| 1147/1148 | R32I/D175L | + |
| 1149/1150 | P21R/K54T/G78T/L190R | + |
| 1151/1152 | K62E | + |
| 1153/1154 | Q55S/K62E/E179P/ | + |
| | L190H/S212N | |
| 1155/1156 | V81L/V135K | + |
| 1157/1158 | P21R/K54T/L190H | + |
| 1159/1160 | P21R/Q55S/R84A/L190H | + |
| 1161/1162 | K62S/R84A | + |
| 1163/1164 | P21R/Q55S/K62E | + |
| 1165/1166 | P21R/K62S/R84A | + |
| 1167/1168 | V135M | + |
| 1169/1170 | R32I/A60S/V81L/F127I | + |
| 1171/1172 | P21R/K54T/Q55S/K62E/G78T | + |
| 1173/1174 | F100Y/V104H | + |
| 1175/1176 | K54T/R84A | + |
| 1177/1178 | Q55S/K62S/E179P/L190R | + |
| 1179/1180 | K54T/Q55S/K62S/R84A/E179P/ | + |
| | L190H/S212N | |
| 1181/1182 | L190R | + |
| 1183/1184 | L190H | + |
| 1185/1186 | K83S | + |
| 1187/1188 | F100Y/V104R | + |
| 1189/1190 | G78T | + |
| 1191/1192 | K54T/K62E/L190R | + |
| 1193/1194 | P21R/Q55S/K62E/E179P/L190H | + |
| 1195/1196 | G78C | + |
| 1197/1198 | R32I/A60S/F127I | + |
| 1199/1200 | K54T/K62E/R84A/L190H/S212N | + |
| 1201/1202 | P21R/K62S/E179P | + |
| 1203/1204 | F100Y/V104Q | + |
| 1205/1206 | F127I/K128N/I225A | + |
| 1207/1208 | R32I/I225A | + |
| 1209/1210 | R32I/A60S/V81L | + |
| 1211/1212 | I225V | + |
| 1213/1214 | V81L | + |
| 1215/1216 | K54T | + |
| 1217/1218 | P21R | + |
| 1219/1220 | A60S/V81L/K128N/D175L | + |
| 1221/1222 | L190Q | + |
| 1223/1224 | R84A | + |
| 1225/1226 | P21R/L190R/S212N | + |
| 1227/1228 | L190C | + |
| 1229/1230 | V135K | + |
| 1231/1232 | A60S/K128N/I225A | + |
| 1233/1234 | I225T | + |
| 1235/1236 | K62G | + |
| 1237/1238 | I225Q | + |
| 1239/1240 | E179P | + |
| 1241/1242 | I225R | + |
| 1243/1244 | F100Y/V104S | + |
| 1245/1246 | K83H | + |
| 1247/1248 | P21R/Q55S/K62E/L190R | + |
| 1249/1250 | F100Y/V104W | + |
| 1251/1252 | L190G | + |
| 1253/1254 | K128N | + |
| 1255/1256 | R84M | + |
| 1257/1258 | A60S | + |
| 1259/1260 | G78N | + |
| 1261/1262 | R84S | + |
| 1263/1264 | K62L | + |
| 1265/1266 | K128E | + |
| 1267/1268 | S212N | + |
| 1269/1270 | Q55S | + |
| 1271/1272 | F127I | + |
| 1273/1274 | P21S | + |
| 1275/1276 | R32I | + |
| 1277/1278 | A80G | + |
| 1279/1280 | R84E | + |
| 1281/1282 | A220V | + |
| 1283/1284 | K62S/G78T/E179P | + |
| 1285/1286 | L190E | + |
| 1287/1288 | Q55S/K62E/R84A | + |
| 1289/1290 | P21R/Q55S/L190H/S212N | + |
| 1291/1292 | R32I/F127I/K128N/I225A | + |
| 1293/1294 | P21R/K54T/Q55S/R84A/E179P | + |
| 1295/1296 | P21R/L190R | + |
| 1297/1298 | R32I/A60S/V81L/K83S/ | + |
| | K128N/V135M | |
| 1299/1300 | F127I/K128N | + |
| 1301/1302 | P21R/Q55S/E179P | + |

Levels of increased activity were determined relative to the reference polypeptide of SEQ ID NO: 874 and defined as follows: “+” 1.11 to 3.06, “++” > 3.06, “+++” > 4.97.

### Example 22

Relative activities of AdyK variants for the Conversion of Nucleosides to Nucleotides

### Shake Flask Characterization of AdyK Variants

AdyK variants SEQ ID NO: 2, SEQ ID NO: 172, SEQ ID NO: 508, SEQ ID NO: 584, SEQ ID NO: 674, SEQ ID NO: 874, and SEQ ID NO: 1032 were expressed and purified as described in Example 3.

To assess activity, each variant was added to a 5 μL reaction at a final concentration of 10 μM. The reaction contained 50 mM Tris (pH 8.0), 50 mM lithium potassium acetyl phosphate, 10 μM ATP, 10 mM MgCl₂, 10 μM AdoK variant SEQ ID NO: 1312, AcK variant 10 μM SEQ ID NO: 1318, and 10 mM nucleoside. Reactions were incubated in a Multitron (Infors) shaker at 30° C. & 400 rpm for 60 min. Reactions were then quenched and diluted 40-fold with 75% methanol and analyzed by HPLC as described in Example 5. Relative activities were normalized to the lowest observed activity by a variant on a given substrate. The results are shown in Table 22.1.

TABLE 22.1

| | Relative Activities of AdyK Variants | | | | | | | | |
|---|---|---|---|---|---|---|---|---|---|
| SEQ ID NO: | on Nucleoside Substrates | | | | | | | | |
| (nt/aa) | A | C | G | U | fA | fC | fG | fU | mA |
| 1/2 | ~ | ~ | ~ | ~ | ~ | − | ~ | − | + |
| 171/172 | ~ | ++ | ++ | +++ | ~ | ~ | + | − | + |
| 507/508 | ~ | ++ | ++ | +++ | ~ | + | ++ | + | + |
| 583/584 | ~ | ++ | ++ | +++ | ~ | + | ++ | ~ | + |
| 673/674 | ~ | ++ | ++ | +++ | ~ | + | ++ | + | + |
| 873/874 | ~ | ++ | ++ | +++ | ~ | + | +++ | + | + |
| 1031/1032 | ~ | ++ | ++ | +++ | ~ | + | +++ | + | ~ |

Levels of relative activity were measured for the listed variants and defined as follows: “−” 0.00 to 0.99, “~” ≥ 1.0, “+” > 1.10, “++” > 4.15, “+++” > 100.

### Example 23

### Improvements Over SEQ ID NO: 1032 in Conversion of Nucleoside Monophosphates to Nucleotides

### HTP Screening for Improved AdyK Variants

Adenylate kinase of SEQ ID NO: 1032 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 23.1.

Reactions were performed as described in Example 4 using conditions summarized in Table 23.1. Data were collected using the TdT-coupled reaction and CE assay described in Example 6.

TABLE 23.1
Reaction conditions

Lysis Buffer - TEoA (pH 7.5), 0.1 g/L lysozyme; Lysis Conditions - 100 μL, 50° C., 60 min; Reaction
buffer - 50 mM Tris, 50 mM LiK(acetylphosphate), 10 μM ATP, 10 mM magnesium chloride, pH 8.0;
Lysate concentration (vol %) - 25; Reaction Conditions - 1 μL, 30° C., 1 hr; Nucleoside substrate -
mUMP; Substrate Concentration - 10 mM; Auxiliary Cascade Enzymes (Ack) - SEQ ID NO: 1318
(10 μM); Dilution into Coupling Reaction - 800X; Substrate Oligonucleotides - SEQ ID NO: 1323,
SEQ ID NO: 1324; Product Oligonucleotides - SEQ ID NO: 1333, SEQ ID NO: 1334.

Activity relative to SEQ ID NO: 1032 (Activity FIOP) was calculated based on the percentage of extension products observed for the variant compared with the percentage observed with SEQ ID NO: 1032 (where the percent product may be set as the average of replicates or else the highest single sample as appropriate). The results are shown in Table 23.2.

TABLE 23.2
Adenylate kinase activity relative to SEQ ID NO: 1032

| | | FIOP activity - | FIOP activity - |
|---|---|---|---|
| SEQ ID NO: | Amino Acid Differences | mUMP relative to | mGMP relative to |
| (nt/aa) | (Relative to SEQ ID NO: 1032) | SEQ ID NO: 1032 | SEQ ID NO: 1032 |
| 1355/1356 | K54T/A80D/I225A | ++ | +++ |
| 1357/1358 | Q55S/A80D/E180H | + | +++ |
| 1359/1360 | K54T/Q55S/A80D/R208E/I225L | | +++ |
| 1361/1362 | R84M/K128E/F155T/L190R/S212N | +++ | +++ |
| 1363/1364 | K62S/F155T/L190H | +++ | +++ |
| 1365/1366 | K83T/R84M/F155T/L190H | ++ | +++ |
| 1367/1368 | K62S/K128E/V135K/L190H | ++ | +++ |
| 1369/1370 | I225A | + | ++ |
| 1371/1372 | R84M/F155T/L190R | +++ | ++ |
| 1373/1374 | K128E/L190R | ++ | ++ |
| 1375/1376 | L190R/S212N | ++ | ++ |
| 1377/1378 | K83T/R84M/F155T | + | ++ |
| 1379/1380 | K54T/R208E/I225A | + | ++ |
| 1381/1382 | R84M/F155T/L190H | ++ | ++ |
| 1383/1384 | R84H/S212N | + | ++ |
| 1385/1386 | R84M/A123S/F155T/S212N | ++ | ++ |
| 1387/1388 | K62S/K128E/L190R | +++ | ++ |
| 1389/1390 | Q55S/A80D/R208E/I225A | | ++ |
| 1391/1392 | F155T/L190R | +++ | ++ |
| 1393/1394 | K83T/R84M/L190R/S212N | + | ++ |
| 1395/1396 | K83T/K128E/F155T/L190H/S212N | + | ++ |
| 1397/1398 | Q55S/E180H/I225A | + | ++ |
| 1399/1400 | R84M/L190R/S212N | ++ | ++ |
| 1401/1402 | K54T/A80D/E180H/R208E/I225A | | ++ |
| 1403/1404 | A80D/R208E | | ++ |
| 1405/1406 | V135K/F155T/L190R | + | ++ |
| 1407/1408 | S212N | + | ++ |
| 1409/1410 | A80D/E180H/R208E | | ++ |
| 1411/1412 | K128E/V135K/L190R/S212N | + | ++ |
| 1413/1414 | K128E/F155T/S212N | + | ++ |
| 1415/1416 | A80D/R208E/I225L | | ++ |
| 1417/1418 | K83T/S212N | | ++ |
| 1419/1420 | K62S/R84M/F155T/L190R | +++ | + |
| 1421/1422 | K54T/E179V/I225A | ++ | + |
| 1423/1424 | R84H | + | + |
| 1425/1426 | K83T/R84M/L190R | ++ | + |
| 1427/1428 | K128E/S212N | + | + |
| 1429/1430 | K54T/Q55S/A80D/R208E | | + |
| 1431/1432 | L190H/S212N | + | + |
| 1433/1434 | Q55S | + | + |
| 1435/1436 | K62S/K128E | ++ | + |
| 1437/1438 | K54T/A80D/R208E/I225A | | + |
| 1439/1440 | K62S/K83T/R84M/L190R | + | + |
| 1441/1442 | E179A/I225A | + | + |
| 1443/1444 | L190R | ++ | + |
| 1445/1446 | A80D/V169D/R208E | | + |
| 1447/1448 | K62S/K128E/S212N | ++ | + |
| 1449/1450 | A80D | | + |
| 1451/1452 | K62S | ++ | + |
| 1453/1454 | K54T/Q55S/A80D/I225L | | + |
| 1455/1456 | K54T/A80D/E180H/R208E | | + |
| 1457/1458 | K83T/R84M/K128E/L190H/S212N | + | + |
| 1459/1460 | K62S/K83T/L190R | +++ | + |
| 1461/1462 | E179V/I225A | ++ | + |
| 1463/1464 | K62S/L190R | +++ | + |
| 1465/1466 | A80D/E179V | | + |
| 1467/1468 | A80D/E180H | | + |
| 1469/1470 | K54T/A80D | | + |
| 1471/1472 | K62S/K83T/R84H/K128E/L190H/S212N | + | + |
| 1473/1474 | K83T/R84M/V135K/S212N | | + |
| 1475/1476 | K54T/A80D/E179A/R208E | | + |
| 1477/1478 | K62S/R84H/K128E | ++ | + |
| 1479/1480 | G11D/K62S/R84M/K128E/L190H/S212N | + | + |
| 1481/1482 | A80D/E179V/R208E | | + |
| 1483/1484 | A80D/I225A | | + |
| 1485/1486 | K62S/R84M/K128E/L190H/S212N | + | + |
| 1487/1488 | K62S/R84H/S212N | ++ | + |
| 1489/1490 | A80D/E179V/E180H/R208E/I225L | | + |
| 1491/1492 | K62S/V135K | ++ | + |
| 1493/1494 | Q55S/R208E | + | + |
| 1495/1496 | K62S/R84H | ++ | + |
| 1497/1498 | K128E/V135K/L190H | + | + |
| 1499/1500 | Q55S/R208E/I225A | + | + |
| 1501/1502 | L190H | + | + |
| 1503/1504 | R84H/K128E/L190H | + | + |
| 1505/1506 | K62S/R84H/K128E/L190R | +++ | + |
| 1507/1508 | K62S/V135K/S212N | + | + |
| 1509/1510 | K54T/E179V/E180H/I225A | + | + |
| 1511/1512 | K128E/L190H | + | + |
| 1513/1514 | K62S/R84M/V135K/S212N | + | + |
| 1515/1516 | V135K | + | + |
| 1517/1518 | K54T/Q55S/I225L | + | + |
| 1519/1520 | Q55S/A80D/E179V/E180H | + | + |
| 1521/1522 | K62S/R84M/S212N | + | + |
| 1523/1524 | R84M/V135K/L190R | + | + |
| 1525/1526 | R84M/K128E | + | |
| 1527/1528 | K54T | + | |
| 1529/1530 | R84M/K128E/V135K | + | |
| 1531/1532 | K54T/E179A/R208E/I225A | + | |
| 1533/1534 | Q55S/E179V/I225L | + | |
| 1535/1536 | K128E | + | |
| 1537/1538 | K62S/K83T/R84M/F155T/L190R | ++ | |
| 1539/1540 | R84M/V135K | + | |
| 1541/1542 | R84H/K128E/F155T | + | |
| 1543/1544 | Q55S/E179V/E180H | + | |
| 1545/1546 | K83T/R84M/K128E/L190R | + | |
| 1547/1548 | K83T/R84M/L190H | + | |
| 1549/1550 | R84M | + | |
| 1551/1552 | K54T/Q55S/E179V/I225A | + | |

Levels of increased activity were determined for FIOP activity - mUMP relative to SEQ ID NO: 1032 and are defined as follows: “+” > 1.1, “++” > 1.6, “+++” > 2.0. Levels of increased activity were determined for FIOP activity - mGMP relative to SEQ ID NO: 1032 and are defined as follows: “+” > 1.1, “++” > 1.65, “+++” > 2.4.

### Example 24

### Improvements Over SEQ ID NO: 1388 in Conversion of Nucleoside Monophosphates to Nucleotides

### HTP Screening for Improved AdyK Variants

Adenylate kinase of SEQ ID NO: 1388 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 24.1.

Reactions were performed as described in Example 4 using conditions summarized in Table 24.1. Data were collected using the TdT-coupled reaction and CE assay described in Example 6.

TABLE 24.1
Reaction conditions

Lysis Buffer - TEoA (pH 7.5), 0.1 g/L lysozyme; Lysis Conditions - 100 μL, 50° C., 60 min; Reaction
buffer - 50 mM Tris, 50 mM LiK(acetylphosphate), 10 μM ATP, 10 mM magnesium chloride, pH 8.0;
Lysate concentration (vol %) - 5; Reaction Conditions - 1 μL, 30° C., 1 hr; Nucleoside substrate - fG;
Substrate Concentration - 10 mM; Auxiliary Cascade Enzymes (AdoK/AcK) - SEQ ID NO: 1338 (10
μM), SEQ ID NO: 1318 (10 μM); Dilution into Coupling Reaction - 800X; Substrate
Oligonucleotides - SEQ ID NO: 1323, SEQ ID NO: 1324; Product Oligonucleotides - SEQ ID NO:
2654, SEQ ID NO: 2655.

Activity relative to SEQ ID NO: 1388 (Activity FIOP) was calculated based on the percentage of extension products observed for the variant compared with the percentage observed with SEQ ID NO: 1388 (where the percent product may be set as the average of replicates or else the highest single sample as appropriate). The results are shown in Table 24.2.

TABLE 24.2
Adenylate kinase activity relative to SEQ ID NO: 1388

| | | FIOP activity - |
|---|---|---|
| SEQ ID NO: | Amino Acid Differences | fG relative to |
| (nt/aa) | (Relative to SEQ ID NO: 1388) | SEQ ID NO: 1388 |
| 1553/1554 | I143S | +++ |
| 1555/1556 | W228G | +++ |
| 1557/1558 | Q136A | +++ |
| 1559/1560 | I143T | +++ |
| 1561/1562 | V82Q | +++ |
| 1563/1564 | I143A | +++ |
| 1565/1566 | L156C | +++ |
| 1567/1568 | I205P | +++ |
| 1569/1570 | V82P | ++ |
| 1571/1572 | L226P | ++ |
| 1573/1574 | W228L | ++ |
| 1575/1576 | I143C | ++ |
| 1577/1578 | I150G | ++ |
| 1579/1580 | I150S | ++ |
| 1581/1582 | V219P | ++ |
| 1583/1584 | I150P | ++ |
| 1585/1586 | N217H | ++ |
| 1587/1588 | L226T | ++ |
| 1589/1590 | I143G | ++ |
| 1591/1592 | V218L | ++ |
| 1593/1594 | E68S | ++ |
| 1595/1596 | L142R | ++ |
| 1597/1598 | S212I | ++ |
| 1599/1600 | S212L | ++ |
| 1601/1602 | W228P | ++ |
| 1603/1604 | Q213S | ++ |
| 1605/1606 | L226S | ++ |
| 1607/1608 | L156T | + |
| 1609/1610 | N79P | + |
| 1611/1612 | D230A | + |
| 1613/1614 | V218A | + |
| 1615/1616 | I143R | + |
| 1617/1618 | W228S | + |
| 1619/1620 | E68L | + |
| 1621/1622 | K207T | + |
| 1623/1624 | W228R | + |
| 1625/1626 | F155A | + |
| 1627/1628 | Q136S | + |
| 1629/1630 | I150L | + |
| 1631/1632 | I150V | + |
| 1633/1634 | G227P | + |
| 1635/1636 | V129L | + |
| 1637/1638 | D230Q | + |
| 1639/1640 | D210V | + |
| 1641/1642 | I205L | + |
| 1643/1644 | E68G | + |
| 1645/1646 | E133W | + |
| 1647/1648 | F155W | + |
| 1649/1650 | S212R | + |
| 1651/1652 | K207A | + |
| 1653/1654 | Y151F | + |
| 1655/1656 | S212P | + |
| 1657/1658 | V218I | + |
| 1659/1660 | V129P | + |
| 1661/1662 | S43A | + |
| 1663/1664 | D230P | + |
| 1665/1666 | V129S | + |
| 1667/1668 | E68A | + |
| 1669/1670 | L142F | + |
| 1671/1672 | G204S | + |
| 1673/1674 | Q136Y | + |
| 1675/1676 | V182S | + |
| 1677/1678 | E133S | + |
| 1679/1680 | W228I | + |
| 1681/1682 | S43C | + |
| 1683/1684 | N217G | + |
| 1685/1686 | V129I | + |

Levels of increased activity were determined FIOP activity - fG relative to SEQ ID NO: 1388 and are defined as follows: “+” > 1.0, “++” > 1.22, “+++” > 1.5.

### Example 25

### Improvements Over SEQ ID NO: 1388 in Conversion of Nucleoside Monophosphates to Nucleotides

### HTP Screening for Improved AdyK Variants

Adenylate kinase of SEQ ID NO: 1388 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 25.1.

Reactions were performed as described in Example 4 using conditions summarized in Table 25.1. Data were collected using the TdT-coupled reaction and CE assay described in Example 6.

TABLE 25.1
Reaction conditions

Lysis Buffer - TEoA (pH 7.5), 0.1 g/L lysozyme; Lysis Conditions - 100 μL, 50° C., 60 min;
Reaction buffer - 50 mM Tris, 50 mM LiK(acetylphosphate), 10 μM ATP, 10 mM magnesium
chloride, pH 8.0; Lysate concentration (vol %) - 2; Reaction Conditions - 1 μL, 30° C., 1 hr;
Nucleoside substrate - mA; Substrate Concentration - 10 mM; Auxiliary Cascade Enzymes
(AdoK/AcK)- SEQ ID NO: 1338 (10 μM), SEQ ID NO: 1318 (10 μM); Dilution into Coupling
Reaction - 800X; Substrate Oligonucleotides - SEQ ID NO: 1323, SEQ ID NO: 1324; Product
Oligonucleotides - SEQ ID NO: 2656, SEQ ID NO: 2657.

Activity relative to SEQ ID NO: 1388 (Activity FIOP) was calculated based on the percentage of extension products observed for the variant compared with the percentage observed with SEQ ID NO: 1388 (where the percent product may be set as the average of replicates or else the highest single sample as appropriate). The results are shown in Table 25.2.

TABLE 25.2
Adenylate kinase activity relative to SEQ ID NO: 1388

| | | FIOP activity - | FIOP activity - |
|---|---|---|---|
| SEQ ID NO: | Amino Acid Differences | mA relative to | fG relative to |
| (nt/aa) | (Relative to SEQ ID NO: 1388) | SEQ ID NO: 1388 | SEQ ID NO: 1388 |
| 1687/1688 | V82Q/Q136L/I150G/N217P | +++ | |
| 1689/1690 | Q136A/I150S | +++ | ++ |
| 1691/1692 | N79P/V81S/I143A/L156C/S212I/W228G | +++ | |
| 1693/1694 | V82Q/Q136A | ++ | ++ |
| 1695/1696 | N79P/V81S/I143A/S212I | ++ | |
| 1697/1698 | N217P | ++ | + |
| 1699/1700 | N79P/E133F/W228L | + | + |
| 1701/1702 | V81S/I143A/L156T/S212I | + | |
| 1703/1704 | Q136A/V219A | + | |
| 1705/1706 | V82Q/Q136A/L226P | + | |
| 1707/1708 | Q136A/I150S/N217P | + | +++ |
| 1709/1710 | Q136A/I150G | + | +++ |
| 1711/1712 | E133F | + | ++ |
| 1713/1714 | E133F/L156C | + | + |
| 1715/1716 | E133F/I143A/W228L | + | + |
| 1717/1718 | Q136L | + | |
| 1719/1720 | I150S/N217P | | ++ |
| 1721/1722 | V82Q/Q136A/I150S | | +++ |
| 1723/1724 | V81S | | +++ |
| 1725/1726 | V81S/1143T | | +++ |
| 1727/1728 | V81S/E133F/W228L | | +++ |
| 1729/1730 | I143A/W228L | | ++ |
| 1731/1732 | S212I/W228G | | ++ |
| 1733/1734 | N79P/V81S | | ++ |
| 1735/1736 | E133F/W228L | | + |
| 1737/1738 | E133F/S212M | | + |
| 1739/1740 | Q136L/N217H | | + |
| 1741/1742 | V82P/Q136L | | + |

and are defined as follows: “+” > 1.0, “++” > 1.4, “+++” > 1.9. Levels of increased activity were determined for FIOP activity - fG relative to SEQ ID NO: 1388 and are defined as follows: “+” > 1.0, “++” > 1.5, “+++” > 1.77.

### Example 26

### Improvements Over SEQ ID NO: 1388 in Conversion of Nucleoside Monophosphates to Nucleotides

### HTP Screening for Improved AdyK Variants

Adenylate kinase of SEQ ID NO: 1388 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 26.1.

Reactions were performed as described in Example 4 using conditions summarized in Table 26.1. Data were collected using the TdT-coupled reaction and CE assay described in Example 6.

TABLE 26.1
Reaction conditions

Lysis Buffer - TEoA (pH 7.5), 0.1 g/L lysozyme; Lysis Conditions - 100 μL, 50° C., 60 min; Reaction
buffer - 50 mM Tris, 50 mM LiK(acetylphosphate), 10 μM ATP, 10 mM magnesium chloride, pH
8.0; Lysate concentration (vol %) - 2; Reaction Conditions - 1 μL, 30° C., 1 hr; Nucleoside substrate -
mA; Substrate Concentration - 10 mM; Auxiliary Cascade Enzymes (AdoK/AcK)- SEQ ID NO:
1340 (10 μM), SEQ ID NO: 1354 (10 μM); Dilution into Coupling Reaction - 800X; Substrate
Oligonucleotides - SEQ ID NO: 1323, SEQ ID NO: 1324; Product Oligonucleotides - SEQ ID NO:
2656, SEQ ID NO: 2657.

Activity relative to SEQ ID NO: 1388 (Activity FIOP) was calculated based on the percentage of extension products observed for the variant compared with the percentage observed with SEQ ID NO: 1388 (where the percent product may be set as the average of replicates or else the highest single sample as appropriate). The results are shown in Table 26.2.

TABLE 26.2
Adenylate kinase activity relative to SEQ ID NO: 1388

| | Amino Acid | | |
|---|---|---|---|
| | Differences | FIOP activity - | FIOP activity - |
| SEQ | (Relative to | mA relative to | mUMP relative to |
| ID NO: | SEQ ID | SEQ ID | SEQ ID |
| (nt/aa) | NO: 1388) | NO: 1388 | NO: 1388 |
| 1553/1554 | I143S | | +++ |
| 1557/1558 | Q136A | | + |
| 1559/1560 | I143T | | + |
| 1575/1576 | I143C | | ++ |
| 1579/1580 | I150S | | ++ |
| 1581/1582 | V219P | ++ | |
| 1589/1590 | I143G | + | +++ |
| 1591/1592 | V218L | + | |
| 1595/1596 | L142R | | +++ |
| 1599/1600 | S212L | + | |
| 1611/1612 | D230A | + | |
| 1619/1620 | E68L | | ++ |
| 1623/1624 | W228R | +++ | ++ |
| 1625/1626 | F155A | + | ++ |
| 1627/1628 | Q136S | + | ++ |
| 1633/1634 | G227P | | + |
| 1641/1642 | I205L | +++ | ++ |
| 1649/1650 | S212R | + | |
| 1651/1652 | K207A | + | + |
| 1653/1654 | Y151F | + | |
| 1657/1658 | V218I | + | |
| 1661/1662 | S43A | + | |
| 1669/1670 | L142F | | ++ |
| 1683/1684 | N217G | + | ++ |
| 1743/1744 | I127P | +++ | |
| 1745/1746 | N217E | ++ | + |
| 1747/1748 | D216H | ++ | |
| 1749/1750 | I215P | ++ | |
| 1751/1752 | I143P | ++ | |
| 1753/1754 | Y151R | ++ | |
| 1755/1756 | P22S | ++ | |
| 1757/1758 | D216P | ++ | + |
| 1759/1760 | S212M | + | |
| 1761/1762 | Y151A | + | |
| 1763/1764 | V219A | + | |
| 1765/1766 | Q213L | + | +++ |
| 1767/1768 | Q213P | + | |
| 1769/1770 | I127S | + | |
| 1771/1772 | I150C | + | |
| 1773/1774 | I215A | + | |
| 1775/1776 | K207L | + | |
| 1777/1778 | E133Q | + | |
| 1779/1780 | S212W | + | |
| 1781/1782 | V219G | + | |
| 1783/1784 | G204R | + | |
| 1785/1786 | V219S | + | |

Levels of increased activity were determined for FIOP activity - mA relative to SEQ ID NO: 1388 and are defined as follows: “+” > 1.1, “++” > 1.4, “+++” > 1.7. Levels of increased activity were determined for FIOP activity - mUMP relative to SEQ ID NO: 1388 and are defined as follows: “+” > 1.1, “++” > 1.25, “+++” > 1.4.

### Example 27

### Improvements Over SEQ ID NO: 1708 in Conversion of Nucleoside Monophosphates to Nucleotides

### HTP Screening for Improved AdyK Variants

Adenylate kinase of SEQ ID NO: 1708 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 27.1.

Reactions were performed as described in Example 4 using conditions summarized in Table 27.1. Data were collected using the TdT-coupled reaction and CE assay described in Example 6.

TABLE 27.1
Reaction conditions

Lysis Buffer - TEoA (pH 7.5), 0.1 g/L lysozyme; Lysis Conditions - 100 μL, 50° C., 60 min; Reaction
buffer - 50 mM Tris, 50 mM LiK(acetylphosphate), 10 μM ATP, 10 mM magnesium chloride, pH
8.0; Lysate concentration (vol %) - 0.5; Reaction Conditions - 1 μL, 30° C., 1 hr; Nucleoside substrate -
mA; Substrate Concentration - 10 mM; Auxiliary Cascade Enzymes (AdoK/AcK) - SEQ ID NO:
1340 (10 μM), SEQ ID NO: 1354 (10 μM); Dilution into Coupling Reaction - 800X; Substrate
Oligonucleotides - SEQ ID NO: 1323, SEQ ID NO: 1324; Product Oligonucleotides - SEQ ID NO:
2656, SEQ ID NO: 2657.

Activity relative to SEQ ID NO: 1708 (Activity FIOP) was calculated based on the percentage of extension products observed for the variant compared with the percentage observed with SEQ ID NO: 1708 (where the percent product may be set as the average of replicates or else the highest single sample as appropriate). The results are shown in Table 27.2.

TABLE 27.2
Adenylate kinase activity relative to SEQ ID NO: 1708

| | | FIOP activity - |
|---|---|---|
| SEQ ID NO: | Amino Acid Differences | mA relative to |
| (nt/aa) | (Relative to SEQ ID NO: 1708) | SEQ ID NO: 1708 |
| 1787/1788 | I127P | ++++ |
| 1789/1790 | I143S/F155A/V219P/W228R | ++++ |
| 1791/1792 | V219P | ++++ |
| 1793/1794 | I127P/L142R | ++++ |
| 1795/1796 | L142R/Y151A/K207A/V218A | ++++ |
| 1797/1798 | I143G/Q213L | ++++ |
| 1799/1800 | Y151A/V218A | ++++ |
| 1801/1802 | I127P/L142R/V218I | ++++ |
| 1803/1804 | L142R/Y151A | +++ |
| 1805/1806 | I143S/F155A/I205L/Q213L/W228R | +++ |
| 1807/1808 | I127P/L142R/K207A | +++ |
| 1809/1810 | I127P/D216P/V218A | +++ |
| 1811/1812 | L142R/Y151A/K207A | +++ |
| 1813/1814 | I143S/V219P | +++ |
| 1815/1816 | I127P/K207A | +++ |
| 1817/1818 | I127P/V218I | +++ |
| 1819/1820 | I143G/V219P/W228R | +++ |
| 1821/1822 | I127P/D216P | +++ |
| 1823/1824 | E68L/I127P/K207L | +++ |
| 1825/1826 | I143G/F155A/S212R/W228R | +++ |
| 1827/1828 | Y151A | +++ |
| 1829/1830 | I143S/V219P/W228R | +++ |
| 1831/1832 | I143S/Q213L/W228R | +++ |
| 1833/1834 | I143G/F155A/I205L | +++ |
| 1835/1836 | Y151A/K207A/D216P | +++ |
| 1837/1838 | I143G/W228R | +++ |
| 1839/1840 | E68L/I127P/K207A | ++ |
| 1841/1842 | E68L/I127P/K207A/V218A | ++ |
| 1843/1844 | I143G/F155A | ++ |
| 1845/1846 | I143G | ++ |
| 1847/1848 | I205L/Q213L/V219P | ++ |
| 1849/1850 | F155A/S212R/V219P/W228R | ++ |
| 1851/1852 | I143S/W228R | ++ |
| 1853/1854 | I205L/V219P/W228R | ++ |
| 1855/1856 | I143G/I205L | ++ |
| 1857/1858 | L142R/K207A | ++ |
| 1859/1860 | I143G/I205L/V219P | ++ |
| 1861/1862 | I143G/S212R/Q213L | ++ |
| 1863/1864 | I127P/K207L/V218I | ++ |
| 1865/1866 | L142R | ++ |
| 1867/1868 | I127P/D216P/V218I | ++ |
| 1869/1870 | I127P/L142R/Y151A/K207A/V218I | ++ |
| 1871/1872 | I143S/I205L/V219P/W228R | ++ |
| 1873/1874 | I205L/Q213L/W228R | ++ |
| 1875/1876 | I143G/V219P | + |
| 1877/1878 | L142R/K207L | + |
| 1879/1880 | E68L/Y151A/K207L | + |
| 1881/1882 | I143S/Q213L | + |
| 1883/1884 | L142R/Y151A/K207L | + |
| 1885/1886 | I127P/L142R/K207L | + |
| 1887/1888 | E68L/I127P/L142R/K207L | + |
| 1889/1890 | I143S/I205L | + |
| 1891/1892 | E68L/I127P | + |
| 1893/1894 | V218A | + |
| 1895/1896 | I127P/L142R/V196I | + |
| 1897/1898 | L142R/K207L/V218A | + |
| 1899/1900 | I143S/F155A | + |
| 1901/1902 | I143S | + |
| 1903/1904 | E68L/L142R/D216P | + |
| 1905/1906 | E68L/I127P/D216P/V218I | + |
| 1907/1908 | E68L/I127P/V218I | + |
| 1909/1910 | Y151A/D216P/V218I | + |
| 1911/1912 | I143S/S212R/W228R | + |
| 1913/1914 | I127P/L142R/Y151A | + |
| 1915/1916 | Y151A/K207L | + |
| 1917/1918 | I127P/L142R/D216P | + |
| 1919/1920 | V129I/K207A/V218A | + |
| 1921/1922 | L142R/K207L/V218I | + |
| 1923/1924 | E68L/L142R/Y151A/K207A | + |
| 1925/1926 | Y151A/K207A | + |
| 1927/1928 | E68L/L142R/Y151A | + |
| 1929/1930 | Y151A/K207L/V218I | + |
| 1931/1932 | W228R | + |
| 1933/1934 | F155A/Q213L | + |
| 1935/1936 | I127P/L142R/Y151A/V218I | + |
| 1937/1938 | I127P/Y151A | + |
| 1939/1940 | F155A/W228R | + |
| 1941/1942 | I143S/F155A/I205L | + |
| 1943/1944 | Y151A/D216P/V218A | + |
| 1945/1946 | E68L/L142R/K207L | + |
| 1947/1948 | A80V/Y151A/K207L | + |
| 1949/1950 | I205L/W228R | + |

Levels of increased activity were determined FIOP activity - mA relative to SEQ ID NO: 1708 and are defined as follows: “+” > 1.8, “++” > 4.7, “+++” > 6.6, “++++” > 9.0.

### Example 28

### Improvements Over SEQ ID NO: 1708 in Conversion of Nucleoside Monophosphates to Nucleotides

### HTP Screening for Improved AdyK Variants

Adenylate kinase of SEQ ID NO: 1708 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 28.1.

Reactions were performed as described in Example 4 using conditions summarized in Table 28.1. Data were collected using the TdT-coupled reaction and CE assay described in Example 6.

TABLE 28.1
Reaction conditions

Lysis Buffer - TEoA (pH 7.5), 0.1 g/L lysozyme; Lysis Conditions - 100 μL, 50° C., 60 min; Reaction
buffer - 50 mM Tris, 50 mM LiK(acetylphosphate), 10 μM ATP, 10 mM magnesium chloride, pH 8.0;
Lysate concentration (vol %) - 2; Reaction Conditions - 1 μL, 30° C., 1 hr; Nucleoside substrate -
mUMP; Substrate Concentration - 10 mM; Auxiliary Cascade Enzymes (AdoK/AcK) - SEQ ID NO:
1340 (10 μM), SEQ ID NO: 1354 (10 μM); Dilution into Coupling Reaction - 800X; Substrate
Oligonucleotides - SEQ ID NO: 1323, SEQ ID NO: 1324; Product Oligonucleotides - SEQ ID NO:
1333, SEQ ID NO: 1334.

Activity relative to SEQ ID NO: 1708 (Activity FIOP) was calculated based on the percentage of extension products observed for the variant compared with the percentage observed with SEQ ID NO: 1708 (where the percent product may be set as the average of replicates or else the highest single sample as appropriate). The results are shown in Table 28.2.

TABLE 28.2
Adenylate kinase activity relative to SEQ ID NO: 1708

| | | FIOP activity - |
|---|---|---|
| SEQ ID NO: | Amino Acid Differences | mUMP relative to |
| (nt/aa) | (Relative to SEQ ID NO: 1708) | SEQ ID NO: 1708 |
| 1951/1952 | E68L/L142R/K207A/D216P/V218I | +++ |
| 1953/1954 | E68L/V218I | +++ |
| 1955/1956 | E68L/K207A | +++ |
| 1957/1958 | D216P/V218I | ++ |
| 1959/1960 | E68L/L142R | ++ |
| 1961/1962 | S212R | + |
| 1963/1964 | E68L/K207L | + |
| 1965/1966 | I205L | + |
| 1967/1968 | L142R/K207A/D216P/V218I | + |

Levels of increased activity were determined FIOP activity - mUMP relative to SEQ ID NO: 1708 and are defined as follows: “+” > 1.1, “++” > 1.3, “+++” > 1.5.

### Example 29

### Improvements Over SEQ ID NO: 1952 in Conversion of Nucleoside Monophosphates to Nucleotides

### HTP Screening for Improved AdyK Variants

Adenylate kinase of SEQ ID NO: 1952 was selected as the parent adenylate kinase enzyme. Libraries of genes were produced from the parent gene using various techniques (e.g., saturation mutagenesis and recombination of previously identified beneficial mutations). The polypeptides encoded by each gene were produced in HTP and prepared as described in Table 29.1.

Reactions were performed as described in Example 4 using conditions summarized in Table 29.1. Data were collected using the TdT-coupled reaction and CE assay described in Example 6.

TABLE 29.1
Reaction conditions

Lysis Buffer - TEoA (pH 7.5), 0.1 g/L lysozyme; Lysis Conditions - 100 μL, 63° C., 60 min; Reaction
buffer - 50 mM Tris, 50 mM LiK(acetylphosphate), 10 μM ATP, 10 mM magnesium chloride, pH
8.0; Lysate concentration (vol %) - 5; Reaction Conditions - 1 μL, 30° C., 1 hr; Nucleoside substrate -
fG; Substrate Concentration - 10 mM; Auxiliary Cascade Enzymes (AdoK/AcK) - SEQ ID NO: 1342
(10 μM), SEQ ID NO: 1354 (10 μM); Dilution into Coupling Reaction - 800X; Substrate
Oligonucleotides - SEQ ID NO: 1323, SEQ ID NO: 1324; Product Oligonucleotides - SEQ ID NO:
2654, SEQ ID NO: 2655.

Stability relative to SEQ ID NO: 1952 (Stability FIOP) was calculated based on the percentage of extension products observed for the variant compared with the percentage observed with SEQ ID NO: 1952 (where the percent product may be set as the average of replicates or else the highest single sample as appropriate). The results are shown in Table 29.2.

TABLE 29.2
Adenylate kinase activity relative to SEQ ID NO: 1952

| | | FIOP stability |
|---|---|---|
| SEQ ID NO: | Amino Acid Differences | relative to |
| (nt/aa) | (Relative to SEQ ID NO: 1952) | SEQ ID NO: 1952 |
| 1969/1970 | F18L/N118G | +++ |
| 1971/1972 | S87M/R119K/K192A | +++ |
| 1973/1974 | R119K/Q173R/K192A | +++ |
| 1975/1976 | N118G | +++ |
| 1977/1978 | S87M/R119K | +++ |
| 1979/1980 | F18L/N118G/G170S | ++ |
| 1981/1982 | F18L | ++ |
| 1983/1984 | R119K | ++ |
| 1985/1986 | K192A | ++ |
| 1987/1988 | S87E | ++ |
| 1989/1990 | S87M | ++ |
| 1991/1992 | K89Q | ++ |
| 1993/1994 | K192I | ++ |
| 1995/1996 | V59L | + |
| 1997/1998 | G170S | + |
| 1999/2000 | K64R | + |
| 2001/2002 | S112N | + |
| 2003/2004 | K93E | + |
| 2005/2006 | H184S | + |
| 2007/2008 | K146N | + |
| 2009/2010 | S112K | + |
| 2011/2012 | I51T | + |
| 2013/2014 | V125I | + |
| 2015/2016 | K93S | + |
| 2017/2018 | K192W | + |
| 2019/2020 | S112A | + |
| 2021/2022 | E191D | + |
| 2023/2024 | I51M | + |
| 2025/2026 | Q117L | + |
| 2027/2028 | K93Y | + |
| 2029/2030 | V172T | + |
| 2031/2032 | L163Q | + |

Levels of increased activity were determined FIOP stability relative to SEQ ID NO: 1952 and are defined as follows: “+” > 1.3, “++” > 2.6, “+++” > 7.5.

### Example 30

### Improvements Over SEQ ID NO: 1980 in Conversion of Nucleoside Monophosphates to Nucleotides

### HTP Screening for Improved AdyK Variants

Adenylate kinase of SEQ ID NO: 1980 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 30.1.

Reactions were performed as described in Example 4 using conditions summarized in Table 30.1. Data were collected using the TdT-coupled reaction and CE assay described in Example 6.

TABLE 30.1
Reaction conditions

Lysis Buffer - TEoA (pH 7.5), 0.1 g/L lysozyme; Lysis Conditions - 100 μL, 68° C., 60 min; Reaction
buffer - 50 mM Tris, 50 mM LiK(acetylphosphate), 10 μM ATP, 10 mM magnesium chloride, pH 8.0;
Lysate concentration (vol %) - 5; Reaction Conditions - 1 μL, 30° C., 1 hr; Nucleoside substrate - fG;
Substrate Concentration - 10 mM; Auxiliary Cascade Enzymes (AdoK/AcK) - SEQ ID NO: 1344 (10
μM), SEQ ID NO: 1354 (10 μM); Dilution into Coupling Reaction - 800X; Substrate
Oligonucleotides - SEQ ID NO: 1323, SEQ ID NO: 1324; Product Oligonucleotides - SEQ ID NO:
2654, SEQ ID NO: 2655.

Stability relative to SEQ ID NO: 1980 (Stability FIOP) was calculated based on the percentage of extension products observed for the variant compared with the percentage observed with SEQ ID NO: 1980 (where the percent product may be set as the average of replicates or else the highest single sample as appropriate). The results are shown in Table 30.2.

TABLE 30.2
Adenylate kinase activity relative to SEQ ID NO: 1980

| | | FIOP stability |
|---|---|---|
| SEQ ID NO: | Amino Acid Differences | relative to |
| (nt/aa) | (Relative to SEQ ID NO: 1980) | SEQ ID NO: 1980 |
| 2033/2034 | S87E/R119K/K192A | +++ |
| 2035/2036 | S87E/K192A | +++ |
| 2037/2038 | S87E/R119K | +++ |
| 2039/2040 | R119K/K192A | +++ |
| 2041/2042 | S87M/R119K | ++ |
| 2043/2044 | K192A | ++ |
| 2045/2046 | R119K | ++ |
| 2047/2048 | K146N/K192A | ++ |
| 2049/2050 | S87E | ++ |
| 2051/2052 | S87M/K192A | + |
| 2053/2054 | S87M | + |
| 2055/2056 | V59L/S87E | + |
| 2057/2058 | V59L/S87E/S112A/K146N | + |
| 2059/2060 | K146N | + |
| 2061/2062 | S87E/S112A/R119K | + |

Levels of increased activity were determined FIOP stability relative to SEQ ID NO: 1980 and are defined as follows: “+” > 1.2, “++” > 2.0, “+++” > 3.1.

### Example 31

### Improvements Over SEQ ID NO: 1980 in Conversion of Nucleoside Monophosphates to Nucleotides

### HTP Screening for Improved AdyK Variants

Adenylate kinase of SEQ ID NO: 1980 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 31.1.

Reactions were performed as described in Example 4 using conditions summarized in Table 31.1. Data were collected using the TdT-coupled reaction and CE assay described in Example 6.

TABLE 31.1
Reaction conditions

Lysis Buffer - TEoA (pH 7.5), 0.1 g/L lysozyme; Lysis Conditions - 100 μL, 50° C., 60 min; Reaction
buffer - 50 mM Tris, 50 mM LiK(acetylphosphate), 10 μM ATP, 10 mM magnesium chloride, pH 8.0;
Lysate concentration (vol %) - 25; Reaction Conditions - 1 μL, 30° C., 1 hr; Nucleoside substrate -
mGMP; Substrate Concentration - 10 mM; Auxiliary Cascade Enzymes (AdoK/AcK) - SEQ ID NO:
1344 (10 μM), SEQ ID NO: 1354 (10 μM); Dilution into Coupling Reaction - 80000X; Substrate
Oligonucleotides - SEQ ID NO: 1323, SEQ ID NO: 1324; Product Oligonucleotides - SEQ ID NO:
T15mGmAmCmG, SEQ ID NO: 2660.

Activity relative to SEQ ID NO: 1980 (Activity FIOP) was calculated based on the percentage of extension products observed for the variant compared with the percentage observed with SEQ ID NO: 1980 (where the percent product may be set as the average of replicates or else the highest single sample as appropriate). The results are shown in Table 31.2.

TABLE 31.2
Adenylate kinase activity relative to SEQ ID NO: 1980

| | | FIOP activity - |
|---|---|---|
| SEQ ID NO: | Amino Acid Differences | mGMP relative to |
| (nt/aa) | (Relative to SEQ ID NO: 1980) | SEQ ID NO: 1980 |
| 2063/2064 | P74G | +++ |
| 2065/2066 | D75L | +++ |
| 2067/2068 | I66A | +++ |
| 2069/2070 | T181V | +++ |
| 2071/2072 | I66W | +++ |
| 2073/2074 | I66G | +++ |
| 2075/2076 | I66Q | ++ |
| 2077/2078 | V81F | ++ |
| 2079/2080 | I66H | ++ |
| 2081/2082 | V73R | ++ |
| 2083/2084 | I66N | ++ |
| 2085/2086 | L77S | ++ |
| 2087/2088 | L77A | ++ |
| 2089/2090 | I66D | ++ |
| 2091/2092 | I66V | ++ |
| 2093/2094 | V188L | ++ |
| 2095/2096 | L77Q | + |
| 2097/2098 | V73I | + |
| 2099/2100 | P216E | + |
| 2101/2102 | V82L | + |
| 2103/2104 | A136L | + |
| 2105/2106 | G214L | + |
| 2107/2108 | V188G | + |
| 2109/2110 | G214T | + |
| 2111/2112 | P21S | + |
| 2113/2114 | I66T | + |
| 2115/2116 | P217E | + |
| 2117/2118 | I154R | + |
| 2119/2120 | P216T | + |
| 2121/2122 | E133V | + |
| 2123/2124 | E71T | + |
| 2125/2126 | G214R | + |
| 2127/2128 | A136I | + |
| 2129/2130 | S212H | + |
| 2131/2132 | G214P | + |
| 2133/2134 | W228Q | + |

Levels of increased activity were determined FIOP activity - mGMP relative to SEQ ID NO: 1980 and are defined as follows: “+” > 1.2, “++” > 1.7, “+++” > 2.5.

### Example 32

### Improvements Over SEQ ID NO: 2072 in Conversion of Nucleoside Monophosphates to Nucleotides

### HTP Screening for Improved AdyK Variants

Adenylate kinase of SEQ ID NO: 2072 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 32.1.

Reactions were performed as described in Example 4 using conditions summarized in Table 32.1. Data were collected using the TdT-coupled reaction and CE assay described in Example 6.

TABLE 32.1
Reaction conditions

Lysis Buffer - TEoA (pH 7.5), 0.1 g/L lysozyme; Lysis Conditions - 100 μL, 50° C., 60 min; Reaction
buffer - 50 mM Tris, 50 mM LiK(acetylphosphate), 10 μM ATP, 10 mM magnesium chloride, pH
8.0; Lysate concentration (vol %) - 25; Reaction Conditions - 1 μL, 30° C., 1 hr; Nucleoside substrate -
mGMP; Substrate Concentration - 10 mM; Auxiliary Cascade Enzymes (AcK) - SEQ ID NO: 1354
(10 μM); Dilution into Coupling Reaction - 160X; Substrate Oligonucleotides - SEQ ID NO: 1323,
SEQ ID NO: 1324; Product Oligonucleotides - SEQ ID NO: 2659, SEQ ID NO: 2660.

Activity relative to SEQ ID NO: 2072 (Activity FlOP) was calculated based on the percentage of extension products observed for the variant compared with the percentage observed with SEQ ID NO: 2072 (where the percent product may be set as the average of replicates or else the highest single sample as appropriate). The results are shown in Table 32.2.

TABLE 32.2
Adenylate kinase activity relative to SEQ ID NO: 2072

| | | FIOP activity - |
|---|---|---|
| SEQ ID NO: | Amino Acid Differences | mGMP relative to |
| (nt/aa) | (Relative to SEQ ID NO: 2072) | SEQ ID NO: 2072 |
| 2135/2136 | S87R | +++ |
| 2137/2138 | S87K | +++ |
| 2139/2140 | S87L | +++ |
| 2141/2142 | Q194R | +++ |
| 2143/2144 | I197L | ++ |
| 2145/2146 | S87Y | ++ |
| 2147/2148 | K93G | ++ |
| 2149/2150 | K202E | ++ |
| 2151/2152 | G38C | ++ |
| 2153/2154 | C91V | ++ |
| 2155/2156 | Q194L | ++ |
| 2157/2158 | R183L | ++ |
| 2159/2160 | K93T | ++ |
| 2161/2162 | I39T | ++ |
| 2163/2164 | C91L | ++ |
| 2165/2166 | K146H | ++ |
| 2167/2168 | K203E | ++ |
| 2169/2170 | K203L | + |
| 2171/2172 | K146N | + |
| 2173/2174 | T37G | + |
| 2175/2176 | Q194Y | + |
| 2177/2178 | T37R | + |
| 2179/2180 | E92L | + |
| 2181/2182 | K36R | + |
| 2183/2184 | K36S | + |
| 2185/2186 | K89T | + |
| 2187/2188 | E131A | + |
| 2189/2190 | D132T | + |
| 2191/2192 | D90F | + |
| 2193/2194 | I197Q | + |
| 2195/2196 | D90V | + |
| 2197/2198 | G148S | + |
| 2199/2200 | C91S | + |
| 2201/2202 | A35E/I197V | + |
| 2203/2204 | D132V | + |
| 2205/2206 | I197A | + |
| 2207/2208 | I197V | + |
| 2209/2210 | K93P | + |
| 2211/2212 | E92S | + |
| 2213/2214 | K36M | + |
| 2215/2216 | I39A | + |
| 2217/2218 | Q194V | + |
| 2219/2220 | S87V | + |
| 2221/2222 | D132G | + |
| 2223/2224 | G38F | + |
| 2225/2226 | D90T | + |
| 2227/2228 | K231R | + |
| 2229/2230 | G148F | + |
| 2231/2232 | C91G | + |
| 2233/2234 | C91A | + |

Levels of increased activity were determined FIOP activity - mGMP relative to SEQ ID NO: 2072 and are defined as follows: “+” > 1.3, “++” > 1.8, “+++” > 2.3.

### Improvements Over SEQ ID NO: 2072 in Conversion of Nucleoside Monophosphates to Nucleotides

### HTP Screening for Improved AdyK Variants

Adenylate kinase of SEQ ID NO: 2072 was selected as the parent adenylate kinase enzyme. Libraries of genes were produced from the parent gene using various techniques (e.g., saturation mutagenesis and recombination of previously identified beneficial mutations). The polypeptides encoded by each gene were produced in HTP and prepared as described in Table 33.1.

Reactions were performed as described in Example 4 using conditions summarized in Table 33.1. Data were collected using the TdT-coupled reaction and CE assay described in Example 6.

TABLE 33.1
Reaction conditions

Lysis Buffer - TEoA (pH 7.5), 0.1 g/L lysozyme; Lysis Conditions - 100 μL, 62° C., 60 min; Reaction
buffer - 50 mM Tris, 50 mM LiK(acetylphosphate), 10 μM ATP, 10 mM magnesium chloride, pH 8.0;
Lysate concentration (vol %) - 5; Reaction Conditions - 1 μL, 30° C., 1 hr; Nucleoside substrate - fG;
Substrate Concentration - 10 mM; Auxiliary Cascade Enzymes (AdoK/AcK) - SEQ ID NO: 1346 (10
μM), SEQ ID NO: 1354 (10 μM); Dilution into Coupling Reaction - 800X; Substrate
Oligonucleotides - SEQ ID NO: 1323, SEQ ID NO: 1324; Product Oligonucleotides - SEQ ID NO:
2654, SEQ ID NO: 2655.

Stability relative to SEQ ID NO: 2072 (Stability FIOP) was calculated based on the percentage of extension products observed for the variant compared with the percentage observed with SEQ ID NO: 2072 (where the percent product may be set as the average of replicates or else the highest single sample as appropriate). The results are shown in Table 33.2.

TABLE 33.2
Adenylate kinase activity relative to SEQ ID NO: 2072

| | | FIOP stability |
|---|---|---|
| SEQ ID NO: | Amino Acid Differences | relative to |
| (nt/aa) | (Relative to SEQ ID NO: 2072) | SEQ ID NO: 2072 |
| 2149/2150 | K202E | + |
| 2201/2202 | A35E/I197V | + |
| 2213/2214 | K36M | +++ |
| 2227/2228 | K231R | ++ |
| 2235/2236 | K36I | +++ |
| 2237/2238 | S87E | +++ |
| 2239/2240 | T37L | +++ |
| 2241/2242 | K231T | ++ |
| 2243/2244 | K231A | ++ |
| 2245/2246 | K89P | ++ |
| 2247/2248 | K202S | ++ |
| 2249/2250 | K36E | ++ |
| 2251/2252 | K231Q | ++ |
| 2253/2254 | S87A | + |
| 2255/2256 | K146V | + |
| 2257/2258 | K146R | + |
| 2259/2260 | N56T | + |
| 2261/2262 | K93A | + |
| 2263/2264 | K203R | + |

Levels of increased activity were determined FIOP stability relative to SEQ ID NO: 2072 and are defined as follows: “+” > 1.5, “++” > 1.8, “+++” > 2.4.

### Example 34

### Improvements Over SEQ ID NO: 2138 in Conversion of Nucleoside Monophosphates to Nucleotides

### HTP Screening for Improved AdyK Variants

Adenylate kinase of SEQ ID NO: 2138 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 34.1.

Reactions were performed as described in Example 4 using conditions summarized in Table 34.1. Data were collected using the TdT-coupled reaction and CE assay described in Example 6.

TABLE 34.1
Reaction conditions

Lysis Buffer - TEoA (pH 7.5), 0.1 g/L lysozyme; Lysis Conditions - 100 μL, 50° C., 60 min; Reaction
buffer - 50 mM Tris, 50 mM LiK(acetylphosphate), 10 μM ATP, 10 mM magnesium chloride, pH 8.0;
Lysate concentration (vol %) - 25; Reaction Conditions - 1 μL, 30° C., 1 hr; Nucleoside substrate - mG;
Substrate Concentration - 10 mM; Auxiliary Cascade Enzymes (AdoK/AcK) - SEQ ID NO: 1348 (10
μM), SEQ ID NO: 1354 (10 μM); Dilution into Coupling Reaction - 160X; Substrate
Oligonucleotides - SEQ ID NO: 1323, SEQ ID NO: 1324; Product Oligonucleotides - SEQ ID NO:
2659, SEQ ID NO: 2660.

Activity relative to SEQ ID NO: 2138 (Activity FIOP) was calculated based on the percentage of extension products observed for the variant compared with the percentage observed with SEQ ID NO: 2138 (where the percent product may be set as the average of replicates or else the highest single sample as appropriate). The results are shown in Table 34.2.

TABLE 34.2
Adenylate kinase activity relative to SEQ ID NO: 2138

| | | FIOP activity - |
|---|---|---|
| SEQ ID NO: | Amino Acid Differences | mG relative to |
| (nt/aa) | (Relative to SEQ ID NO: 2138) | SEQ ID NO: 2138 |
| 2265/2266 | D161G | +++ |
| 2267/2268 | E162G | +++ |
| 2269/2270 | M153C | +++ |
| 2271/2272 | P216L | ++ |
| 2273/2274 | S212Q | + |
| 2275/2276 | P216H | + |
| 2277/2278 | L156V | + |
| 2279/2280 | D161L | + |
| 2281/2282 | G214W | + |
| 2283/2284 | S229C | + |
| 2285/2286 | P216M | + |
| 2287/2288 | P216V | + |
| 2289/2290 | G214A | + |
| 2291/2292 | G214M | + |

Levels of increased activity were determined FIOP activity - mG relative to SEQ ID NO: 2138 and are defined as follows: “+” > 1.1, “++” > 1.25, “+++” > 1.5.

### Example 35

### Improvements Over SEQ ID NO: 2138 in Conversion of Nucleoside Monophosphates to Nucleotides

### HTP Screening for Improved AdyK Variants

Adenylate kinase of SEQ ID NO: 2138 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 35.1.

Reactions were performed as described in Example 4 using conditions summarized in Table 35.1. Data were collected using the TdT-coupled reaction and CE assay described in Example 6.

TABLE 35.1
Reaction conditions

Lysis Buffer - TEoA (pH 7.5), 0.1 g/L lysozyme; Lysis Conditions - 100 μL, 50° C., 60 min; Reaction
buffer - 50 mM Tris, 50 mM LiK(acetylphosphate), 10 μM ATP, 10 mM magnesium chloride, pH 8.0;
Lysate concentration (vol %) - 10; Reaction Conditions - 1 μL, 30° C., 1 hr; Nucleoside substrate - mG;
Substrate Concentration - 10 mM; Auxiliary Cascade Enzymes (AdoK/AcK) - SEQ ID NO: 1350 (10
μM), SEQ ID NO: 1354 (10 μM); Dilution into Coupling Reaction - 160X; Substrate
Oligonucleotides - SEQ ID NO: 1323, SEQ ID NO: 1324; Product Oligonucleotides - SEQ ID NO:
2659, SEQ ID NO: 2660.

Activity relative to SEQ ID NO: 2138 (Activity FIOP) was calculated based on the percentage of extension products observed for the variant compared with the percentage observed with SEQ ID NO: 2138 (where the percent product may be set as the average of replicates or else the highest single sample as appropriate). The results are shown in Table 35.2.

TABLE 35.2
Adenylate kinase activity relative to SEQ ID NO: 2138

| | | FIOP activity - |
|---|---|---|
| SEQ ID NO: | Amino Acid Differences | mG relative to |
| (nt/aa) | (Relative to SEQ ID NO: 2138) | SEQ ID NO: 2138 |
| 2293/2294 | L77I/I143V/G214E/P216R | +++ |
| 2295/2296 | I143V/L156V/P216R | +++ |
| 2297/2298 | L77I/I143V | +++ |
| 2299/2300 | L77I/I143V/P216R | +++ |
| 2301/2302 | L77I/G214E/P216R | +++ |
| 2303/2304 | L77I/P216L | ++ |
| 2305/2306 | I143V/L156V | ++ |
| 2307/2308 | L156V/P216R | ++ |
| 2309/2310 | I143V | ++ |
| 2311/2312 | L77I/G214E/P216L | ++ |
| 2313/2314 | L77I | ++ |
| 2315/2316 | L77I/G214E | ++ |
| 2317/2318 | L77I/I143V/D161L/E162G/ | ++ |
| | S212Q/G214E | |
| 2319/2320 | L77I/E162G/G214E/P216L | + |
| 2321/2322 | L77I/I143V/L156V/G214E/P216L | + |
| 2323/2324 | L77I/I143V/L156V/E162G/ | + |
| | S212Q/P216R | |
| 2325/2326 | L77I/I143V/L156V/D161L/ | + |
| | G214E/P216R | |
| 2327/2328 | L77I/P216R | + |
| 2329/2330 | L77I/L156V/E162G/P216R | + |
| 2331/2332 | L156V/D161L/G214E/P216L | + |
| 2333/2334 | L77I/I143V/E162G/G214W/P216L | + |
| 2335/2336 | I143V/P216L | + |
| 2337/2338 | G214E/P216L | + |
| 2339/2340 | I143V/L156V/G214E/P216R | + |
| 2341/2342 | I143V/G214E/P216L | + |
| 2343/2344 | I143V/G214E/P216R | + |
| 2345/2346 | G214E | + |
| 2347/2348 | L77I/I143V/S212Q/P216R | + |
| 2349/2350 | I143V/L156V/D161L/E162G/P216R | + |
| 2351/2352 | L156V/E162G/G214E | + |
| 2353/2354 | L77I/I143V/L156V/D161L/E162G | + |
| 2355/2356 | I143V/D161L/G214E/P216R | + |
| 2357/2358 | L77I/D161L/P216R | + |
| 2359/2360 | I143V/S212Q/G214E/P216R | + |
| 2361/2362 | L77I/I143V/G214W/P216R | + |
| 2363/2364 | I143V/P216R | + |
| 2365/2366 | P216R | |

Levels of increased activity were determined FIOP activity - mG relative to SEQ ID NO: 2138 and are defined as follows: “+” > 1.3, “++” > 2.3, “+++” > 2.9.

### Example 36

### Improvements Over SEQ ID NO: 2294 in Conversion of Nucleoside Monophosphates to Nucleotides

### HTP Screening for Improved AdyK Variants

Adenylate kinase of SEQ ID NO: 2294 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 36.1.

Reactions were performed as described in Example 4 using conditions summarized in Table 36.1. Data were collected using the TdT-coupled reaction and CE assay described in Example 6.

TABLE 36.1
Reaction conditions

Lysis Buffer - TEoA (pH 7.5), 0.1 g/L lysozyme; Lysis Conditions - 100 μL, 50° C., 60 min; Reaction
buffer - 50 mM Tris, 50 mM LiK(acetylphosphate), 10 μM ATP, 10 mM magnesium chloride, pH 8.0;
Lysate concentration (vol %) - 10; Reaction Conditions - 1 μL, 30° C., 1 hr; Nucleoside substrate - mG;
Substrate Concentration - 10 mM; Auxiliary Cascade Enzymes (AdoK/AcK) - SEQ ID NO: 1350 (10
μM), SEQ ID NO: 1354 (10 μM); Dilution into Coupling Reaction - 160X; Substrate
Oligonucleotides - SEQ ID NO: 1323, SEQ ID NO: 1324; Product Oligonucleotides - SEQ ID NO:
2659, SEQ ID NO: 2660.

Activity relative to SEQ ID NO: 2294 (Activity FIOP) was calculated based on the percentage of extension products observed for the variant compared with the percentage observed with SEQ ID NO: 2294 (where the percent product may be set as the average of replicates or else the highest single sample as appropriate). The results are shown in Table 36.2.

TABLE 36.2
Adenylate kinase activity relative to SEQ ID NO: 2294

| | | FIOP activity - |
|---|---|---|
| SEQ ID NO: | Amino Acid Differences | mG relative to |
| (nt/aa) | (Relative to SEQ ID NO: 2294) | SEQ ID NO: 2294 |
| 2367/2368 | A80P | +++ |
| 2369/2370 | R105K | ++++ |
| 2371/2372 | W228S | ++++ |
| 2373/2374 | D50Q | ++++ |
| 2375/2376 | L175F | ++++ |
| 2377/2378 | S170R | ++++ |
| 2379/2380 | V82L | +++ |
| 2381/2382 | Q173S | +++ |
| 2383/2384 | E65T | +++ |
| 2385/2386 | A53M | +++ |
| 2387/2388 | H184V | +++ |
| 2389/2390 | T122S | +++ |
| 2391/2392 | P195M | +++ |
| 2393/2394 | W228L | +++ |
| 2395/2396 | L68S | +++ |
| 2397/2398 | K89A | +++ |
| 2399/2400 | S34E | +++ |
| 2401/2402 | G118S | +++ |
| 2403/2404 | A53S | +++ |
| 2405/2406 | R119F | +++ |
| 2407/2408 | K87A | ++ |
| 2409/2410 | E179C | ++ |
| 2411/2412 | Q117S | ++ |
| 2413/2414 | F113E | ++ |
| 2415/2416 | D50C | ++ |
| 2417/2418 | A80G | ++ |
| 2419/2420 | T122H | ++ |
| 2421/2422 | K89V | ++ |
| 2423/2424 | A80R | ++ |
| 2425/2426 | K54I | ++ |
| 2427/2428 | R190Q | ++ |
| 2429/2430 | R216M | ++ |
| 2431/2432 | E88R | ++ |
| 2433/2434 | A80D | ++ |
| 2435/2436 | K36Q | ++ |
| 2437/2438 | R105S | ++ |
| 2439/2440 | P195G | ++ |
| 2441/2442 | L68V | ++ |
| 2443/2444 | R105L | ++ |
| 2445/2446 | L175D | + |
| 2447/2448 | H184T | + |
| 2449/2450 | D166L | + |
| 2451/2452 | K89H | + |
| 2453/2454 | G38R | + |
| 2455/2456 | K54G | + |
| 2457/2458 | G38F | + |
| 2459/2460 | K89I | + |
| 2461/2462 | V169I | + |
| 2463/2464 | W66C | + |
| 2465/2466 | R142W | + |
| 2467/2468 | Q117L | + |
| 2469/2470 | A136Y | + |
| 2471/2472 | E131V | + |
| 2473/2474 | K187Y | + |
| 2475/2476 | P40L | + |
| 2477/2478 | Q173T | + |
| 2479/2480 | P74C | + |
| 2481/2482 | W228V | + |
| 2483/2484 | I127L | + |
| 2485/2486 | A80L | + |
| 2487/2488 | Q55G | + |
| 2489/2490 | H184M | + |
| 2491/2492 | A136V | + |
| 2493/2494 | W228T | + |
| 2495/2496 | G148R | + |
| 2497/2498 | E88T | + |
| 2499/2500 | W228Q | + |
| 2501/2502 | W66S | + |
| 2503/2504 | I215V | + |
| 2505/2506 | P195R | + |
| 2507/2508 | R190G | + |
| 2509/2510 | K64R | + |
| 2511/2512 | H184R | + |
| 2513/2514 | T122A | + |
| 2515/2516 | D50S | + |
| 2517/2518 | R142M | + |
| 2519/2520 | F155W | + |
| 2521/2522 | A80S | + |
| 2523/2524 | R216G | + |

Levels of increased activity were determined FIOP activity - mG relative to SEQ ID NO: 2294 and are defined as follows: “+” > 1.3, “++” > 1.9, “+++” > 2.7, “++++” > 3.5.

### Example 37

### Improvements Over SEQ ID NO: 2294 in Conversion of Nucleoside Monophosphates to Nucleotides

### HTP Screening for Improved AdyK Variants

Adenylate kinase of SEQ ID NO: 2294 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 37.1.

Reactions were performed as described in Example 4 using conditions summarized in Table 37.1. Data were collected using the TdT-coupled reaction and CE assay described in Example 6.

TABLE 37.1
Reaction conditions

Lysis Buffer - TEoA (pH 7.5), 0.1 g/L lysozyme; Lysis Conditions - 100 μL, 66° C., 60 min; Reaction
buffer - 50 mM Tris, 50 mM LiK(acetylphosphate), 10 μM ATP, 10 mM magnesium chloride, pH
8.0; Lysate concentration (vol %) - 5; Reaction Conditions - 1 μL, 30° C., 1 hr; Nucleoside substrate -
mU; Substrate Concentration - 10 mM; Auxiliary Cascade Enzymes (AdoK/AcK) - SEQ ID NO:
1352 (10 μM), SEQ ID NO: 1354 (10 μM); Dilution into Coupling Reaction - 800X; Substrate
Oligonucleotides - SEQ ID NO: 1323, SEQ ID NO: 1324; Product Oligonucleotides - SEQ ID NO:
1333, SEQ ID NO: 1334.

Stability relative to SEQ ID NO: 2294 (Stability FIOP) was calculated based on the percentage of extension products observed for the variant compared with the percentage observed with SEQ ID NO: 2294 (where the percent product may be set as the average of replicates or else the highest single sample as appropriate). The results are shown in Table 37.2.

TABLE 37.2
Adenylate kinase activity relative to SEQ ID NO: 2294

| | | FIOP stability |
|---|---|---|
| SEQ ID NO: | Amino Acid Differences | relative to |
| (nt/aa) | (Relative to SEQ ID NO: 2294) | SEQ ID NO: 2294 |
| 2371/2372 | W228S | + |
| 2375/2376 | L175F | + |
| 2383/2384 | E65T | + |
| 2385/2386 | A53M | + |
| 2407/2408 | K87A | + |
| 2419/2420 | T122H | + |
| 2421/2422 | K89V | + |
| 2445/2446 | L175D | + |
| 2461/2462 | V169I | + |
| 2507/2508 | R190G | + |
| 2509/2510 | K64R | + |
| 2511/2512 | H184R | + |
| 2525/2526 | S116A | +++ |
| 2527/2528 | D90C | +++ |
| 2529/2530 | D132K | +++ |
| 2531/2532 | R105G | +++ |
| 2533/2534 | A60W | +++ |
| 2535/2536 | G94Y | ++ |
| 2537/2538 | S112T | ++ |
| 2539/2540 | K87S | ++ |
| 2541/2542 | K187G | ++ |
| 2543/2544 | Q120T | ++ |
| 2545/2546 | K87I | ++ |
| 2547/2548 | L68R | ++ |
| 2549/2550 | T37Y | + |
| 2551/2552 | Q173R | + |
| 2553/2554 | R105M | + |
| 2555/2556 | E108V | + |
| 2557/2558 | A35L | + |
| 2559/2560 | L68I | + |
| 2561/2562 | D166F | + |
| 2563/2564 | A35F | + |
| 2565/2566 | Q120S | + |
| 2567/2568 | H184S | + |
| 2569/2570 | V169A | + |
| 2571/2572 | I51L | + |
| 2573/2574 | S61V | + |
| 2575/2576 | S116F | + |
| 2577/2578 | E65D | + |
| 2579/2580 | K54H | + |
| 2581/2582 | S212C | + |
| 2583/2584 | V172S | + |
| 2585/2586 | Q55L | + |
| 2587/2588 | V59L | + |
| 2589/2590 | A60C | + |
| 2591/2592 | W228F | + |
| 2593/2594 | N56Q | + |

Levels of increased activity were determined FIOP stability relative to SEQ ID NO: 2294 and are defined as follows: “+” > 1.07, “++” > 1.6, “+++” > 2.5.

### Example 38

### Improvements Over SEQ ID NO: 2368 in Conversion of Nucleoside Monophosphates to Nucleotides

### HTP Screening for Improved AdyK Variants

Adenylate kinase of SEQ ID NO: 2368 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 38.1.

Reactions were performed as described in Example 4 using conditions summarized in Table 38.1. Data were collected using the TdT-coupled reaction and CE assay described in Example 6.

TABLE 38.1
Reaction conditions

Lysis Buffer - TEoA (pH 7.5), 0.1 g/L lysozyme; Lysis Conditions - 100 μL, 50° C., 60 min; Reaction
buffer - 50 mM Tris, 50 mM LiK(acetylphosphate), 10 μM ATP, 10 mM magnesium chloride, pH 8.0;
Lysate concentration (vol %) - 5; Reaction Conditions - 1 μL, 30° C., 1 hr; Nucleoside substrate - mU;
Substrate Concentration - 10 mM; Auxiliary Cascade Enzymes (AdoK/AcK) - SEQ ID NO: 1352 (10
μM), SEQ ID NO: 1354 (10 μM); Dilution into Coupling Reaction - 800X; Substrate Oligonucleotides -
SEQ ID NO: 1323, SEQ ID NO: 1324; Product Oligonucleotides - SEQ ID NO: 1333, SEQ ID NO: 1334.

Activity relative to SEQ ID NO: 2368 (Activity FIOP) was calculated based on the percentage of extension products observed for the variant compared with the percentage observed with SEQ ID NO: 2368 (where the percent product may be set as the average of replicates or else the highest single sample as appropriate). The results are shown in Table 38.2.

TABLE 38.2
Adenylate kinase activity relative to SEQ ID NO: 2368

| | | FIOP activity - |
|---|---|---|
| SEQ ID NO: | Amino Acid Differences | mG relative to |
| (nt/aa) | (Relative to SEQ ID NO: 2368) | SEQ ID NO: 2368 |
| 2595/2596 | A136Y/W228V | +++ |
| 2597/2598 | V82L/R142M/Q173T/H184T/R216M | +++ |
| 2599/2600 | H184T/R216M | +++ |
| 2601/2602 | D50Q/H184M/R216M | +++ |
| 2603/2604 | I127L/Q173R/H184M | +++ |
| 2605/2606 | R105K/A136V/S170R/L175F/W228T | ++ |
| 2607/2608 | R105K/L175F | ++ |
| 2609/2610 | I127L/Q173T/H184M | ++ |
| 2611/2612 | D50Q/H184T | ++ |
| 2613/2614 | R105K/S170R/L175F/W228V | ++ |
| 2615/2616 | D50Q/I127L/Q173R/H184M | ++ |
| 2617/2618 | D50Q/R142M/H184M/R216M | ++ |
| 2619/2620 | D50Q/V82L/I127L/R216M | ++ |
| 2621/2622 | D50Q | + |
| 2623/2624 | D50Q/V82L/Q173T/R216M | + |
| 2625/2626 | D50Q/I127L/R142W/H184M/R216M | + |
| 2627/2628 | D50Q/V82L/I127L | + |
| 2629/2630 | A136V/W228V | + |
| 2631/2632 | R105K/A136V | + |
| 2633/2634 | R105K/W228V | + |
| 2635/2636 | R105K/A136V/L175F/W228V | + |
| 2637/2638 | Q173T/R216M | + |
| 2639/2640 | D50Q/Q173T | + |
| 2641/2642 | W228V | + |
| 2643/2644 | I127L/Q173R | + |
| 2645/2646 | R105K/A136V/S170R/P195G | + |
| 2647/2648 | D50Q/V82L/R216M | + |
| 2649/2650 | L175F/W228V | + |
| 2651/2652 | D50Q/V82L/I127L/R142M/ | + |
| | H184M/R216M | |

Levels of increased activity were determined FIOP activity - mG relative to SEQ ID NO: 2368 and are defined as follows: “+” > 1.2, “++” > 1.4, “+++” > 1.7.

### Example 39

### Biosynthetic Cascade Reactions for Production of Nucleotide Triphosphates (NTPs) Using Pyruvate Oxidase (POx)

### NTP Biosynthetic Reaction Setup

Reactions were performed in 384-well format 40 μL BioRad PCR plates. AdoK variants were assayed in the presence of adenylate kinase (AdoK), acetate kinase (AcK), and pyruvate oxidase (POx) variants to enable direct conversion of nucleosides to the corresponding triphosphate using potassium phosphate and sodium pyruvate as POx substrates. These enzyme were added as either purified enzymes at a specific molarity or as g/L lyophilized lysate powder which was prepared by lyophilizing clarified shake flask lysate prepared as in Example 3. The reactions were set up as follows: (i) all reaction components, except for the nucleoside substrate, pyruvate, phosphate and the AdoK lysate, were premixed in a single solution and were aliquoted into each well of the 384-well plates, (ii) AdoK lysate solution was then added into the wells, and (iii) an aliquot of the substrate nucleoside in DMSO and with an aliquot of equimolar sodium pyruvate and potassium phosphate was added to initiate the reaction. The reaction plate was heat-sealed with a peelable aluminum seal and incubated in a thermocycler at the indicated temperature and reaction time, then held at 10° C. prior to analysis.

### Example 40

### Improvements Over SEQ ID NO: 2368 in Conversion of Nucleoside Monophosphates to Nucleotides

### HTP Screening for Improved AdvK Variants

Adenylate kinase of SEQ ID NO: 2368 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 40.1.

Reactions were performed as described in Example 39 using conditions summarized in Table 40.1. Data were collected using the CE assay described in Example 6.

TABLE 40.1
Reaction conditions

Lysis Buffer - TEoA (pH 7.5), 0.1 g/L lysozyme; Lysis Conditions - 100 μL, 50.0° C., 60 min;
Reaction buffer - 50.0 mM Tris, 50.0 mM sodium pyruvate, 50.0 mM dibasic potassium phosphate,
10.0 μM ATP, 10 mM magnesium chloride, pH 8.0; Lysate concentration (vol %) - 10.0; Reaction
Conditions - 1.0 μL, 30.0° C., 2 hr; Nucleoside substrate - fG; Substrate Concentration - 10.0 mM;
Auxiliary Cascade Enzymes - SEQ ID NO: 1352 (10.0 μM), SEQ ID NO: 1354 (10.0 μM), SEQ ID
NO: 2670 (2.0 μM); Dilution into Coupling Reaction - 800X; Substrate Oligonucleotides - SEQ ID
NO: 3194, SEQ ID NO: 3193; Product Oligonucleotides - SEQ ID NO: 3196, SEQ ID NO: 3195.

Activity relative to SEQ ID NO: 2368 (Activity FIOP) was calculated based on the percentage of extension products observed for the variant compared with the percentage observed with SEQ ID NO: 2368 (where the percent product may be set as the average of replicates or else the highest single sample as appropriate). The results are shown in Table 40.2.

TABLE 40.2
Adenylate kinase activity relative to SEQ ID NO: 2368

| | | FIOP % yield fGTP |
|---|---|---|
| SEQ ID NO: | Amino Acid Differences | relative to |
| (nt/aa) | (Relative to SEQ ID NO: 2368) | SEQ ID NO: 2368 |
| 2675/2676 | L48F | +++ |
| 2677/2678 | L138V | +++ |
| 2679/2680 | P21V | +++ |
| 2681/2682 | P21L | +++ |
| 2683/2684 | S43G | ++ |
| 2685/2686 | D46E | ++ |
| 2687/2688 | P22A | ++ |
| 2689/2690 | T181C | ++ |
| 2691/2692 | V182A | ++ |
| 2693/2694 | L138M | ++ |
| 2695/2696 | T44S | ++ |
| 2697/2698 | T181V | + |
| 2699/2700 | D46M | + |
| 2701/2702 | D46L | + |
| 2703/2704 | P21N | + |
| 2705/2706 | G20A | + |
| 2707/2708 | S43A | + |
| 2709/2710 | S43H | + |
| 2711/2712 | D46Y | + |
| 2713/2714 | D46F | + |

Levels of increased activity were determined for FIOP % yield fGTP relative to SEQ ID NO: 2368 and are defined as follows: “+” > 1.1, “++”> 1.3, “+++” > 1.5.

### Example 41

### Improvements Over SEQ ID NO: 2602 in Conversion of Nucleoside Monophosphates to Nucleotides

### HTP Screening for Improved AdyK Variants

Adenylate kinase of SEQ ID NO: 2602 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 41.1.

Reactions were performed as described in Example 39 using conditions summarized in Table 41.1. Data were collected using the CE assay described in Example 6.

TABLE 41.1
Reaction conditions

Lysis Buffer - TEoA (pH 7.5), 0.1 g/L lysozyme; Lysis Conditions - 100 μL, 50.0° C., 60 min;
Reaction buffer - 50.0 mM Tris, 50.0 mM sodium pyruvate, 50.0 mM dibasic potassium phosphate,
10.0 μM ATP, 10 mM magnesium chloride, pH 8.0; Lysate concentration (vol %) - 5; Reaction
Conditions - 1.0 μL, 30.0° C., 1.0 hr; Nucleoside substrate - mU; Substrate Concentration - 10.0
mM; Auxiliary Cascade Enzymes - SEQ ID NO: 1352 (10.0 μM), SEQ ID NO: 1354 (10.0 μM),
SEQ ID NO: 2670 (2.0 μM); Dilution into Coupling Reaction - 800X; Substrate Oligonucleotides -
SEQ ID NO: 3194, SEQ ID NO: 3193; Product Oligonucleotides - SEQ ID NO: 3198, SEQ ID NO:
3197.

Activity relative to SEQ ID NO: 2602 (Activity FIOP) was calculated based on the percentage of extension products observed for the variant compared with the percentage observed with SEQ ID NO: 2602 (where the percent product may be set as the average of replicates or else the highest single sample as appropriate). The results are shown in Table 41.2.

TABLE 41.2
Adenylate kinase activity relative to SEQ ID NO: 2602

| | | FIOP % yield mUTP |
|---|---|---|
| SEQ ID NO: | Amino Acid Differences | relative to |
| (nt/aa) | (Relative to SEQ ID NO: 2602) | SEQ ID NO: 2602 |
| 2715/2716 | M216P | +++ |
| 2717/2718 | Q50R | +++ |
| 2719/2720 | P21T | +++ |
| 2721/2722 | Q173F | +++ |
| 2723/2724 | G20R | +++ |
| 2725/2726 | L156A | ++ |
| 2727/2728 | M216L | ++ |
| 2729/2730 | K69F | ++ |
| 2731/2732 | S43N | ++ |
| 2733/2734 | G148P | ++ |
| 2735/2736 | G30L | ++ |
| 2737/2738 | R149L | ++ |
| 2739/2740 | P21R | ++ |
| 2741/2742 | V143T | ++ |
| 2743/2744 | P21L | ++ |
| 2745/2746 | M216W | ++ |
| 2747/2748 | M153V | ++ |
| 2749/2750 | G20P | ++ |
| 2751/2752 | M216G | ++ |
| 2753/2754 | I42T | ++ |
| 2755/2756 | D98G | ++ |
| 2757/2758 | G30Y | ++ |
| 2759/2760 | W66P | + |
| 2761/2762 | G99C | + |
| 2763/2764 | G99A | + |
| 2765/2766 | M216D | + |
| 2767/2768 | D98Q | + |
| 2769/2770 | R102C | + |
| 2771/2772 | P80S | + |
| 2773/2774 | M216N | + |
| 2775/2776 | G27C | + |
| 2777/2778 | G148S | + |
| 2779/2780 | R102N | + |
| 2781/2782 | M67R | + |
| 2783/2784 | P80G | + |
| 2785/2786 | L18E | + |
| 2787/2788 | L18C/N56T | + |
| 2789/2790 | L139H | + |
| 2791/2792 | G70R | + |
| 2793/2794 | M216E | + |
| 2795/2796 | M184N | + |
| 2797/2798 | R142L | + |
| 2799/2800 | K26L | + |
| 2801/2802 | I32L | + |
| 2803/2804 | D98A | + |
| 2805/2806 | N152F | + |
| 2807/2808 | G30S | + |
| 2809/2810 | R141V | + |
| 2811/2812 | G20T | + |
| 2813/2814 | R102A | + |
| 2815/2816 | G20V | + |
| 2817/2818 | V135I | + |
| 2819/2820 | R141G | + |
| 2821/2822 | R137L | + |
| 2823/2824 | Q29R | + |

Levels of increased activity were determined for FIOP % yield fGTP relative to SEQ ID NO: 2368 and are defined as follows: “+” > 1.1, “++” > 1.3, “+++” > 1.5.

### Example 42

### Improvements Over SEQ ID NO: 2602 in Conversion of Nucleoside Monophosphates to Nucleotides

### HTP Screening for Improved AdyK Variants

Adenylate kinase of SEQ ID NO: 2602 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 42.1.

Reactions were performed as described in Example 39 using conditions summarized in Table 42.1. Data were collected using the CE assay described in Example 6.

TABLE 42.1
Reaction conditions

Lysis Buffer - TEoA (pH 7.5), 0.1 g/L lysozyme; Lysis Conditions - 100 μL, 50.0° C., 60 min;
Reaction buffer - 50.0 mM Tris, 50.0 mM sodium pyruvate, 50.0 mM dibasic potassium phosphate,
10.0 μM ATP, 10 mM magnesium chloride, pH 8.0; Lysate concentration (vol %) - 10.0; Reaction
Conditions - 1.0 μL, 30.0° C., 1.0 hr; Nucleoside substrate - mG or mU; Substrate Concentration -
10.0 mM; Auxiliary Cascade Enzymes - SEQ ID NO: 1352 (10.0 μM), SEQ ID NO: 1354 (10.0
μM), SEQ ID NO: 2670 (2.0 μM); Dilution into Coupling Reaction - 200X; Substrate
Oligonucleotides - SEQ ID NO: 3194, SEQ ID NO: 3193; Product Oligonucleotides - SEQ ID NO:
3200, SEQ ID NO: 3199 or SEQ ID NO: 3198, SEQ ID NO: 3197.

Activity relative to SEQ ID NO: 2602 (Activity FIOP) was calculated based on the percentage of extension products observed for the variant compared with the percentage observed with SEQ ID NO: 2602 (where the percent product may be set as the average of replicates or else the highest single sample as appropriate). The results are shown in Table 42.2.

TABLE 42.2
Adenylate kinase activity relative to SEQ ID NO: 2602

| | | FIOP % yield | FIOP % yield |
|---|---|---|---|
| | | mUTP relative | mGTP relative |
| SEQ ID | Amino Acid Differences | to SEQ ID | to SEQ ID NO: |
| NO: (nt/aa) | (Relative to SEQ ID NO: 2602) | NO: 2602 | 2602 |
| 2825/2826 | R105K/I127L/A136V/P195G | + | +++ |
| 2827/2828 | E65T/R105K/I127L/L175F | | +++ |
| 2829/2830 | E65T/I127L/S170R | ++ | +++ |
| 2831/2832 | V104A/I127L/A136V/L175F | + | ++ |
| 2833/2834 | E65T/V104A/A136V | | ++ |
| 2835/2836 | E65T/R105K/I127L/A136V | + | ++ |
| 2837/2838 | V104A/R105K/I127L/A136V/L175F/P195G | ++ | ++ |
| 2839/2840 | E65T/V104A/A136V/S170R | | ++ |
| 2841/2842 | R105K/S116A/I127L/A136Y/S170R/L175F | + | + |
| 2843/2844 | E65T | | + |
| 2845/2846 | E65T/R105K/I127L/S170R | | + |
| 2847/2848 | E65T/R105K/A136V | + | + |
| 2849/2850 | E65T/V104A/R105K/S116A/I127L | | + |
| 2851/2852 | R105K | | + |
| 2853/2854 | E65T/V104A/R105K/I127L/A136V | | + |
| 2855/2856 | V104A/I127L/A136V/S170R | | + |
| 2857/2858 | V104A/R105K/A136V/S170R | +++ | |
| 2859/2860 | D132K | +++ | |
| 2861/2862 | E65T/V104A | +++ | |
| 2863/2864 | R105K/A136V/P195G | +++ | |
| 2865/2866 | V104A/I127L | ++ | |
| 2867/2868 | E65T/S116A | ++ | |
| 2869/2870 | I127L/P195G | ++ | |
| 2871/2872 | R105K/A136V | ++ | |
| 2873/2874 | S116A/I127L/A136Y/L175F | ++ | |
| 2875/2876 | E65T/I127L/A136V/S170R | ++ | |
| 2877/2878 | E65T/R105K/S116A/A136V/S170R | ++ | |
| 2879/2880 | E65T/I127L/A136Y | ++ | |
| 2881/2882 | S170R | + | |
| 2883/2884 | E65T/R105K/I127L/A136V/L175F | + | |
| 2885/2886 | E65T/R105K/A136V/S170R | + | |
| 2887/2888 | V104A/R105K | + | |
| 2889/2890 | A136V | + | |
| 2891/2892 | R105K/I127L/A136V | + | |
| 2893/2894 | E65T/R105K/S170R | + | |
| 2895/2896 | G94Y/Q173R | + | |
| 2897/2898 | V104A/R105K/I127L | + | |
| 2899/2900 | G94Y/K187G | + | |

Levels of increased activity were determined for FIOP % yield mUTP relative to SEQ ID NO: 2602 and are defined as follows: “+” > 2.7, “++” > 4.5, “+++” > 7.0. Levels of increased activity were determined for FIOP % yield mGTP relative to SEQ ID NO: 2602 and are defined as follows: “+” > 2.0, “++” > 3.0, “+++” > 4.5.

### Example 43

### Improvements Over SEQ ID NO: 2602 in Conversion of Nucleoside Monophosphates to Nucleotides

### HTP Screening for Improved AdyK Variants

Adenylate kinase of SEQ ID NO: 2602 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 43.1.

Reactions were performed as described in Example 39 using conditions summarized in Table 43.1. Data were collected using the CE assay described in Example 6.

TABLE 43.1
Reaction conditions

Lysis Buffer - TEoA (pH 7.5), 0.1 g/L lysozyme; Lysis Conditions - 100 μL, 50.0° C., 60 min;
Reaction buffer - 50.0 mM Tris, 50.0 mM sodium pyruvate, 50.0 mM dibasic potassium phosphate,
10.0 μM ATP, 10 mM magnesium chloride, pH 8.0; Lysate concentration (vol %) - 2.5; Reaction
Conditions - 1.0 μL, 30.0° C., 1.0 hr; Nucleoside substrate - mU; Substrate Concentration - 10.0
mM; Auxiliary Cascade Enzymes - SEQ ID NO: 2662 (10.0 μM), SEQ ID NO: 1354 (10.0 μM),
SEQ ID NO: 2672 (2.0 μM); Dilution into Coupling Reaction - 800X; Substrate Oligonucleotides -
SEQ ID NO: 3194, SEQ ID NO: 3193; Product Oligonucleotides - SEQ ID NO: 3198, SEQ ID NO:
3197.

Activity relative to SEQ ID NO: 2602 (Activity FIOP) was calculated based on the percentage of extension products observed for the variant compared with the percentage observed with SEQ ID NO: 2602 (where the percent product may be set as the average of replicates or else the highest single sample as appropriate). The results are shown in Table 43.2.

TABLE 43.2
Adenylate kinase activity relative to SEQ ID NO: 2602

| | | FIOP % yield UTP |
|---|---|---|
| SEQ ID NO: | Amino Acid Differences | relative to |
| (nt/aa) | (Relative to SEQ ID NO: 2602) | SEQ ID NO: 2602 |
| 2901/2902 | T37L | ++ |
| 2903/2904 | T37I | ++ |
| 2905/2906 | Q117L | ++ |
| 2907/2908 | V59Y | ++ |
| 2909/2910 | K89P | ++ |
| 2911/2912 | P40S | + |
| 2913/2914 | Q50A | + |
| 2915/2916 | V59T | + |
| 2917/2918 | Q120L | + |
| 2919/2920 | K36V | + |
| 2921/2922 | E128I | + |
| 2923/2924 | G168L | + |
| 2925/2926 | K203L | + |

Levels of increased activity were determined for FIOP % yield UTP relative to SEQ ID NO: 2602 and are defined as follows: “+” > 1.1, “++” > 1.25.

### Example 44

### Improvements Over SEQ ID NO: 2602 in Conversion of Nucleoside Monophosphates to Nucleotides

### HTP Screening for Improved AdyK Variants

Adenylate kinase of SEQ ID NO: 2602 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 44.1.

Reactions were performed as described in Example 39 using conditions summarized in Table 44.1. Data were collected using the CE assay described in Example 6.

TABLE 44.1
Reaction conditions

Lysis Buffer - TEoA (pH 7.5), 0.1 g/L lysozyme; Lysis Conditions - 100 μL, 50.0° C., 60 min;
Reaction buffer - 50.0 mM Tris, 50.0 mM sodium pyruvate, 50.0 mM dibasic potassium phosphate,
10.0 μM ATP, 10 mM magnesium chloride, pH 8.0; Lysate concentration (vol %) - 1; Reaction
Conditions - 1.0 μL, 30.0° C., 1.0 hr; Nucleoside substrate - fG; Substrate Concentration - 10.0 mM;
Auxiliary Cascade Enzymes - SEQ ID NO: 2662 (10.0 μM), SEQ ID NO: 1354 (10.0 μM), SEQ ID
NO: 2672 (2.0 μM); Dilution into Coupling Reaction - 800X; Substrate Oligonucleotides - SEQ ID
NO: 3194, SEQ ID NO: 3193; Product Oligonucleotides - SEQ ID NO: 3196, SEQ ID NO: 3195.

Stability relative to SEQ ID NO: 2602 (Stability FIOP) was calculated based on the percentage of extension products observed for the variant compared with the percentage observed with SEQ ID NO: 2602 (where the percent product may be set as the average of replicates or else the highest single sample as appropriate). The results are shown in Table 44.2.

TABLE 44.2
Adenylate kinase stability relative to SEQ ID NO: 2602

| | | FIOP stability |
|---|---|---|
| SEQ ID NO: | Amino Acid Differences | relative to |
| (nt/aa) | (Relative to SEQ ID NO: 2602) | SEQ ID NO: 2602 |
| 2927/2928 | D90S | +++ |
| 2929/2930 | I32F | +++ |
| 2931/2932 | S61E | +++ |
| 2933/2934 | K36S | ++ |
| 2935/2936 | S61N | ++ |
| 2937/2938 | S170G | ++ |
| 2939/2940 | V172A | ++ |
| 2941/2942 | D166S | ++ |
| 2943/2944 | E128R | ++ |
| 2945/2946 | E58Y | ++ |
| 2947/2948 | E128C | ++ |
| 2949/2950 | G94M | + |
| 2951/2952 | E131G | + |
| 2953/2954 | D111R | + |
| 2955/2956 | Q50N | + |
| 2957/2958 | D111P | + |
| 2959/2960 | Q50V | + |
| 2961/2962 | V104R | + |
| 2963/2964 | P195I | + |
| 2965/2966 | T37F | + |
| 2967/2968 | R119P | + |
| 2969/2970 | G94V | + |
| 2971/2972 | D166L | + |
| 2973/2974 | V169Y | + |
| 2975/2976 | D132L | + |
| 2977/2978 | K192H | + |
| 2979/2980 | L97I | + |
| 2981/2982 | K89L | + |
| 2983/2984 | Y200A | + |
| 2985/2986 | L110F | + |
| 2987/2988 | G118L | + |

Levels of increased activity were determined for FIOP stability relative to SEQ ID NO: 2602 and are defined as follows: “+” > 1.6, “++” > 2.2, “+++” > 3.0.

### Example 45

### Improvements Over SEQ ID NO: 2832 in Conversion of Nucleoside Monophosphates to Nucleotides

### HTP Screening for Improved AdyK Variants

Adenylate kinase of SEQ ID NO: 2832 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 45.1.

Reactions were performed as described in Example 39 using conditions summarized in Table 45.1. Data were collected using the CE assay described in Example 6.

TABLE 45.1
Reaction conditions

Lysis Buffer - TEoA (pH 7.5), 0.1 g/L lysozyme; Lysis Conditions - 100 μL, 66° C., 60 min; Reaction
buffer - 50.0 mM Tris, 50.0 mM sodium pyruvate, 50.0 mM dibasic potassium phosphate, 10.0 μM
ATP, 10 mM magnesium chloride, pH 8.0; Lysate concentration (vol %) - 5; Reaction Conditions -
1.0 μL, 30.0° C., 16 hr; Nucleoside substrate - fG; Substrate Concentration - 10.0 mM; Auxiliary
Cascade Enzymes - SEQ ID NO: 2662 (10.0 μM), SEQ ID NO: 1354 (10.0 μM), SEQ ID NO: 2672
(2.0 μM); Dilution into Coupling Reaction - 800X; Substrate Oligonucleotides - SEQ ID NO: 3194,
SEQ ID NO: 3193; Product Oligonucleotides - SEQ ID NO: 3196, SEQ ID NO: 3195.

Stability relative to SEQ ID NO: 2832 (Stability FIOP) was calculated based on the percentage of extension products observed for the variant compared with the percentage observed with SEQ ID NO: 2832 (where the percent product may be set as the average of replicates or else the highest single sample as appropriate). The results are shown in Table 45.2.

TABLE 45.2
Adenylate kinase activity relative to SEQ ID NO: 2832

| | | FIOP stability |
|---|---|---|
| SEQ ID NO: | Amino Acid Differences | relative to |
| (nt/aa) | (Relative to SEQ ID NO: 2832) | SEQ ID NO: 2832 |
| 2989/2990 | Q50N | +++ |
| 2991/2992 | K36S | +++ |
| 2993/2994 | Y151F/P157V | ++ |
| 2995/2996 | M216D | ++ |
| 2997/2998 | Q50N/K93V | ++ |
| 2999/3000 | D90S/A104R | ++ |
| 3001/3002 | K36S/Q50N/K89L/K93V/L139R | ++ |
| 3003/3004 | K36S/Q50N/K89L/K93V/L110F | ++ |
| 3005/3006 | K36S/Q50N | ++ |
| 3007/3008 | A104R/Y151F/I154Q | + |
| 3009/3010 | Q50N/K89L/K93V/L139R | + |
| 3011/3012 | K36S/Q50N/K89L/L139R/S170G | + |
| 3013/3014 | K36S/Q50N/K89L/V172A | + |
| 3015/3016 | A104R/Y151F/I154Q/P157V | + |
| 3017/3018 | Y151F/M216E | + |
| 3019/3020 | K36S/K89L | + |
| 3021/3022 | Q50N/K89L/K93V | + |
| 3023/3024 | Q50N/S170G | + |
| 3025/3026 | A104R/I154Q/P157V/M216E | + |
| 3027/3028 | K36S/S170G | + |
| 3029/3030 | D90S/Y151F/P157V | + |
| 3031/3032 | K89L/S170G/V172A | + |

Levels of increased activity were determined for FIOP stability relative to SEQ ID NO: 2832 and are defined as follows: “+” > 1.1, “++” > 1.3, “+++” > 1.5.

### Example 46

### Improvements Over SEQ ID NO: 2994 in Conversion of Nucleoside Monophosphates to Nucleotides

### HTP Screening for Improved AdyK Variants

Adenylate kinase of SEQ ID NO: 2994 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 46.1.

Reactions were performed as described in Example 39 using conditions summarized in Table 46.1. Data were collected using the CE assay described in Example 6.

TABLE 46.1
Reaction conditions

Lysis Buffer - TEoA (pH 7.5), 0.1 g/L lysozyme; Lysis Conditions - 100 μL, 50.0° C., 60 min;
Reaction buffer - 50.0 mM Tris, 50.0 mM sodium pyruvate, 50.0 mM dibasic potassium phosphate,
10.0 μM ATP, 10 mM magnesium chloride, pH 8.0; Lysate concentration (vol %) - 10.0; Reaction
Conditions - 1.0 μL, 30.0° C., 1.0 hr; Nucleoside substrate - fG; Substrate Concentration - 10.0 mM;
Auxiliary Cascade Enzymes - SEQ ID NO: 2664 (10.0 μM), SEQ ID NO: 2668 (10.0 μM), SEQ ID
NO: 2672 (2.0 μM); Dilution into Coupling Reaction - 800X; Substrate Oligonucleotides - SEQ ID
NO: 3194, SEQ ID NO: 3193; Product Oligonucleotides - SEQ ID NO: 3196, SEQ ID NO: 3195.

Activity relative to SEQ ID NO: 2994 (Activity FIOP) was calculated based on the percentage of extension products observed for the variant compared with the percentage observed with SEQ ID NO: 2994 (where the percent product may be set as the average of replicates or else the highest single sample as appropriate). The results are shown in Table 46.2.

TABLE 46.2
adenylate kinase activity relative to SEQ ID NO: 2994

| | | FIOP % yield fGTP |
|---|---|---|
| SEQ ID NO: | Amino Acid Differences | relative to |
| (nt/aa) | (Relative to SEQ ID NO: 2994) | SEQ ID NO: 2994 |
| 3033/3034 | L226R | +++ |
| 3035/3036 | P80W | +++ |
| 3037/3038 | P217T | +++ |
| 3039/3040 | S170P | ++ |
| 3041/3042 | N79P | ++ |
| 3043/3044 | N79K | ++ |
| 3045/3046 | S170R | ++ |
| 3047/3048 | G224S | ++ |
| 3049/3050 | G224T | ++ |
| 3051/3052 | E76G | ++ |
| 3053/3054 | N79A | ++ |
| 3055/3056 | S112C | ++ |
| 3057/3058 | V172M | ++ |
| 3059/3060 | L68A | ++ |
| 3061/3062 | V182G | ++ |
| 3063/3064 | W228D | ++ |
| 3065/3066 | P74Q | + |
| 3067/3068 | S170H | + |
| 3069/3070 | L226K | + |
| 3071/3072 | V59W | + |
| 3073/3074 | F113W | + |
| 3075/3076 | R119S | + |
| 3077/3078 | V182L | + |
| 3079/3080 | N79L | + |
| 3081/3082 | Q50L | + |
| 3083/3084 | E76L | + |
| 3085/3086 | N79W | + |
| 3087/3088 | V182Q | + |
| 3089/3090 | D90K | + |
| 3091/3092 | D230M | + |
| 3093/3094 | E58C | + |
| 3095/3096 | K231R | + |
| 3097/3098 | L68E | + |
| 3099/3100 | L68V | + |
| 3101/3102 | M184R | + |

Levels of increased activity were determined for FIOP activity relative to SEQ ID NO: 2994 and are defined as follows: “+” > 2.5, “++” > 3.0, “+++” > 4.0.

### Example 47

### Improvements Over SEQ ID NO: 2994 in Conversion of Nucleoside Monophosphates to Nucleotides

### HTP Screening for Improved AdyK Variants

Adenylate kinase of SEQ ID NO: 2994 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 47.1.

Reactions were performed as described in Example 39 using conditions summarized in Table 47.1. Data were collected using the CE assay described in Example 6.

TABLE 47.1
Reaction conditions

Lysis Buffer - TEoA (pH 7.5), 0.1 g/L lysozyme; Lysis Conditions - 100 μL, 50.0° C., 60 min; Reaction
buffer - 50.0 mM Tris, 50.0 mM sodium pyruvate, 50.0 mM dibasic potassium phosphate, 10.0 μM
ATP, 10 mM magnesium chloride, pH 8.0; Lysate concentration (vol %) - 10.0; Reaction Conditions -
1.0 μL, 30.0° C., 1.0 hr; Nucleoside substrate - mG; Substrate Concentration - 10.0 mM; Auxiliary
Cascade Enzymes - SEQ ID NO: 2666 (10.0 μM), SEQ ID NO: 2668 (10.0 μM), SEQ ID NO: 2672
(2.0 μM); Dilution into Coupling Reaction - 800X; Substrate Oligonucleotides - SEQ ID NO: 3194,
SEQ ID NO: 3193; Product Oligonucleotides - SEQ ID NO: 3200, SEQ ID NO: 3199.

Activity relative to SEQ ID NO: 2994 (Activity FIOP) was calculated based on the percentage of extension products observed for the variant compared with the percentage observed with SEQ ID NO: 2994 (where the percent product may be set as the average of replicates or else the highest single sample as appropriate). The results are shown in Table 47.2.

TABLE 47.2
adenylate kinase activity relative to SEQ ID NO: 2994

| | | FIOP % yield mGTP |
|---|---|---|
| SEQ ID NO: | Amino Acid Differences | relative to |
| (nt/aa) | (Relative to SEQ ID NO: 2994) | SEQ ID NO: 2994 |
| 3103/3104 | K83L/R190Q/S201A/M216E | +++ |
| 3105/3106 | K83L/M216E | +++ |
| 3107/3108 | N79W/W228V | ++ |
| 3109/3110 | V157P/R190Q/M216E | ++ |
| 3111/3112 | K83L/A104R/F151Y/G168S/ | ++ |
| | Q173T/R190Q | |
| 3113/3114 | K83L | ++ |
| 3115/3116 | S229I | ++ |
| 3117/3118 | A104R/V157P | ++ |
| 3119/3120 | V157P/R190Q | ++ |
| 3121/3122 | V157P/W228V | + |
| 3123/3124 | V157P/Q173T/R190Q/M216E | + |
| 3125/3126 | K83L/Q173V/R190Q | + |
| 3127/3128 | K83L/Q173T/S201A | + |
| 3129/3130 | N79W/V157P/W228V/S229I | + |
| 3131/3132 | K83L/F113Y | + |
| 3133/3134 | S201F/M216E | + |
| 3135/3136 | K83L/A104R | + |
| 3137/3138 | N79W/V157P/S229I | + |
| 3139/3140 | V157P | + |
| 3141/3142 | Q173T/M216E | + |
| 3143/3144 | K83L/Q173V/R190Q/S201F | + |
| 3145/3146 | V157P/R183L | + |
| 3147/3148 | K83L/R190Q | + |
| 3149/3150 | N79W/A104R/V157P/W228V | + |

Levels of increased activity were determined for FIOP % yield mGTP relative to SEQ ID NO: 2994 and are defined as follows: “+” > 2.5, “++” > 4.0, “+++” > 6.5.

### Example 48

### Improvements Over SEQ ID NO: 3104 in Conversion of Nucleoside Monophosphates to Nucleotides

### HTP Screening for Improved AdyK Variants

Adenylate kinase of SEQ ID NO: 3104 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 48.1.

Reactions were performed as described in Example 39 using conditions summarized in Table 48.1. Data were collected using the CE assay described in Example 6.

TABLE 48.1
Reaction conditions

Lysis Buffer - TEoA (pH 7.5), 0.1 g/L lysozyme; Lysis Conditions - 100 μL, 55° C., 60 min;
Reaction buffer - 50 mM Tris, 50 mM sodium pyruvate, 50 mM dibasic potassium phosphate, 10
uM ATP, 10 mM magnesium chloride, pH 8.0; Lysate concentration (vol %) - 10; Reaction
Conditions - 1 μL, 30° C., 16 hr; Nucleoside substrate - mU; Substrate Concentration - 10 mM;
Auxiliary Cascade Enzymes - SEQ ID NO: 2666 (10 μM), SEQ ID NO: 2668 (0.25 g/L lyophilized
lysate), SEQ ID NO: 2674 (0.25 μM); Dilution into Coupling Reaction - 800X; Substrate
Oligonucleotides - SEQ ID NO: 3194, SEQ ID NO: 3193; Product Oligonucleotides - SEQ ID NO:
3198, SEQ ID NO: 3197.

Activity relative to SEQ ID NO: 3104 (Activity FIOP) was calculated based on the percentage of extension products observed for the variant compared with the percentage observed with SEQ ID NO: 3104 (where the percent product may be set as the average of replicates or else the highest single sample as appropriate). The results are shown in Table 48.2.

TABLE 48.2
adenylate kinase activity relative to SEQ ID NO: 3104

| | | FIOP activity |
|---|---|---|
| SEQ ID NO: | Amino Acid Differences | relative to |
| (nt/aa) | (Relative to SEQ ID NO: 3104) | SEQ ID NO: 3104 |
| 3151/3152 | A104R/V157P/W228V/S229I | +++ |
| 3153/3154 | A104R | +++ |
| 3155/3156 | A104R/V157P/S229I | +++ |
| 3157/3158 | V157P/W228V/S229I | +++ |
| 3159/3160 | A104R/S170P/Q190R/W228V | ++ |
| 3161/3162 | A104R/V157P | ++ |
| 3163/3164 | Q190R | ++ |
| 3165/3166 | A201S/W228V | + |
| 3167/3168 | Q190R/S229I | + |
| 3169/3170 | S170P/Q190R/W228V | + |
| 3171/3172 | S170P/Q190R/W228V/S229I | + |
| 3173/3174 | V157P/W228V | + |
| 3175/3176 | A104R/A201S/W228V | + |

Levels of increased activity were determined for FIOP activity relative to SEQ ID NO: 3104 and are defined as follows: “+” > 1.5, “++” > 2.0, “+++” > 3.0.

### Example 49

### Improvements Over SEQ ID NO: 3104 in Conversion of Nucleoside Monophosphates to Nucleotides

### HTP Screening for Improved AdyK Variants

Adenylate kinase of SEQ ID NO: 3104 was selected as the parent adenylate kinase enzyme. Libraries of genes were produced from the parent gene using various techniques (e.g. saturation mutagenesis and recombination of previously identified beneficial mutations). The polypeptides encoded by each gene were produced in HTP and prepared as described in Table 49.1.

Reactions were performed as described in Example 39 using conditions summarized in Table 49.1. Data were collected using the CE assay described in Example 6.

TABLE 49.1
Reaction conditions

Lysis Buffer - TEoA (pH 7.5), 0.1 g/L lysozyme; Lysis Conditions - 100 μL, 60° C., 60 min;
Reaction buffer - 50 mM Tris, 50 mM sodium pyruvate, 50 mM dibasic potassium phosphate, 10
uM ATP, 10 mM magnesium chloride, pH 8.0; Lysate concentration (vol %) - 10; Reaction
Conditions - 1 μL, 30° C., 16 hr; Nucleoside substrate - mU; Substrate Concentration - 10 mM;
Auxiliary Cascade Enzymes - SEQ ID NO: 2666 (10 μM), SEQ ID NO: 2668 (0.25 g/L lyophilized
lysate), SEQ ID NO: 2674 (0.25 μM); Dilution into Coupling Reaction - 800X; Substrate
Oligonucleotides - SEQ ID NO: 3194, SEQ ID NO: 3193; Product Oligonucleotides - SEQ ID NO:
3198, SEQ ID NO: 3197.

Stability relative to SEQ ID NO: 3104 (Stability FIOP) was calculated based on the percentage of extension products observed for the variant compared with the percentage observed with SEQ ID NO: 3104 (where the percent product may be set as the average of replicates or else the highest single sample as appropriate). The results are shown in Table 49.2.

TABLE 49.2
Adenylate kinase activity relative to SEQ ID NO: 3104

| | | FIOP stability |
|---|---|---|
| SEQ ID NO: | Amino Acid Differences | relative to |
| (nt/aa) | (Relative to SEQ ID NO: 3104) | SEQ ID NO: 3104 |
| 3153/3154 | A104R | ++ |
| 3177/3178 | A104R/Q190R/A201S | +++ |
| 3179/3180 | A104R/Q190R/W228V | ++ |
| 3181/3182 | W228V | ++ |
| 3183/3184 | A104R/Q190R | + |
| 3185/3186 | A104R/V157P/Q190R/W228V | + |
| 3187/3188 | A104R/S170P/Q190R | + |
| 3189/3190 | A104R/W228V/S229I | + |
| 3191/3192 | A104R/S170P/W228V | + |

Levels of increased activity were determined for FIOP stability relative to SEQ ID NO: 3104 and are defined as follows: “+” > 1.5, “++” > 2.5, “+++” > 3.5.

### Example 50

### Relative Activities of AdyK Variants for the Conversion of Nucleosides to Nucleotides

### Shake Flask Characterization of AdoK Variants

AdyK variants SEQ ID NO: 2, SEQ ID NO: 1032, SEQ ID NO: 1708, SEQ ID NO: 1980, SEQ ID NO: 2138, SEQ ID NO: 2368, SEQ ID NO: 2832 and SEQ ID NO: 3152 were expressed as described in Example 3 and either purified or lyophilized.

To assess activity, each variant was added to a 5 μL reaction at a final concentration of 10 μM. The reaction contained 50 mM Tris (pH 8.0), 50 mM sodium pyruvate, 50 mM potassium phosphate, 10 μM ATP, 10 mM MgCl₂, 0.4 g/L lyophilized lysate SEQ ID NO: 2666, 0.25 g/L lyophilized lysate SEQ ID NO: 2668, 0.25 g/L lyophilized lysate SEQ ID NO: 2674 and 10 mM nucleoside. Reactions were incubated in a Multitron (Infors) shaker at 30° C. & 600 rpm for 16 hours. Reactions were then quenched and diluted 40-fold with 75% methanol and analyzed by HPLC as described in Example 5. Relative activities were normalized to the lowest observed activity by a variant on a given substrate. The results are shown in Table 50.1.

TABLE 50.1
Adenylate kinase activity relative to SEQ ID NO: 2

| | fA | fC | fG | fU | mA | mC | mG | mU |
|---|---|---|---|---|---|---|---|---|
| | relative | relative | relative | relative | relative | relative | relative | relative |
| SEQ ID | to SEQ | to SEQ | to SEQ | to SEQ | to SEQ | to SEQ | to SEQ | to SEQ |
| NO: | ID NO: | ID NO: | ID NO: | ID NO: | ID NO: | ID NO: | ID NO: | ID NO: |
| (nt/aa) | 2 | 2 | 2 | 2 | 2 | 2 | 2 | 2 |
| 1/2 | ++ | ++ | + | + | ++ | + | + | + |
| 1031/1032 | ++ | +++ | + | ++ | ++ | +++ | + | +++ |
| 1707/1708 | ++ | +++ | ++ | ++ | ++ | +++ | + | +++ |
| 1979/1980 | ++ | +++ | ++ | ++ | ++ | +++ | + | +++ |
| 2137/2138 | ++ | +++ | ++ | ++ | ++ | +++ | + | +++ |
| 2367/2368 | ++ | +++ | + | ++ | +++ | +++ | ++ | +++ |
| 2831/2832 | ++ | +++ | + | ++ | ++ | +++ | ++ | +++ |
| 3151/3152 | +++ | +++ | ++ | ++ | +++ | +++ | ++ | +++ |

Percent yield of NTP were determined for fA relative to SEQ ID NO: 2 and are defined as follows: “+” > 0.1, “++” > 2.0, “+++” > 20.0. Percent yield of NTP were determined for fC relative to SEQ ID NO: 2 and are defined as follows: “+” > 0.1, “++” > 2.0, “+++” > 20.0. Percent yield of NTP were determined for fG relative to SEQ ID NO: 2 and are defined as follows: “+” > 0.1, “++” > 2.0, “+++” > 20.0. Percent yield of NTP were determined for fU relative to SEQ ID NO: 2 and are defined as follows: “+” > 0.1, “++” > 2.0, “+++” > 20.0. Percent yield of NTP were determined for mA relative to SEQ ID NO: 2 and are defined as follows: “+” > 0.1, “++” > 2.0, “+++” > 20.0. Percent yield of NTP were determined for mC relative to SEQ ID NO: 2 and are defined as follows: “+” > 0.1, “++” > 2.0, “+++” > 20.0. Percent yield of NTP were determined for mG relative to SEQ ID NO: 2 and are defined as follows: “+” > 0.1, “++” > 2.0, “+++” > 20.0. Percent yield of NTP were determined for mU relative to SEQ ID NO: 2 and are defined as follows: “+” > 0.1, “++” > 2.0, “+++” > 20.0.

While the invention has been described with reference to the specific embodiments, various changes can be made and equivalents can be substituted to adapt to a particular situation, material, composition of mailer, process, process step or steps, without departing from the spirit and scope of the invention.

For all purposes, each and every publication and patent document cited in this disclosure is incorporated herein by reference as if each such publication or document was specifically and individually indicated to be incorporated herein by reference. Citation of publications and patent documents is not intended as an indication that any such document is pertinent prior art, nor does it constitute an admission as to its contents or date.

1. An engineered adenylate kinase comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12-231 of an even-numbered SEQ ID NO. of SEQ ID NOs: 2-1302, 1356-2652, and 2676-3192, or to a reference sequence corresponding to an even-numbered SEQ ID NO. of SEQ ID NOs: 2-1302, 1356-2652, and 2676-3192, wherein the amino acid sequence comprises one or more substitutions relative to a reference sequence corresponding to amino acid residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or relative to a reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

2. The engineered adenylate kinase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or to a reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, wherein the amino acid sequence comprises one or more substitutions relative to the reference sequence corresponding to amino acid residues 12-231 of SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 2, 4, 172, 376, 508, 524, 584, 610, 674, 816, 874, 1032, 1388, 1708, 1952, 1980, 2072, 2138, 2294, 2368, 2602, 2832, 2994, or 3104.

3. The engineered adenylate kinase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12-231 of SEQ ID NO: 2, or to the reference sequence corresponding to SEQ ID NO: 2, wherein the amino acid sequence comprises one or more substitutions relative to the reference sequence corresponding to amino acid residues 12-231 of SEQ ID NO: 2, or relative to the reference sequence corresponding to SEQ ID NO: 2.

4. The engineered adenylate kinase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to amino acid residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, wherein the amino acid sequence comprises one or more substitutions relative to the reference sequence corresponding to amino acid residues 12-231 of SEQ ID NO: 2, or relative to the reference sequence corresponding to SEQ ID NO: 2.

5. The engineered adenylate kinase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to a reference sequence corresponding to amino acid residues 12-231 of an even-numbered SEQ ID NO. of SEQ ID NOs: 4-1302, 1356-2652, and 2676-3192, or to a reference sequence corresponding to an even-numbered SEQ ID NO. of SEQ ID NOs: 4-1302, 1356-2652, and 2676-3192, wherein the amino acid sequence comprises one or more substitutions relative to the reference sequence corresponding to amino acid residues 12-231 of SEQ ID NO: 2, or relative to the reference sequence corresponding to SEQ ID NO: 2.

6. The engineered adenylate kinase of claim 1, wherein the amino acid sequence of the engineered adenylate kinase comprises at least a substitution at amino acid position 11, 13, 14, 15, 16, 18, 20, 21, 22, 26, 27, 29, 30, 31, 32, 34, 35, 36, 37, 38, 39, 40, 42, 43, 44, 46, 48, 50, 51, 52, 53, 54, 55, 56, 57, 58, 59, 60, 61, 62, 63, 64, 65, 66, 67, 68, 69, 70, 71, 73, 74, 75, 76, 77, 78, 79, 80, 81, 82, 83, 84, 87, 88, 89, 90, 91, 92, 93, 94, 97, 98, 99, 100, 101, 102, 104, 105, 108, 109, 110, 111, 112, 113, 115, 116, 117, 118, 119, 120, 122, 123, 124, 125, 126, 127, 128, 129, 131, 132, 133, 134, 135, 136, 137, 138, 139, 140, 141, 142, 143, 146, 148, 149, 150, 151, 152, 153, 154, 155, 156, 157, 161, 162, 163, 166, 168, 169, 170, 172, 173, 175, 178, 179, 180, 181, 182, 183, 184, 186, 187, 188, 190, 191, 192, 194, 195, 196, 197, 198, 199, 200, 201, 202, 203, 204, 205, 206, 207, 208, 210, 211, 212, 213, 214, 215, 216, 217, 218, 219, 220, 221, 222, 223, 224, 225, 226, 227, 228, 229, 230, or 231, or combinations thereof, wherein the amino acid positions are relative to the reference sequence corresponding to amino acid residues 12-231 of SEQ ID NO: 2, or relative to the reference sequence corresponding to SEQ ID NO: 2.

7. The engineered adenylate kinase of claim 1, wherein the amino acid sequence of the engineered adenylate kinase comprises at least a substitution or amino acid residue 11D, 13G/S, 14C/E, 15F, 16M, 18C/E/L, 20A/P/R/T/V, 21L/N/R/S/T/V, 22A/S, 26L, 27C/S, 29Q/R, 30G/L/S/Y, 31R, 32F/I/L/N, 34A/E/S/V, 35A/E/F/L/S, 36A/E/I/L/M/Q/R/S/V/Y, 37F/G/I/L/R/Y, 38C/F/L/R, 39A/M/Q/T, 40C/F/L/S/T, 42T, 43A/C/G/H/N, 445, 46E/F/L/M/Y, 48F/L, 50A/C/L/N/Q/R/S/V, 51F/G/K/L/M/R/T, 52A/H, 53A/M/S, 54G/H/I/M/Q/R/T, 55G/L/Q/S, 56E/Q/T, 57H/L/P/S, 58C/G/Y, 59E/G/H/M/P/R/T/V/W/Y, 60A/C/P/S/W, 61A/E/N/P/S/V, 62A/E/G/I/L/S, 63S, 64R, 65A/D/F/K/P/T, 66A/C/D/E/F/G/H/N/P/Q/R/S/T/V/W, 67R, 68A/D/E/G/I/L/Q/R/S/T/V/W/Y, 69F/L/Q/R/Y, 70R, 71T, 73I/R, 74C/G/Q/S, 75L, 76G/L/P, 77A/I/M/Q/S, 78C/G/N/T, 79A/K/L/P/W, 80A/D/G/L/N/P/R/S/V/W, 81F/I/L/Q/S, 82I/L/P/Q/T, 83G/H/L/S/T, 84A/E/H/M/S, 87A/E/I/K/L/M/R/V/Y, 88N/R/T, 89A/H/I/L/P/Q/T/V, 90C/E/F/K/S/T/V, 91A/F/G/L/S/V, 92L/S, 93A/E/G/P/S/T/V/Y, 94A/C/F/M/Q/S/T/V/Y, 97I, 98A/G/Q, 99A/C, 100F/S/V, 101-, 102A/C/N, 104A/F/H/I/L/Q/R/S/W, 105G/K/L/M/R/S, 108M/R/S/V, 109E/Y, 110C/F, 111E/P/R, 112A/C/E/K/M/N/Q/R/T, 113E/W/Y, 115G/K/R, 116A/E/F/L, 117L/N/S, 118A/G/L/R/S, 119F/K/L/P/R/S, 120G/K/L/R/S/T, 122A/H/S, 123S, 124V, 125I, 126A/E/S/V, 127I/L/P/S, 128C/E/I/K/N/R/S, 129I/L/P/S, 131A/G/V, 132G/K/L/T/V, 133A/E/F/L/Q/S/V/W, 134L, 135I/K/M/P/R/S, 136A/I/L/S/V/Y, 137L, 138C/I/M/V, 139A/H/L/R, 140G, 141G/V, 142F/L/M/W, 143A/C/G/P/R/S/T/V, 146D/H/N/R/V, 148F/H/M/P/Q/R/S/T, 149L, 150C/GIL/P/S/V, 151A/F/R/Y, 152F/H, 153C/K/S/V, 154Q/R, 155A/F/T/W, 156A/C/N/T/V, 157P/V, 161G/L, 162G, 163G/Q/S, 166C/F/L/P/S, 168G/L/N/Q/S, 169A/D/I/Y, 170A/G/H/P/R/S, 172A/H/M/S/T, 173F/K/R/S/T/V, 175F/L/S, 178E/G/N/R, 179A/C/G/I/L/P/V, 180G/H/P, 181C/I/V, 182A/G/I/L/Q/S, 183A/L/Q, 184I/K/M/N/R/S/T/V, 186L, 187G/Y, 188G/L, 190A/C/E/G/H/N/Q/R, 191D/K, 192A/H/I1W, 194F/L/R/V/Y, 195G/I/M/R, 196I, 197A/L/Q/V, 198G, 199R, 200A, 201A/F/K/L/S, 202E/G/M/S, 203A/E/L/R, 204R/S, 205L/P, 206W, 207A/L/T, 208E, 210V, 211A, 212C/H/I/L/M/N/P/Q/R/S/W, 213L/P/Q/S, 214A/E/L/M/P/R/T/W, 215A/P/V, 216A/D/E/G/H/L/M/N/P/R/S/T/V, 217E/G/H/P/T, 218A/I/L, 219A/F/G/L/P/S/T/W, 220E/V, 221D, 222I/T, 223T, 224G/S/T, 225A/L/Q/R/T/V, 226K/L/P/R/S/T, 227P, 228D/F/G/I/L/P/Q/R/S/T/V, 229C/I, 230A/M/P/Q, or 231A/Q/R/T, or combinations thereof, wherein the amino acid positions are relative to the reference sequence corresponding to amino acid residues 12-231 of SEQ ID NO: 2, or relative to the reference sequence corresponding to SEQ ID NO: 2.

8. The engineered adenylate kinase of claim 1, wherein the amino acid sequence of the engineered adenylate kinase comprises at least a substitution at amino acid position 13, 18, 29, 30, 31, 32, 34, 35, 48, 50, 53, 55, 59, 60, 61, 62, 66, 68, 77, 78, 80, 83, 87, 100, 104, 105, 109, 118, 119, 127, 128, 133, 136, 139, 142, 143, 150, 151, 155, 157, 168, 170, 175, 184, 186, 190, 201, 207, 212, 213, 214, 216, 217, 218, 224, or 226, or combinations thereof, wherein the amino acid positions are relative to the reference sequence corresponding to amino acid residues 12-231 of SEQ ID NO: 2, or relative to the reference sequence corresponding to SEQ ID NO: 2.

9. The engineered adenylate kinase of claim 1, wherein the amino acid sequence of the engineered adenylate kinase comprises at least a substitution or amino acid residue 13G/S, 18C/E/L, 29R/Q, 30G/L/S/Y, 31R, 32F/I/L/N, 34A/E/S/V, 35A/E/F/L/S, 48F, 50A/C/L/N/Q/R/S/V, 53A/M/S, 55G/L/Q/S, 59E/G/H/L/M/P/R/T/V/W/Y, 60A/C/P/S/W, 61A/E/N/P/S/V, 62A/E/G/I/L/S, 66A/C/D/E/F/G/H/N/P/Q/R/S/T/V/W, 68A/D/E/G/I/L/Q/R/S/T/V/W/Y, 77A/I/M/Q/S, 78C/G/N/T, 80A/D/G/L/N/P/R/S/V/W, 83G/H/L/S/T, 87A/E/I/K/L/M/R/V/Y, 100F/S/V, 104A/F/H/I/L/Q/R/S/W, 105G/K/L/M/R/S, 109E/Y, 118A/G/L/R/S, 119F/K/L/P/R/S, 127I/L/P/S, 128C/E/I/K/N/R/S, 133A/E/F/L/Q/S/V/W, 136A/I/L/S/V/Y, 139A/H/L/R, 142F/L/M/W, 143A/C/G/P/R/S/T/V, 150C/G/L/P/S/V, 151A/F/R/Y, 155A/F/T/W, 157P/V, 168G/L/N/Q/S, 170A/G/H/P/R/S, 175F/L/S, 184I/K/M/N/R/S/T/V, 186L, 190A/C/E/G/H/N/Q/R, 201A/F/K/L/S, 207A/L/T, 212C/H/I/L/M/N/P/Q/R/S/W, 213L/P/Q/S, 214A/E/L/M/P/R/T/W, 216A/E/G/H/L/M/N/P/R/S/T/V/W, 217E/G/H/P/T, 218A/I/L, 224G/S/T, or 226K/L/P/R/S/T, or combinations thereof, wherein the amino acid positions are relative to the reference sequence corresponding to amino acid residues 12-231 of SEQ ID NO: 2, or relative to the reference sequence corresponding to SEQ ID NO: 2.

10-17. (canceled)

18. The engineered adenylate kinase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, wherein the amino acid sequence comprises one or more substitutions relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

19. The engineered adenylate kinase of claim 1, comprising an amino acid sequence having at least 70%, 75%, 80%, 81%, 82%, 83%, 84%, 85%, 86%, 87%, 88%, 89%, 90%, 91%, 92%, 93%, 94%, 95%, 96%, 97%, 98%, 99%, or more sequence identity to the reference sequence corresponding to residues 12-231 of an even-numbered SEQ ID NO. of SEQ ID NOs: 4-1302, 1356-2652, and 2676-3192, or to the reference sequence corresponding to an even-numbered SEQ ID NO. of SEQ ID NOs: 4-1302, 1356-2652, and 2676-3192, wherein the amino acid sequence comprises one or more substitutions relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

20. The engineered adenylate kinase of claim 18, wherein the amino acid sequence of the engineered adenylate kinase comprises at least a substitution at amino acid position 11, 13, 14, 15, 16, 18, 20, 21, 22, 26, 27, 29, 30, 31, 32, 34, 35, 36, 37, 38, 39, 40, 42, 43, 44, 46, 48, 50, 51, 52, 53, 54, 55, 56, 57, 58, 59, 60, 61, 62, 63, 64, 65, 66, 67, 68, 69, 70, 71, 73, 74, 75, 76, 77, 78, 79, 80, 81, 82, 83, 84, 87, 88, 89, 90, 91, 92, 93, 94, 97, 98, 99, 100, 101, 102, 104, 105, 108, 109, 110, 111, 112, 113, 115, 116, 117, 118, 119, 120, 122, 123, 124, 125, 126, 127, 128, 129, 131, 132, 133, 134, 135, 136, 137, 138, 139, 140, 141, 142, 143, 146, 148, 149, 150, 151, 152, 153, 154, 155, 156, 157, 161, 162, 163, 166, 168, 169, 170, 172, 173, 175, 178, 179, 180, 181, 182, 183, 184, 186, 187, 188, 190, 191, 192, 194, 195, 196, 197, 198, 199, 200, 201, 202, 203, 204, 205, 206, 207, 208, 210, 211, 212, 213, 214, 215, 216, 217, 218, 219, 220, 221, 222, 223, 224, 225, 226, 227, 228, 229, 230, or 231, or combinations thereof, wherein the amino acid positions are relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

21. The engineered adenylate kinase of claim 18, wherein the amino acid sequence of the engineered adenylate kinase comprises at least a substitution or amino acid residue 11D, 13G/S, 14C/E, 15F, 16M, 18C/E/L, 20A/P/R/T/V, 21L/N/R/S/T/V, 22A/S, 26L, 27C/S, 29Q/R, 30G/L/S/Y, 31R, 32F/I/L/N, 34A/E/S/V, 35A/E/F/L/S, 36A/E/I/L/M/Q/R/S/V/Y, 37F/G/I/L/R/Y, 38C/F/L/R, 39A/M/Q/T, 40C/F/L/S/T, 42T, 43A/C/G/H/N, 44S, 46E/F/L/M/Y, 48F/L, 50A/C/L/N/Q/R/S/V, 51F/G/K/L/M/R/T, 52A/H, 53A/M/S, 54G/H/I/M/Q/R/T, 55G/L/Q/S, 56E/Q/T, 57H/L/P/S, 58C/G/Y, 59E/G/H/M/P/R/T/V/W/Y, 60A/C/P/S/W, 61A/E/N/P/S/V, 62A/E/G/I/L/S, 63S, 64R, 65A/D/F/K/P/T, 66A/C/D/E/F/G/H/N/P/Q/R/S/T/V/W, 67R, 68A/D/E/G/I/L/Q/R/S/T/V/W/Y, 69F/L/Q/R/Y, 70R, 71T, 73I/R, 74C/G/Q/S, 75L, 76G/L/P, 77A/I/M/Q/S, 78C/G/N/T, 79A/K/L/P/W, 80A/D/G/L/N/P/R/S/V/W, 81F/I/L/Q/S, 82I/L/P/Q/T, 83G/H/L/S/T, 84A/E/H/M/S, 87A/E/I/K/L/M/R/V/Y, 88N/R/T, 89A/H/I/L/P/Q/T/V, 90C/E/F/K/S/T/V, 91A/F/G/L/S/V, 92L/S, 93A/E/G/P/S/T/V/Y, 94A/C/F/M/Q/S/T/V/Y, 97I, 98A/G/Q, 99A/C, 100F/S/V/Y, 101-, 102A/C/N, 104A/F/H/I/L/Q/R/S/W, 105G/K/L/M/R/S, 108M/R/S/V, 109E/Y, 110C/F, 111E/P/R, 112A/C/E/K/M/N/Q/R/T, 113E/W/Y, 115G/K/R, 116A/E/F/L, 117L/N/S, 118A/G/L/R/S, 119F/K/L/P/R/S, 120G/K/L/R/S/T, 122A/H/S, 1235, 124V, 125I, 126A/E/S/V, 127I/L/P/S, 128C/E/I/K/N/R/S, 129I/L/P/S, 131A/G/V, 132G/K/L/T/V, 133F/Q/S/V/W, 133A/E/F/L/Q/S/V/W, 134L, 135I/K/M/P/R/S, 136A/I/L/S/V/Y, 137L, 138C/I/M/V, 139A/H/L/R, 140G, 141G/V, 142F/L/M/W, 143A/C/G/P/R/S/T/V, 146D/H/N/R/V, 148F/H/M/P/Q/R/S/T, 149L, 150C/GIL/P/S/V, 151A/F/R/Y, 152F/H, 153C/K/S/V, 154Q/R, 155A/F/T/W, 156A/C/N/T/V, 157P/V, 161G/L, 162G, 163G/Q/S, 166C/F/L/P/S, 168G/L/N/Q/S, 169A/D/I/Y, 170A/G/H/P/R/S, 172A/H/M/S/T, 173F/K/R/S/T/V, 175F/L/S, 178E/G/N/R, 179A/C/G/I/L/P/V, 180G/H/P, 181C/I/V, 182A/G/I/L/Q/S, 183A/L/Q, 184I/K/M/N/R/S/T/V, 186L, 187G/Y, 188G/L, 190A/C/E/G/H/N/Q/R, 191D/K, 192A/H/I/W, 194F/L/R/V/Y, 195G/I/M/R, 196I, 197A/L/Q/V, 198G, 199R, 200A, 201A/F/K/L/S, 202E/G/M/S, 203A/E/L/R, 204R/S, 205L/P, 206W, 207A/L/T, 208E, 210V, 211A, 212C/H/I/L/M/N/P/Q/R/S/W, 213L/P/Q/S, 214A/E/L/M/P/R/T/W, 215A/P/V, 216A/D/E/G/H/L/M/N/P/R/S/T/V, 217E/G/H/P/T, 218A/I/L, 219A/F/G/L/P/S/T/W, 220E/V, 221D, 222I/T, 223T, 224G/S/T, 225A/L/Q/R/T/V, 226K/L/P/R/S/T, 227P, 228D/F/G/I/L/P/Q/R/S/T/V, 229C/I, 230A/M/P/Q, or 231A/Q/R/T, or combinations thereof, wherein the amino acid positions are relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

22. The engineered adenylate kinase of claim 18, wherein the amino acid sequence of the engineered adenylate kinase comprises at least a substitution at amino acid position 13, 18, 29, 30, 31, 32, 34, 35, 48, 50, 53, 55, 59, 60, 61, 62, 66, 68, 77, 78, 80, 83, 87, 100, 104, 105, 109, 118, 119, 127, 128, 133, 136, 139, 142, 143, 150, 151, 155, 157, 168, 170, 175, 184, 186, 190, 201, 207, 212, 213, 214, 216, 217, 218, 224, or 226, or combinations thereof, wherein the amino acid positions are relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

23. The engineered adenylate kinase of claim 18, wherein the amino acid sequence of the engineered adenylate kinase comprises at least a substitution, or amino acid residue 13G/S, 18C/E/L, 29R/Q, 30G/L/S/Y, 31R, 32F/I/L/N, 34A/E/S/V, 35A/E/F/L/S, 48F, 50A/C/L/N/Q/R/S/V, 53A/M/S, 55G/L/Q/S, 59E/G/H/L/M/P/R/T/V/W/Y, 60A/C/P/S/W, 61A/E/N/P/S/V, 62A/E/G/I/L/S, 66A/C/D/E/F/G/H/N/P/Q/R/S/T/V/W, 68A/D/E/G/I/L/Q/R/S/T/V/W/Y, 77A/I/M/Q/S, 78C/G/N/T, 80A/D/G/L/N/P/R/S/V/W, 83G/H/L/S/T, 87A/E/I/K/L/M/R/V/Y, 100F/S/V, 104A/F/H/I/L/Q/R/S/W, 105G/K/L/M/R/S, 109E/Y, 118A/G/L/R/S, 119F/K/L/P/R/S, 127I/L/P/S, 128C/E/I/K/N/R/S, 133A/E/F/L/Q/S/V/W, 136A/I/L/S/V/Y, 139A/H/L/R, 142F/L/M/W, 143A/C/G/P/R/S/T/V, 150C/G/L/P/S/V, 151A/F/R/Y, 155A/F/T/W, 157P/V, 168G/L/N/Q/S, 170A/G/H/P/R/S, 175F/L/S, 184I/K/M/N/R/S/T/V, 186L, 190A/C/E/G/H/N/Q/R, 201A/F/K/L/S, 207A/L/T, 212C/H/I/L/M/N/P/Q/R/S/W, 213L/P/Q/S, 214A/E/L/M/P/R/T/W, 216A/E/G/H/L/M/N/P/R/S/T/V/W, 217E/G/H/P/T, 218A/I/L, 224G/S/T, or 226K/L/P/R/S/T, or combinations thereof, wherein the amino acid positions are relative to the reference sequence corresponding to residues 12-231 of SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104, or relative to the reference sequence corresponding to SEQ ID NO: 4, 172, 376, 508, 524, 584, 610, 674, 816, 874, 1032, 1388, 1708, 1952, 1980, 2072, 2138, 2294, 2368, 2602, 2832, 2994, or 3104.

24-134. (canceled)

135. The engineered adenylate kinase of claim 1, having adenylate kinase activity and at least one improved property as compared to a reference adenylate kinase, wherein the improved property is selected from i) increased activity on unmodified nucleoside monophosphate (NMP), ii) increased stability, iii) increased thermostability, iv) increased activity on 2′-fluoro modified nucleoside monophosphate, and v) increased activity on 2′-O-methyl modified nucleoside monophosphate, or any combinations of i), ii), iii), iv), and v) as compared to a reference adenylate kinase, and wherein the reference adenylate kinase has an amino acid sequence corresponding to residues 12-231 of SEQ ID NO: 2, or an amino acid sequence corresponding to SEQ ID NO: 2.

136. (canceled)

137. (canceled)

138. A recombinant polynucleotide comprising a polynucleotide sequence encoding an engineered adenylate kinase of claim 1.

139-143. (canceled)

144. An expression vector comprising a recombinant polynucleotide of claim 138.

145. (canceled)

146. (canceled)

147. A host cell comprising an expression vector of claim 144.

148. (canceled)

149. (canceled)

150. A method of producing an engineered adenylate kinase polypeptide, comprising culturing a host cell of claim 147 under suitable culture conditions such that the engineered adenylate kinase polypeptide is produced.

151. (canceled)

152. (canceled)

153. A composition comprising an engineered adenylate kinase of claim 1.

154. (canceled)

155. (canceled)

156. A method of converting a nucleoside or a modified nucleoside monophosphate to the corresponding nucleoside diphosphate (NMP), comprising contacting a nucleoside monophosphate with an engineered adenylate kinase of claim 1 in the presence of phosphate donor NTP under suitable reaction conditions to convert the nucleoside monophosphate to the corresponding nucleoside diphosphate (NMP).

157-183. (canceled)
